(19) United States
(12) Patent Application Publication
(10) Pub. No.: US 2024/0277276 A1
(43) Pub. Date: Aug. 22, 2024

(54) Bio-Electrode, And Method For Manufacturing The Same

(71) Applicant: Shin-Etsu Chemical Co., Ltd., Tokyo (JP)

(72) Inventors: Jun Hatakeyama, Joetsu-shi (JP); Joe Ikeda, Joetsu-shi (JP)

(73) Assignee: Shin-Etsu Chemical Co., Ltd., Tokyo (JP)

(21) Appl. No.: 18/407,117

(22) Filed: Jan. 8, 2024

(30) Foreign Application Priority Data
Jan. 20, 2023 (JP) 2023-7511

(51) Int. Cl. A61B 5/268; C08F 212/14; C08F 220/18; C08F 220/28; C08F 220/38; C08F 220/58; C09D 5/24; C09D 125/18; C09D 133/06; C09D 133/16; C09D 133/24

(52) U.S. Cl. CPC A61B 5/268; C08F 212/30; C08F 220/1808; C08F 220/282; C08F 220/382; C08F 220/387; C08F 220/585; C09D 5/24; C09D 125/18; C09D 133/064; C09D 133/066; C09D 133/16; C09D 133/24; A61B 2562/125; C08F 2800/10

(57) ABSTRACT

The present invention is a bio-electrode having layers on a substrate, wherein the layers include: (A) an electro-conductive layer including an electro-conductive wiring having a width of 200 μm or less; and (B) an ionic polymer containing layer including a polymer including a repeating unit-a having at least one selected from fluorosulfonic acid, fluorosulfonimide, and N-carbonyl-fluorosulfonamide, and having the weight-average molecular weight in a range of 1,000 to 500,000. This provides: a bio-electrode that allows thin, highly transparent, highly sensitive to a biological signal, excellent in biocompatibility, light-weight, manufacturable at low cost, capable of preventing significant reduction in the sensitivity to biological signals even when attached on the skin for a long time and when wetted with water or dried, and comfortable without itching, reddening, nor rash of skin; and a method for manufacturing a bio-electrode.

### TECHNICAL FIELD

The present invention relates to a bio-electrode that is used in contact with the skin of a living body and capable of detecting physical conditions such as heart rate by an electric signal transmitted from the skin, and a method for manufacturing the bio-electrode.

### BACKGROUND ART

A recent growing popularity of Internet of Things (IoT) has accelerated the development of wearable devices. In the fields of medicine and sports, wearable devices for constantly monitoring the user's physical state are demanded, and such technological development is expected to be further encouraged. In particular, because the global spread of the new coronavirus (COVID-19) causes a serious burden on medical care, home medical care for people who are not infected with the virus are strongly needed and its accelerated development is desired.

In the field of medicine, wearable devices for monitoring the state of human organs by sensing extremely weak current are sold, for example, like electrocardiogram measuring which detects the motion of the heart by electric signals. The electrocardiogram measurement is conducted by attaching electrodes coated with a hydrated gel to a body, but this is a single, short-time measurement. In contrast to this, development of the above medical wearable device is aimed at devices for continuously monitoring the health condition for a few weeks. Accordingly, the bio-electrode used for the medical wearable device is required to be able to collect biological data, not to cause an itchiness nor a skin allergy, and to be used comfortably, even over extended periods of use in daily life which involves showering, bathing, sweating, etc. In addition to these, the bio-electrode is also required to be so lightweight and thin to give no feeling when attached, and can be produced with a good productivity at low cost.

It is possible to measure an electrocardiogram by using a watch-type device represented by Apple Watch or by non-contact sensing using Rader. However, accurate electrocardiogram measuring for a medical use needs an electrocardiogramonitor which requires to apply bio-electrodes onto several parts on a body.

Medical wearable devices are classified into two types: one type is attached on the body, and another type is incorporated into clothing. For the attached type, for example, the bio-electrode using a hydrated gel such as hydrophilic gel containing water and electrolytes disclosed in patent document 1 is widely used. The hydrophilic gel contains sodium, potassium, or calcium as electrolytes in a hydrophilic polymer for retaining water, and converts a change in ion concentration from the skin into an electric signal by a reductive reaction of silver chloride in contact with the hydrophilic gel. When the gel gets dry, the bio-electrode has problems: the bio-electrode loses electrical conductivity and a function as electrode, and it swells during bathing or showering and falls off.

Meanwhile, for the electrode incorporated into closing, a method to use fabric as an electrode is proposed in patent document 2, wherein electro-conductive polymer such as PEDOT-PSS (Poly-3,4-ethylenedioxythiophene-Polystyrenesulfonate) or silver paste are incorporated in fibers.

A bio-electrode sheet with a stretchability and high electric conductivity is under development (non patent document 1). In the development, silver nanowires are coated on a polyurethane film, and are heated to 500° C. or higher on its surface instantaneously by a flash lamp anneal treatment to be made into fusion splicing with each other. Decreasing a density of the nanowires in a silver nanowire layer makes the bio-electrode more transparent. However, a bioelectrode consisting metals only has a problem having bigger noise to cause a drift in base line.

A bio-electrode consisting of metal thin film of gold, silver, stainless, etc., or a fiber coated with a metal or conductive polymer is not transparent. When a transparent bio-electrode which can observe skin in a see-through manner is developed, it produces an advantage of not visually disconcerting when attached to the skin.

For a transparent electro-conductive film for organic EL as an alternative for ITO, PEDOT-PSS has been studied. Non patent document 2 combines a silver nanowire and PEDOT-PSS. The combination of a silver nanowire and PEDOT-PSS can be applied for a bio-electrode. However, PEDOT-PSS is colored in blue, and not transparent.

Patent documents 3 to 5 propose a bio-electrode having, as a base material, a polymer containing a repeating unit having fluorosulfonic acid, fluorosulfonimide, and N-carbonyl-fluorosulfonamide. Such polymers have a high polarizability and high electric conductivity of an ion released from skin, and is transparent in itself. In Examples of these applications, bio-electrodes using the aforementioned polymer as a base material are formed on a highly conductive substrate, and the bio-electrode itself is not transparent.

According to heartbeats, changes in electrical potential and ion are released from skin. Because a metal bio-electrode detects only a change in electrical potential, its sensitivity of the detected signal is low, and there is a problem of causing a drift of baseline. In order to enhance the sensitivity to signals, it is preferable to detect the change not only in electrical potential but also in ion.

### CITATION LIST

### Patent Literature

Patent Document 1: WO 2013/039151 A1
Patent Document 2: JP 2015-100673 A
Patent Document 3: JP 2018-099504 A
Patent Document 4: JP 2018-110845 A
Patent Document 5: JP 2018-130533 A
Non Patent Literature
Non Patent Document 1: Nano Res. 9, 401 (2016)
Non Patent Document 2: J. Photopolymer Sci. and Tech. Vol. 32 No. 3 p 429 (2019)

### SUMMARY OF INVENTION

### Technical Problem

The present invention has been made to solve the problems, and has an object to provide a bio-electrode which is thin, highly transparent, highly sensitive to a biological signal, excellent in biocompatibility, light-weight, manufacturable at low cost, capable of preventing significant reduction in the sensitivity to biological signals even when attached on the skin for a long time and when wetted with water or dried, and comfortable without itching, reddening, nor rash of skin; and a method for manufacturing the bio-electrode.

### Solution to Problem

The present invention has been made in view of the above-described problem. An object of the present invention is to provide a bio-electrode having layers on a substrate, wherein the layers include:
(A) an electro-conductive layer including an electro-conductive wiring having a width of 200 μm or less; and
(B) an ionic polymer containing layer including a polymer including a repeating unit-a having at least one selected from fluorosulfonic acid, fluorosulfonimide, and N-carbonyl-fluorosulfonamide, and having the weight-average molecular weight in a range of 1,000 to 500,000.

Such a bio-electrode can be thin, highly transparent, highly sensitive to a biological signal, excellent in biocompatibility, light-weight, manufacturable at low cost, capable of preventing significant reduction in sensitivity to biological signals even when attached on the skin for a long time and when wetted with water or dried, and comfortable without itching, reddening, nor rash of skin.

Further, in the present invention, a laminate film, being a combination of the layer (A) and the layer (B), has a visible light transmittance of 50% or more preferably.

Such a bio-electrode is preferable because the skin can be observed through the bio-electrode.

Furthermore, in the present invention, the electro-conductive wiring having a width of 200 μm or less is preferably a printed pattern formed by an electro-conductive paste containing a particle of gold, silver, copper, or nickel, or by a fusion layer of metal nanowires containing gold, silver, copper, nickel, or alloy thereof.

Such an electro-conductive wiring can be used preferably.

Furthermore, in the present invention, the repeating unit-a preferably has a partial structure shown by any of the following general formulae (1)-1 to (1)-4,

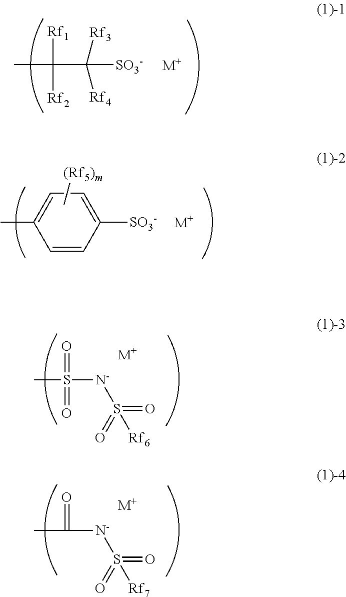

wherein Rf₁ and Rf₂ each represent a hydrogen atom, a fluorine atom, an oxygen atom, a methyl group, or a trifluoromethyl group, provided that when Rf₁ and Rf₂ represent an oxygen atom, the single oxygen atom represented by Rf₁ and Rf₂ bonds to a single carbon atom to form a carbonyl group; Rf₃ and Rf₄ each represent a hydrogen atom, a fluorine atom, or a trifluoromethyl group, and at least one of Rf₁ to Rf₄ is a fluorine atom or a trifluoromethyl group; Rf₅, Rf₆, and Rf₇ each represent a fluorine atom, a trifluoromethyl group, a linear or branched alkyl group having 1 to 4 carbon atoms, or an aryl group having 6 to 10 carbon atoms, and have at least one fluorine atom or trifluoromethyl group; and M⁺ represents an ion selected from the group consisting of an ammonium ion, a sodium ion, and a potassium ion; “m” represents an integer of 1 to 4.

When the repeating unit-a has such structures, the bio-electrode is excellent in electric conductivity and biocompatibility.

In this event, the repeating unit-a preferably includes at least one selected from the group consisting of repeating units A1 to A7 shown by the following general formula (2),

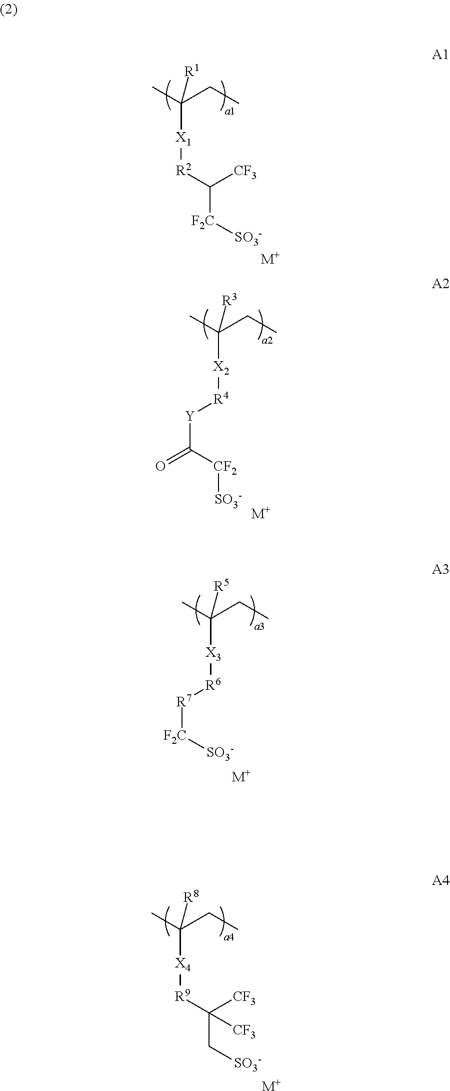

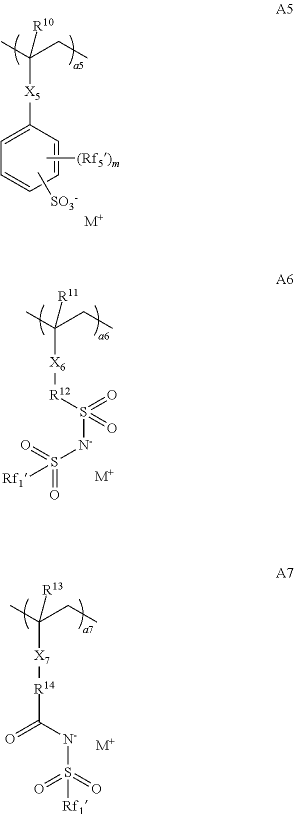

wherein, R¹, R³, R⁵, R⁸, R¹⁰, R¹¹, and R¹³ each independently represent a hydrogen atom or a methyl group; R², R⁴, R⁶, R⁹, R¹², and R¹⁴ each independently represent a single bond or a linear, branched, or cyclic hydrocarbon group having 1 to 13 carbon atoms, the hydrocarbon group optionally having either or both of an ester group and an ether group; R⁷ represents a linear or branched alkylene group having 1 to 4 carbon atoms, and one or two hydrogen atoms in R⁷ are optionally substituted with a fluorine atom; X₁, X₂, X₃, X₄, X₆, and X₇ each independently represent any of a single bond, a phenylene group, a naphthylene group, an ether group, an ester group, and an amide group; X₅ represents any of a single bond, an ether group, and an ester group; Y represents an oxygen atom or a —NR¹⁹— group; R¹⁹ represents any of a hydrogen atom, a linear, branched, or cyclic alkyl group having 1 to 12 carbon atoms, and phenyl group, optionally include one or more group selected from the group consisting of ether group, carbonyl group, ester group, and amide group; and Y forms a ring together with R⁴; Rf₁′ and Rf₅′ each represent a fluorine atom, a trifluoromethyl group, or a linear or branched alkyl group having 1 to 4 carbon atoms, and have at least one fluorine atom; “m” represents an integer of 1 to 4; a1, a2, a3, a4, a5, a6, and a7 satisfy 0≤a1≤1.0, 0≤a2≤1.0, 0≤a3≤1.0, 0≤a4≤1.0, 0≤a5≤1.0, 0≤a6≤1.0, 0≤a7≤1.0, and 0<a1+a2+a3+a4+a5+a6+a7≤1.0; and M⁺ represents an ion selected from the group consisting of an ammonium ion, a sodium ion, and a potassium ion.

The repeating unit-a having such structures enables further improvement of electric conductivity and biocompatibility.

In this event, the repeating unit-a preferably includes an ammonium ion shown by the following general formula (3) as an ammonium ion for forming an ammonium salt,

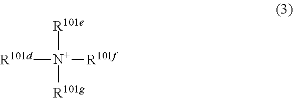

wherein, R^{101d}, R^{101e}, R^{101f}, and R^{101g} each represent a hydrogen atom, a linear, branched, or cyclic alkyl group having 1 to 15 carbon atoms, a linear, branched, or cyclic alkenyl group or alkynyl group having 2 to 12 carbon atoms, or an aromatic group having 4 to 20 carbon atoms, and optionally have one or more selected from the group consisting of an ether group, a carbonyl group, an ester group, a hydroxy group, a carboxy group, an amino group, a nitro group, a sulfonyl group, a sulfinyl group, a halogen atom, and a sulfur atom; and R^{101d} and R^{101e}, or R^{101d}, R^{101e}, and R^{101f}, are optionally bonded to each other together with a nitrogen atom bonded therewith to form a ring in which R^{101d} and R^{101e}, or R^{101d}, R^{101e}, and R^{101f}, represent an alkylene group having 3 to 10 carbon atoms, or to form a heteroaromatic ring having the nitrogen atom in the general formula (3) within the ring.

The polymer compound containing such an ammonium ion can be further excellent in electric conductivity and biocompatibility.

Furthermore, in the present invention, the bio-electrode further includes one or more resin (C) selected from the group consisting of (meth)acrylate resin, (meth)acrylamide resin, urethane resin, polyurethane (meth)acrylate, polyvinyl alcohol, polyvinylpyrrolidone, polyoxazoline, polyglycerin, polyglycerin-modified silicone, polyglycerin(meth)acrylate, cellulose, polyethylene glycol, and polypropylene glycol, as a component of the layer (B).

The layer (B) can contain such a resin (C).

Furthermore, the present invention provides a method for manufacturing the bio-electrode including:
forming the layer (A) on a substrate by applying a solution including a metal nanowire, or by printing an electro-conductive paste containing a conductive particle; and forming the layer (B) on the layer (A) by coating a bio-electrode composition including the polymer.

According to such a manufacturing method, it is possible to manufacture the bio-electrode which is thin, highly transparent, highly sensitive to a biological signal, excellent in biocompatibility, light-weight, manufacturable at low cost, capable of preventing significant reduction in the sensitivity to biological signals even when attached on the skin for a long time and when wetted with water or dried, and comfortable without itching, reddening, nor rash of skin.

### Advantageous Effects of Invention

As described above, the inventive bio-electrode and the inventive method for manufacturing the bio-electrode make it possible to provide a bio-electrode which is highly sensitive to a biological signal, excellent in biocompatibility, thin, light-weight, highly transparent, manufacturable at low cost, capable of preventing significant reduction in the sensitivity to biological signals even when wetted with water or dried and when attached on the skin for a long time, and comfortable without itching, reddening, nor rash of skin.

### BRIEF DESCRIPTION OF DRAWINGS

FIG. 1 is a schematic sectional view showing after an electro-conductive layer is formed on a substrate.

FIG. 2 is a schematic sectional view showing the inventive bio-electrode after an ionic polymer containing layer is formed on the electro-conductive layer.

FIG. 3 is a schematic sectional view showing the inventive bio-electrode attached on a human skin to measure a biological signal.

FIG. 4 is a schematic top view showing the electro-conductive wiring having a width of 200 μm or less of the inventive bio-electrode, which is formed in the shape of a straight line by printing.

FIG. 5 is a schematic top view showing the electro-conductive wiring having a width of 200 μm or less of the inventive bio-electrode, which is formed in the shape of zigzag line by printing.

FIG. 6 is a schematic top view showing the electro-conductive wiring having a width of 200 μm or less of the inventive bio-electrode, which is formed in the shape of waves by printing.

FIG. 7 is a schematic top view showing the electro-conductive wiring having a width of 200 μm or less of the inventive bio-electrode, which is formed in the shape of a small zigzag line on a large zigzag line by printing.

FIG. 8 is a schematic top view showing the electro-conductive wiring having a width of 200 μm or less of the inventive bio-electrode, which is formed in an aslant lattice pattern by printing.

FIG. 9 is a schematic top view showing the electro-conductive wiring having a width of 200 μm or less of the inventive bio-electrode, which is formed in a hexagonal pattern by printing.

FIG. 10 is a schematic top view showing the electro-conductive wiring having a width of 200 μm or less of the inventive bio-electrode, which is formed in a pattern of repeating circles by printing.

FIG. 11 is a schematic top view showing the electro-conductive wiring having a width of 200 μm or less of the inventive bio-electrode, which is formed in another pattern of repeating circles by printing.

FIG. 12 is a schematic top view showing the electro-conductive wiring having a width of 200 μm or less of the inventive bio-electrode, which is formed in a mesh pattern by printing.

FIG. 13 is a schematic top view showing the coated nanowires.

FIG. 14 is a schematic view showing the bio-electrodes prepared in Examples of the present invention.

FIG. 15 shows locations where electrodes are attached on a human body in measuring biological signals in Examples of the present invention.

### DESCRIPTION OF EMBODIMENTS

As noted above, in order to detect biological signals with high sensitivity and low noise, it has been desired to develop: the bio-electrode which is excellent in high electric conductivity and biocompatibility, highly transparent, light-weight, thin, manufacturable at low cost, capable of measuring biological signals even when wetted with water or dried, and causing no skin damage nor rash when attached on the skin for a long time; and a method for manufacturing such a bio-electrode.

The surface of skin releases ions of sodium, potassium, and calcium in accordance with heartbeats. The bio-electrode needs to convert the increase and decrease of the ions released from skin to electric signals. Accordingly, the bio-electrode requires a material that is excellent in ionic conductivity to transmit the increase and decrease of ions. In accordance with heartbeats, electric potential of the surface of skin also changes. The change of the electric potential is small, and thus electric conductivity is also required in order to transmit such small current to a device.

A hydrophilic gel containing sodium chloride or potassium chloride has high ionic conductivity, but it loses the electric conductivity when dried. Further, the electric conductivity decreases also when sodium chloride or potassium chloride is eluted outside the bio-electrode by bathing or showering.

Because the bio-electrode using metals such as gold or silver detects only small current and has low ionic conductivity, and thus its sensitivity as a bio-electrode is low. Carbon has electron conductivity like metals, but its electron conductivity is lower than metals, and its sensitivity as a bio-electrode is lower than metals.

Conductive polymers represented by PEDOT-PSS have both electron conductivity and ionic conductivity, but ionic conductivity is low due to its low polarization. Further, because a film coated by PEDOT-PSS has absorption in the red region, and it is tinted blue, its complementary color. When used by people with white skin color, blue areas where bio-electrodes are attached is a visually disconcerting.

Salts of fluorosulfonic acid, fluorosulfonimide, and N-carbonyl-fluorosulfonamide have high polarizability and high ionic conductivity. A combination of these polymers and a narrow conductive paste pattern or metal wires having less reduction of transparency, can produce both properties of high ionic conductivity with high transparency and high electron conductivity.

A polymer film having salts of fluorosulfonic acid, fluorosulfonimide, and N-carbonyl-fluorosulfonamide is transparent to a visible light. If with high transparency, the bio-electrodes are less visually disconcerting when they are attached on the skin.

As a result of their diligent study of the above problems, the inventors found a bioelectrode of the following configuration and a manufacturing method thereof, and have completed the present invention.

That is, the present invention relates to a bio-electrode which has layers on a substrate, the layers including:
(A) an electro-conductive layer including an electro-conductive wiring having a width of 200 μm or less; and
(B) an ionic polymer containing layer including a polymer including a repeating unit-a having at least one selected from fluorosulfonic acid, fluorosulfonimide, and N-carbonyl-fluorosulfonamide, and having the weight-average molecular weight in a range of 1,000 to 500,000. The present invention is described below in detail; however, the present invention is not limited to the examples described below.

### <Bio-Electrode>

The inventive bio-electrode has layers on a substrate, the layers including:
(A) an electro-conductive layer including an electro-conductive wiring having a width of 200 μm or less; and
(B) an ionic polymer containing layer including a polymer including a repeating unit-a having at least one selected from fluorosulfonic acid, fluorosulfonimide, and N-carbonyl-fluorosulfonamide, and having the weight-average molecular weight in a range of 1,000 to 500,000.

A laminate film, being a combination of the layer (A) and the layer (B), preferably has a visible light transmittance of 50% or more. More preferably, the visible light transmittance is 60% or more.

When the visible light transmittance is 50% or more, skin can be observed through the bio-electrode. When its visible light transmittance is 60% or more, difference in color between the bio-electrode and skin is small and presence of the bio-electrode disappears when attached on skin.

Configuration of highly sensitive bio-electrode requires not only high ionic conductivity, but also high electron conductivity. The electron conductivity is secured by electro-conductive wiring.

The electro-conductive wiring is preferably formed by printing electro-conductive ink (paste). Thickness of a hair is said to 100 to 200 μm, and a line having the same thickness as this or less is not visible by the naked eye. Accordingly, electro-conductive wiring having width of more than 200 μm has poor transparency and not preferable. The width of the electro-conductive wiring is 200 μm or less, preferably 100 μm or less, more preferably 80 μm or less, and further preferably 50 μm or less. The electro-conductive wiring may be parallel to the direction of expansion and contraction, but accordion-typed one can prevent wire breaking due to its expansion and contraction and can have advantage of less reduction in electro-conductivity.

The electro-conductive ink is preferably electro-conductive paste having a conductive particle including of gold, silver, copper, nickel, or alloy thereof. A size of the particle is preferably in the range of 1 to 200 nm in diameter. In addition to the conductive particle and the solvent medium, a resin may be added. The resin includes poly(meth)acrylate, polyurethane, polystyrene butadiene, polyacrylonitrile, polyvinyl alcohol, polyvinylpyrrolidone, cellulose, or silicone.

Furthermore, the electro-conductive wiring can be the fusion layer of metal nanowire. The metal nanowire preferably includes gold, silver, copper, nickel, or alloy thereof. The size of the metal nanowire is preferably in the range of 1 to 200 nm in diameter, and in the range of 0.1 to 500 μm in length. Using metal nanowire enables to form the electro-conductive wiring with higher transparency.

The inventive transparent bio-electrode can measure not only electrocardiogram, but also electromyogram, brain wave, and breathing rate. Additionally, not only by measuring signals released from skin but also by providing electric signals with skin, it is possible to send signals to muscles or control brain wave. For example, it is considered to be applied for enhancing performance, stimulating muscles to reduce fatigue during swimming, and enhancing relaxation in bathing.

### <Composition of Ionic Polymer Containing Layer (Bio-Electrode Composition)>

Below, each component to form the ionic polymer containing layer of the present invention will be described in further detail.

### [Ionic Polymer (Salt)]

Ionic polymer which is a material to form the ionic polymer containing layer used for the inventive bio-electrode can contain, as an ionic material, a polymer having ionic repeating unit-a selected from the group consisting of salts of ammonium, lithium, sodium, and potassium formed with any of fluorosulfonic acid, fluorosulfonimide, and N-carbonyl-fluorosulfonamide.

The repeating unit-a can have a partial structure shown by any of the following general formulae (1)-1 to (1)-4,

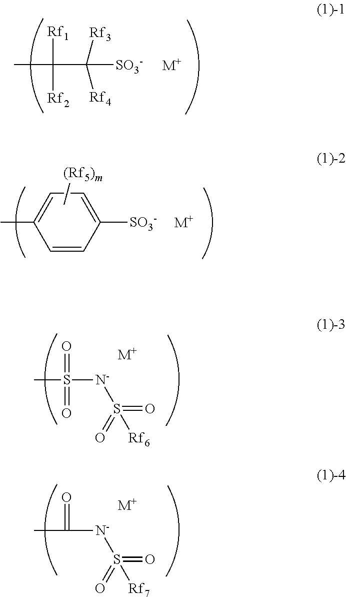

wherein Rf₁ and Rf₂ each represent a hydrogen atom, a fluorine atom, an oxygen atom, a methyl group, or a trifluoromethyl group, provided that when Rf₁ and Rf₂ represent an oxygen atom, the single oxygen atom represented by Rf₁ and Rf₂ bonds to a single carbon atom to form a carbonyl group; Rf₃ and Rf₄ each represent a hydrogen atom, a fluorine atom, or a trifluoromethyl group, and at least one of Rf₁ to Rf₄ is a fluorine atom or a trifluoromethyl group; Rf₅, Rf₆, and Rf₇ each represent a fluorine atom, a trifluoromethyl group, a linear or branched alkyl group having 1 to 4 carbon atoms, or an aryl group having 6 to 10 carbon atoms, and have at least one fluorine atom or trifluoromethyl group; and M⁺ represents an ion selected from the group consisting of an ammonium ion, a sodium ion, and a potassium ion; “m” represents an integer of 1 to 4.

One or more repeating unit-a selected from the group consisting of salts of ammonium, sodium, and potassium formed with any of fluorosulfonic acid shown by the above general formulae (1)-1 and (1)-2, sulfonimide shown by the general formulae (1)-3, or N-carbonyl-fluorosulfonamide shown by the general formulae (1)-4 preferably includes at least one selected from the group consisting of repeating units A1 to A7 shown by the following general formula (2),

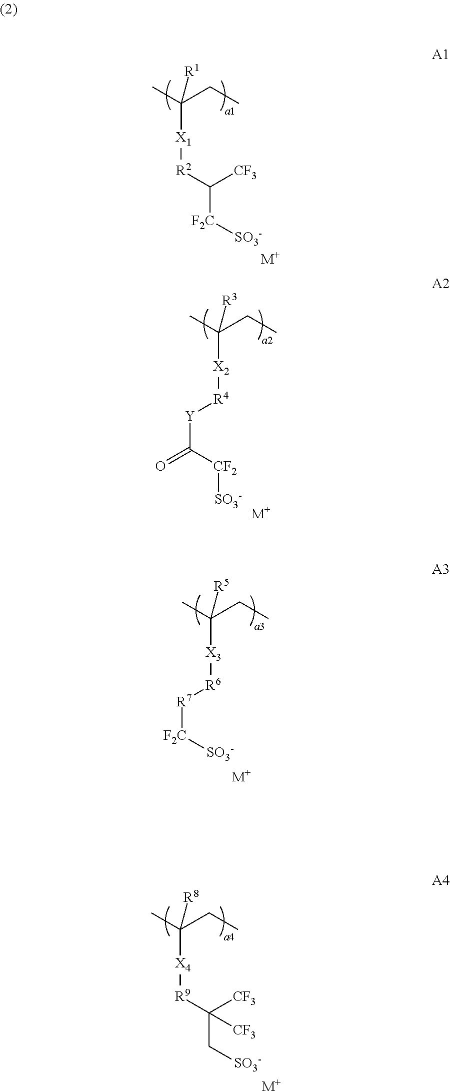

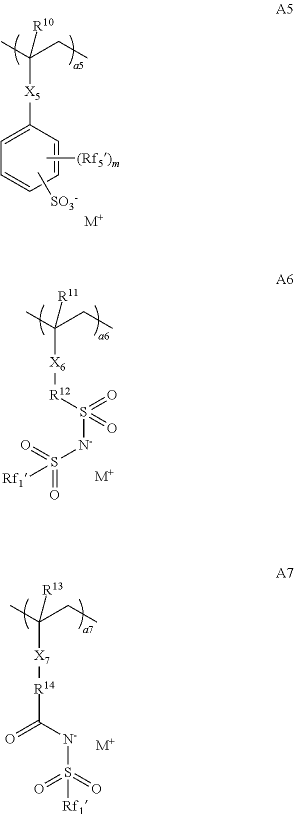

wherein, R¹, R³, R⁵, R⁸, R¹⁰, R¹¹, and R¹³ each independently represent a hydrogen atom or a methyl group; R², R⁴, R⁶, R⁹, R¹², and R¹⁴ each independently represent a single bond or a linear, branched, or cyclic hydrocarbon group having 1 to 13 carbon atoms, the hydrocarbon group optionally having either or both of an ester group and an ether group; R⁷ represents a linear or branched alkylene group having 1 to 4 carbon atoms, and one or two hydrogen atoms in R⁷ are optionally substituted with a fluorine atom; X₁, X₂, X₃, X₄, X₆, and X₇ each independently represent any of a single bond, a phenylene group, a naphthylene group, an ether group, an ester group, and an amide group; X₅ represents any of a single bond, an ether group, and an ester group; Y represents an oxygen atom or a —NR¹⁹— group; R¹⁹ represents any of a hydrogen atom, a linear, branched, or cyclic alkyl group having 1 to 12 carbon atoms, and phenyl group, optionally include one or more group selected from the group consisting of ether group, carbonyl group, ester group, and amide group; and Y forms a ring together with R⁴; Rf₁′ and Rf₅′ each represent a fluorine atom, a trifluoromethyl group, or a linear or branched alkyl group having 1 to 4 carbon atoms, and have at least one fluorine atom; “m” represents an integer of 1 to 4; a1, a2, a3, a4, a5, a6, and a7 satisfy 0≤a1≤1.0, 0≤a2≤1.0, 0≤a3≤1.0, 0≤a4≤1.0, 0≤a5≤1.0, 0≤a6≤1.0, 0≤a7≤1.0, and 0<a1+a2+a3+a4+a5+a6+a7≤1.0; and M⁺ represents an ion selected from the group consisting of an ammonium ion, a sodium ion, and a potassium ion.

In the above general formula (2), a1 to a7 are ratios of repeating units-A1 to -A7 respectively.

### (Repeating Unit A)

Among the repeating units-A1 to -A7 shown by the above general formula (2), the repeating units-A1 to -A5 can be obtained from fluorosulfonic acid salt monomers specifically exemplified below.

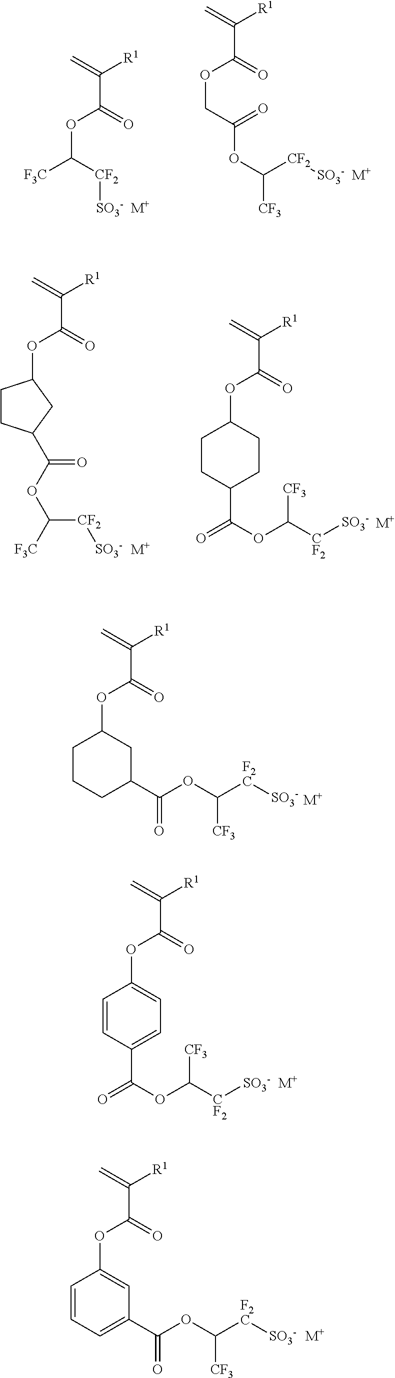

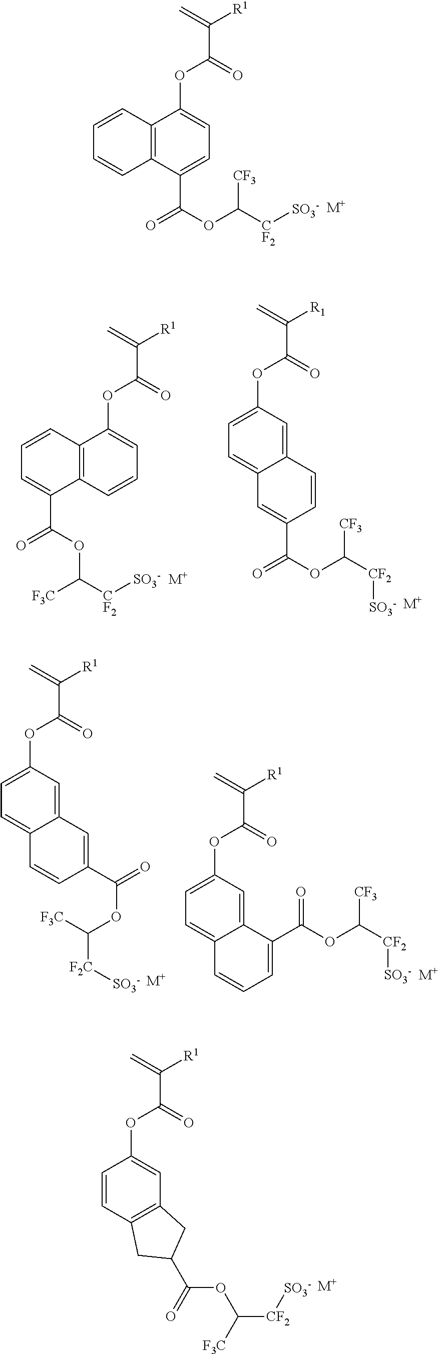

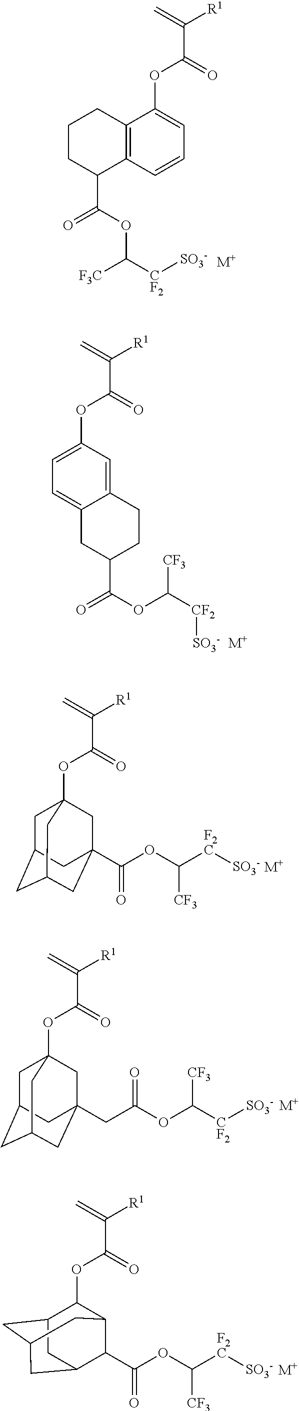

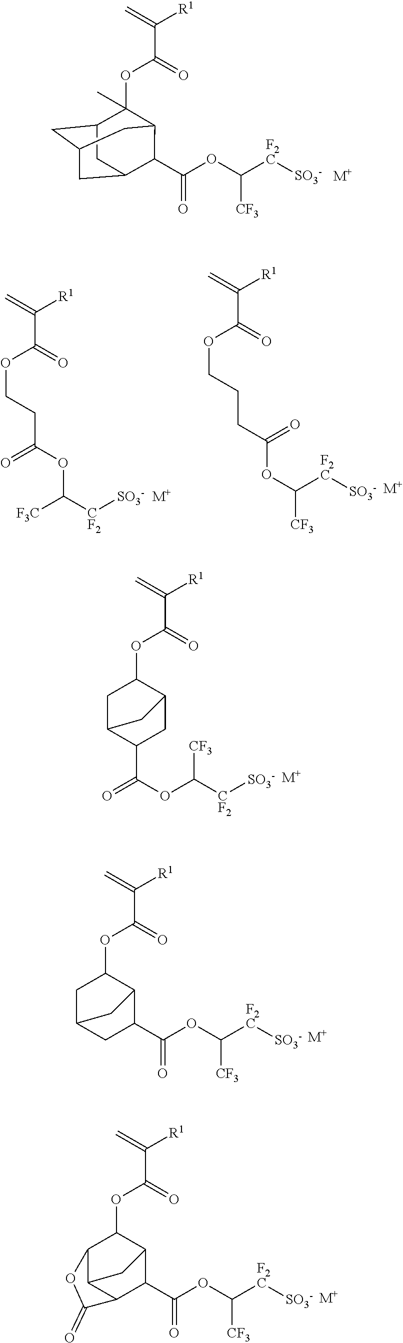

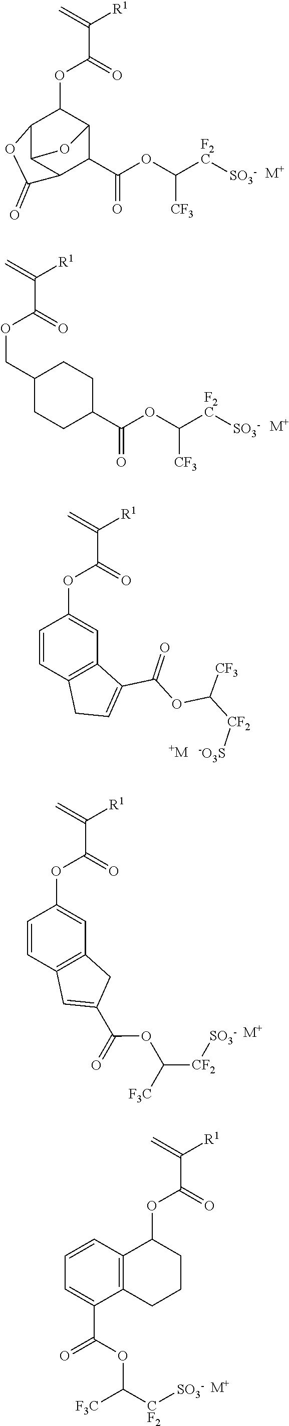

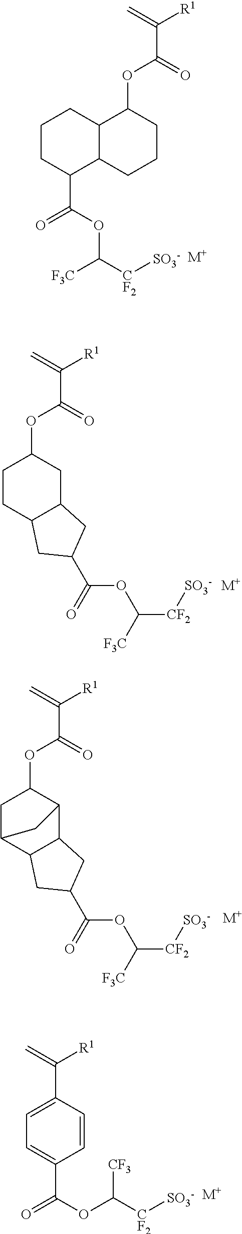

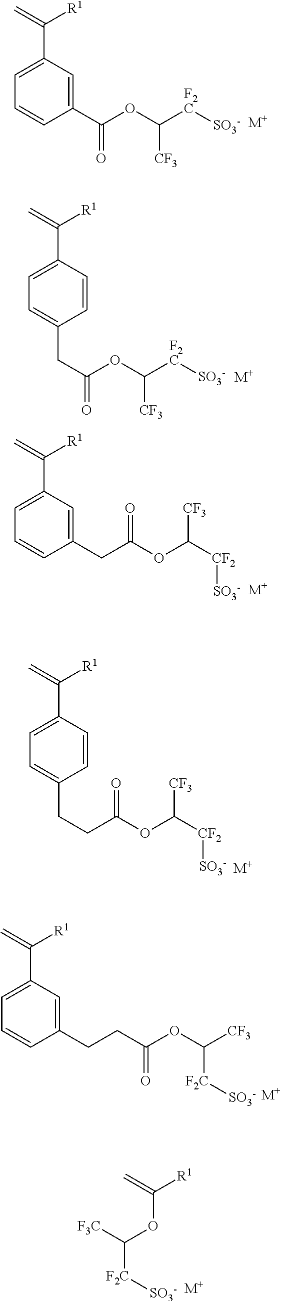

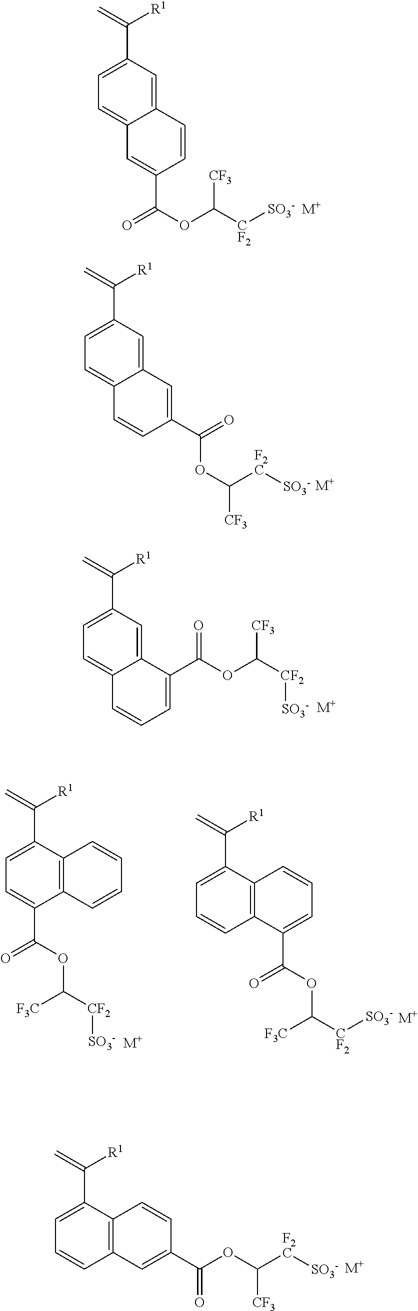

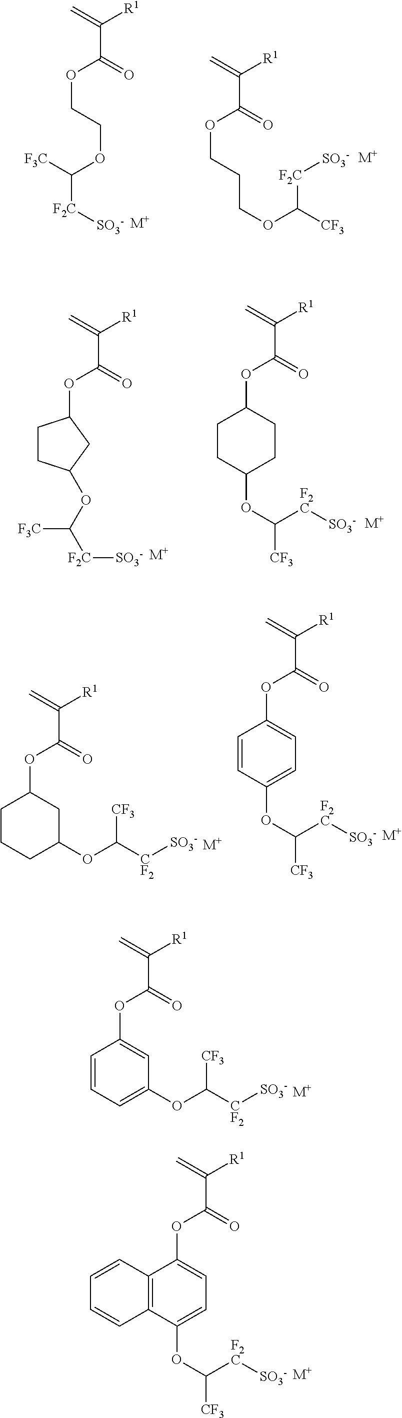

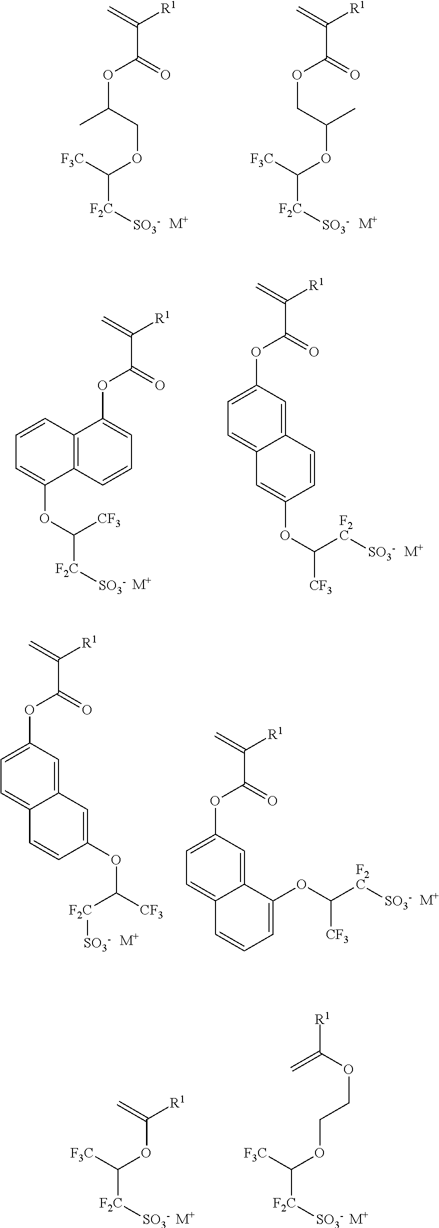

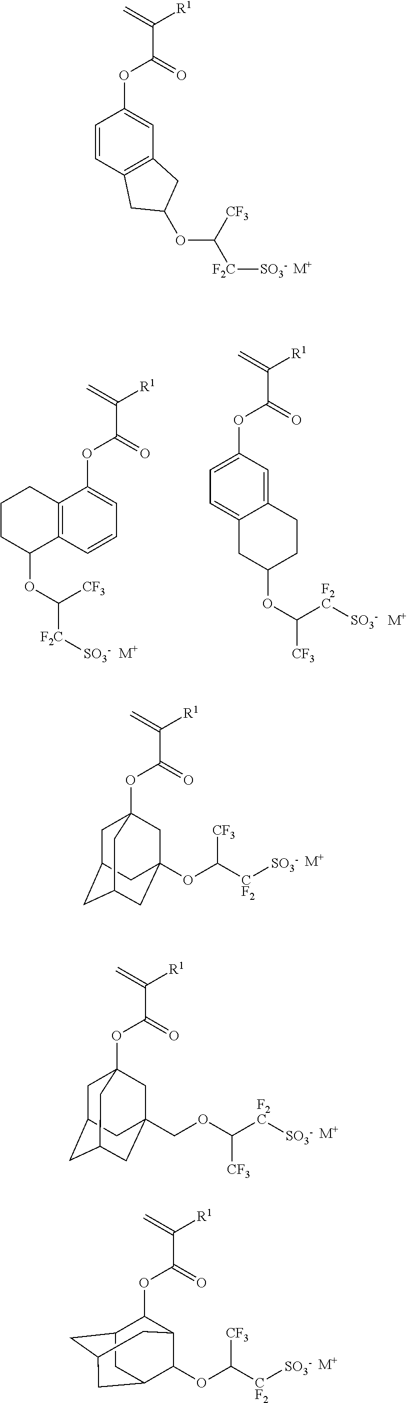

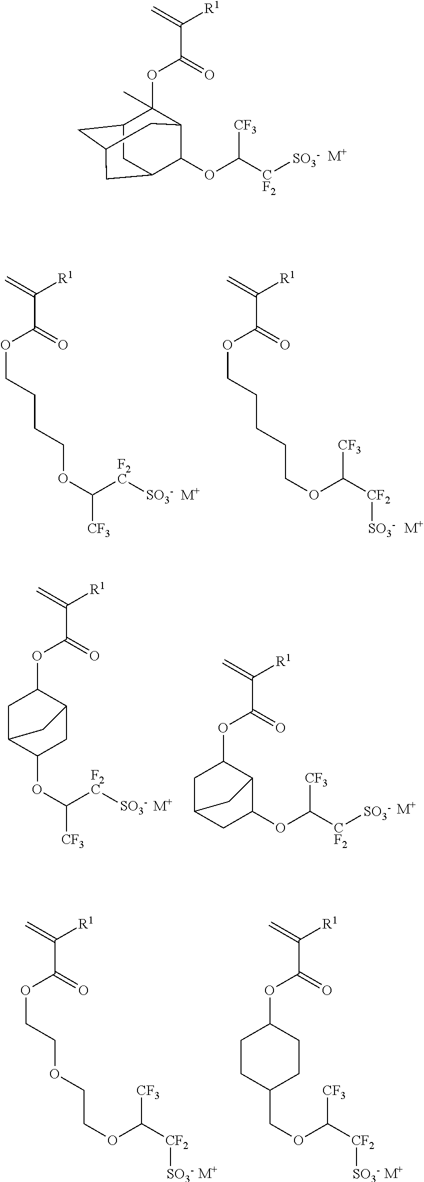

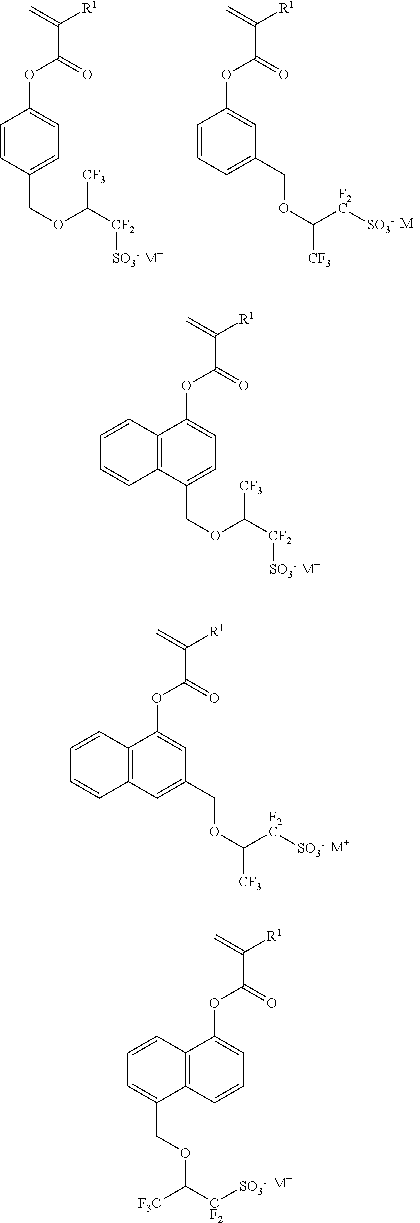

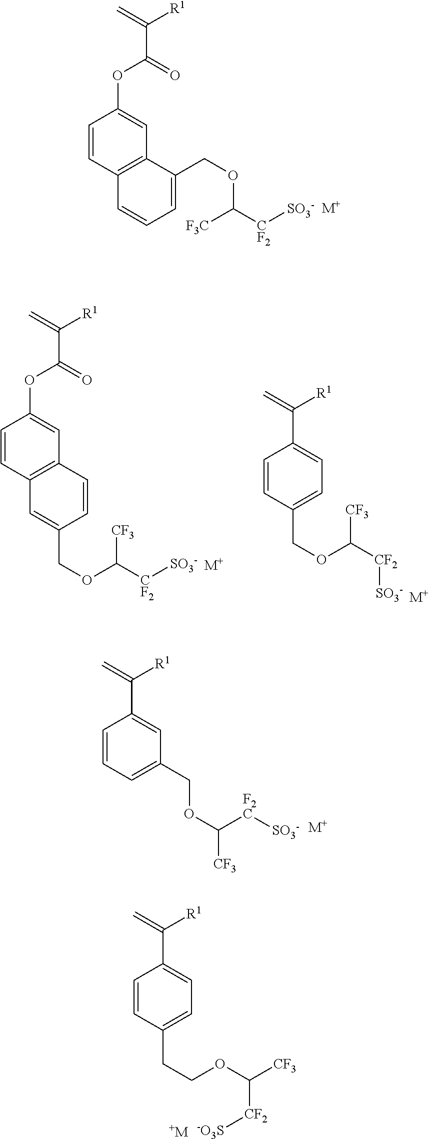

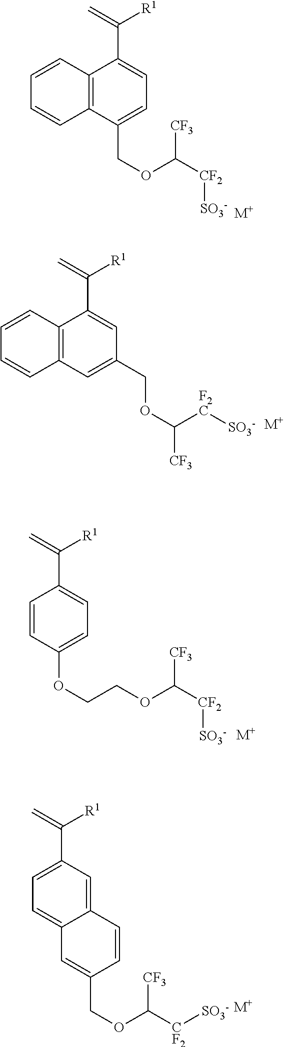

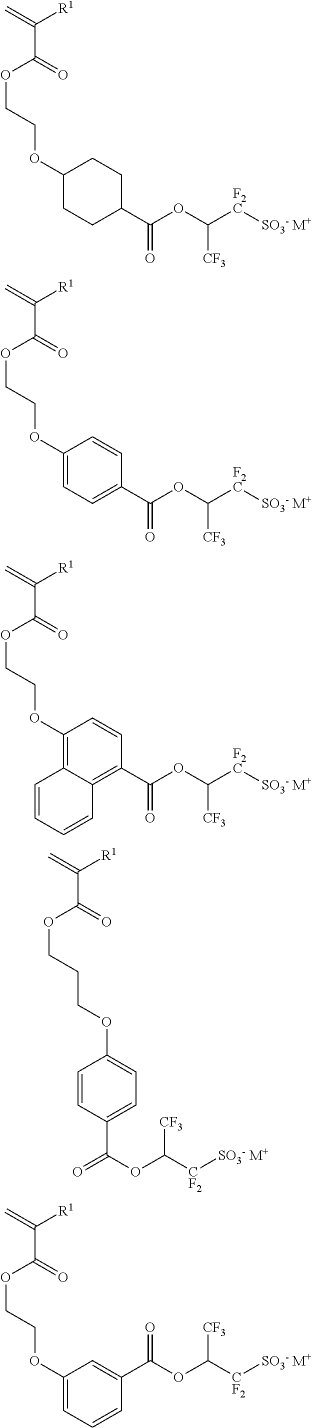

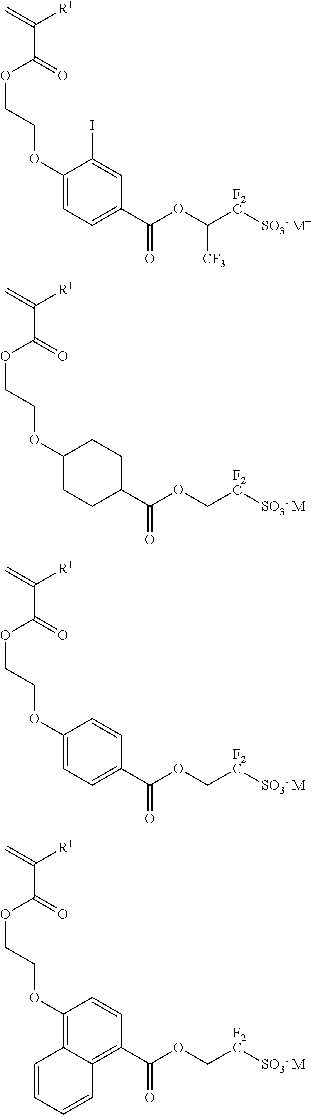

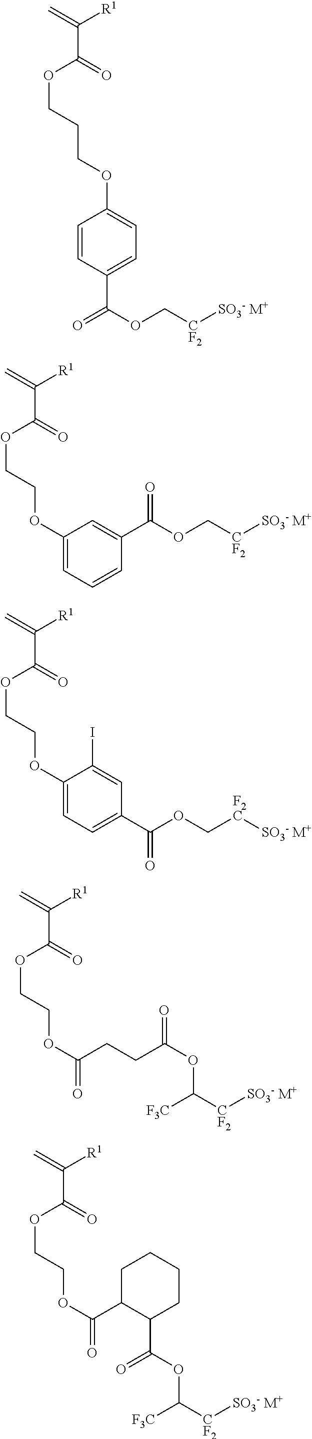

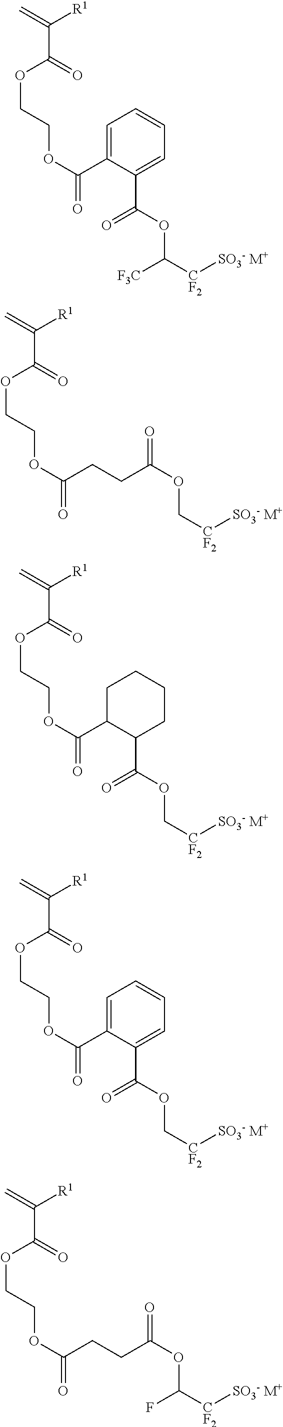

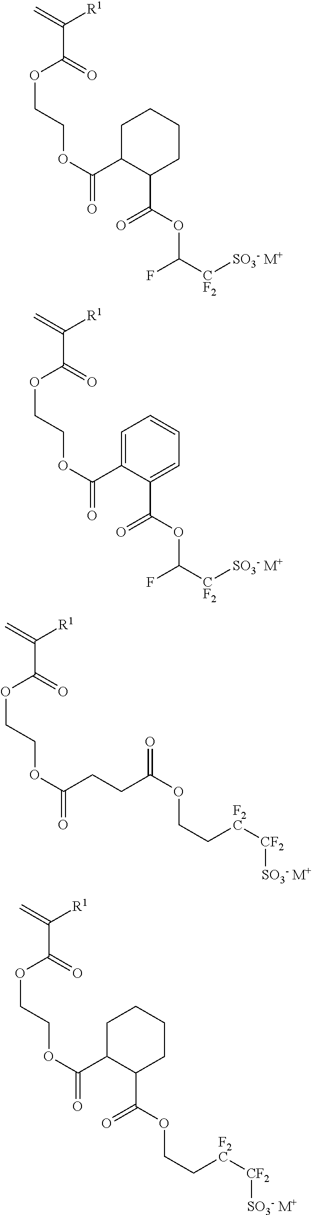

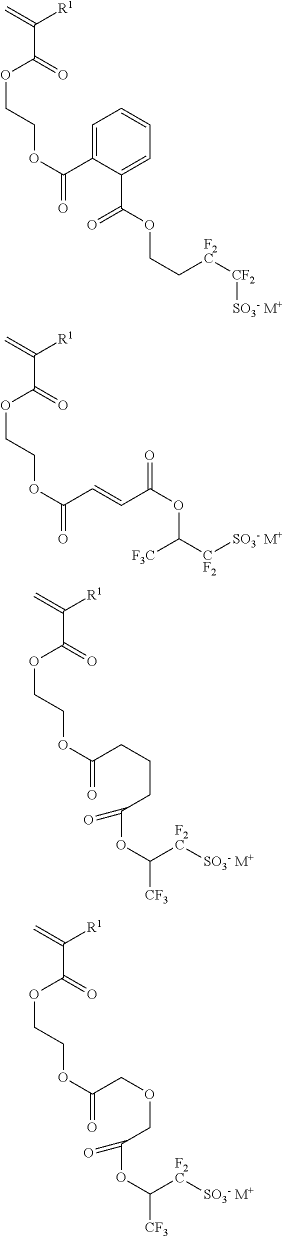

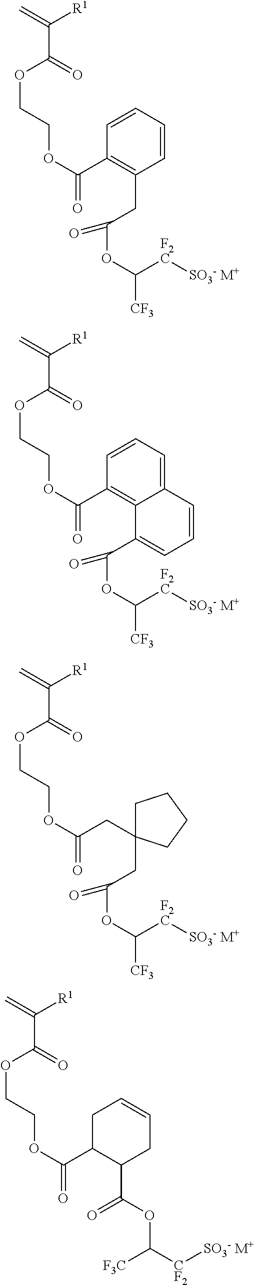

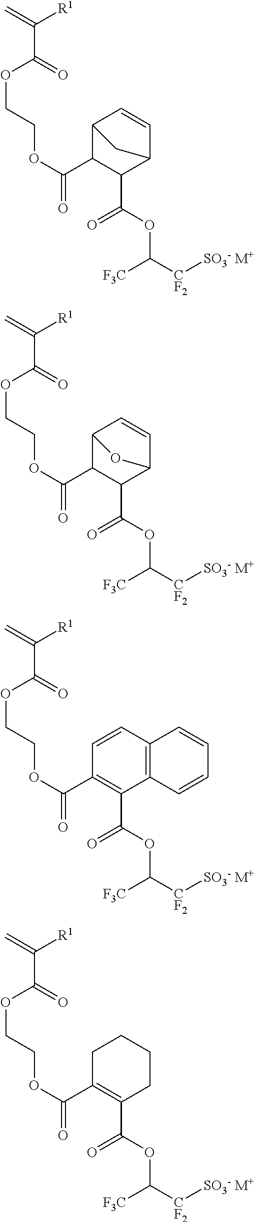

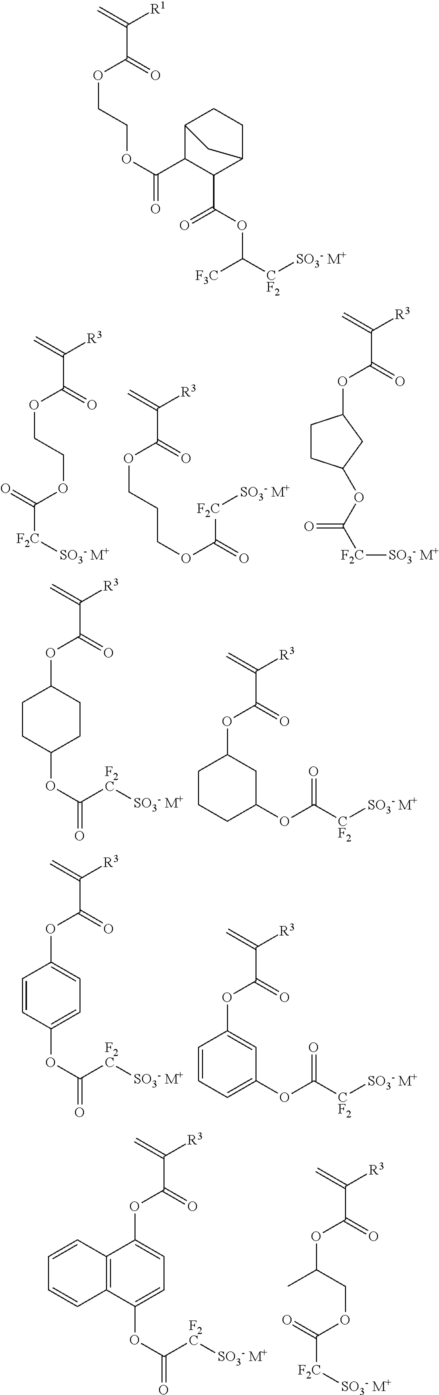

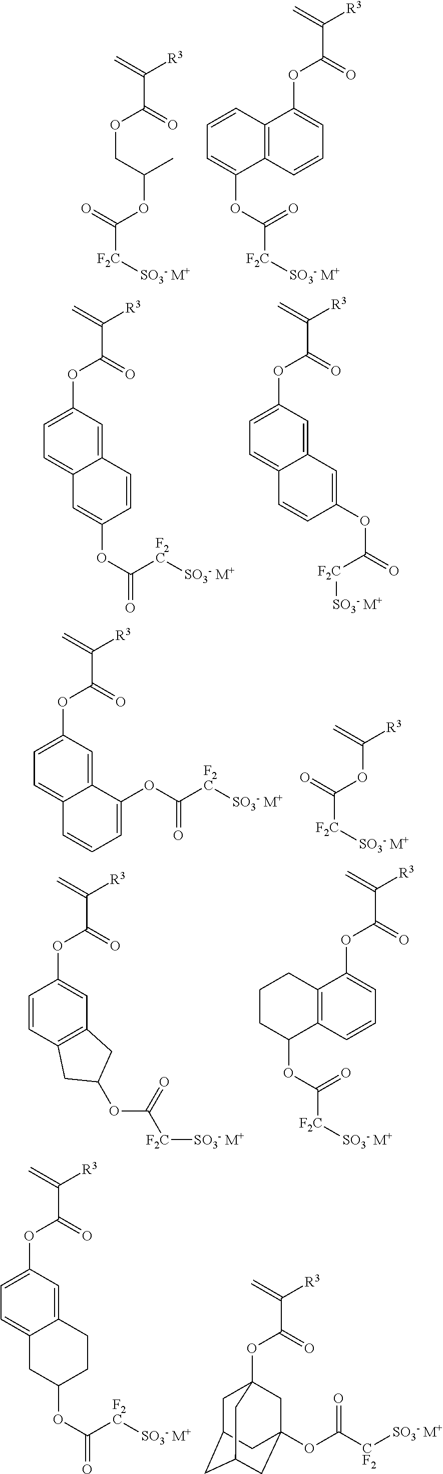

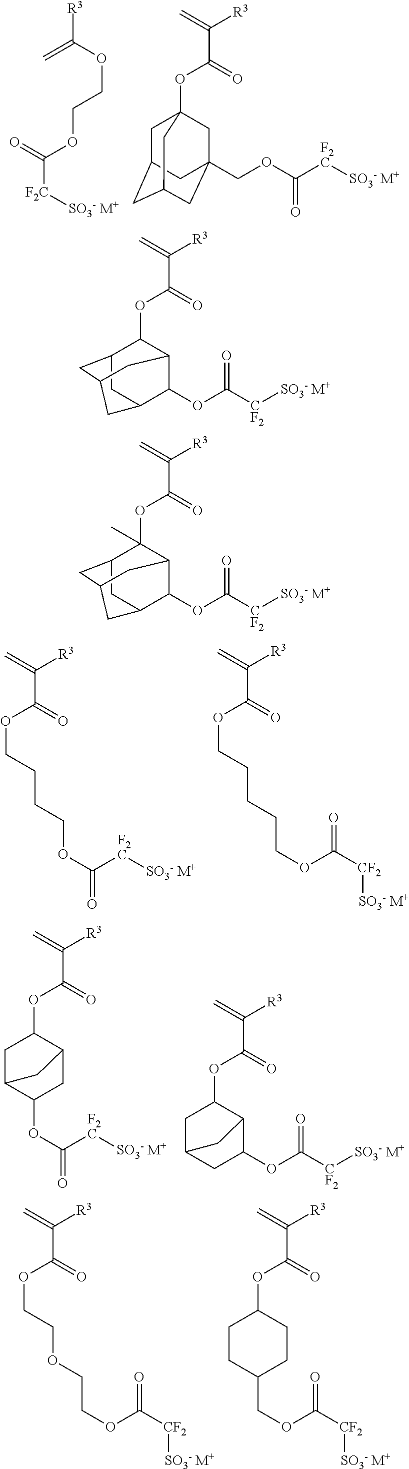

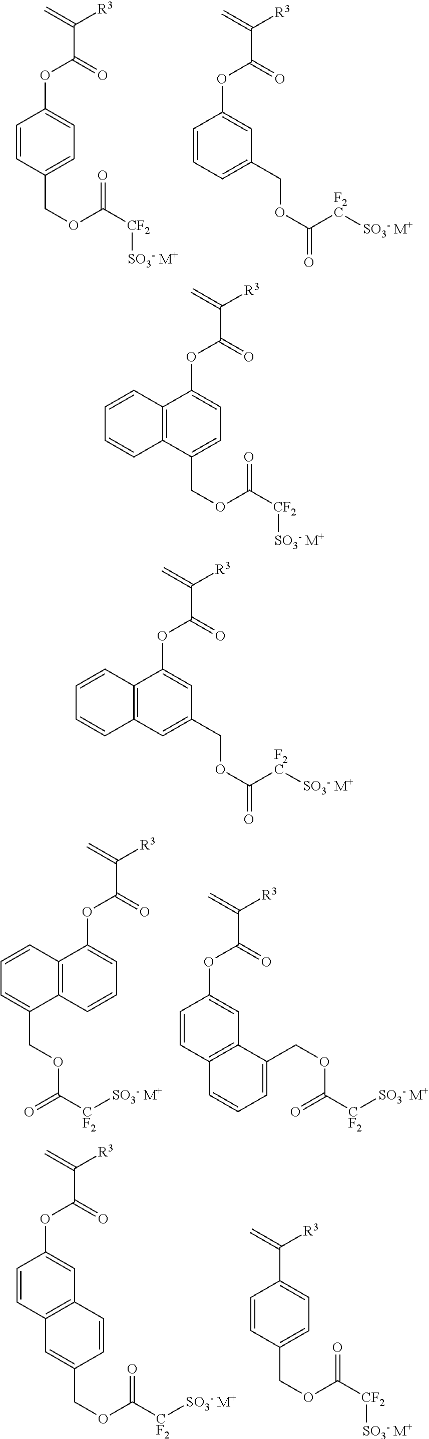

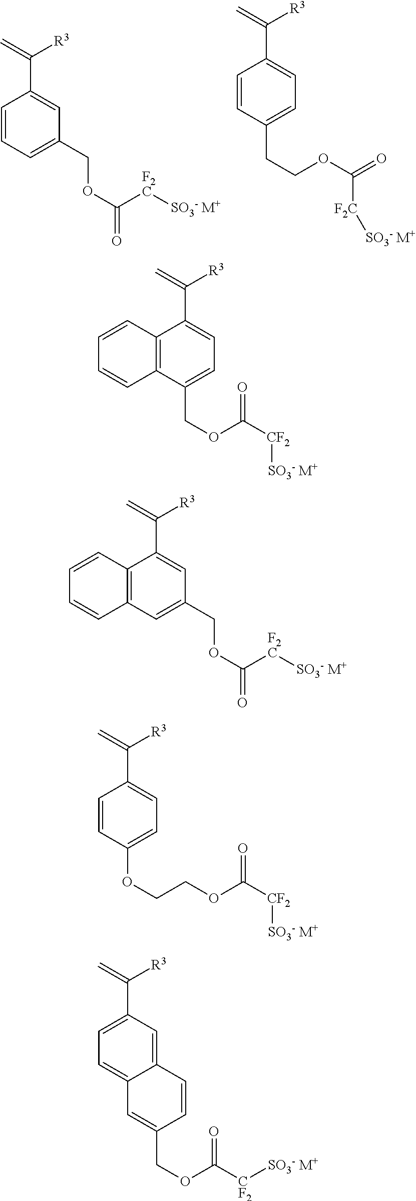

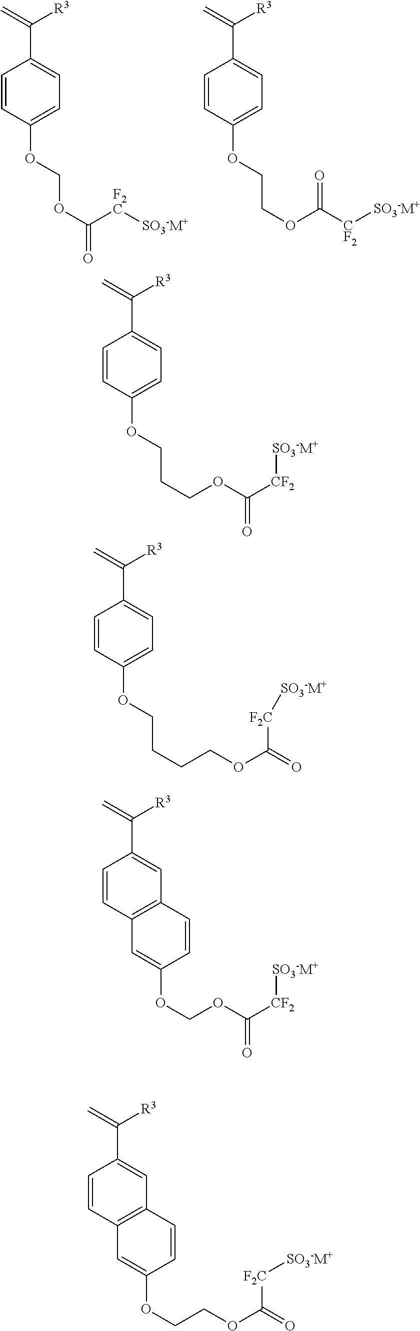

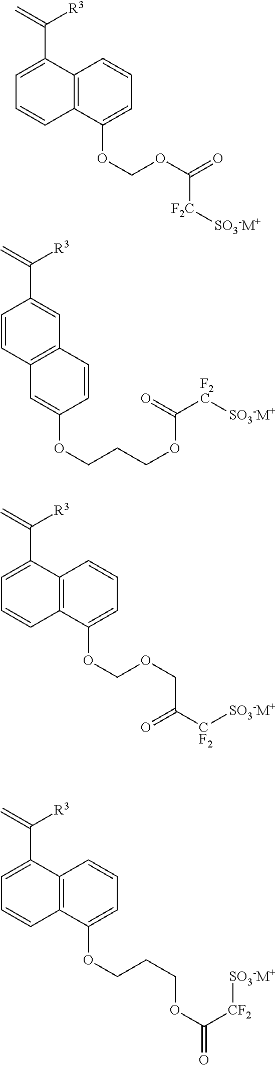

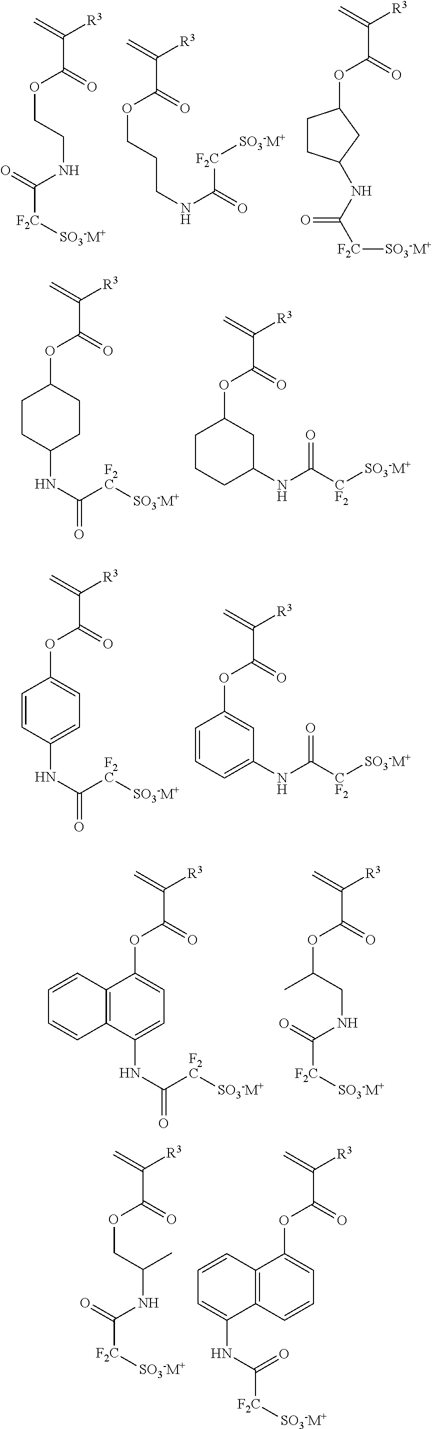

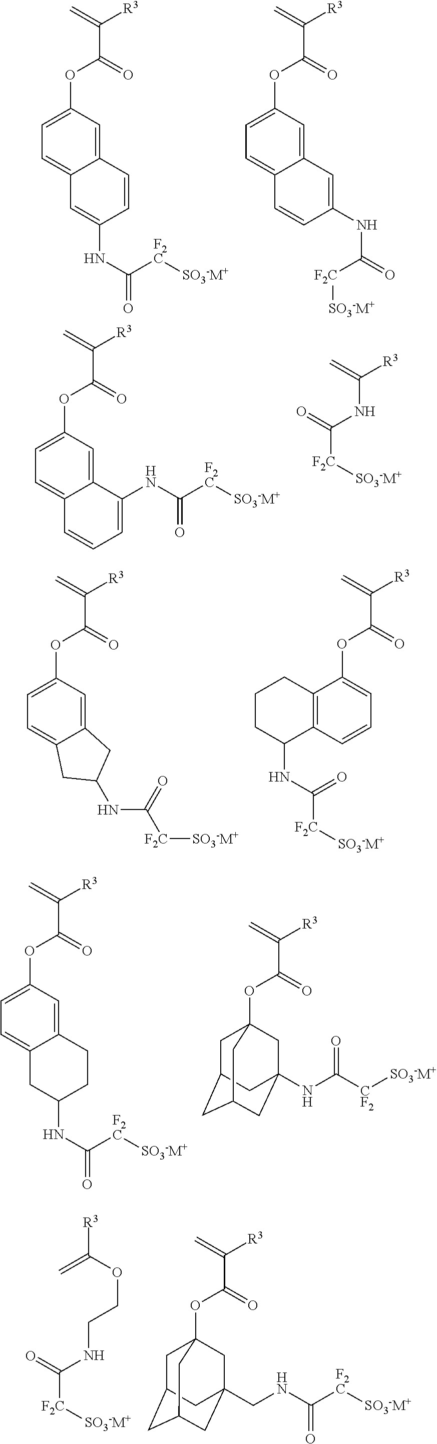

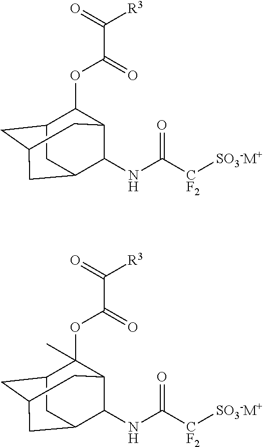

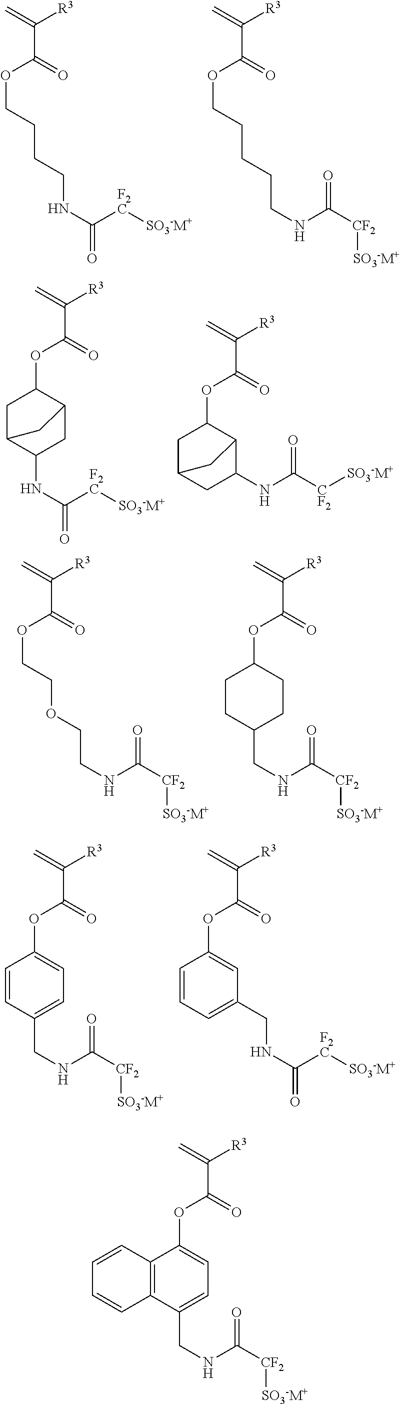

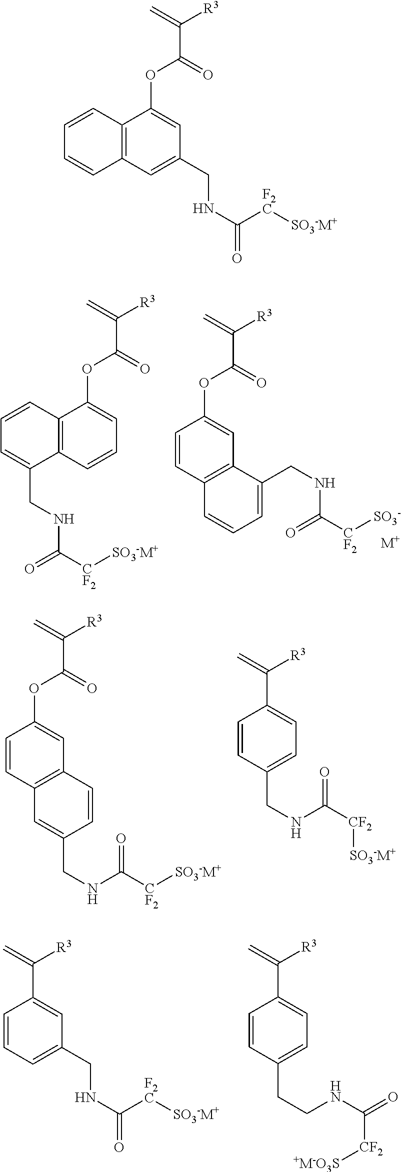

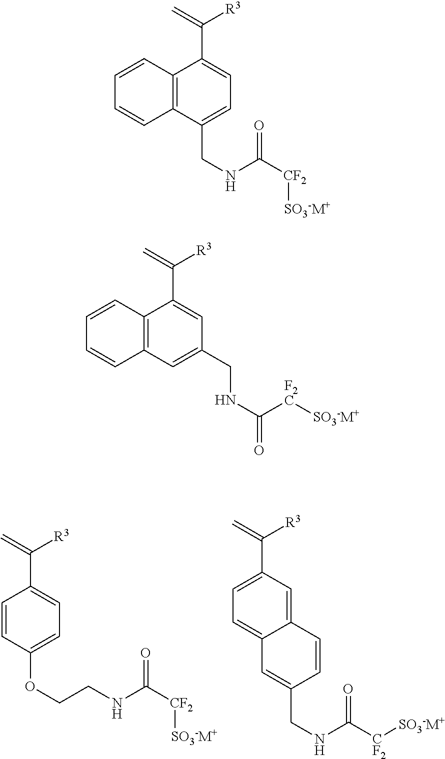

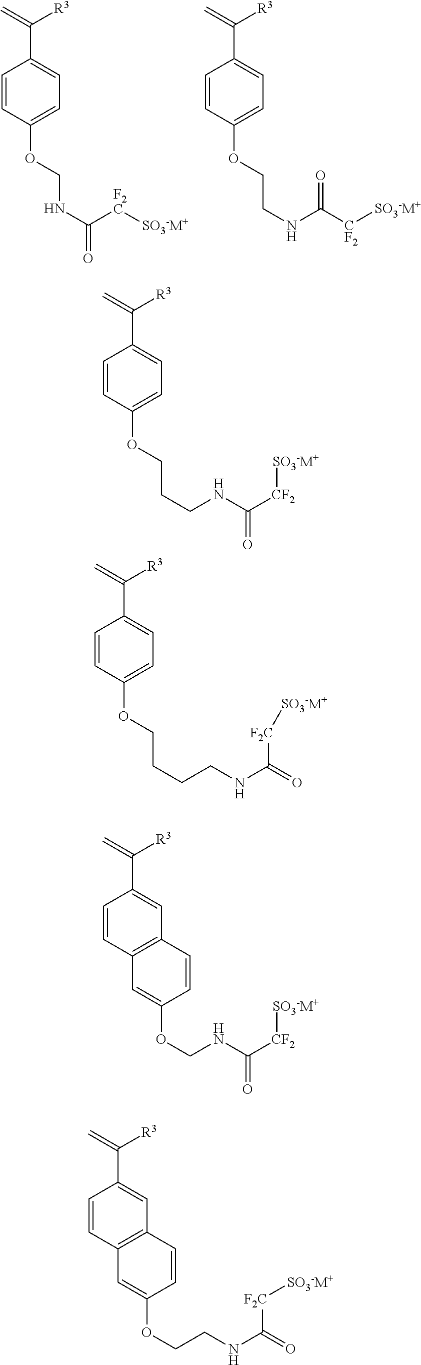

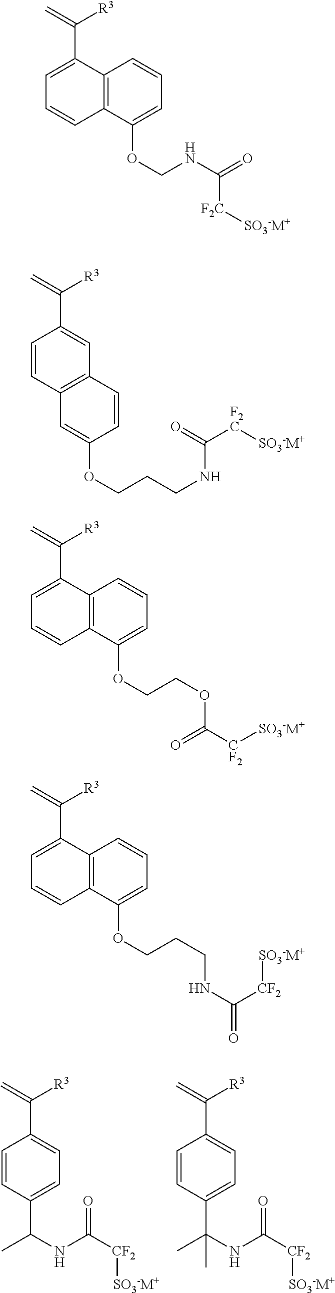

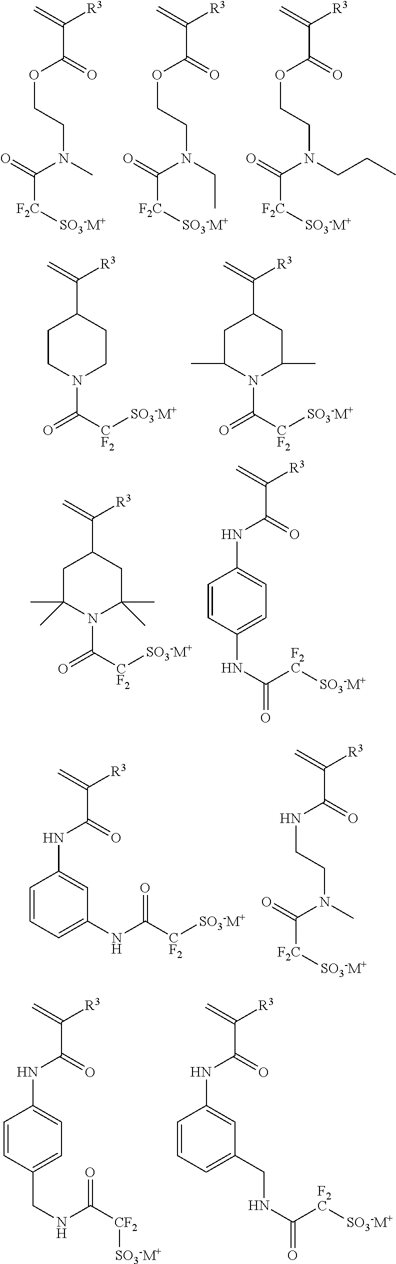

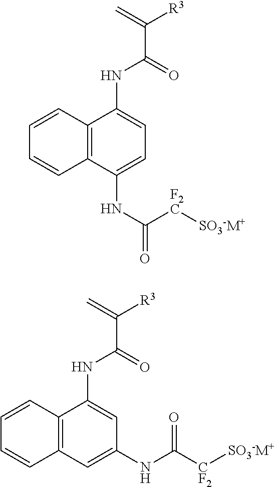

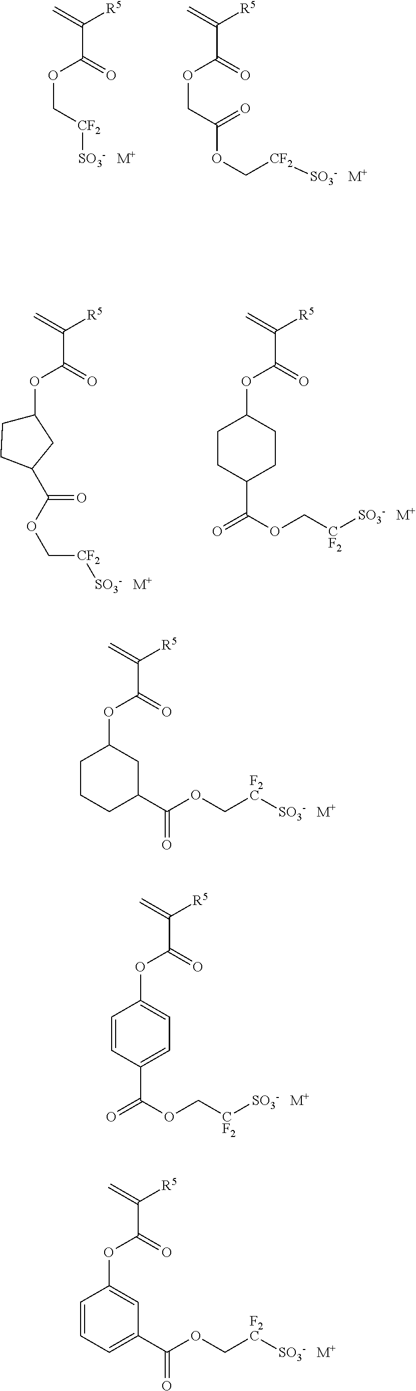

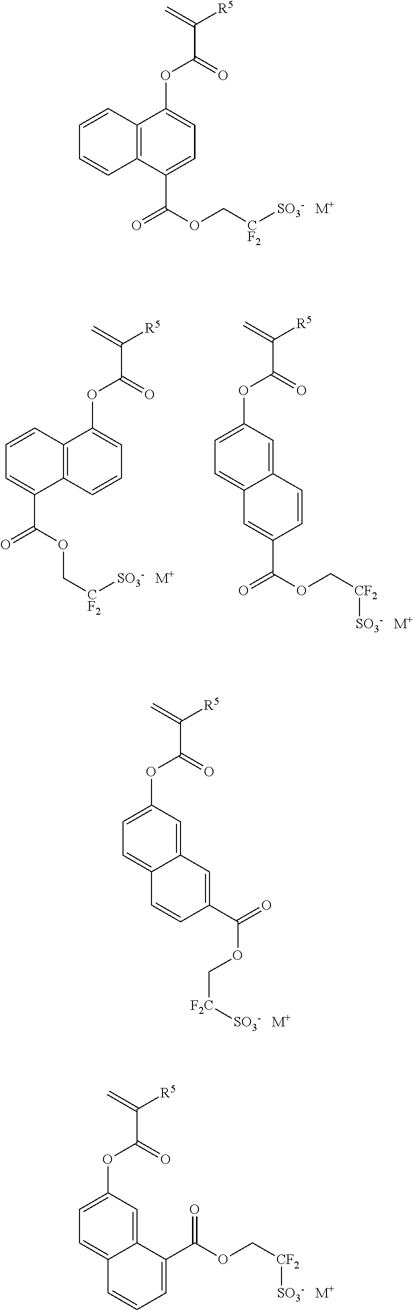

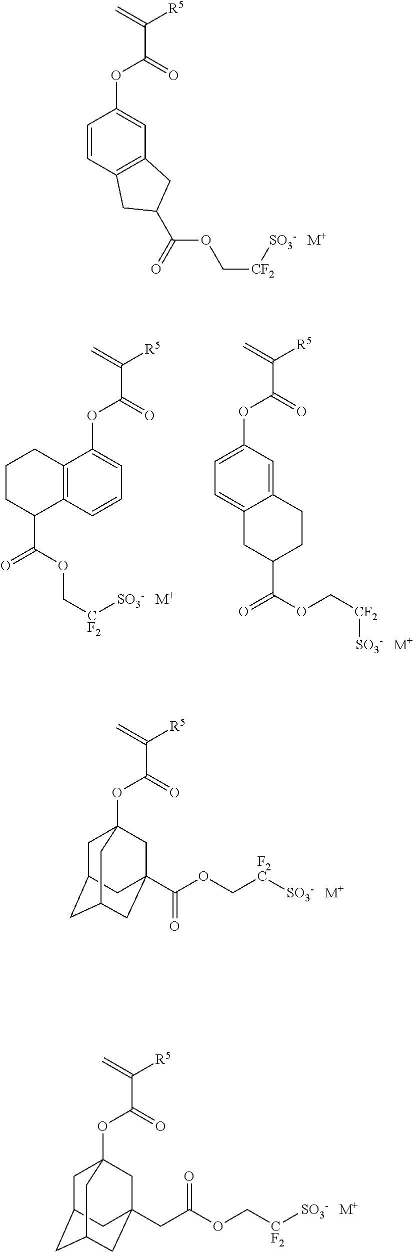

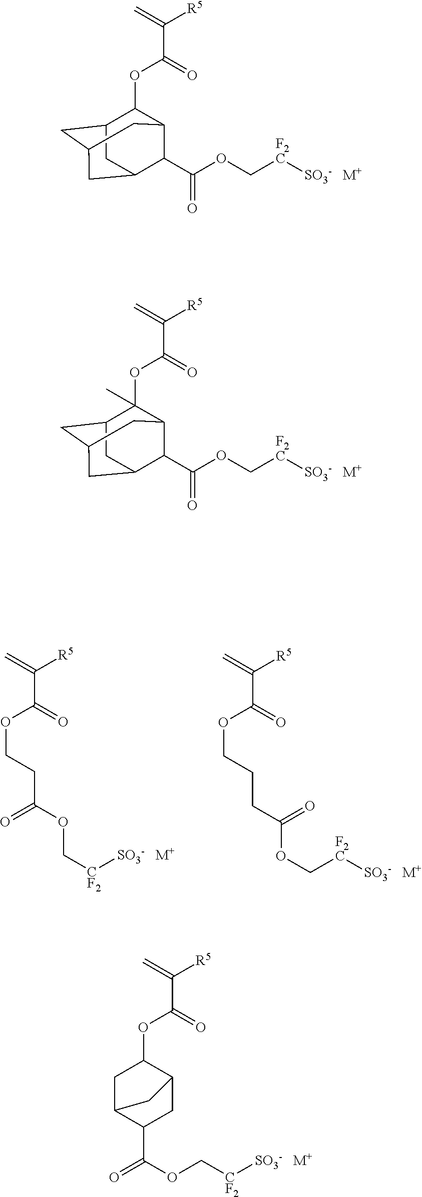

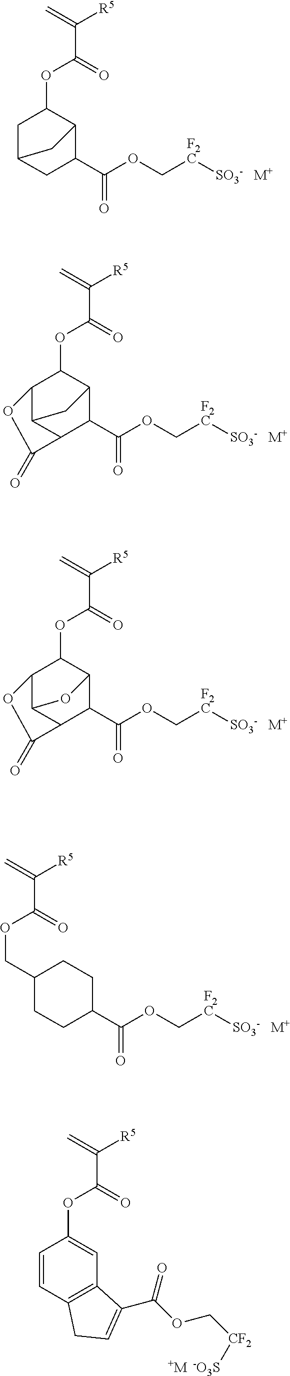

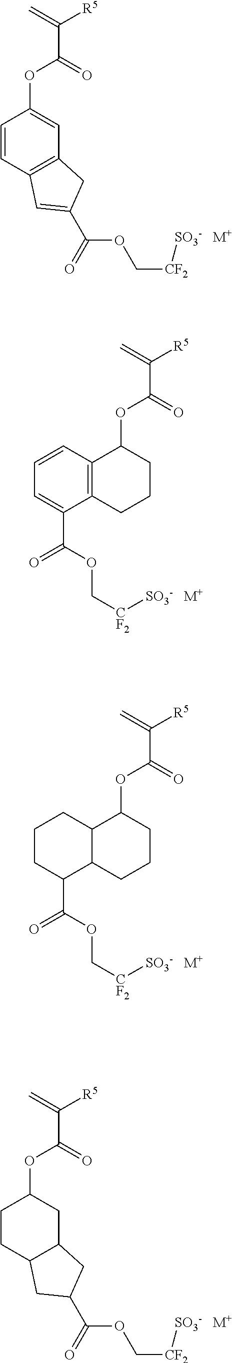

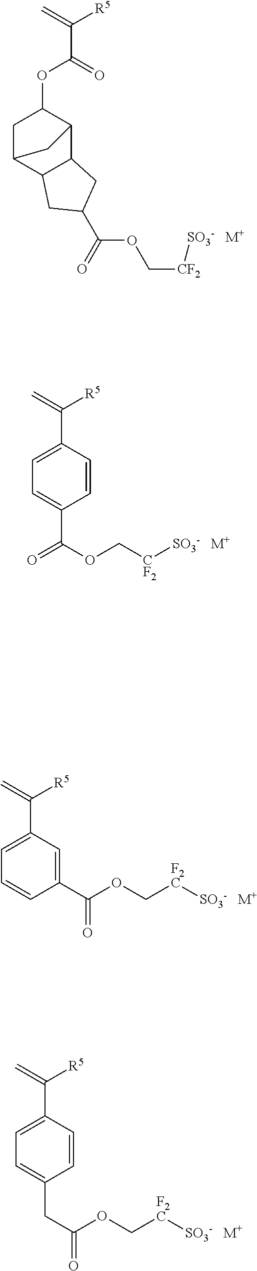

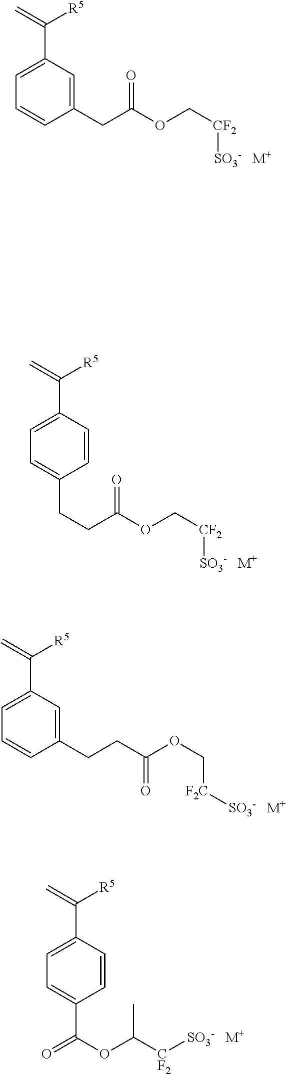

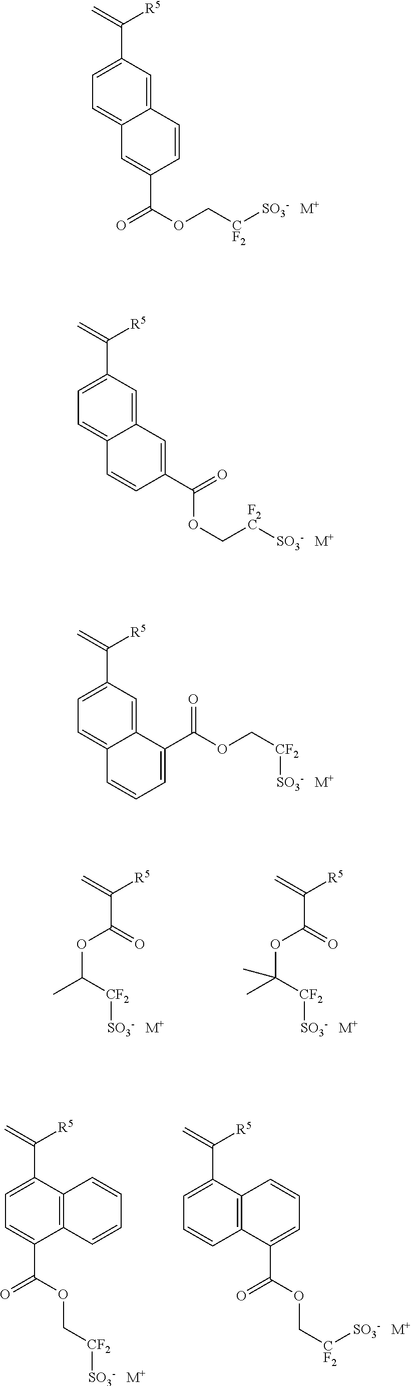

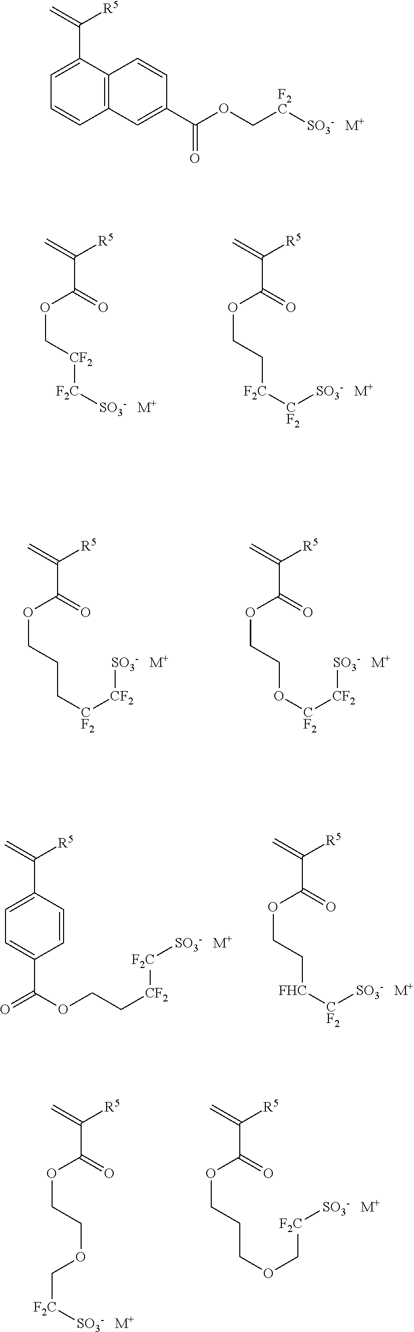

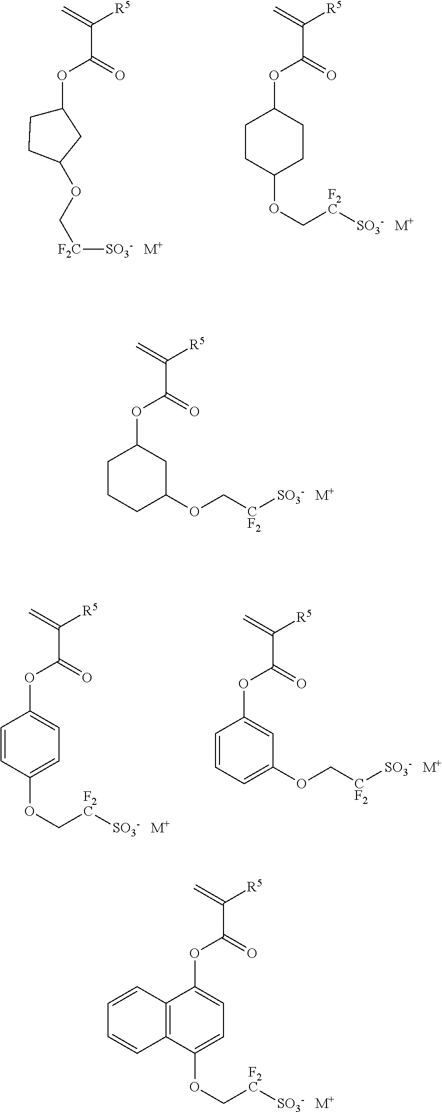

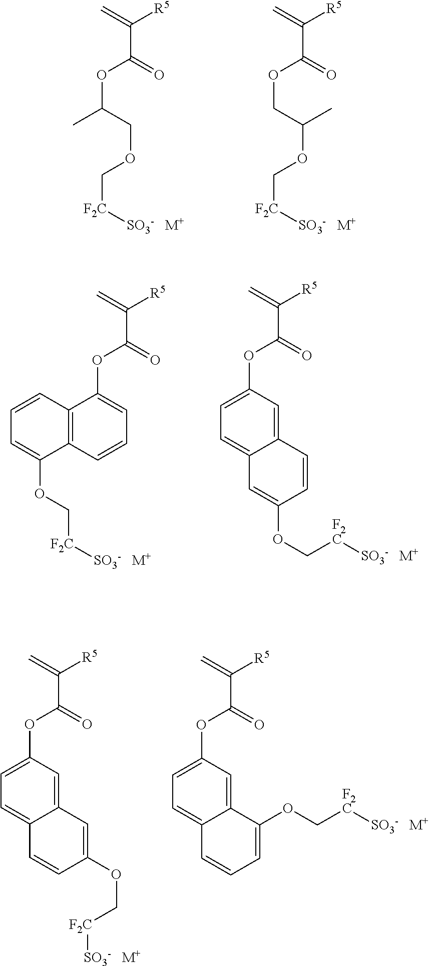

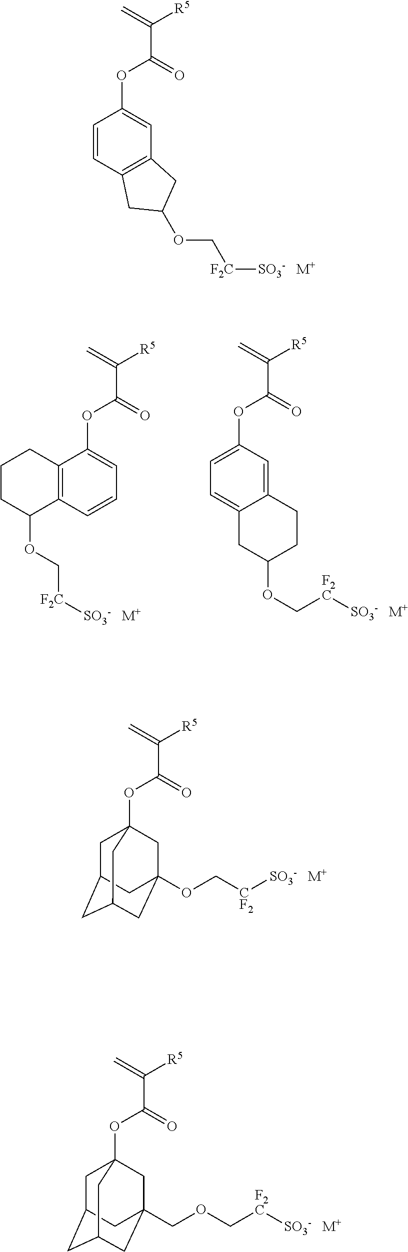

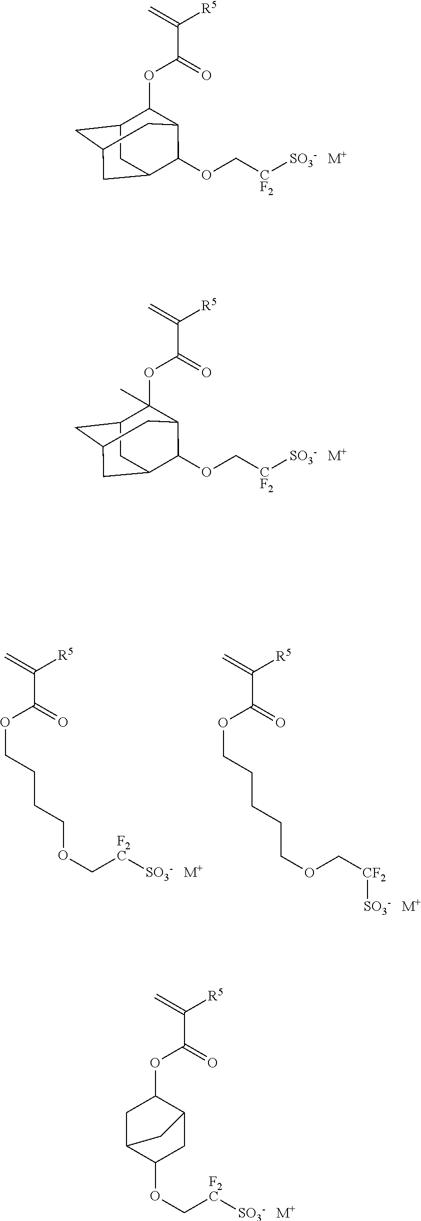

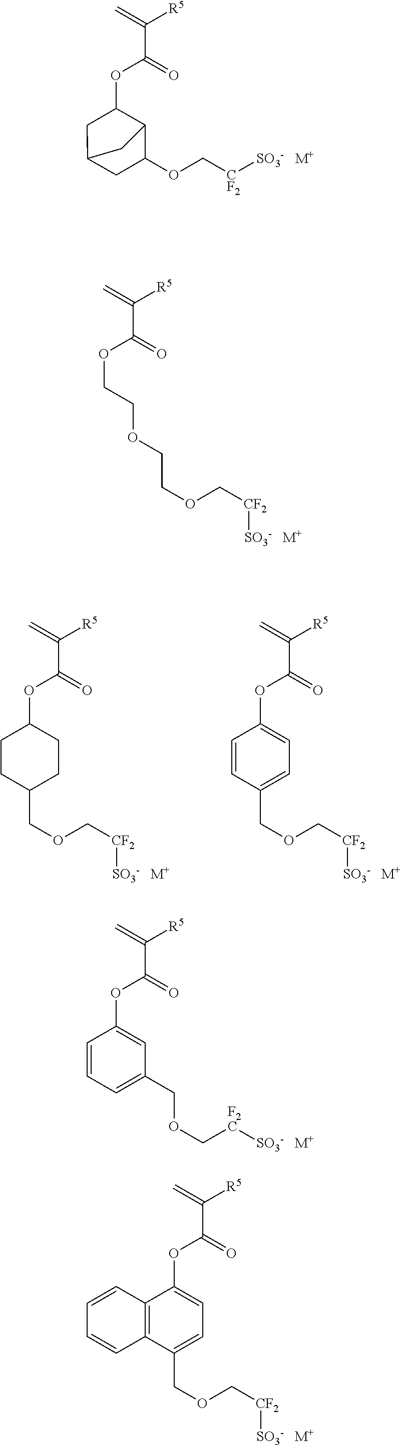

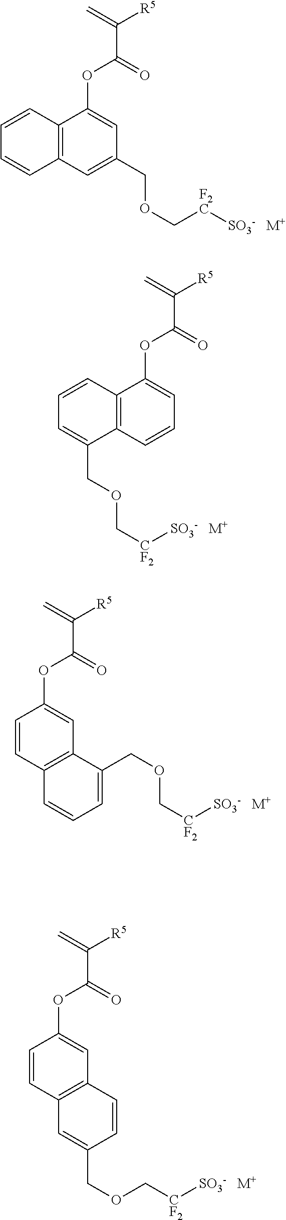

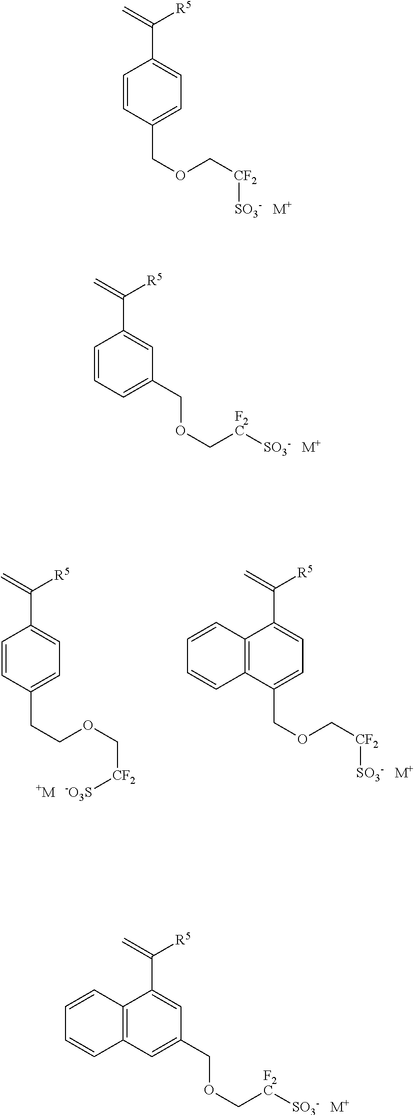

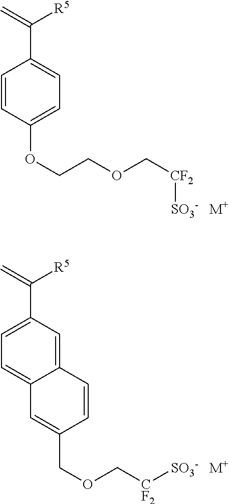

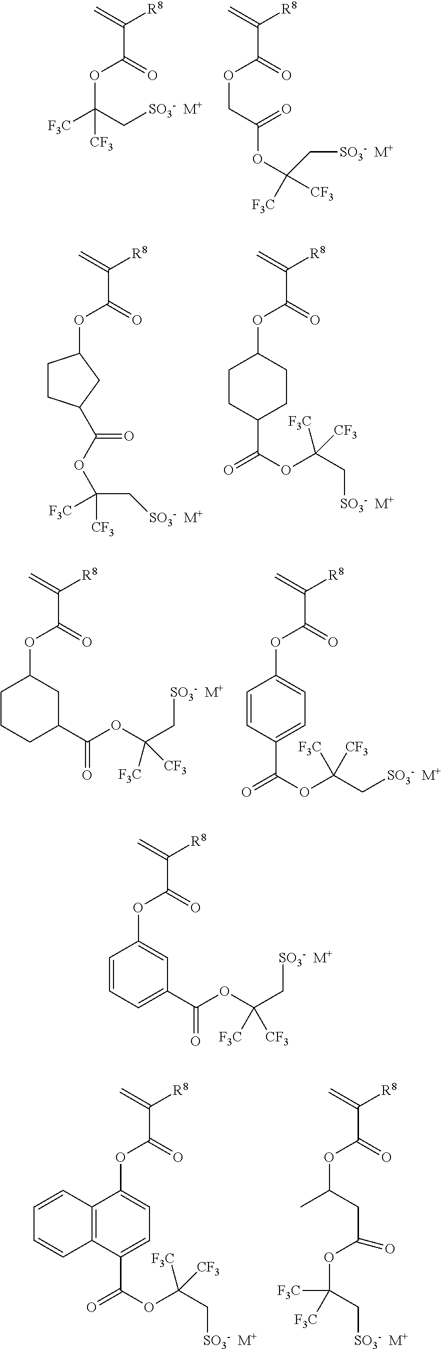

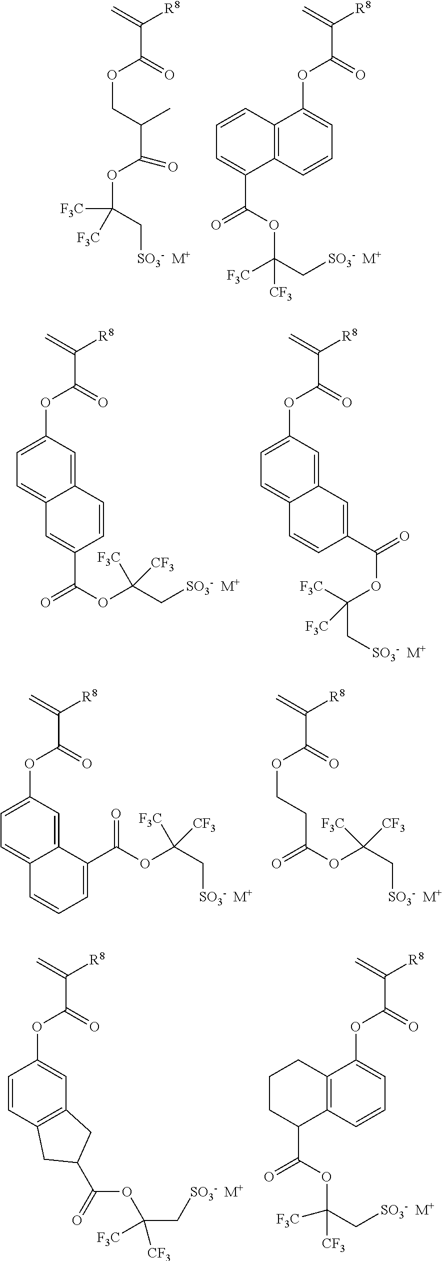

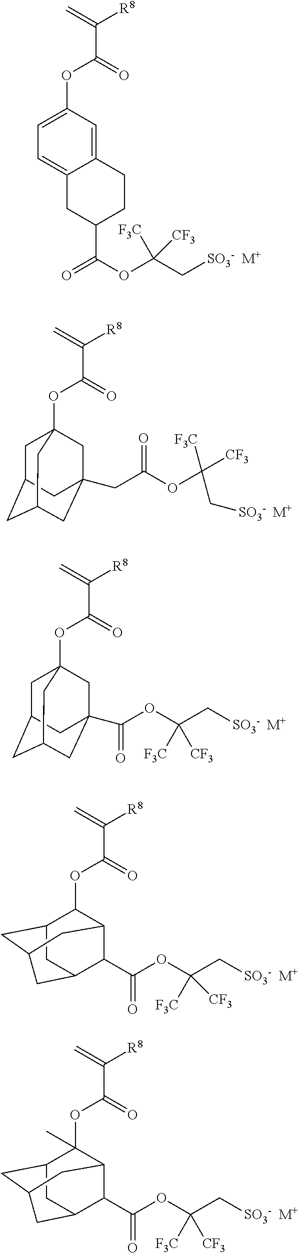

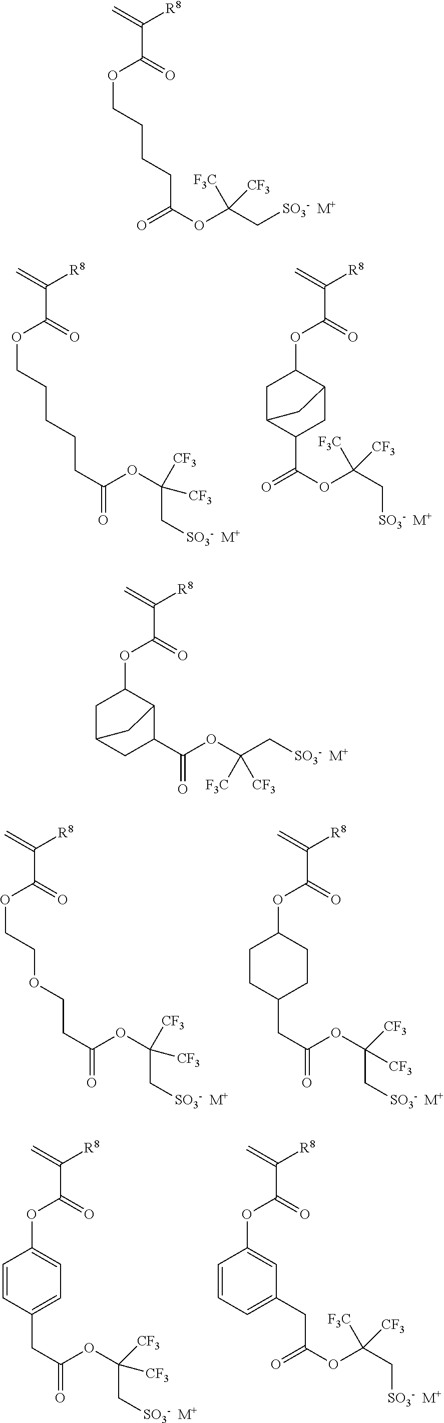

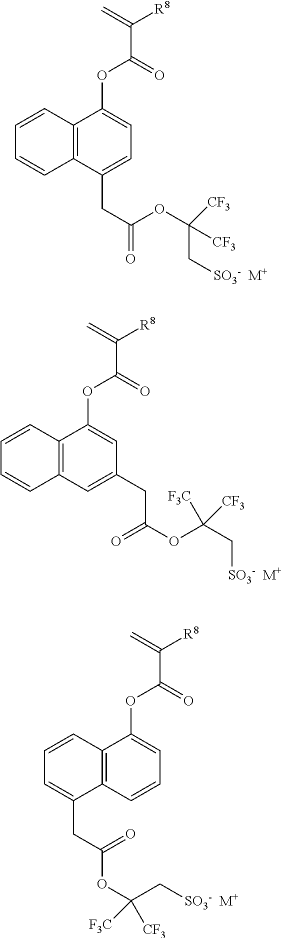

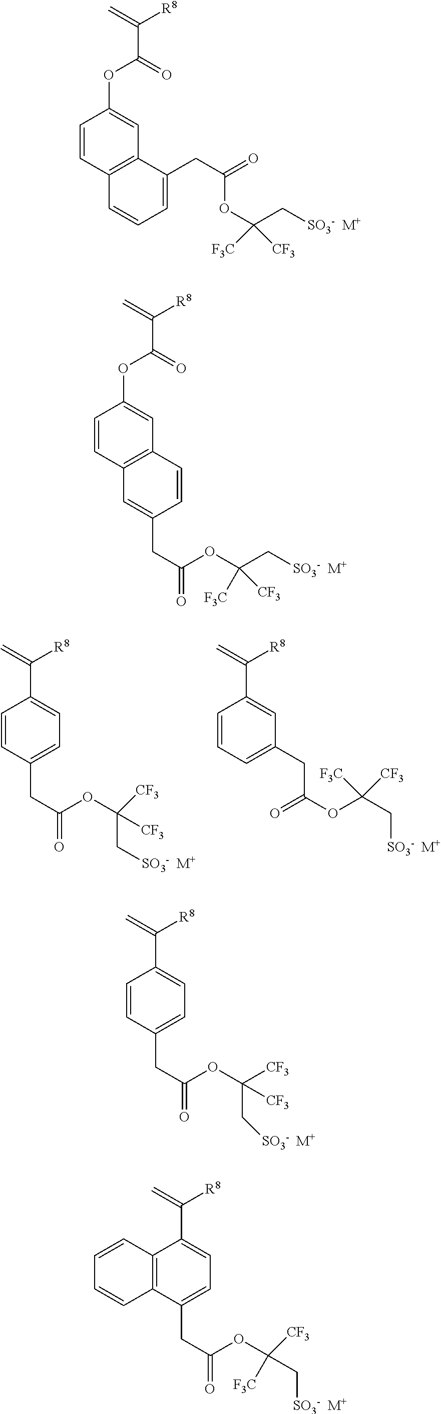

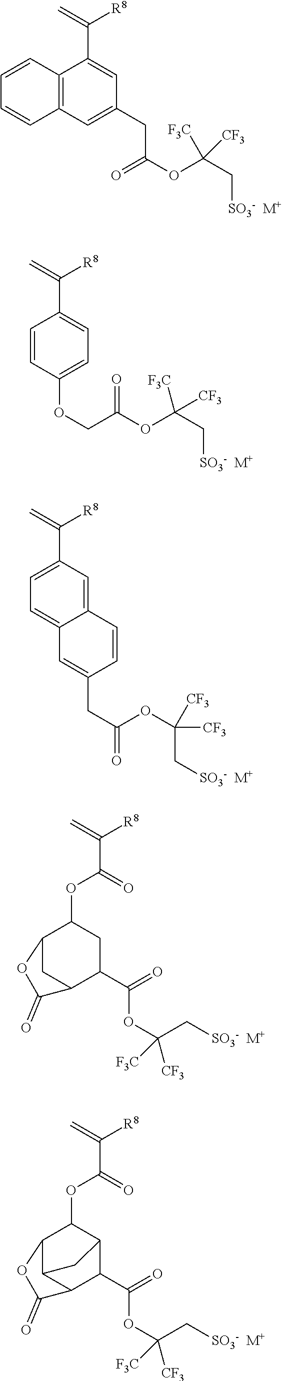

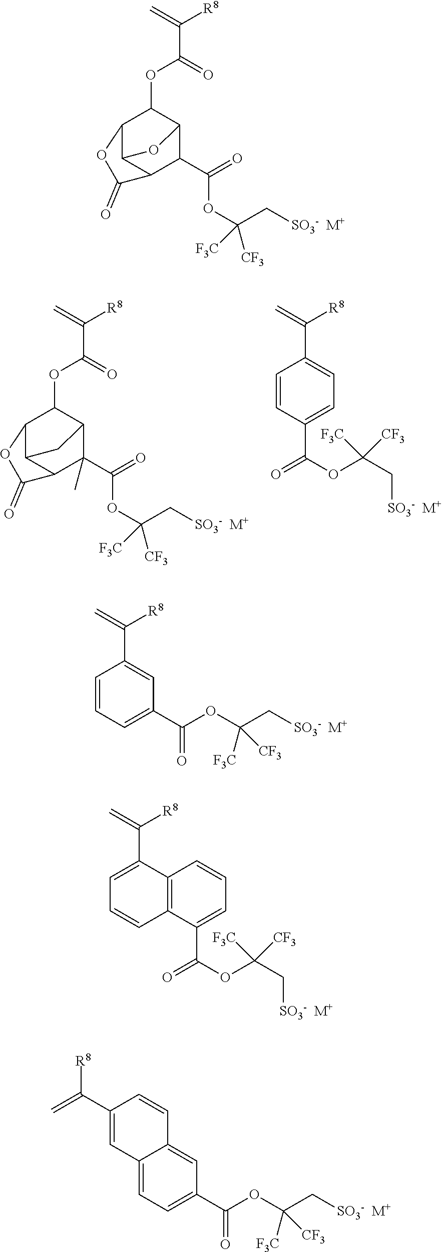

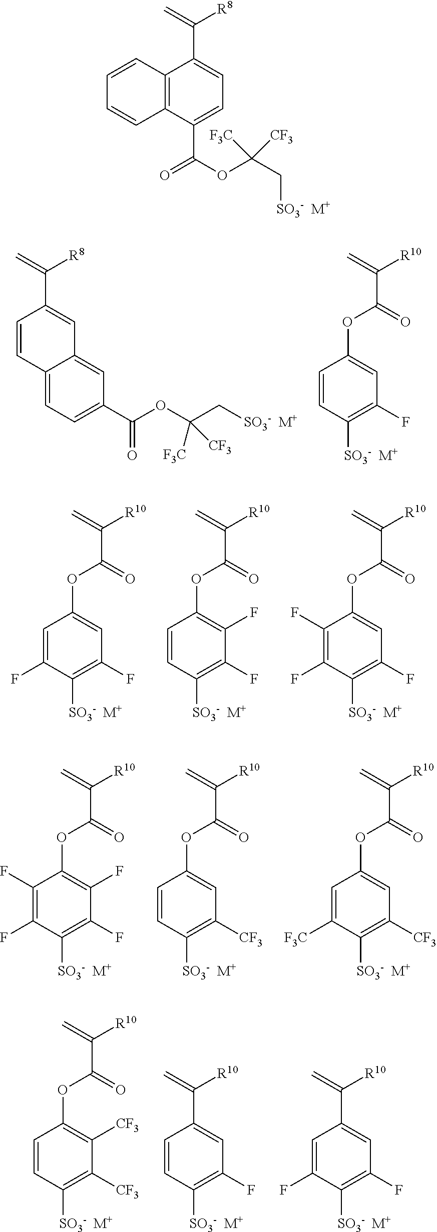

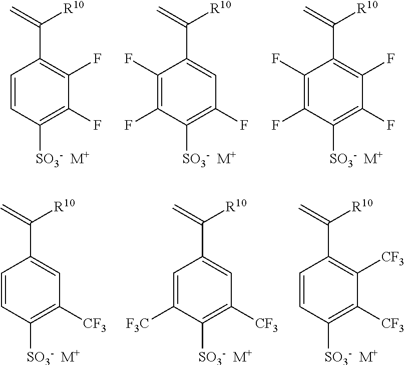

Among the repeating units-A1 to -A7 shown by the general formula (2), the repeating unit-A6 can be obtained from the monomers specifically exemplified below.

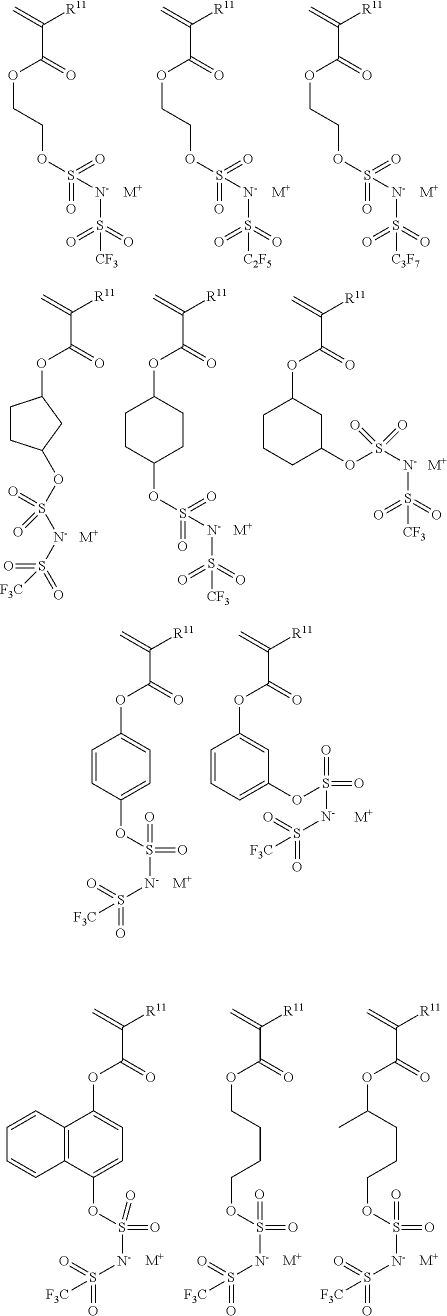

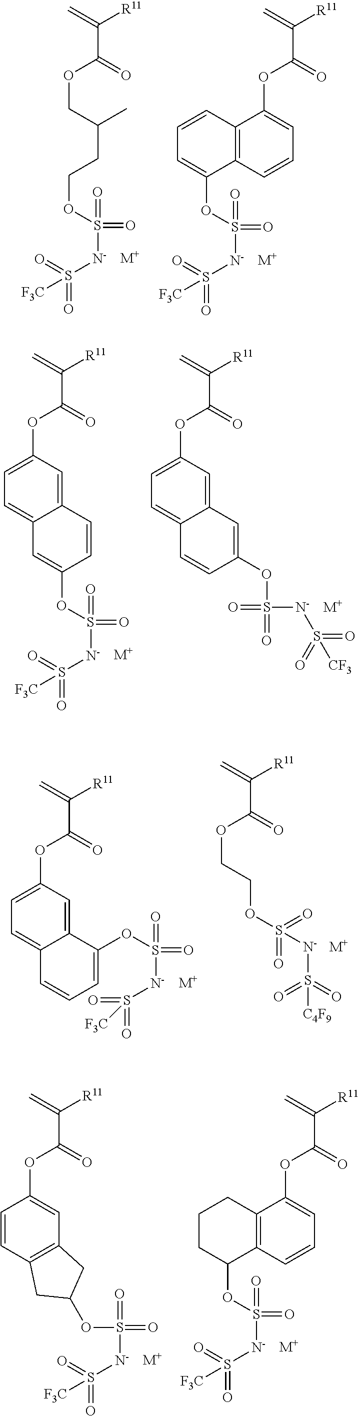

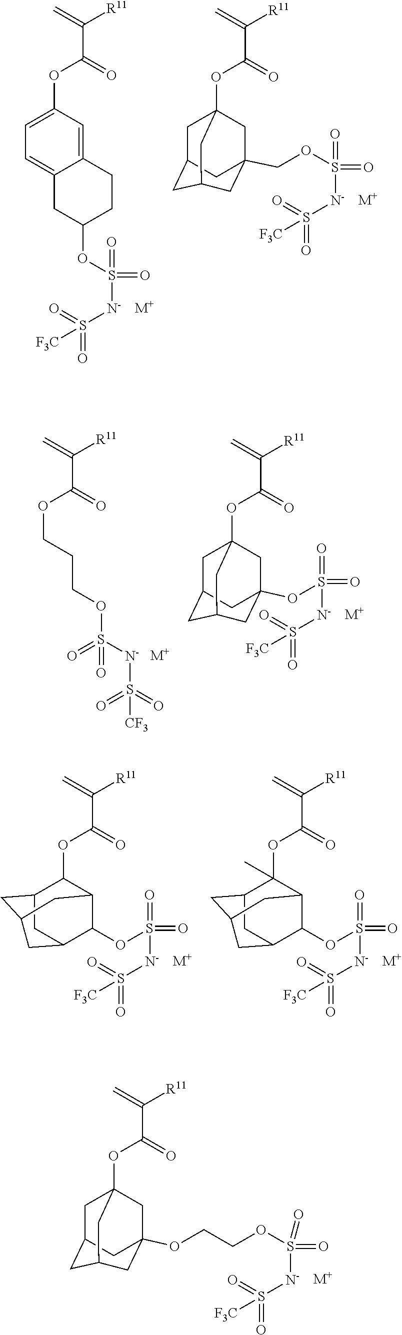

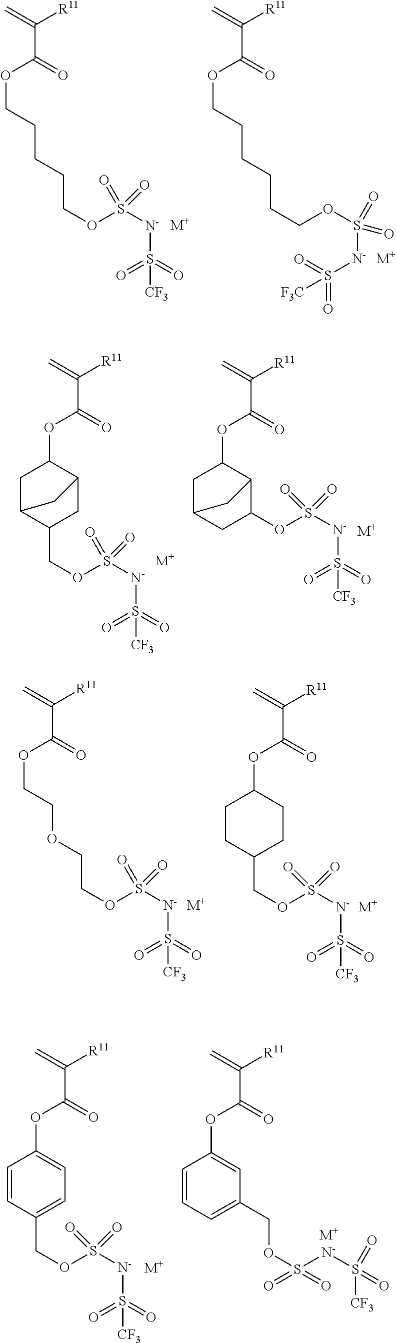

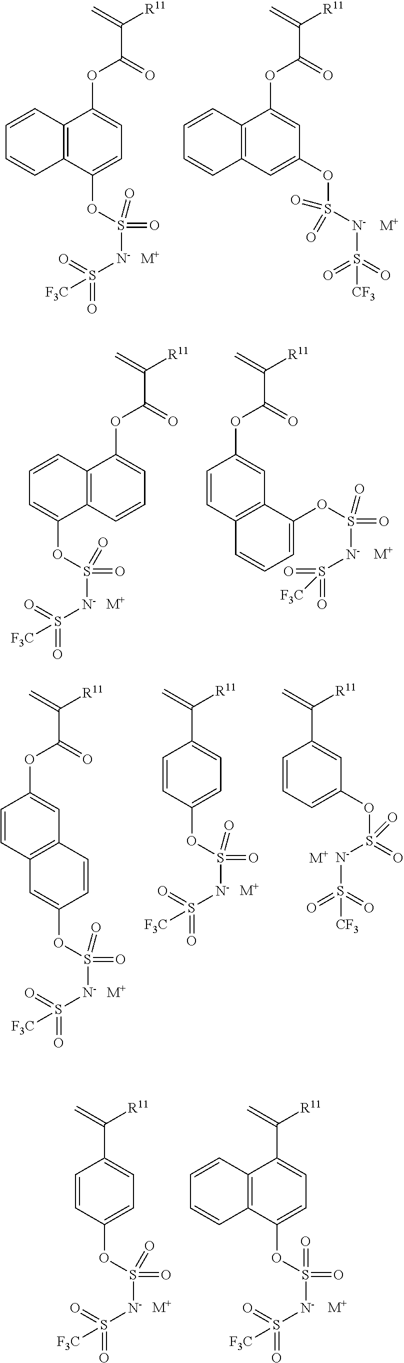

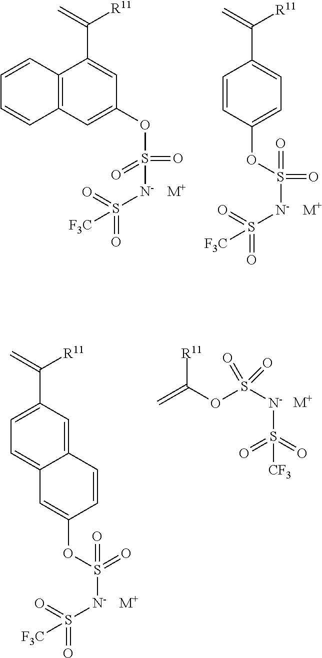

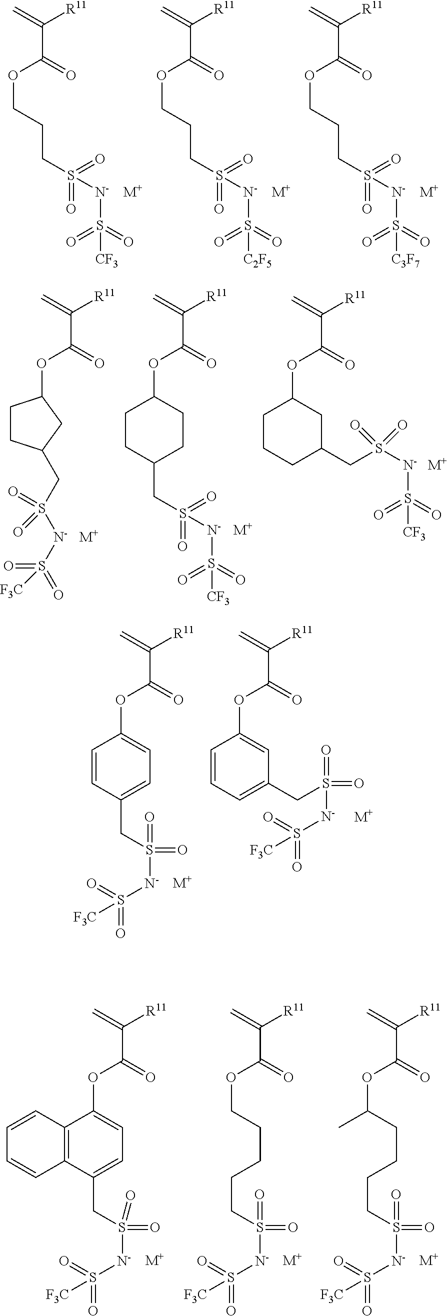

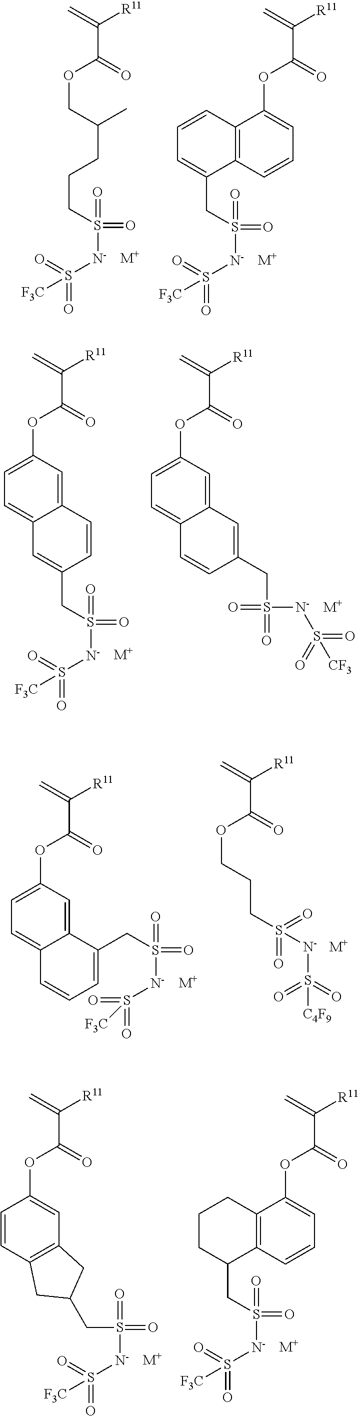

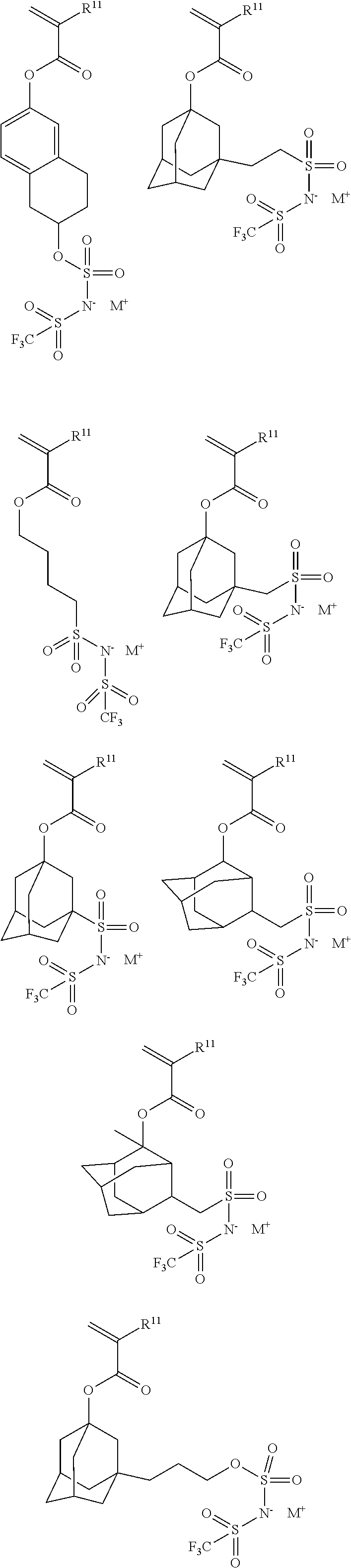

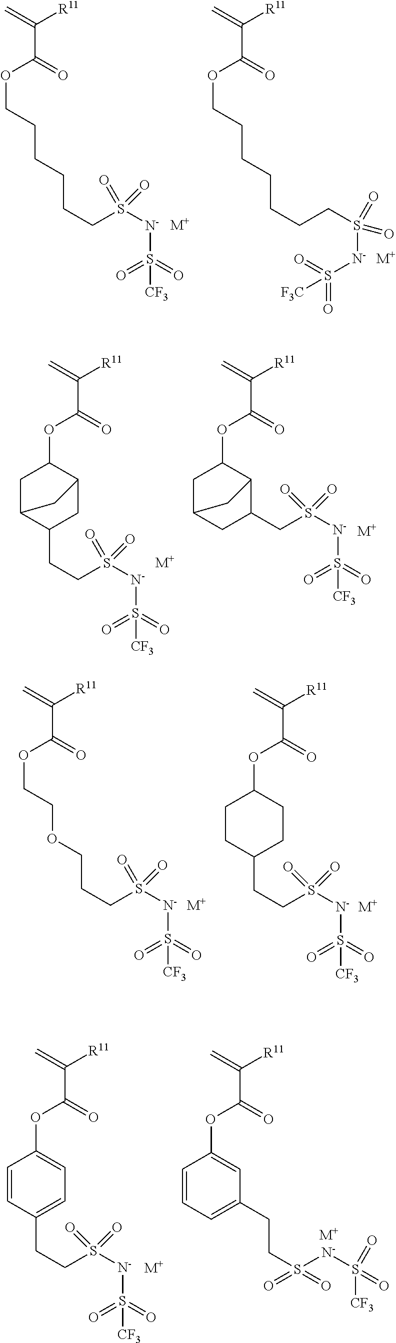

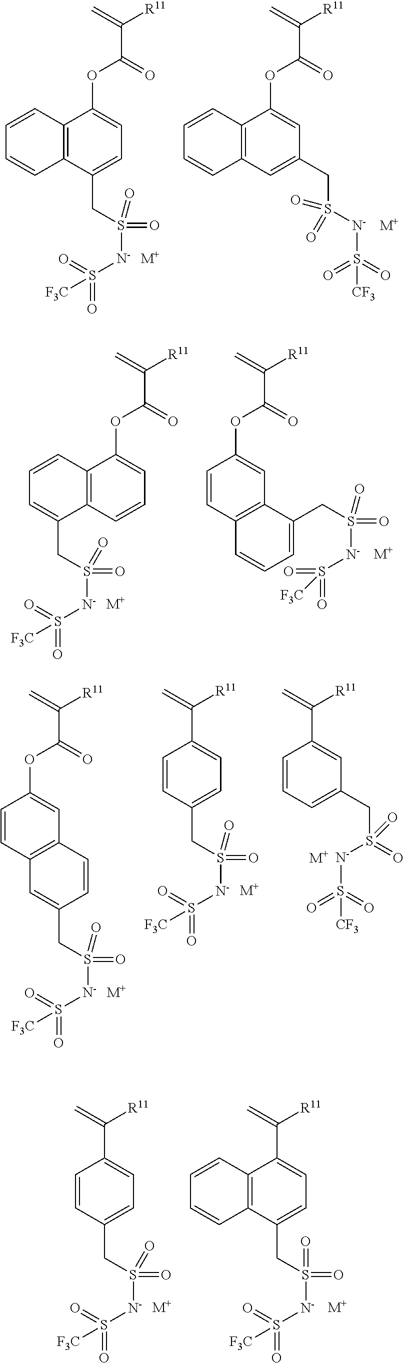

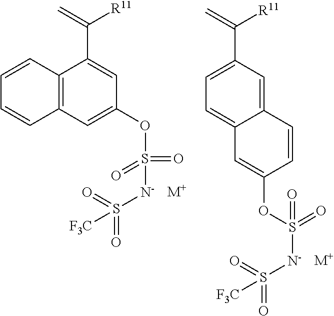

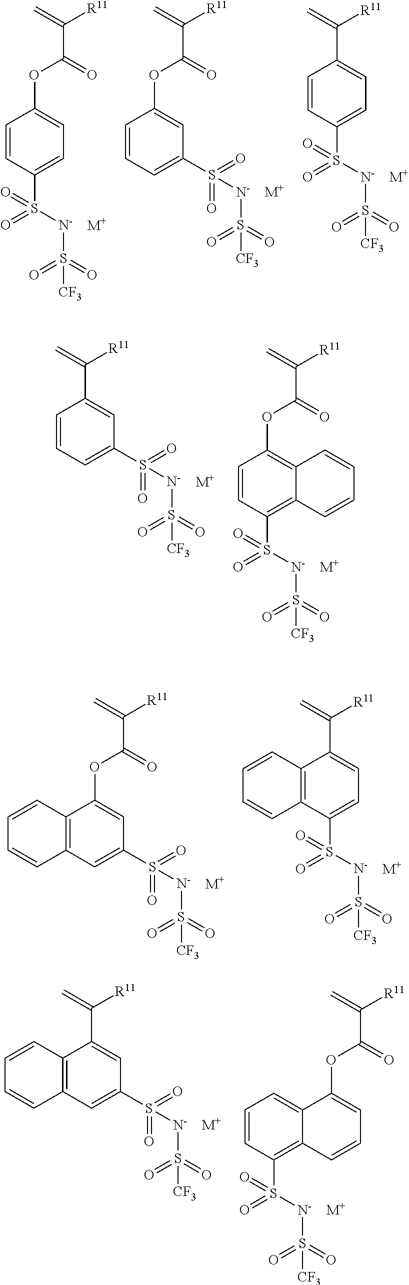

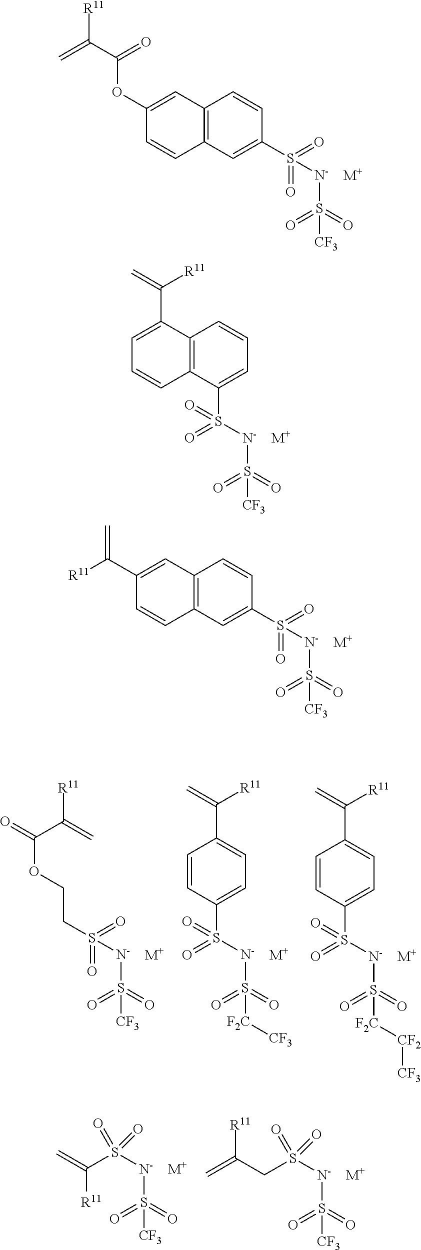

Among the repeating units-A1 to -A7 shown by the general formula (2), the repeating unit-A7 can be obtained from N-carbonyl-sulfonamide salt monomers specifically exemplified below.

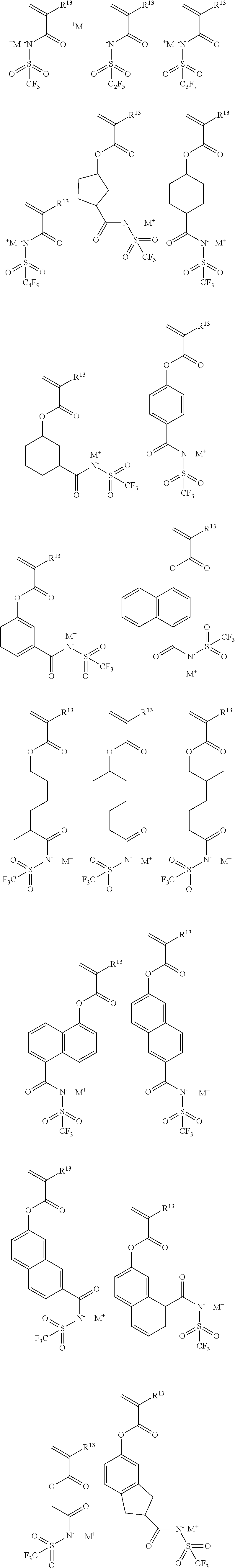

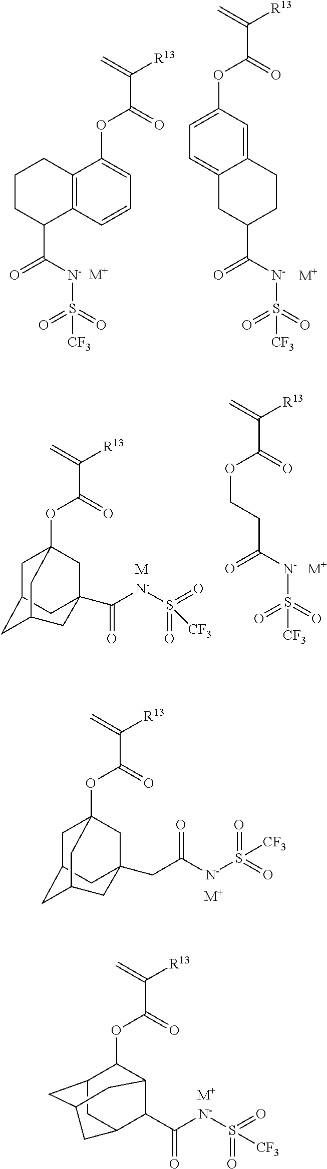

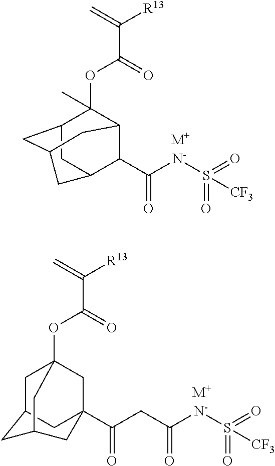

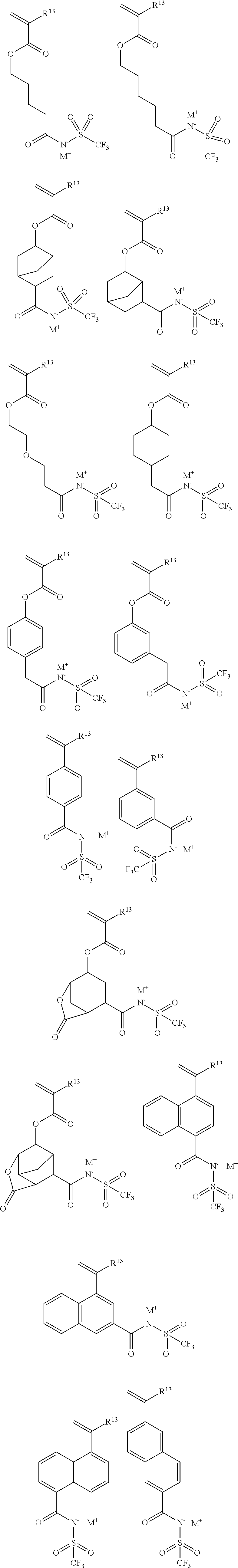

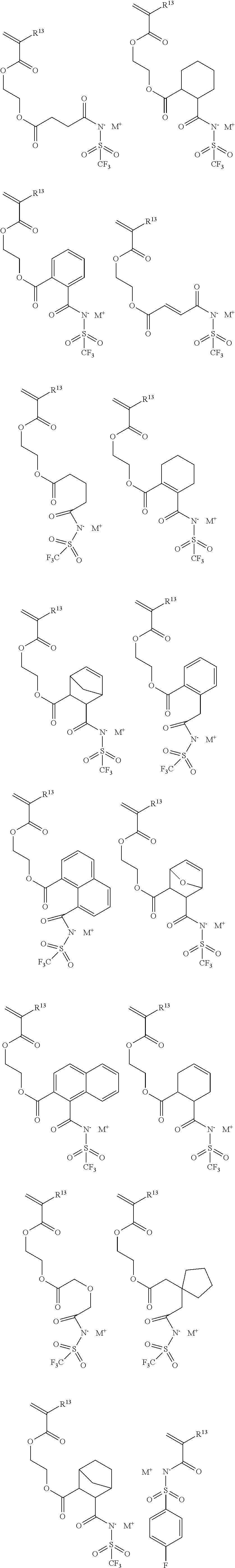

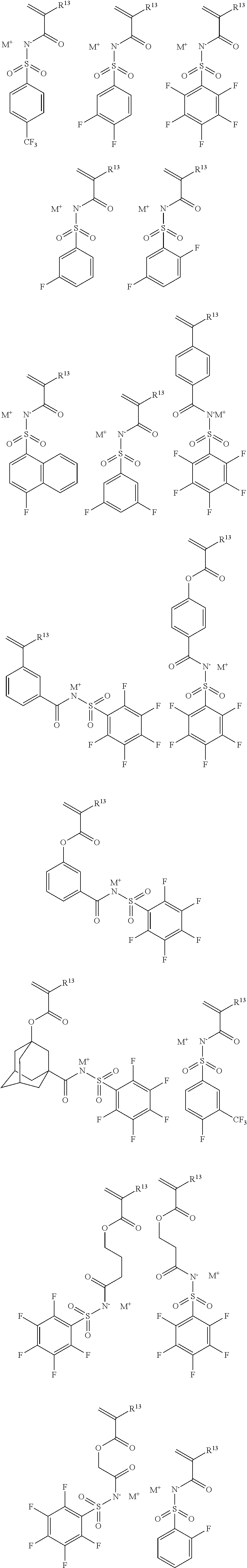

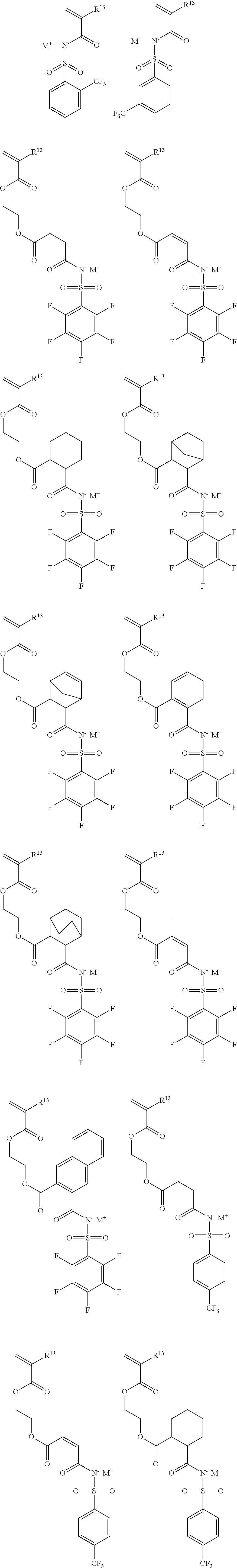

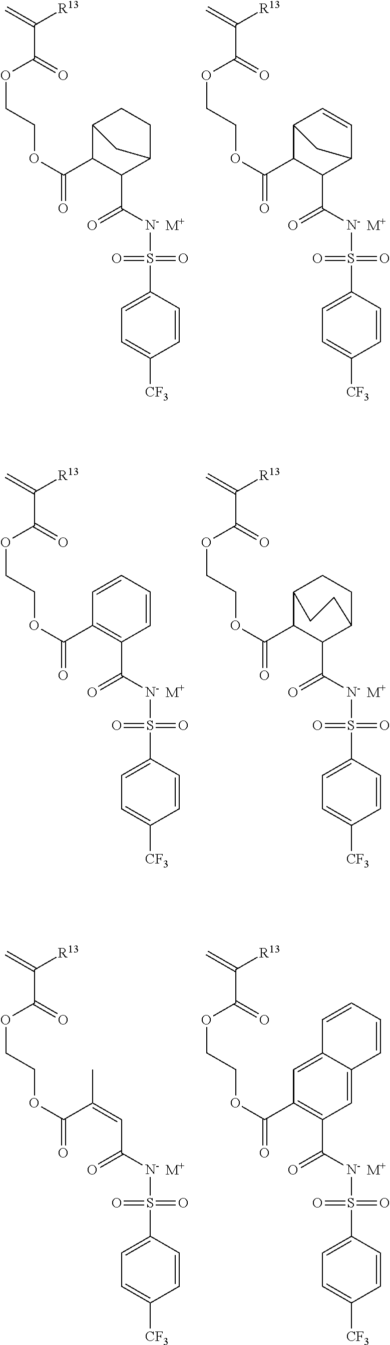

In the formulae, R¹, R³, R⁵, R⁸, R¹⁰, R¹¹, and R¹³ are as defined above.

### (Repeating Unit-b)

For ionic polymer components which are materials to form the ionic polymer containing layer used for the inventive bio-electrode, in addition to the repeating units-A1 to -A7, a repeating unit-b having a glyme chain can also be copolymerized in the ionic polymer in order to enhance the ionic conductivity. Specific examples of a monomer to give the repeating unit-b having a glyme chain include the following. The copolymerization with the repeating unit-b having a glyme chain facilitates the movement of ions released from the skin in a dry electrode film, and thus can increase the sensitivity of a dry electrode.

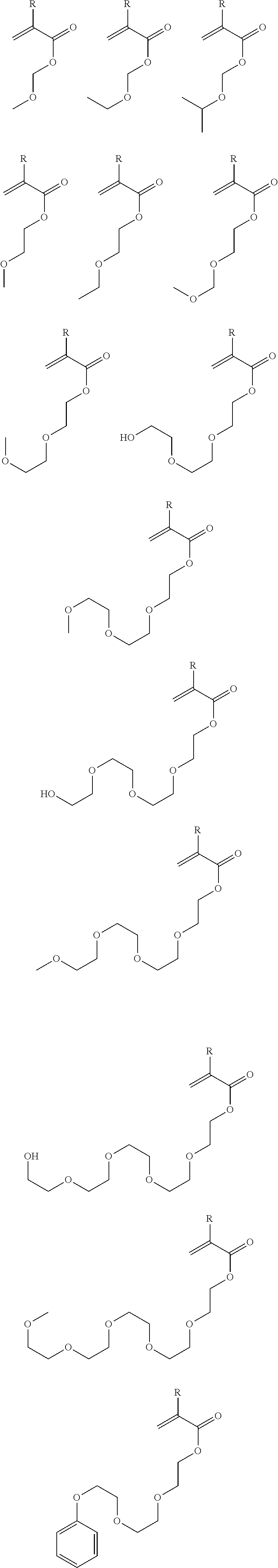

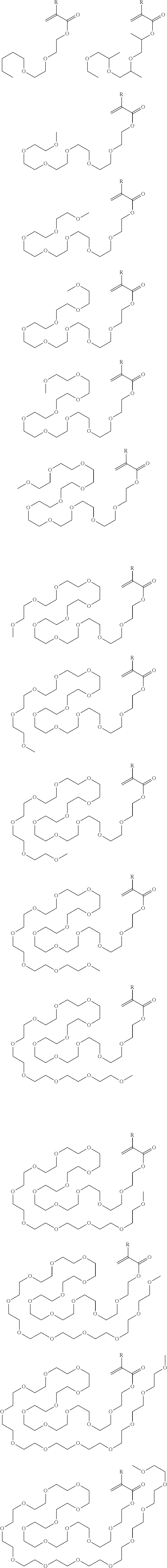

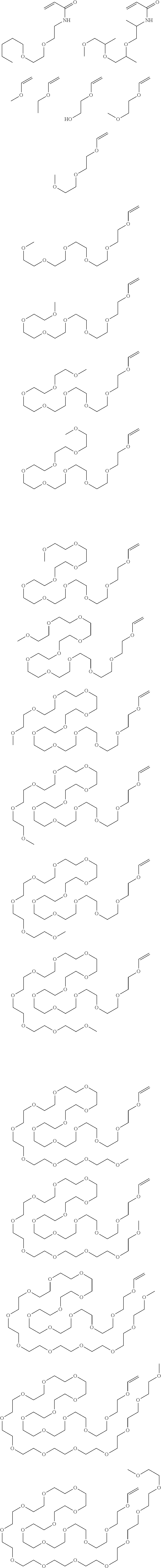

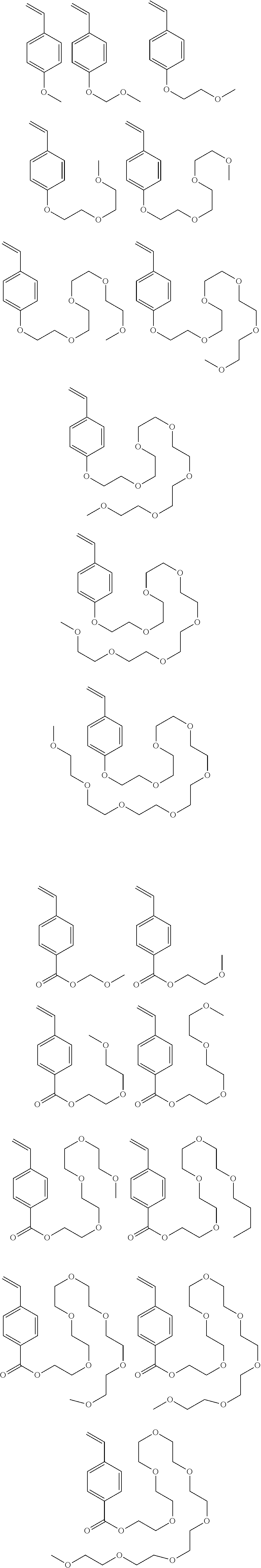

In the formulae, R represents a hydrogen atom or a methyl group.

### (Repeating Unit-c)

For ionic polymer components which are materials to form the ionic polymer containing layer used for the inventive bio-electrode, in addition to the repeating units-A1 to -A7 and -b, it is also possible to copolymerize a hydrophilic repeating unit-c having a hydroxy group, a carboxy group, an ammonium salt, a betaine, an amide group, pyrrolidone, a lactone ring, a lactam ring, a sultone ring, sulfonic acid, a sodium salt of sulfonic acid, or a potassium salt of sulfonic acid in order to enhance electric conductivity. Specific examples of a monomer to give the hydrophilic repeating unit-c include the following. The copolymerization with the repeating unit containing such hydrophilic groups can increase the sensitivity of the dry electrode by increasing the sensitivity to ions released from the skin.

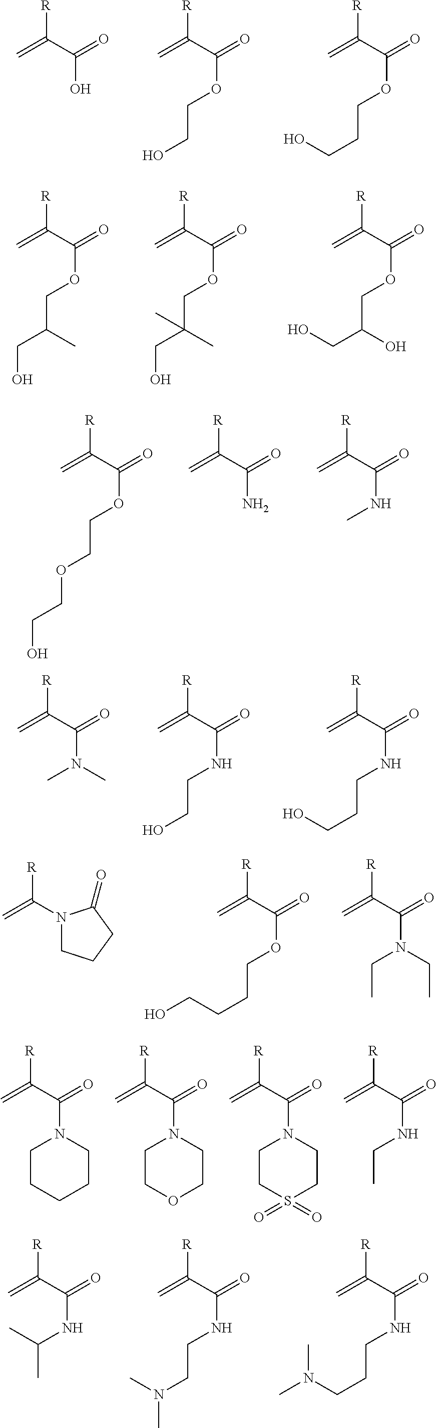

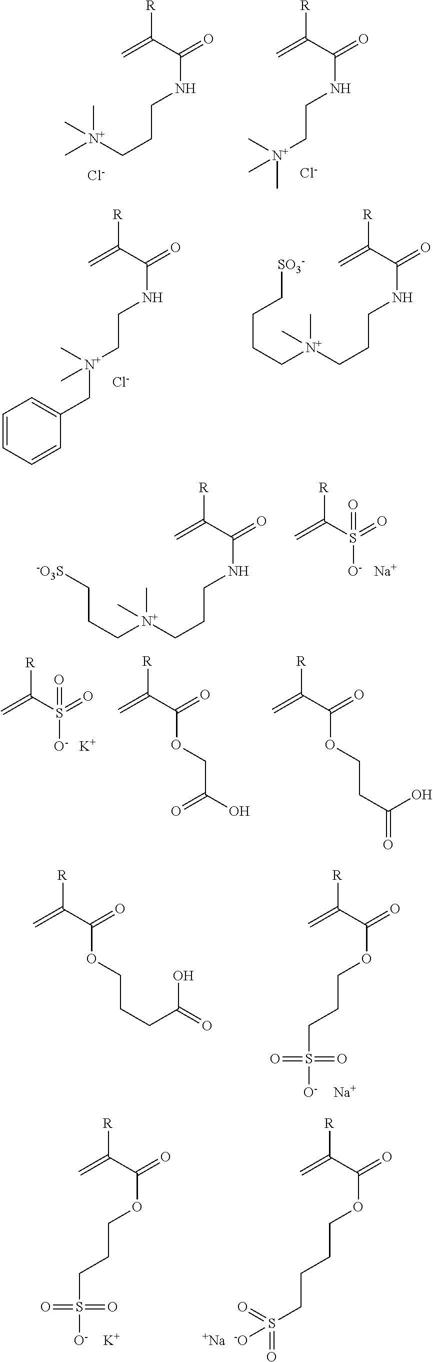

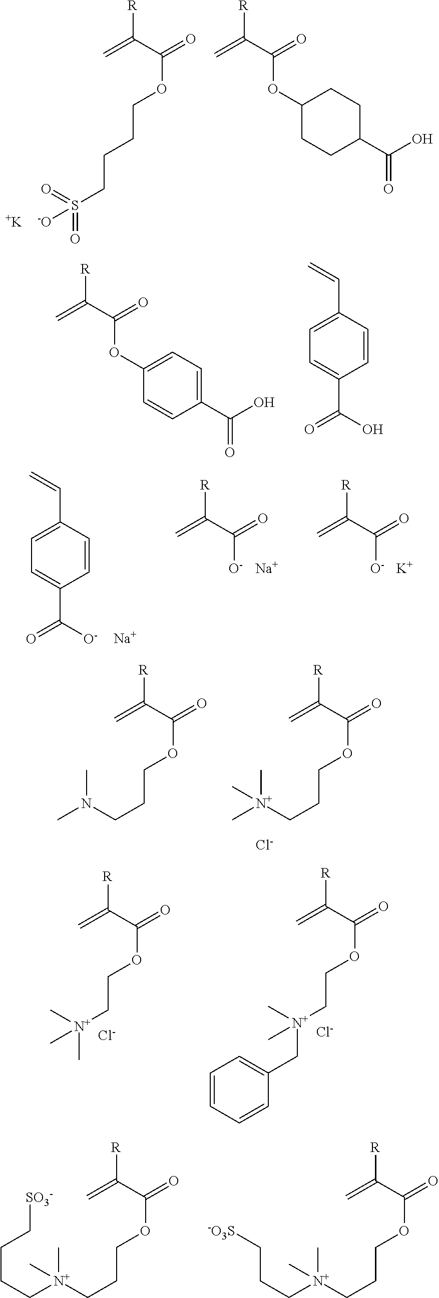

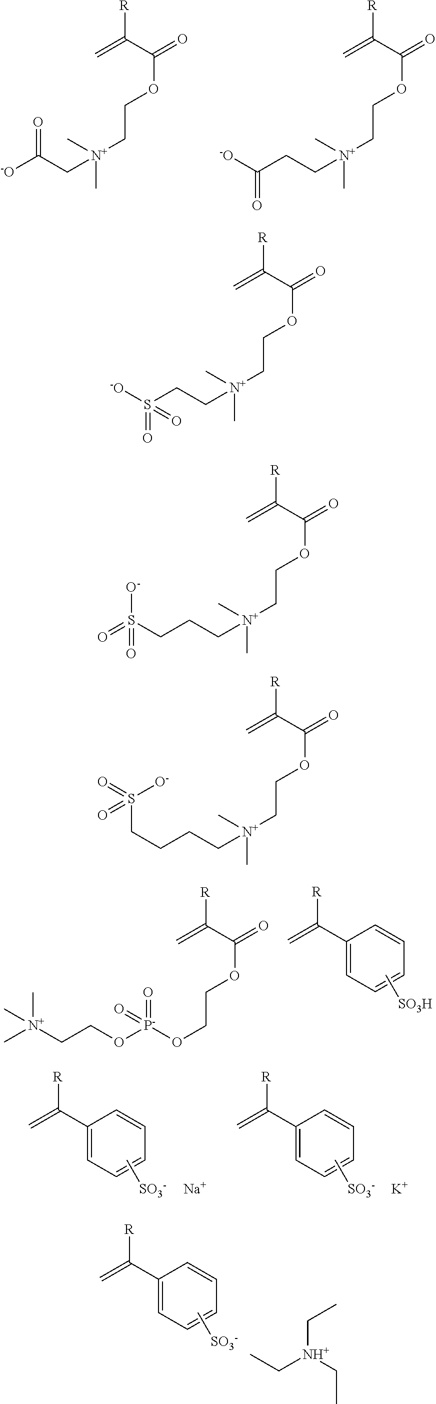

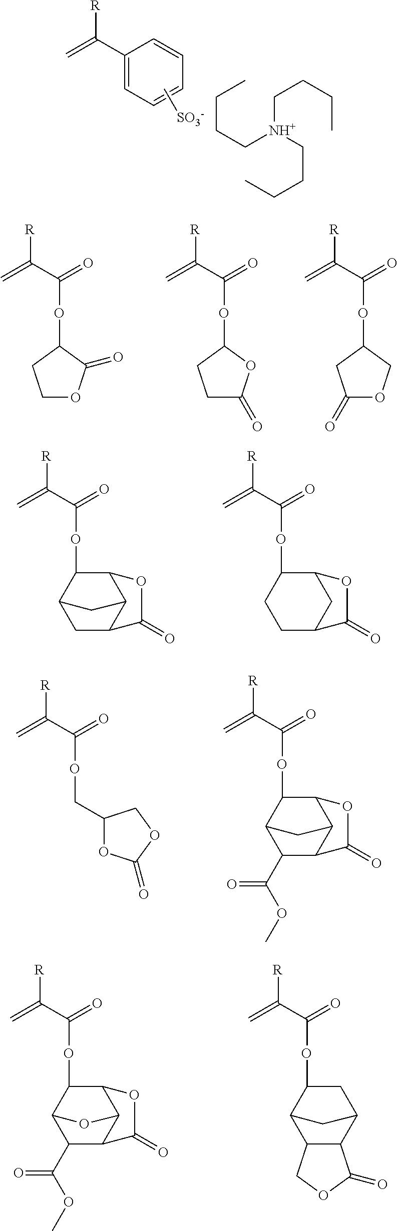

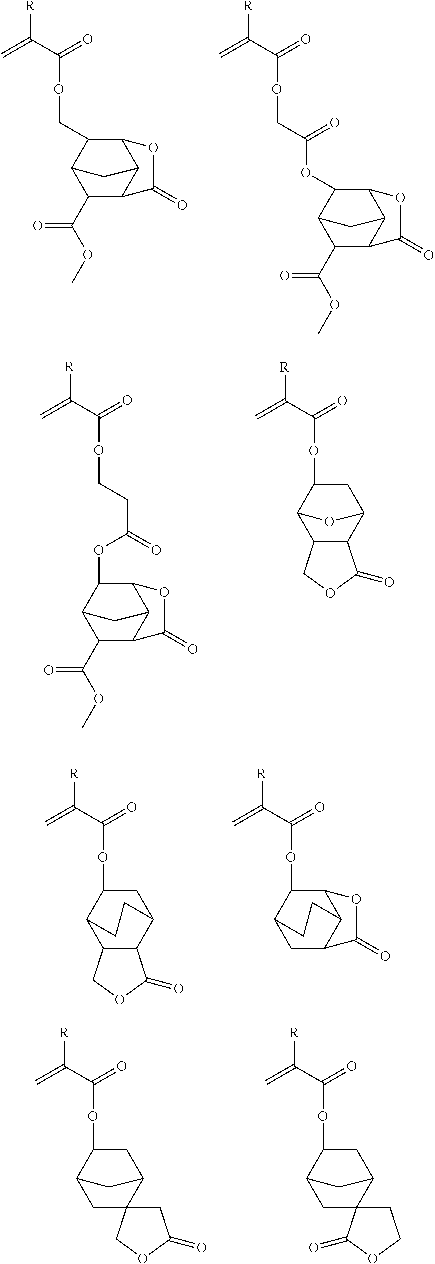

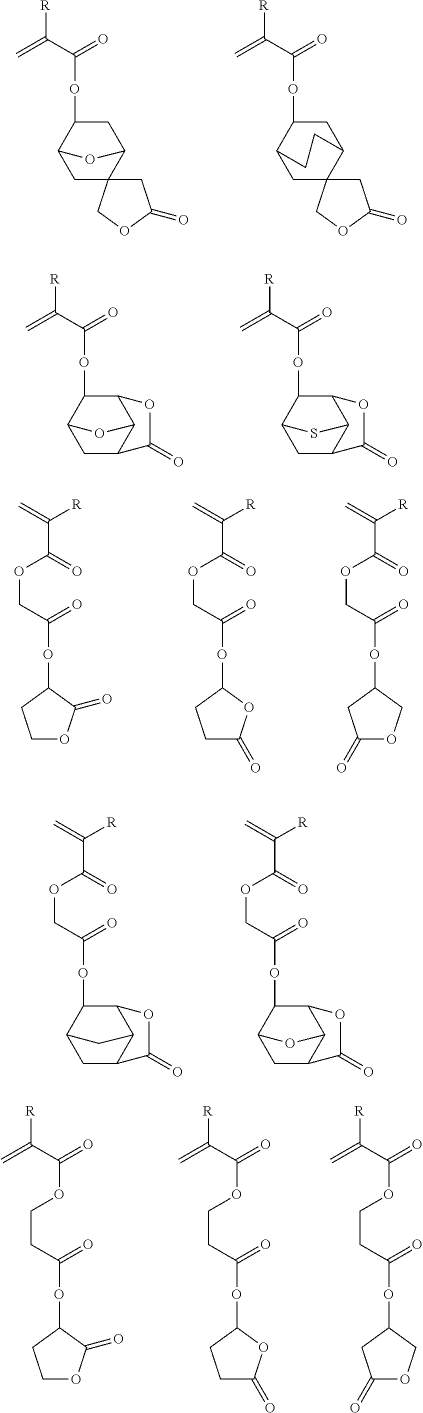

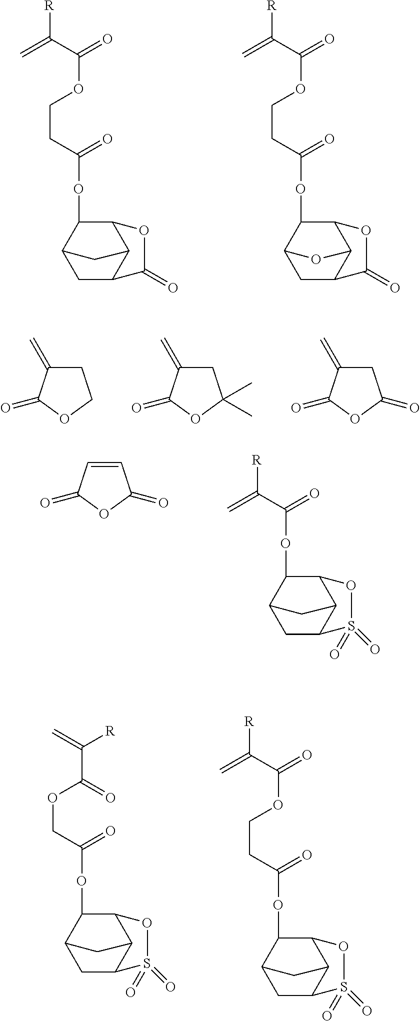

In the formulae, R represents a hydrogen atom or a methyl group.

### (Repeating Unit-d)

Ionic polymer components which are materials to form the ionic polymer containing layer used for the inventive bio-electrode can have a repeating unit-d having fluorine, in addition to the repeating units selected from the above repeating units-A1 to -A7, -b and -c.

The repeating unit-d having fluorine can be obtained from the monomers specifically exemplified below.

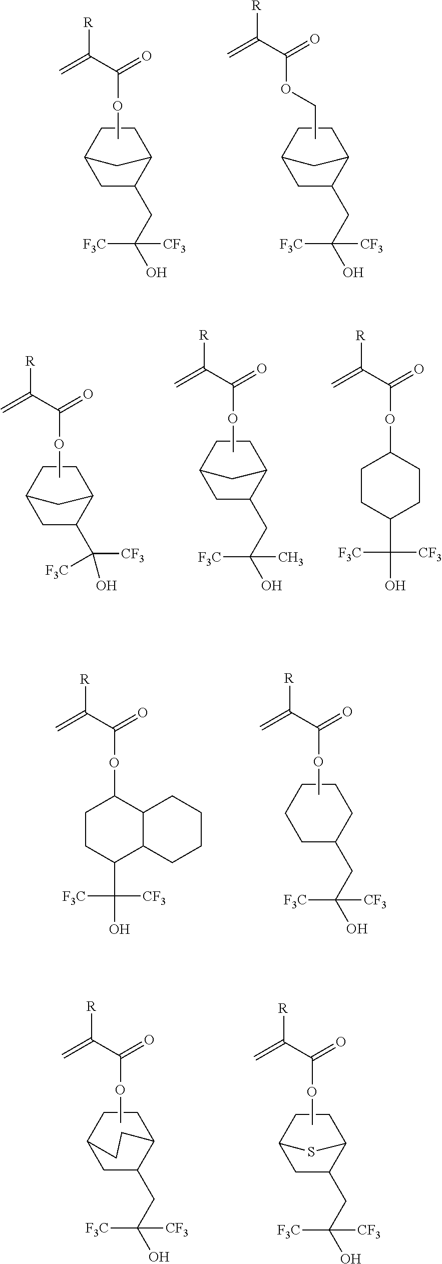

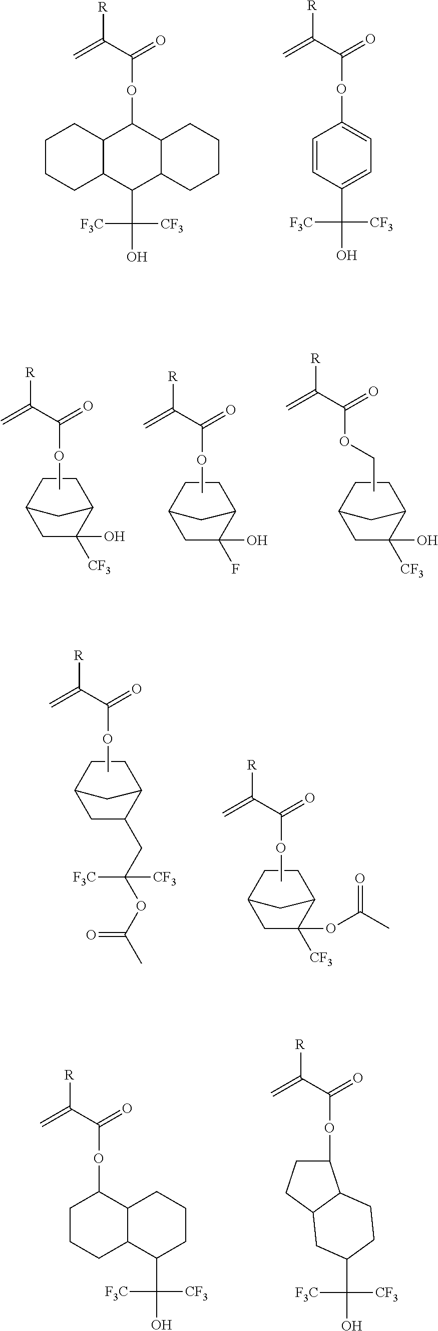

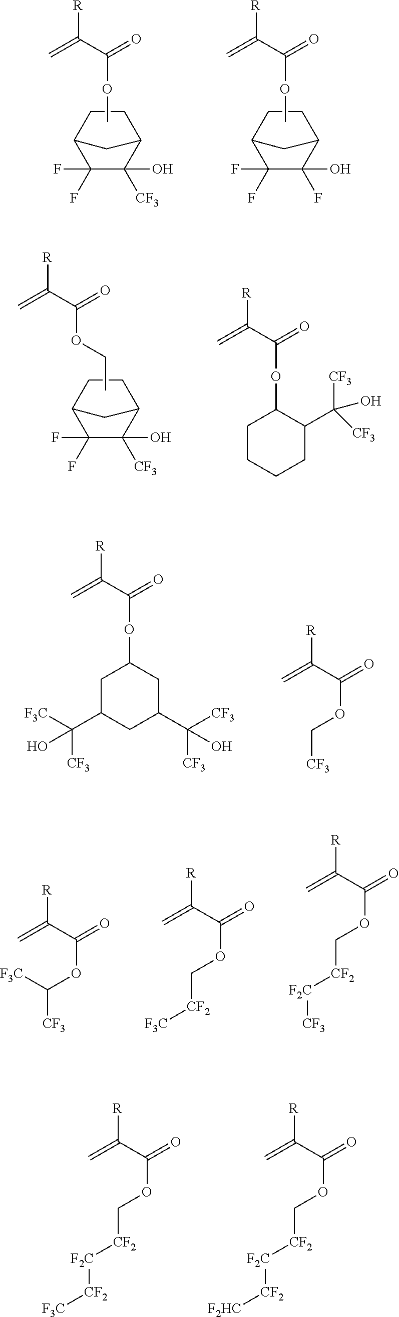

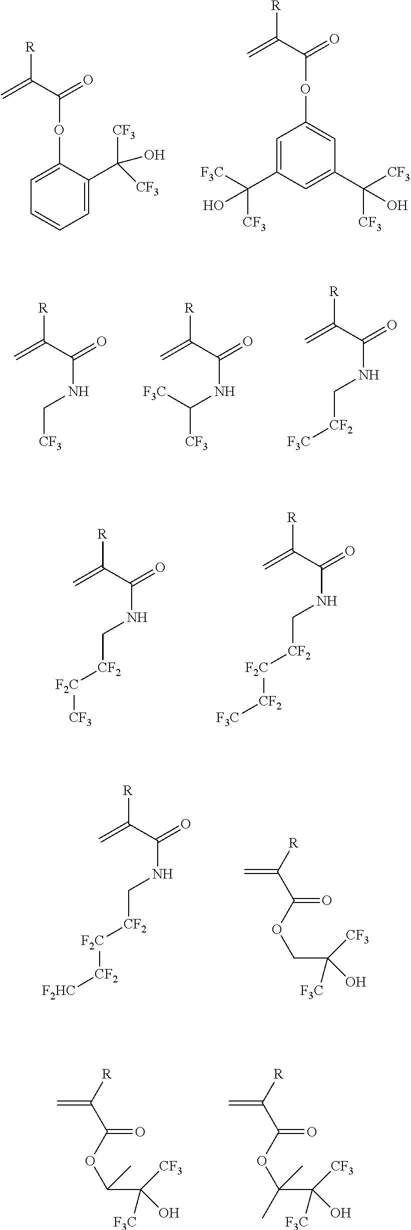

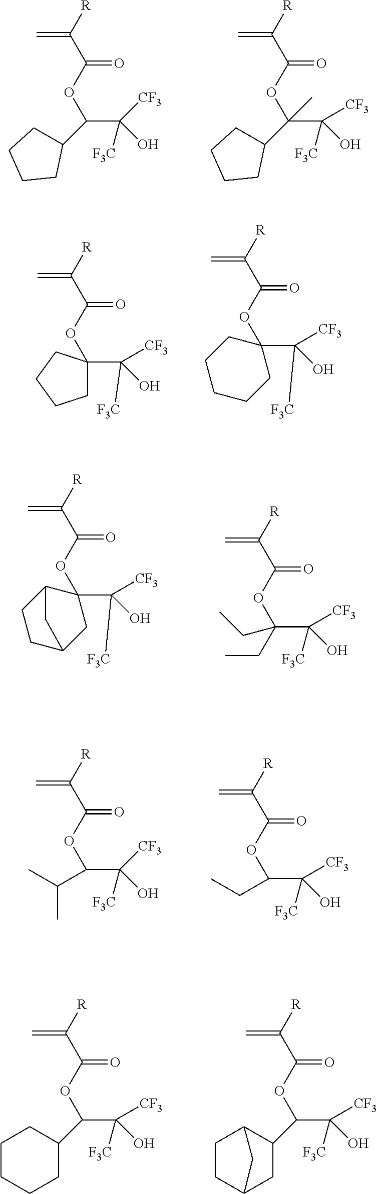

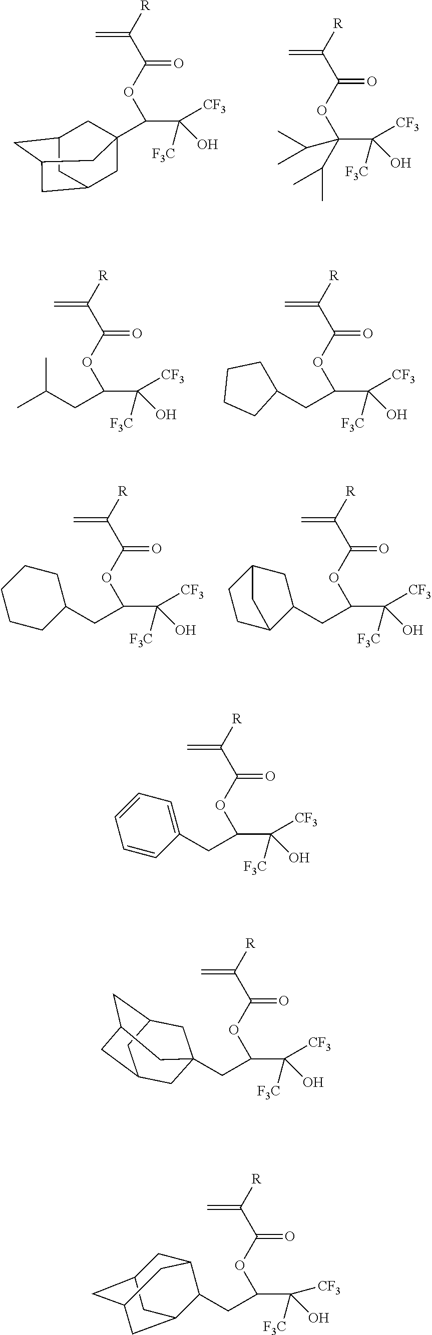

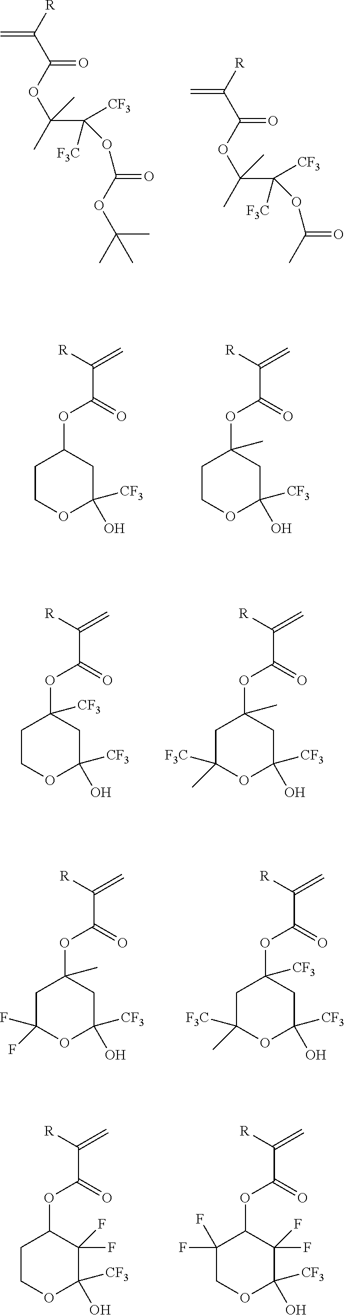

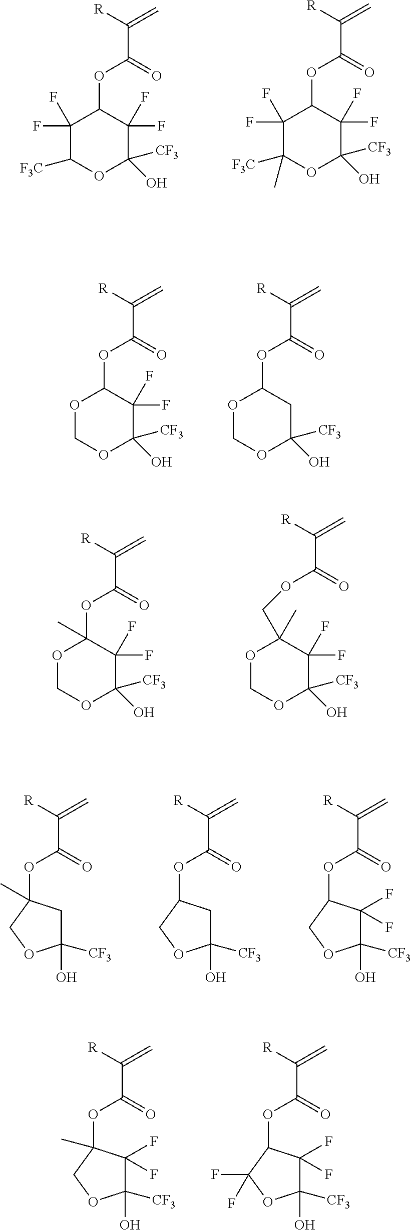

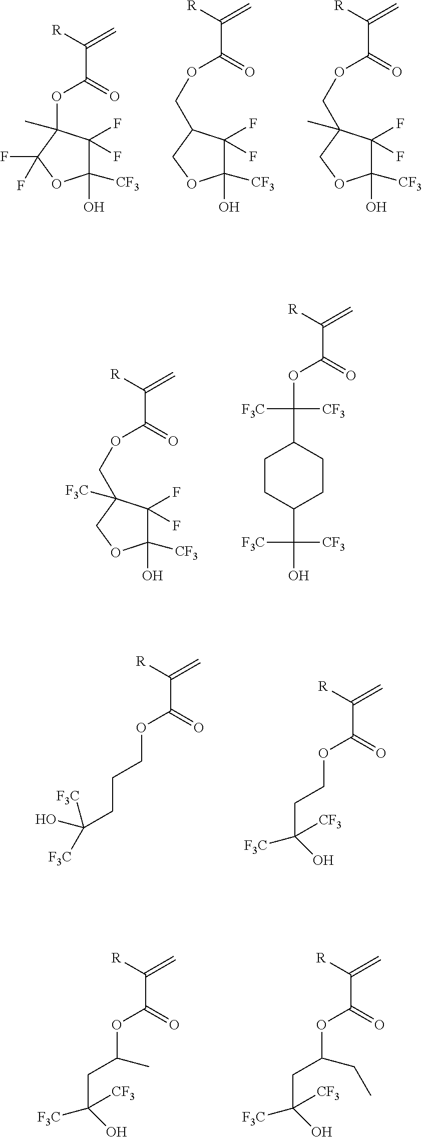

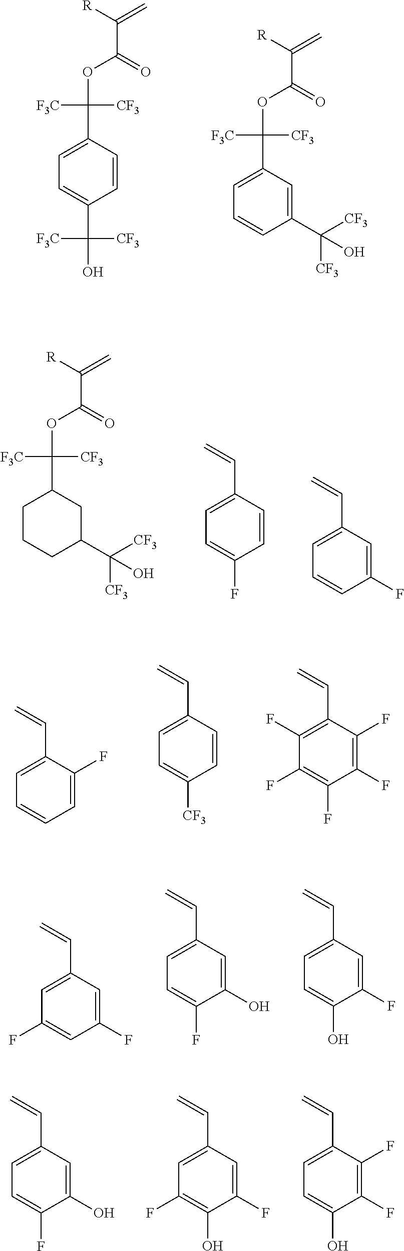

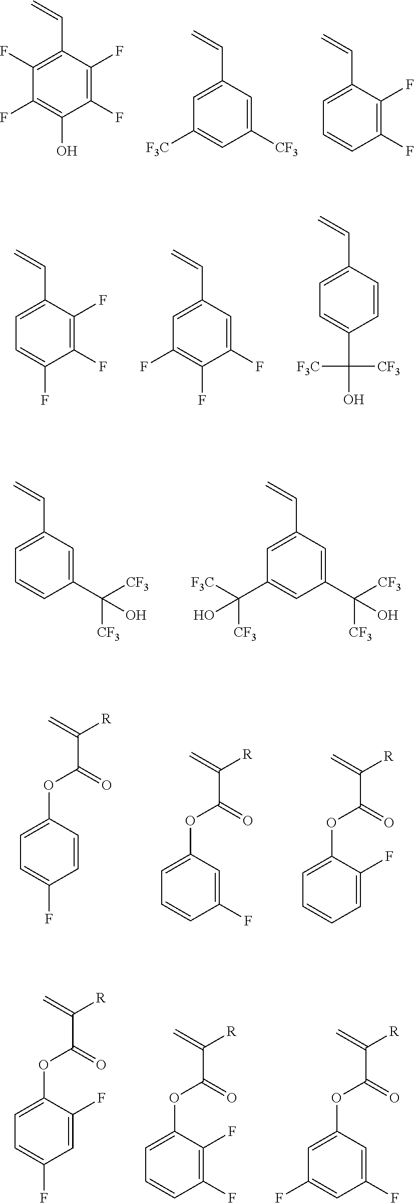

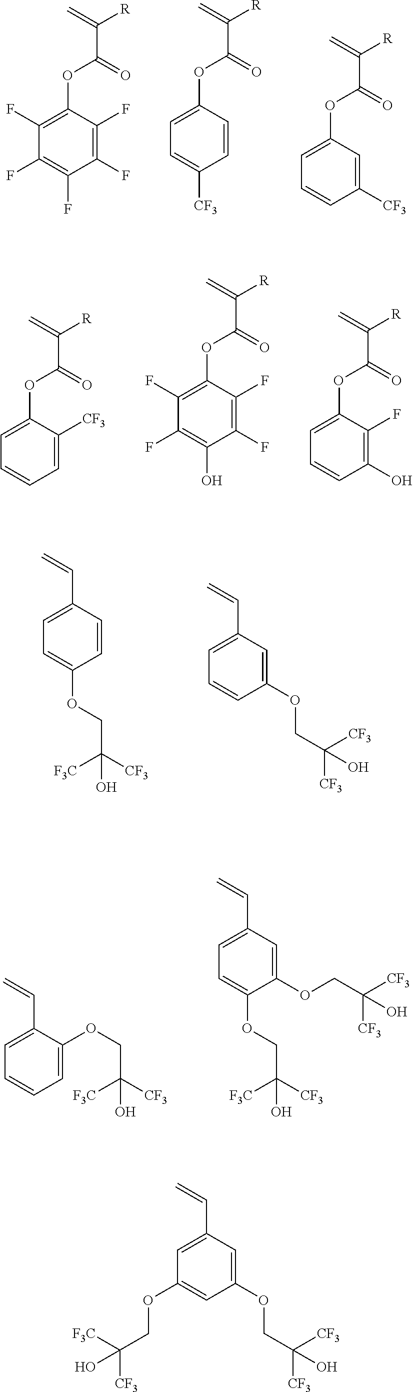

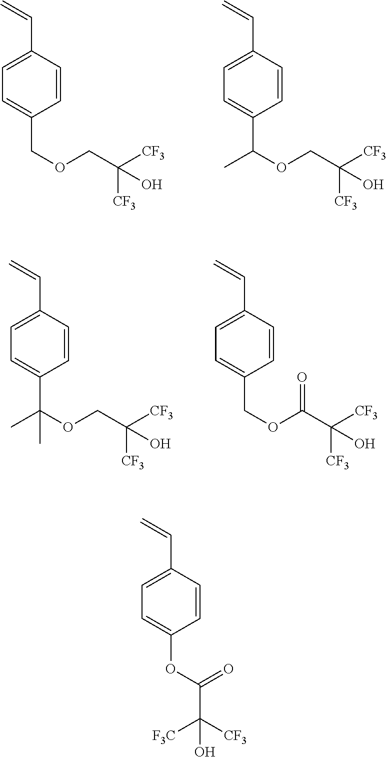

In the formulae, R represents a hydrogen atom or a methyl group.

### (Repeating Unit-e)

Ionic polymer components which are materials to form the ionic polymer containing layer used for the inventive bio-electrode can have a repeating unit-e having a nitro group, in addition to the repeating units selected from the above repeating units-A1 to -A7, -b, -c, and -d.

The repeating unit-e having a nitro group can be obtained from the monomers specifically exemplified below.

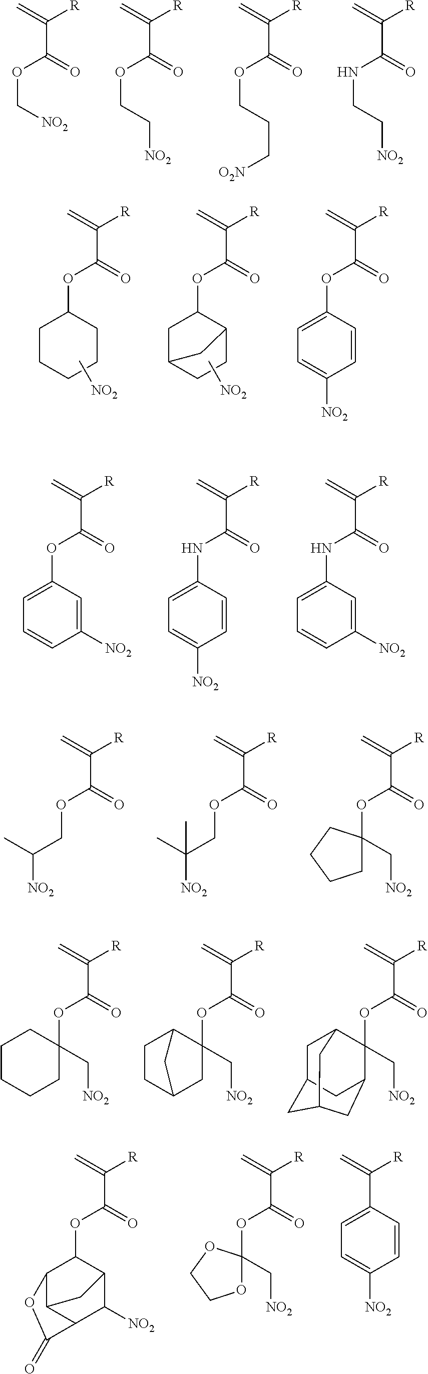

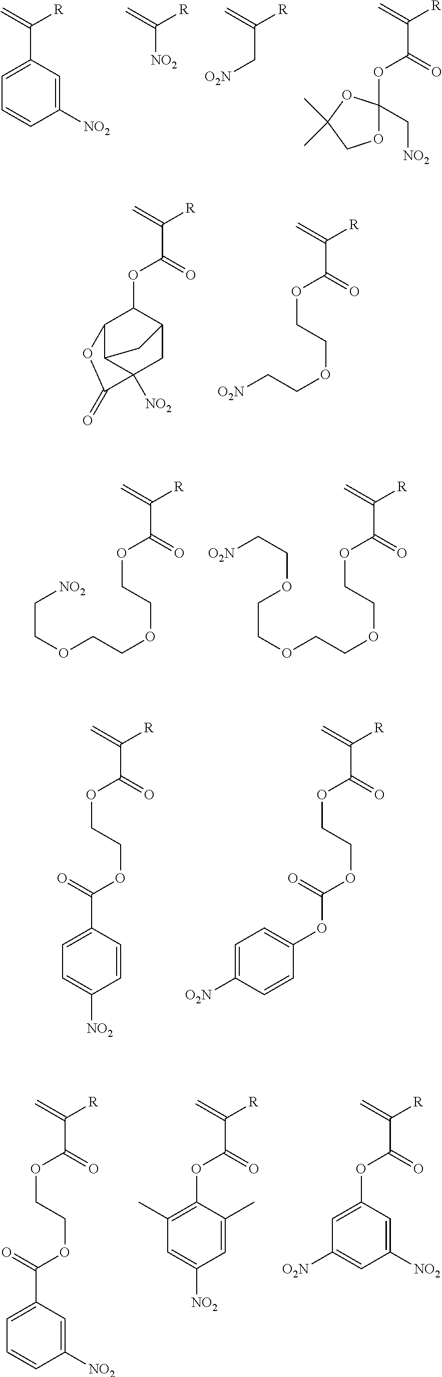

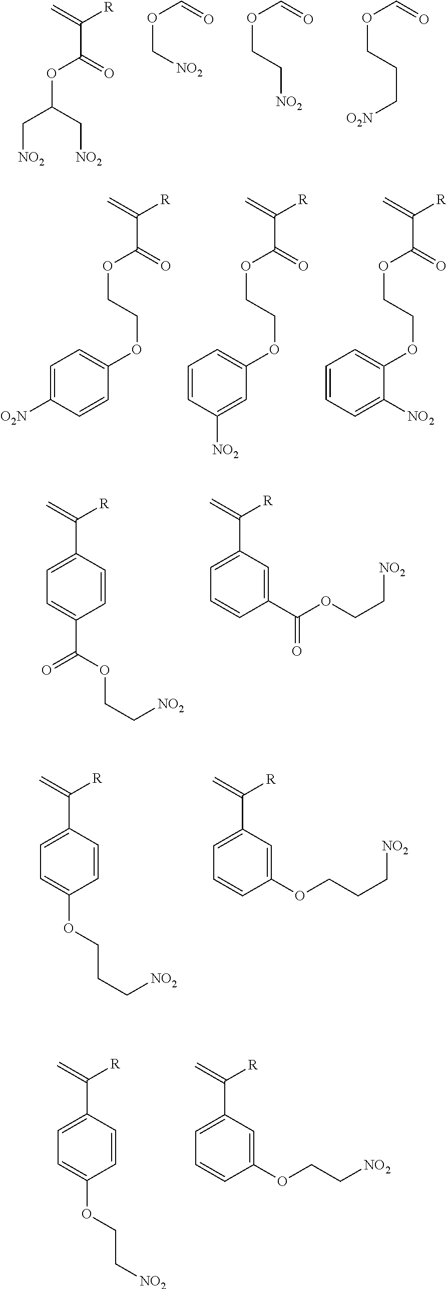

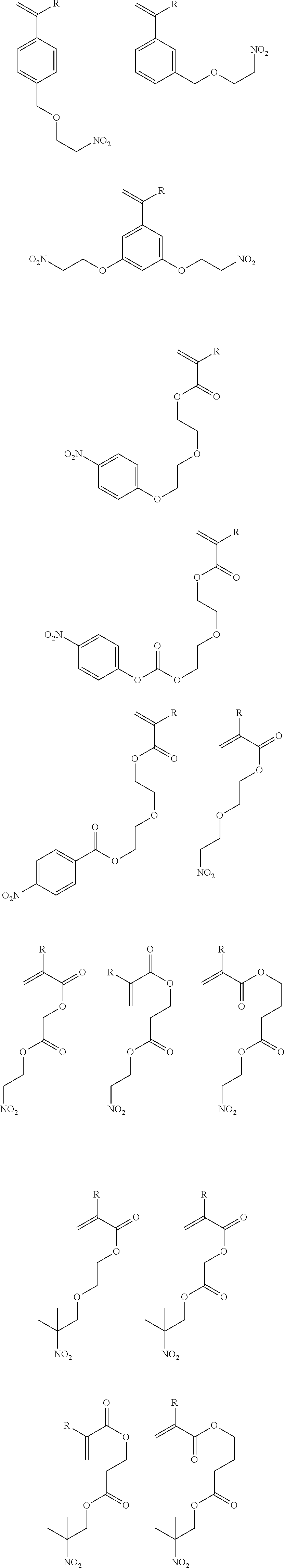

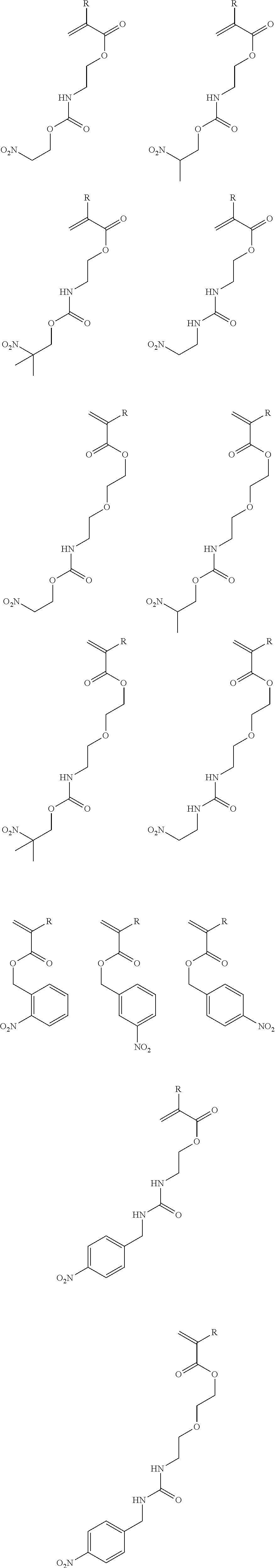

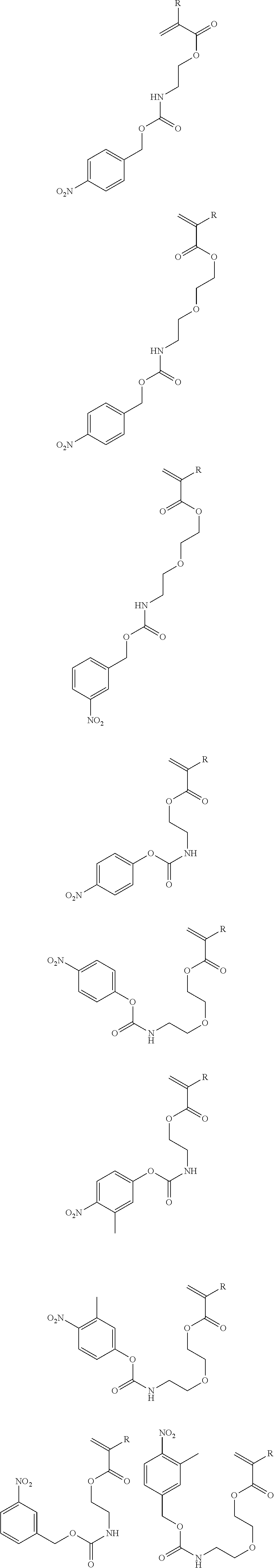

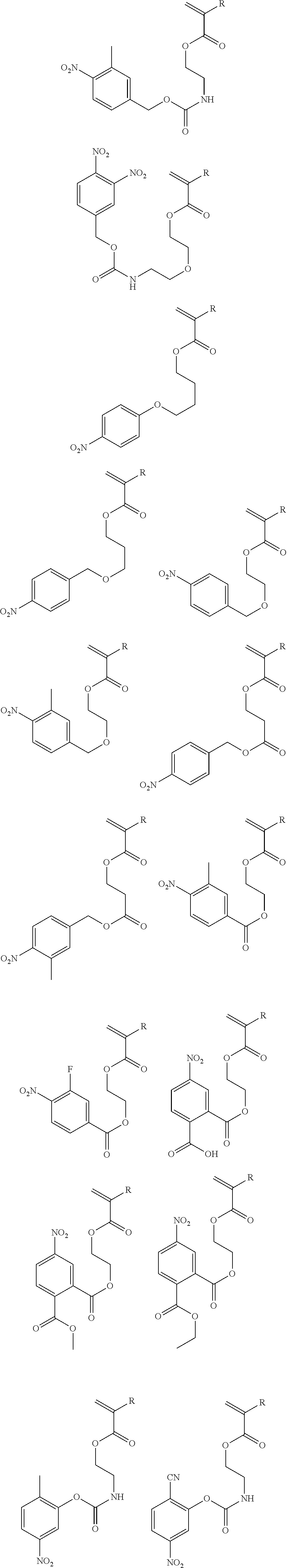

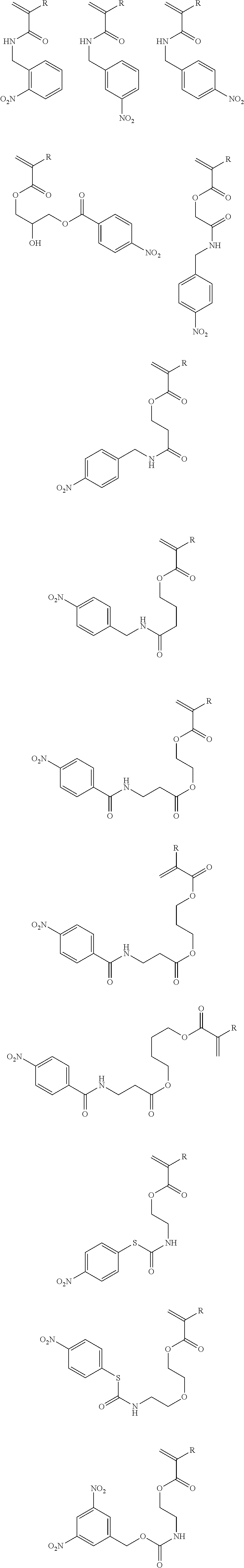

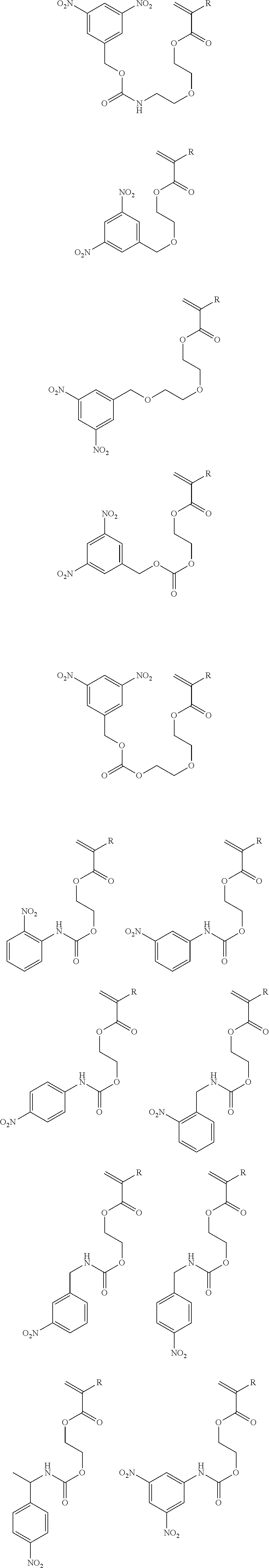

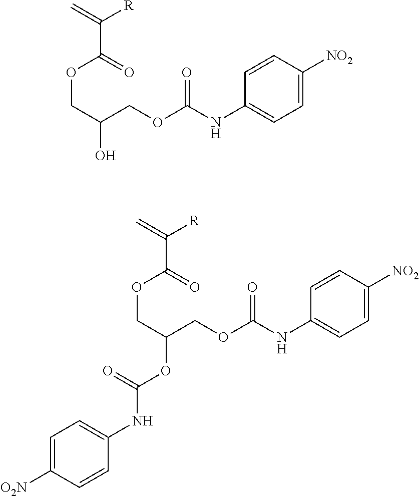

In the formulae, R represents a hydrogen atom or a methyl group.

### (Repeating Unit-f)

Ionic polymer components which are materials to form the ionic polymer containing layer used for the inventive bio-electrode can have a repeating unit-f having a cyano group, in addition to the repeating units selected from the above repeating units-A1 to -A7, -b, -c, -d, and -e.

The repeating unit-f having a cyano group can be obtained from the monomers specifically exemplified below.

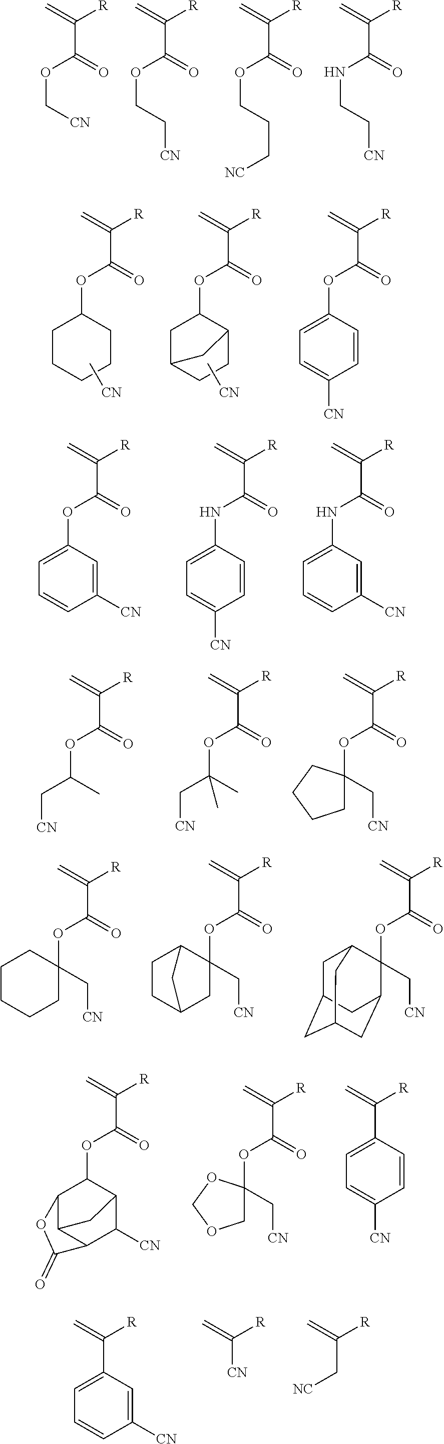

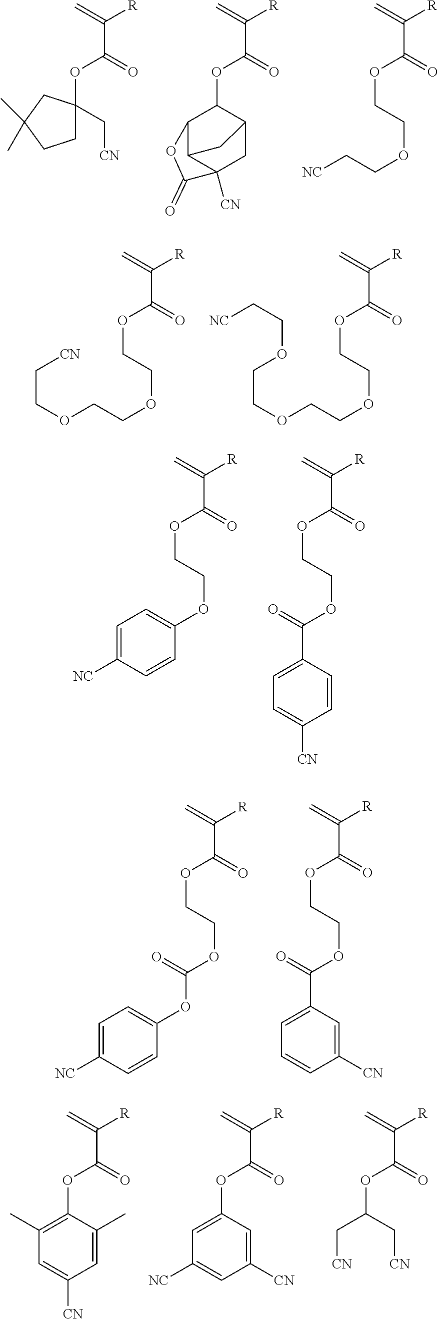

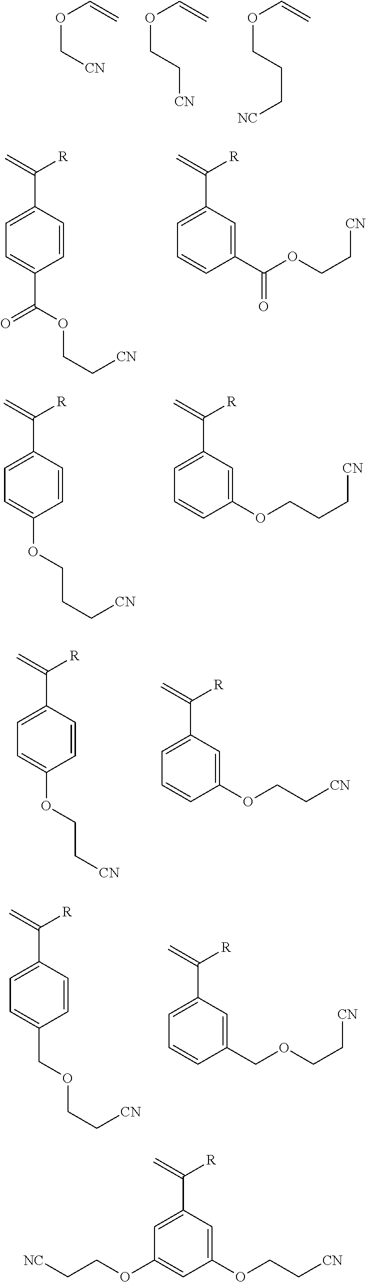

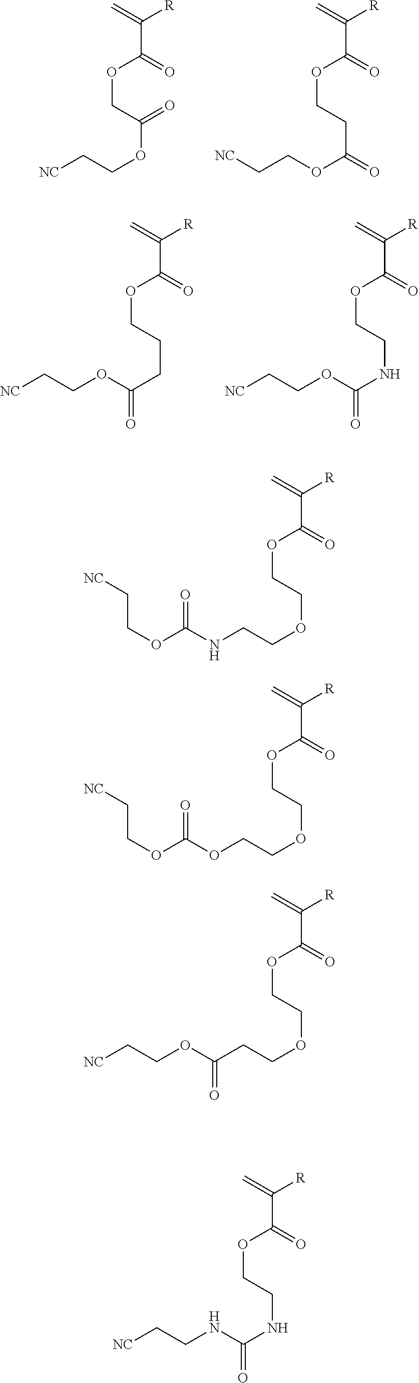

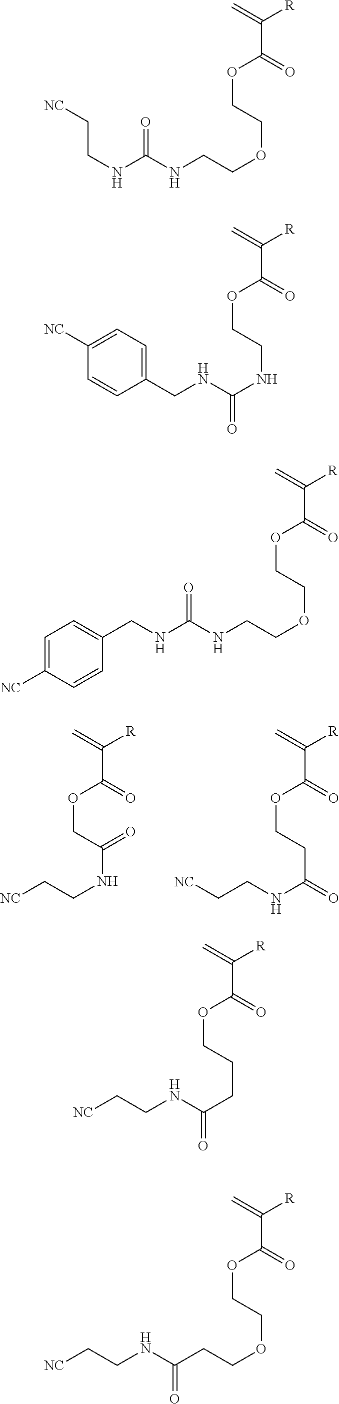

In the formulae, R represents a hydrogen atom or a methyl group.

### (Repeating Unit-g)

Ionic polymer components which are materials to form the ionic polymer containing layer used for the inventive bio-electrode can have a repeating unit-g having an oxirane group or an oxetane group, in addition to the repeating units selected from the above repeating units-A1 to -A7, -b, -c, -d, -e and -f.

The repeating unit-g having an oxirane group or an oxetane group can be obtained from the monomers specifically exemplified below.

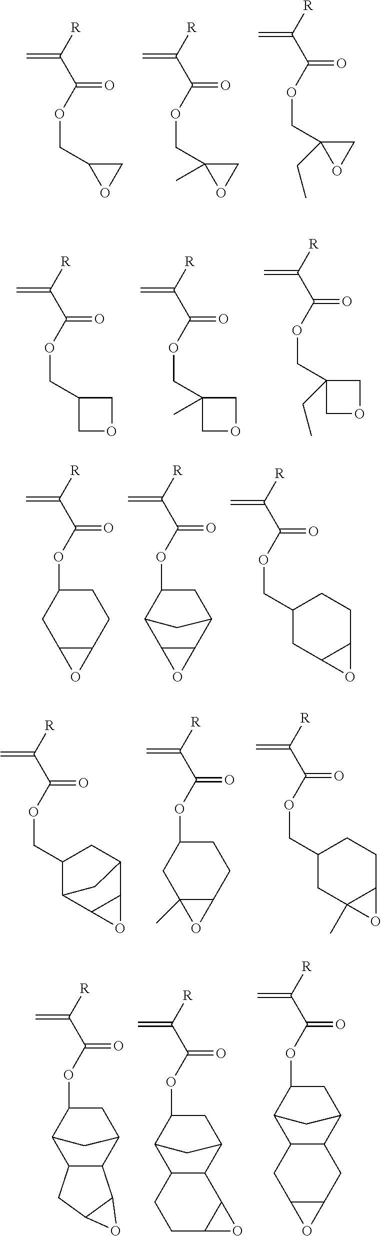

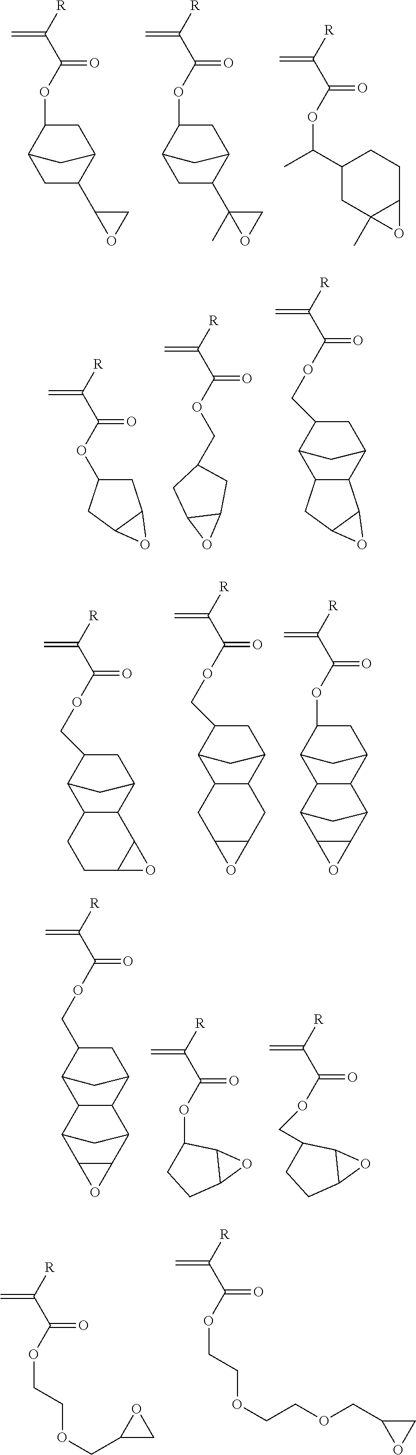

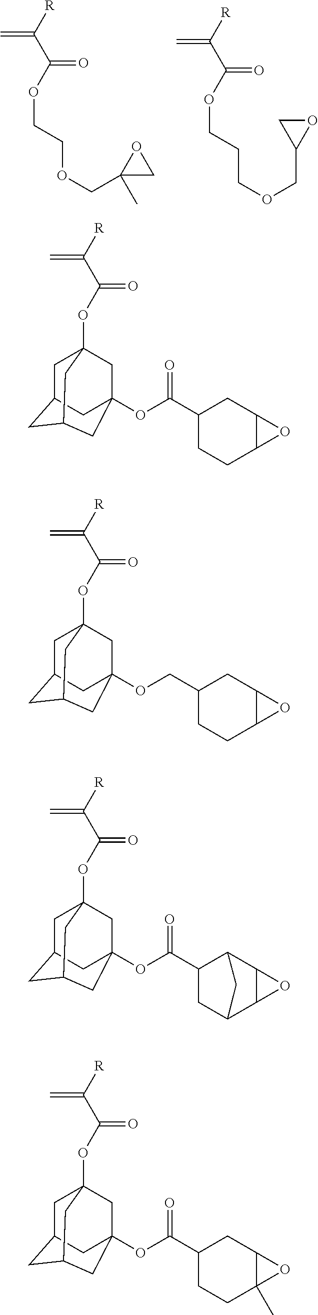

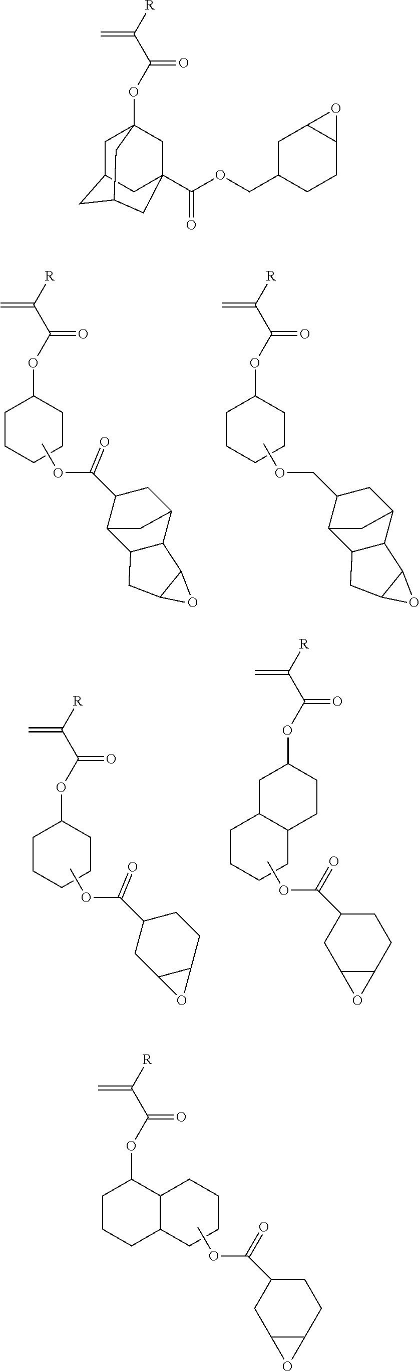

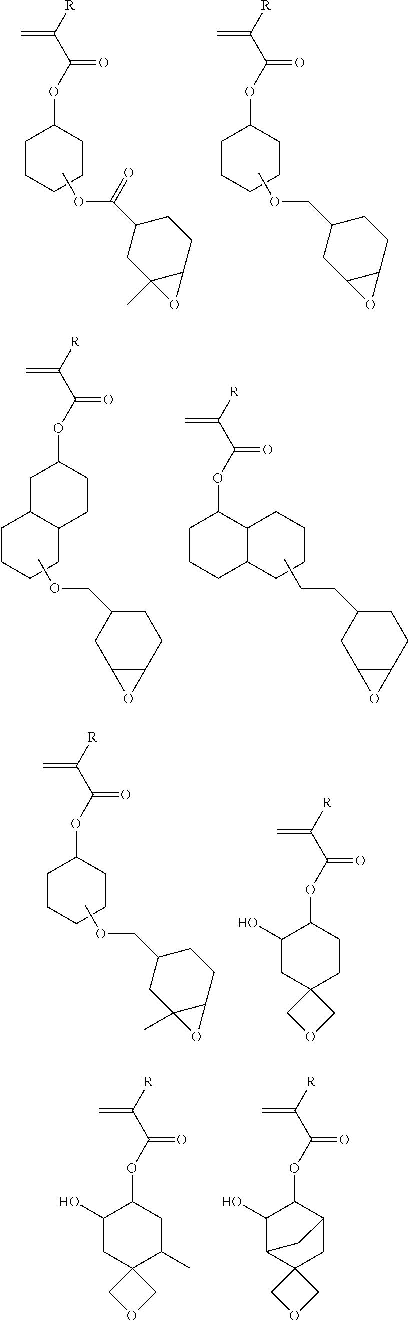

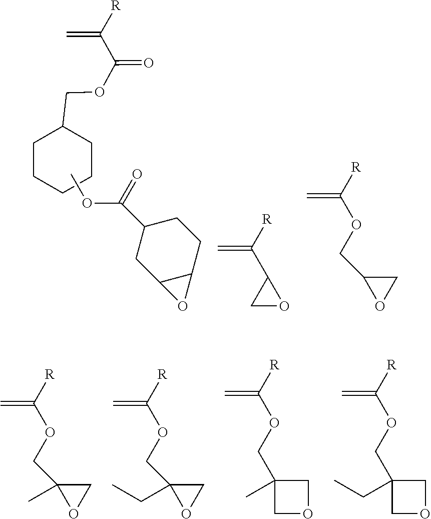

In the formulae, R represents a hydrogen atom or a methyl group.

### (Repeating Unit-h)

Ionic polymer components which are materials to form the ionic polymer containing layer used for the inventive bio-electrode can have a repeating unit-h having an isocyanate group or a blocked isocyanate group, in addition to the repeating units selected from the above repeating units-A1 to -A7, -b, -c, -d, -e, -f and -g.

The repeating unit-h having an isocyanate group or a blocked isocyanate group can be obtained from the monomers specifically exemplified below.

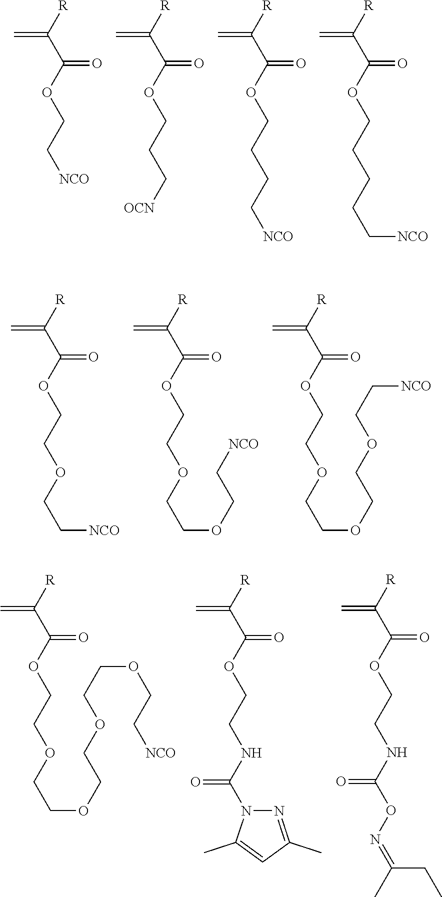

In the formulae, R represents a hydrogen atom or a methyl group.

Further, the ionic repeating unit-a which the ionic polymer has preferably includes an ammonium ion shown by the following general formula (3) as an ammonium ion for forming an ammonium salt,

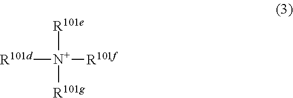

wherein, R^{101d}, R^{101e}, R^{101f}, and R^{101g} each represent a hydrogen atom, a linear, branched, or cyclic alkyl group having 1 to 15 carbon atoms, a linear, branched, or cyclic alkenyl group or alkynyl group having 2 to 12 carbon atoms, or an aromatic group having 4 to 20 carbon atoms, and optionally have one or more selected from the group consisting of an ether group, a carbonyl group, an ester group, a hydroxy group, a carboxy group, an amino group, a nitro group, a sulfonyl group, a sulfinyl group, a halogen atom, and a sulfur atom; and R^{101d} and R^{101e}, or R^{101d}, R^{101e}, and R^{101f}, are optionally bonded to each other together with a nitrogen atom bonded therewith to form a ring in which R^{101d} and R^{101e}, or R^{101d}, R^{101e}, and R^{101f}, represent an alkylene group having 3 to 10 carbon atoms, or to form a heteroaromatic ring having the nitrogen atom in the general formula (3) within the ring.

The ammonium ion shown by the above general formula (3) is specifically exemplified below.

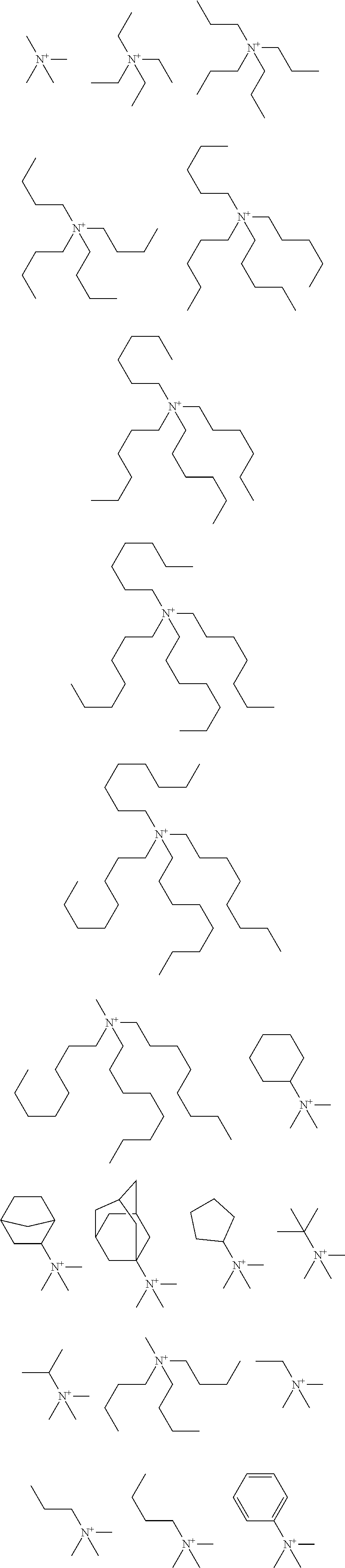

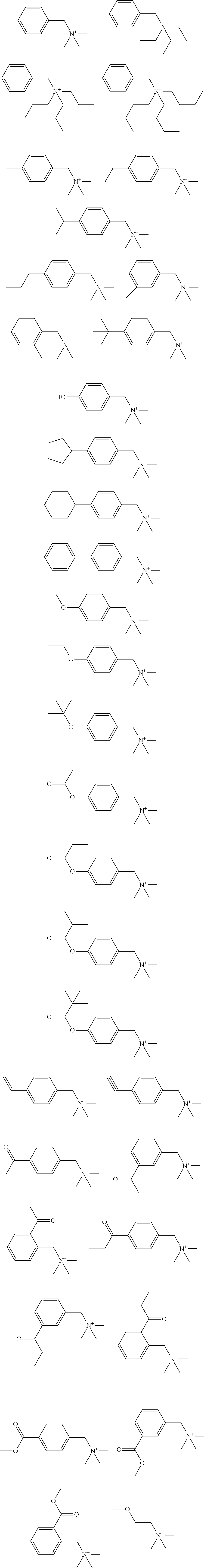

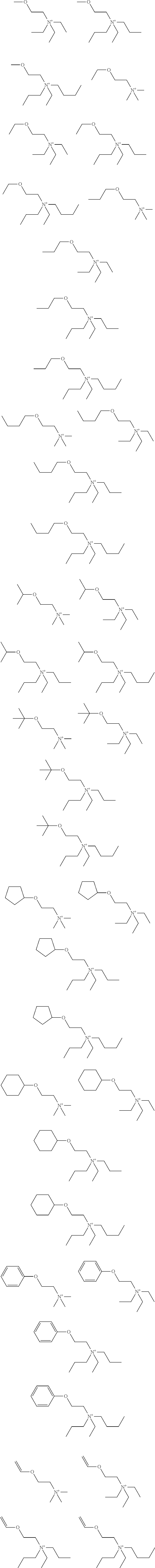

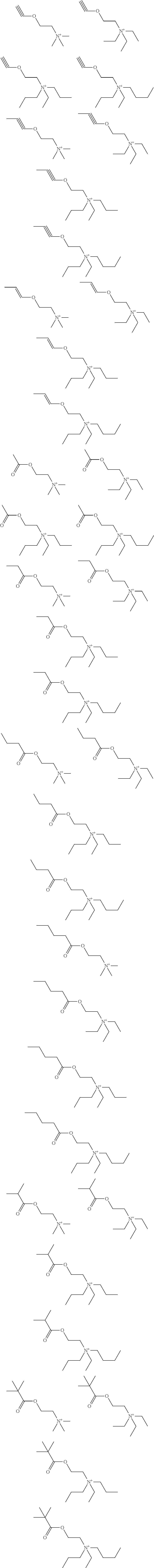

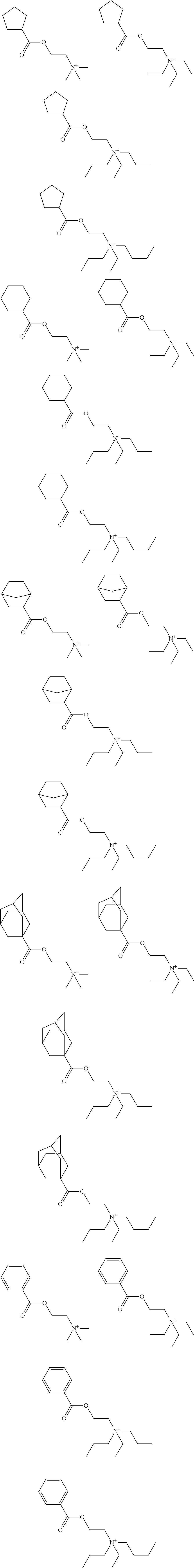

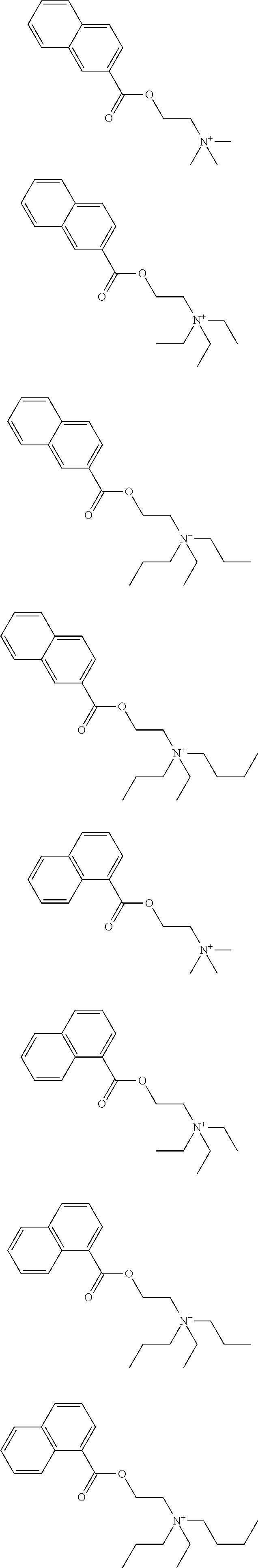

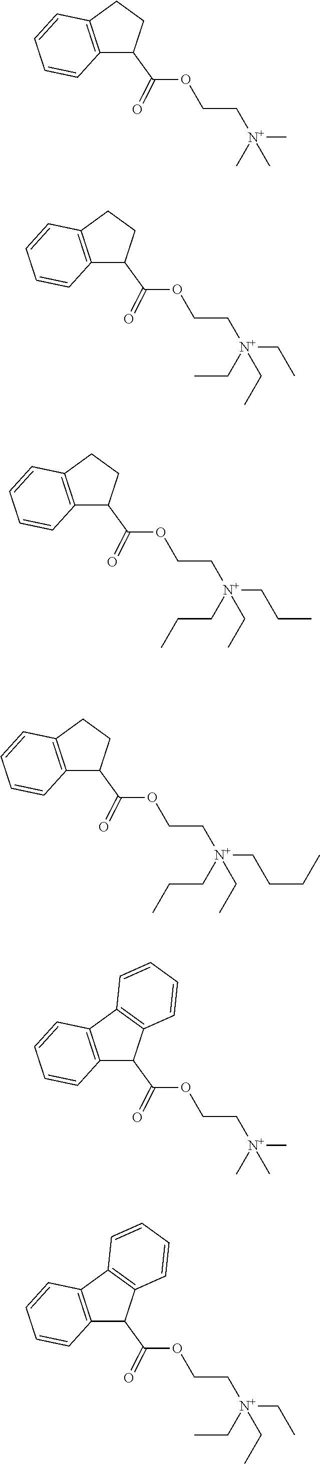

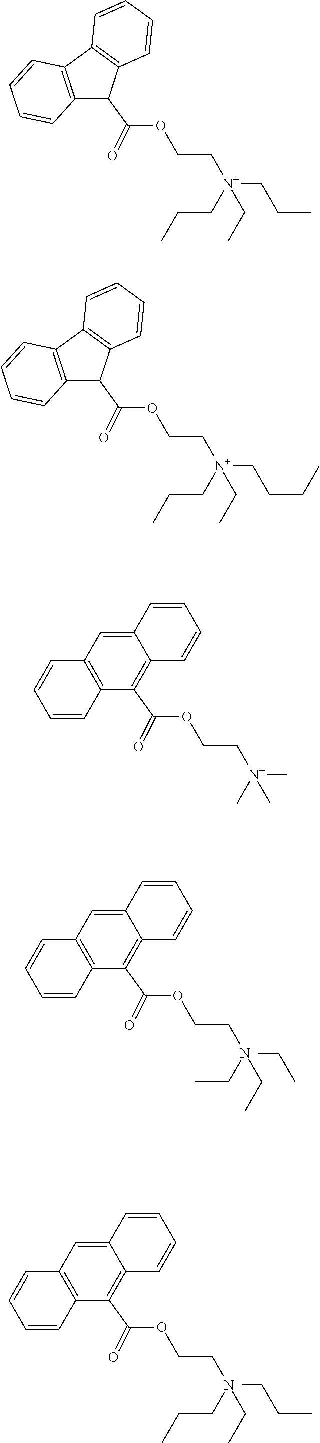

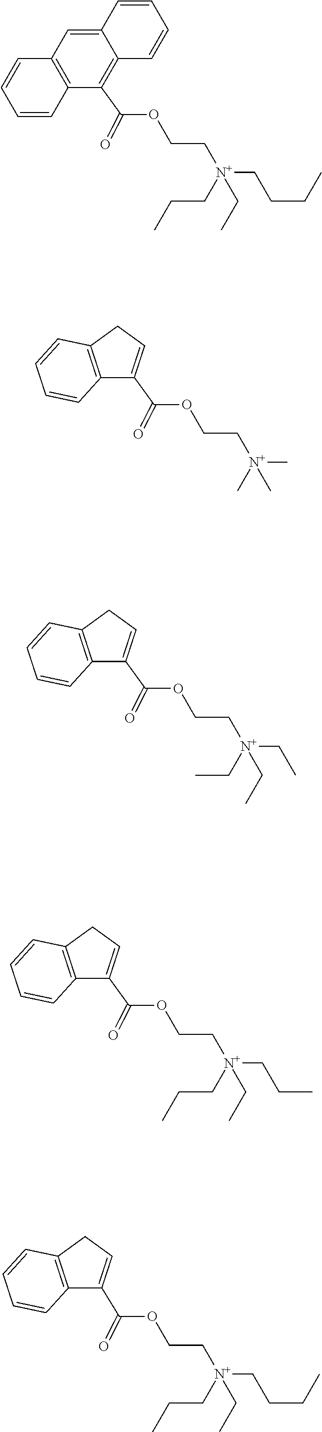

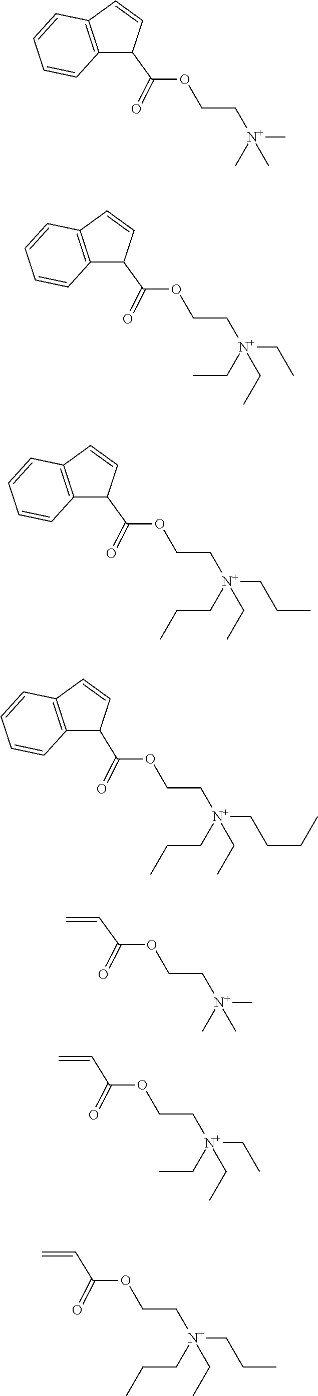

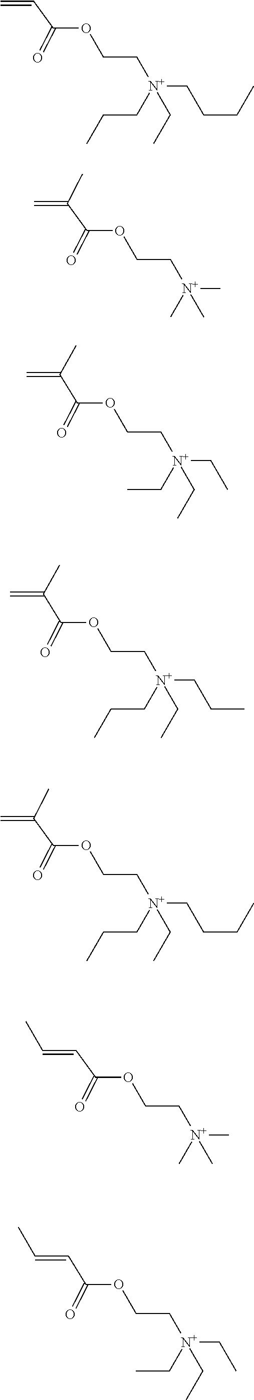

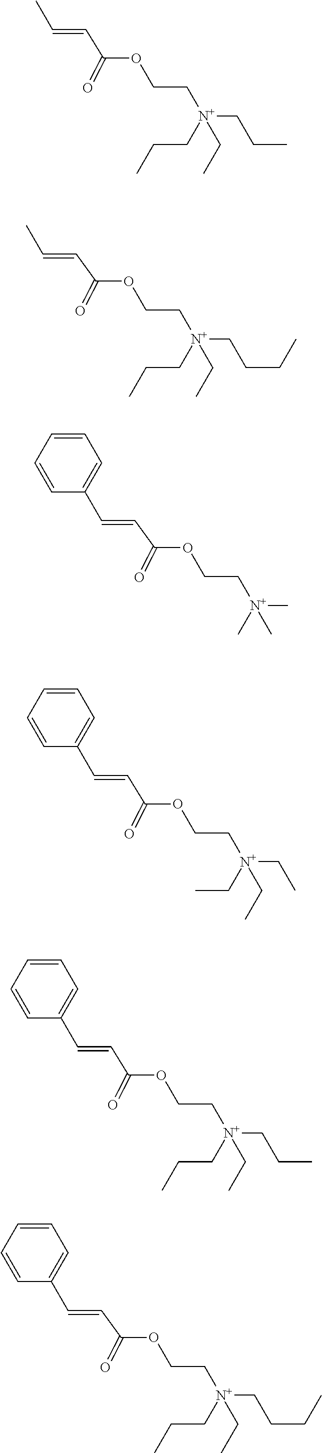

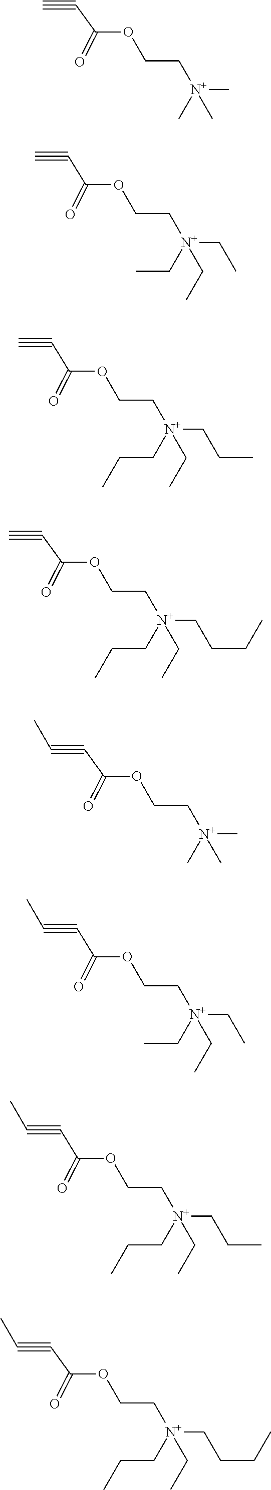

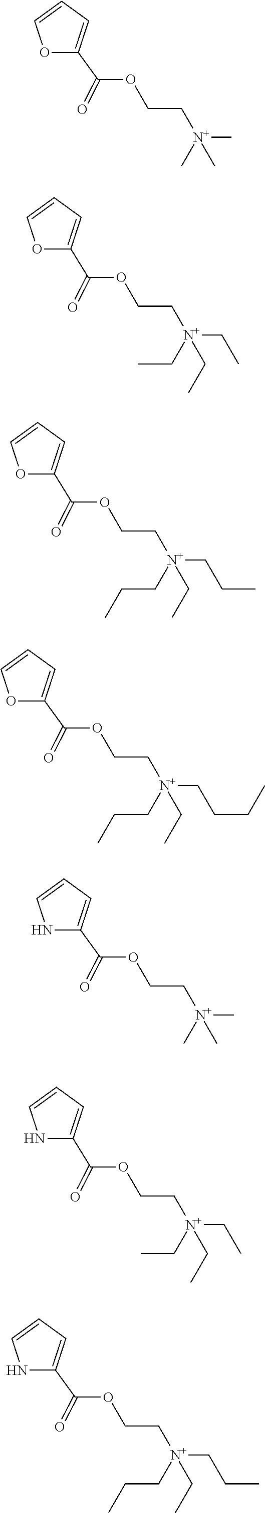

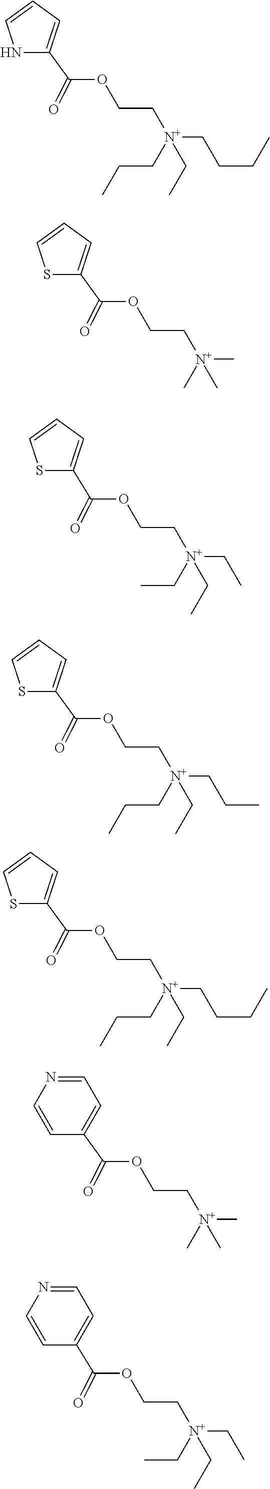

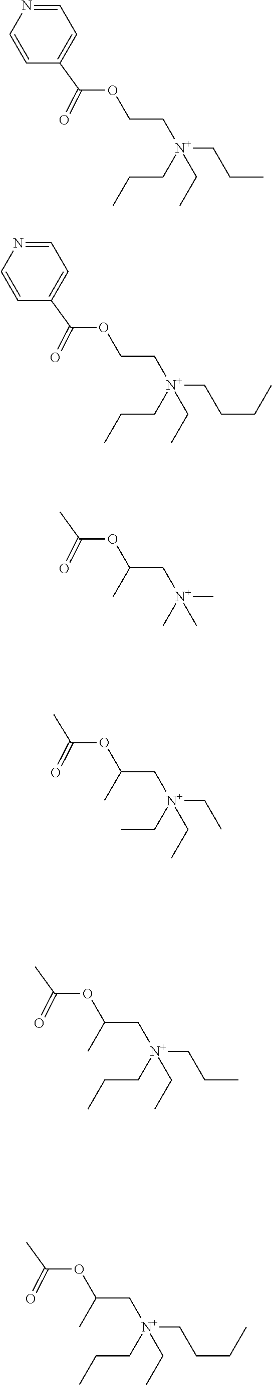

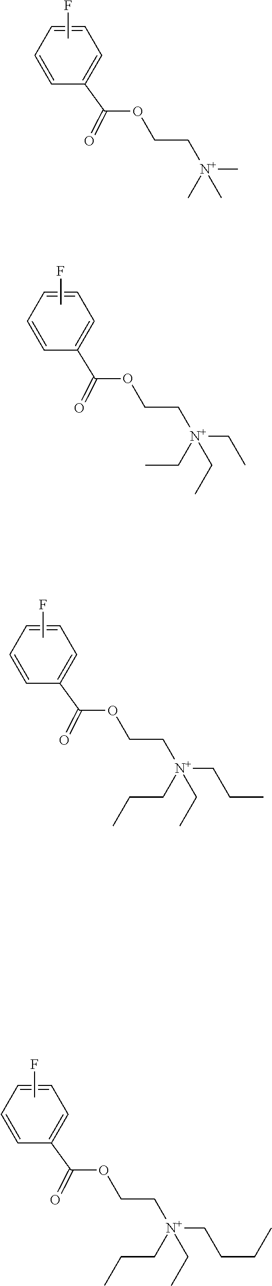

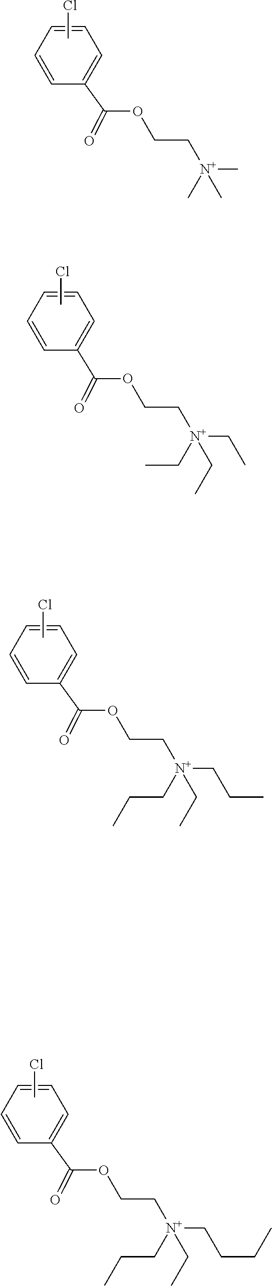

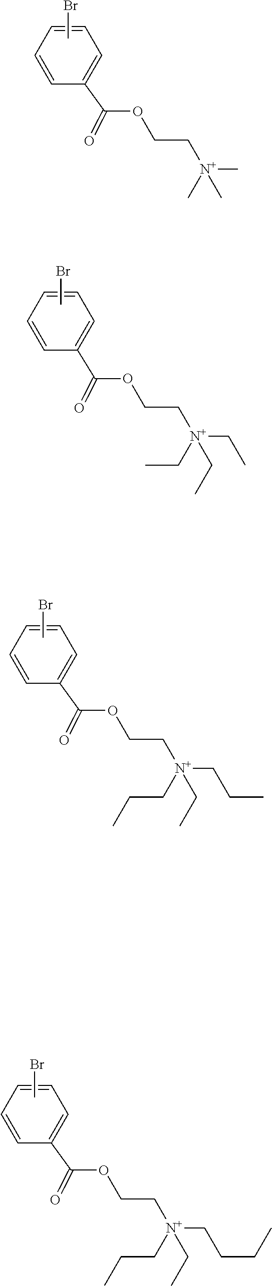

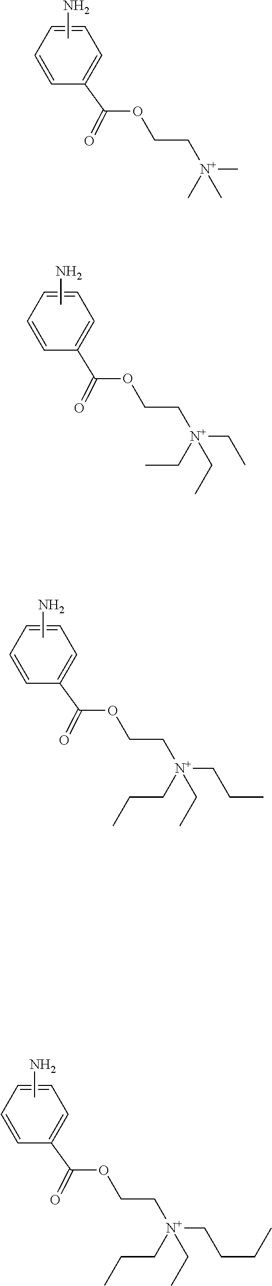

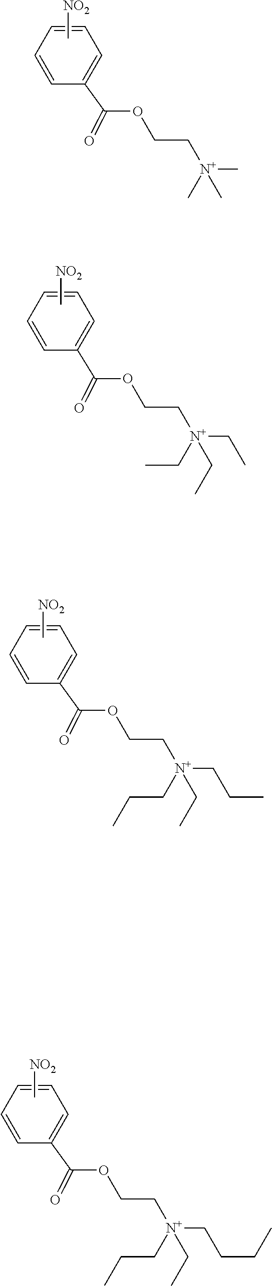

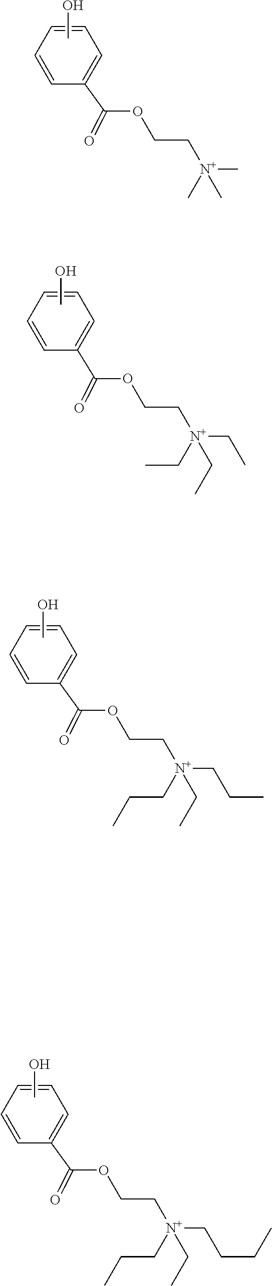

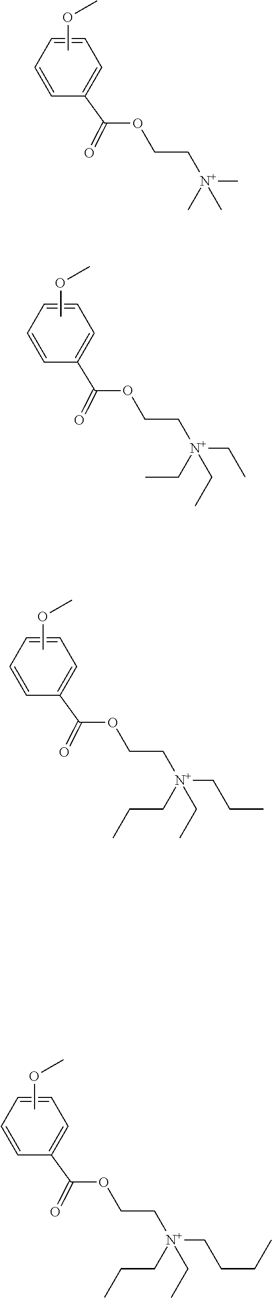

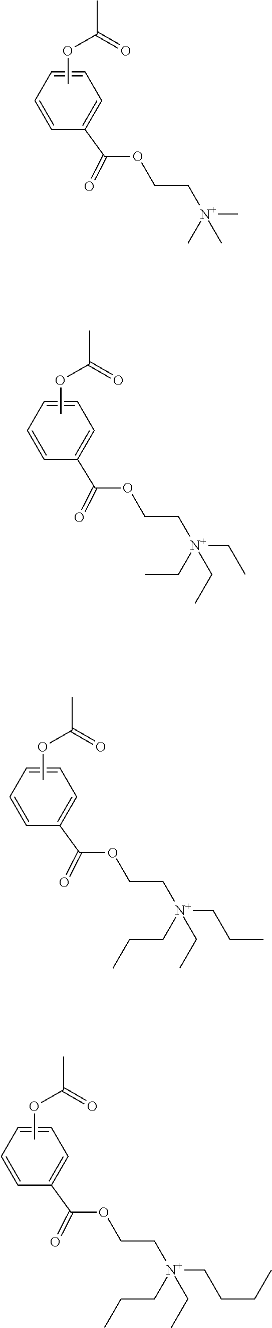

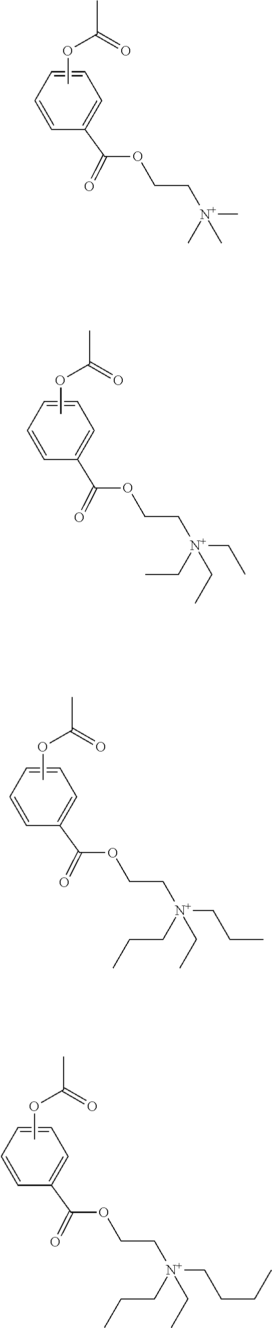

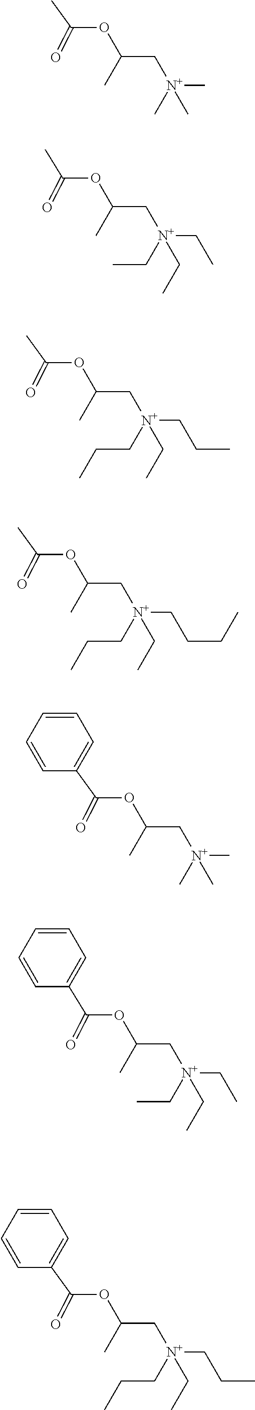

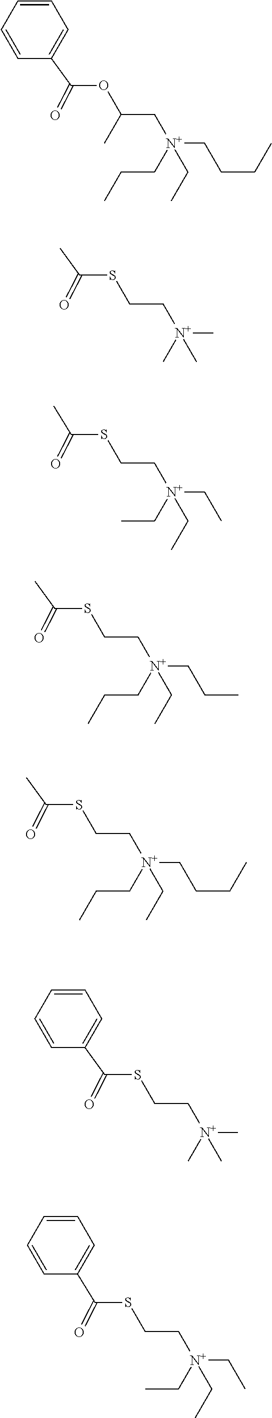

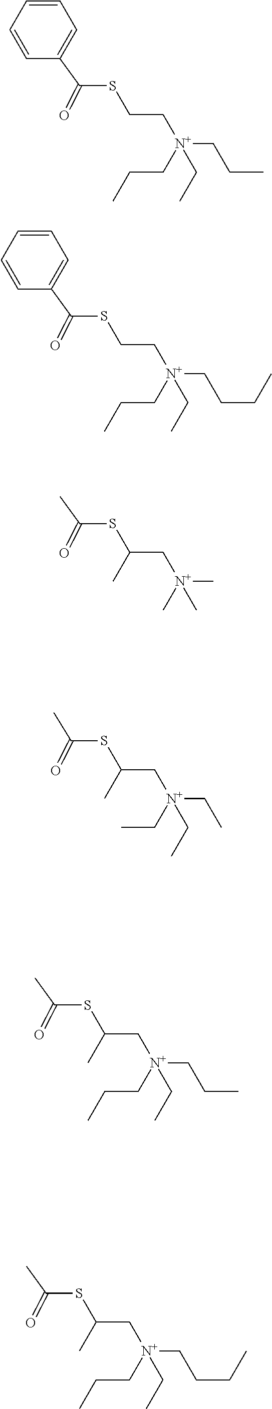

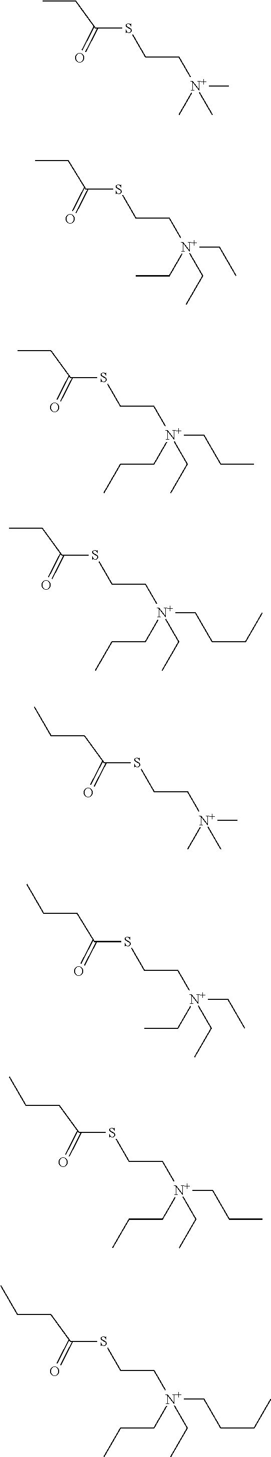

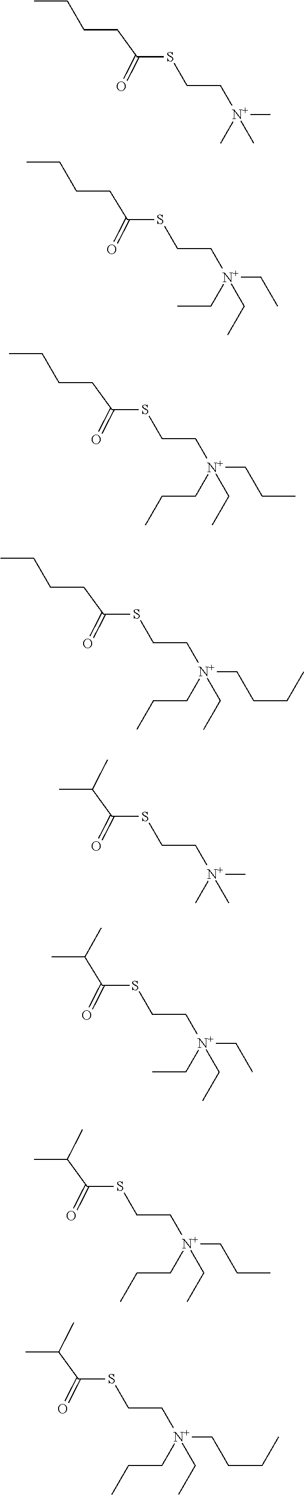

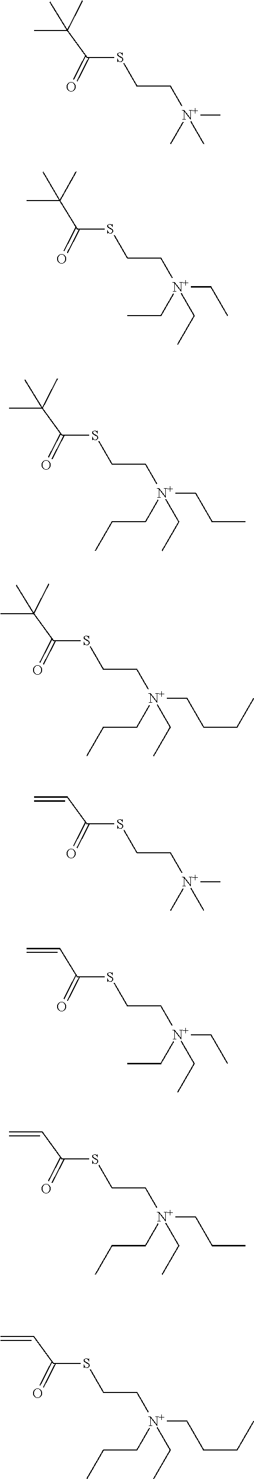

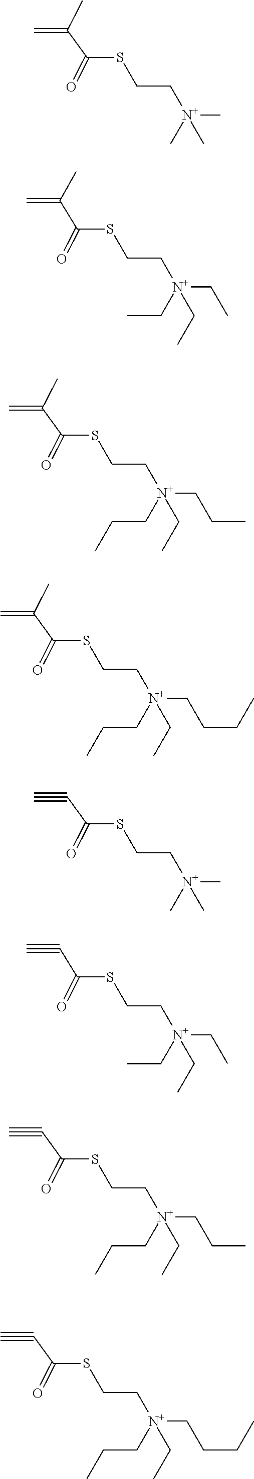

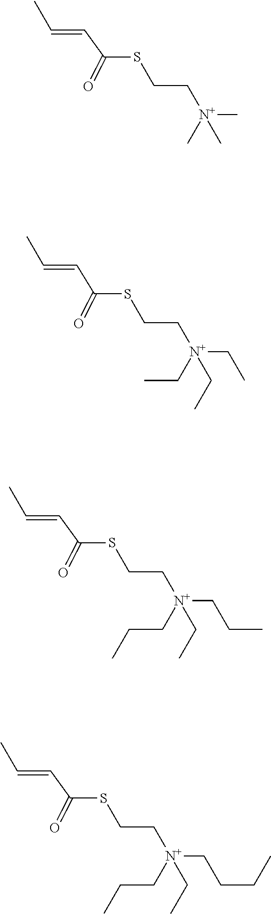

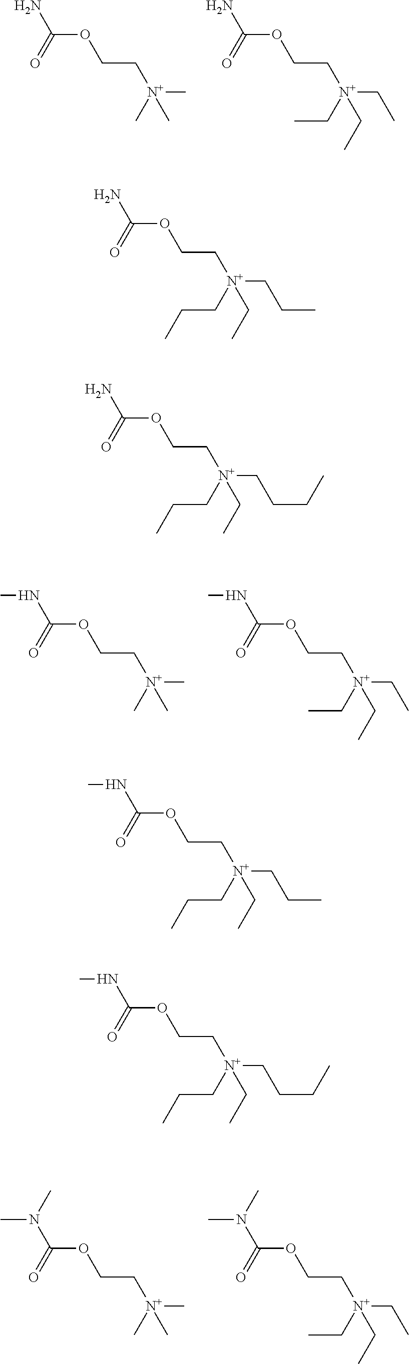

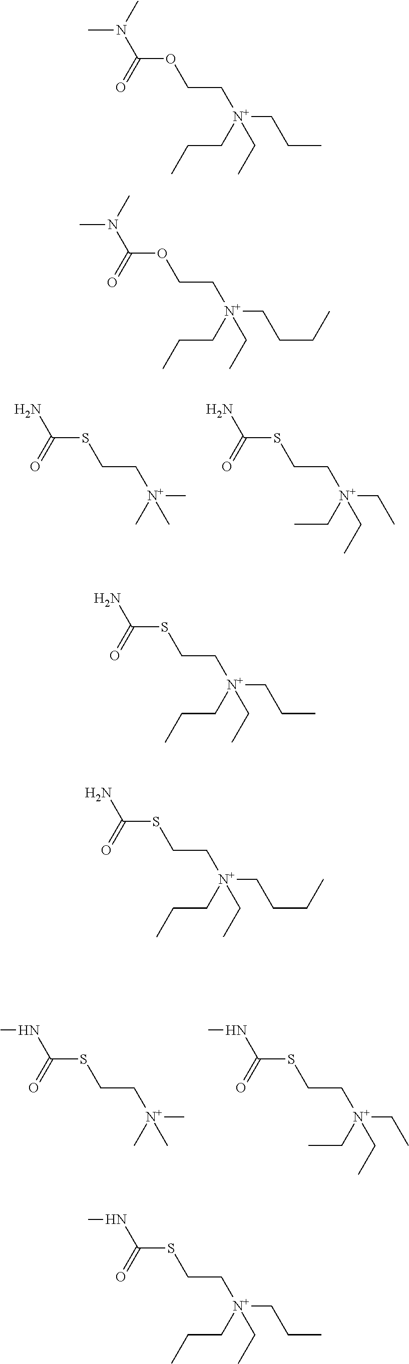

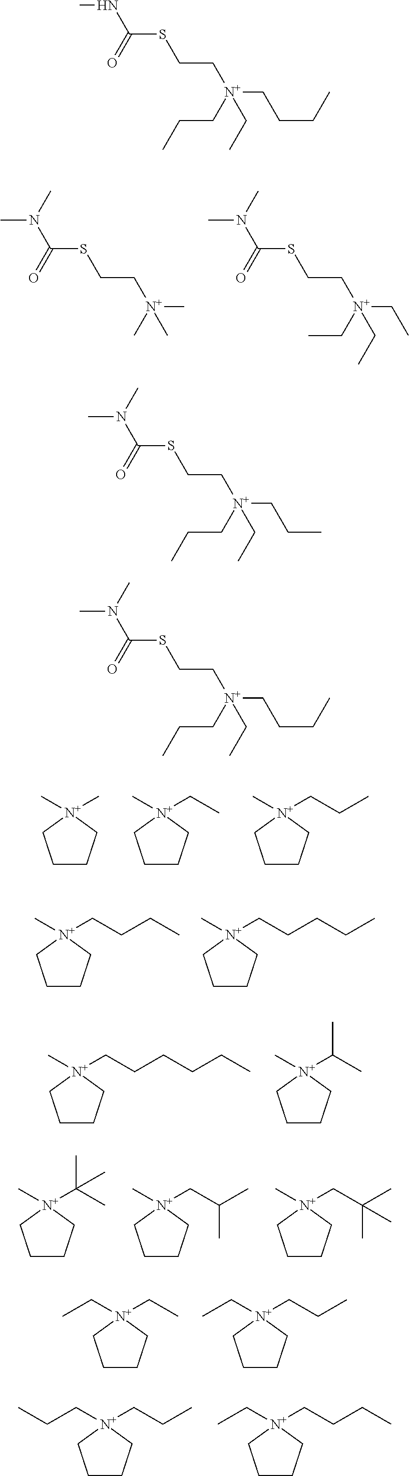

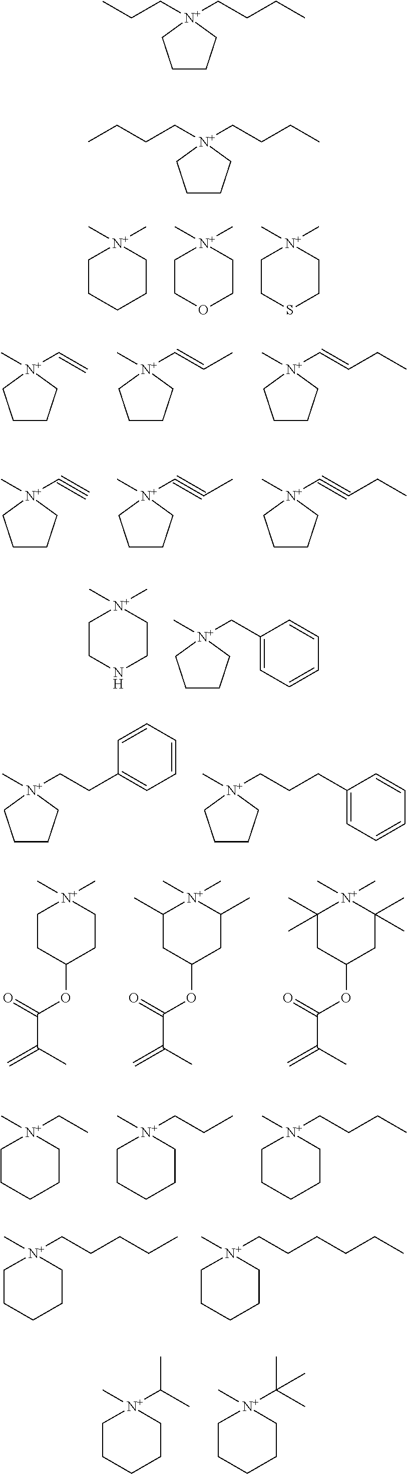

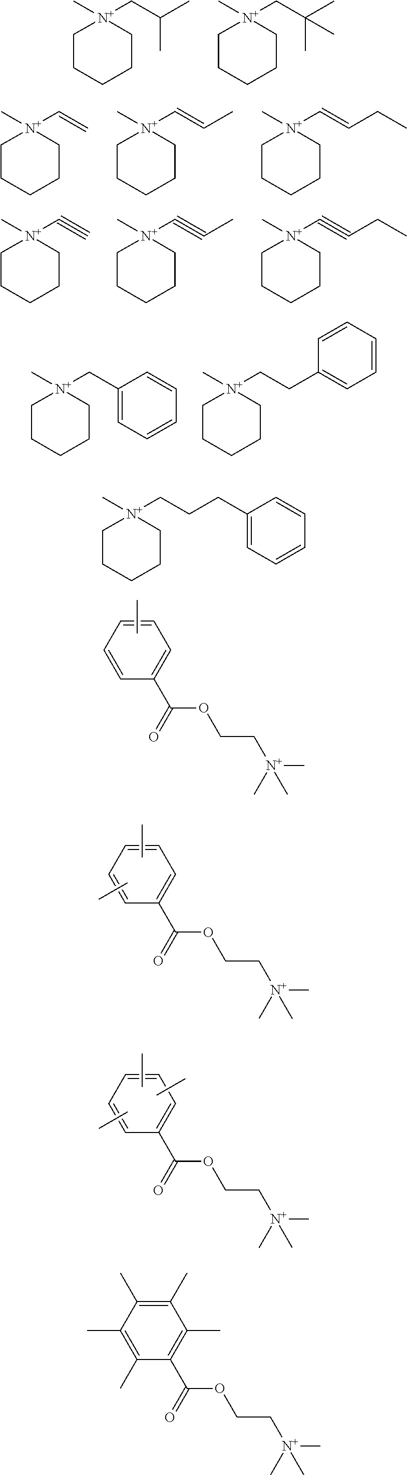

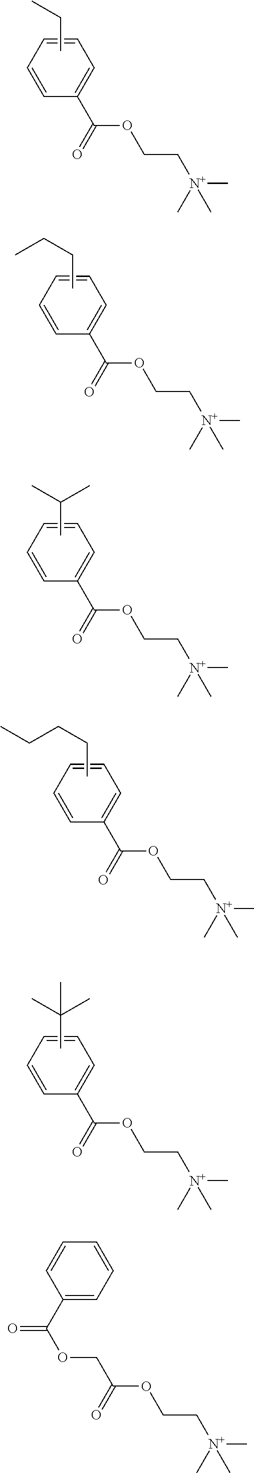

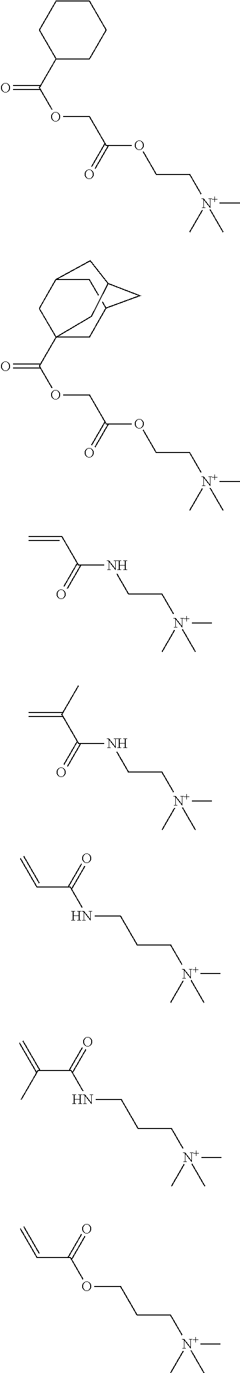

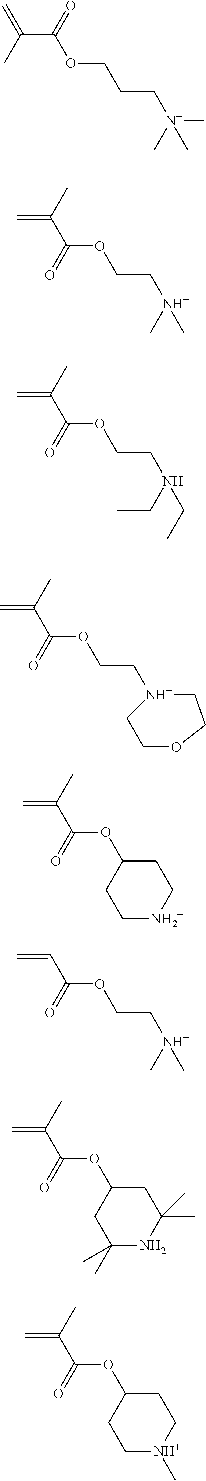

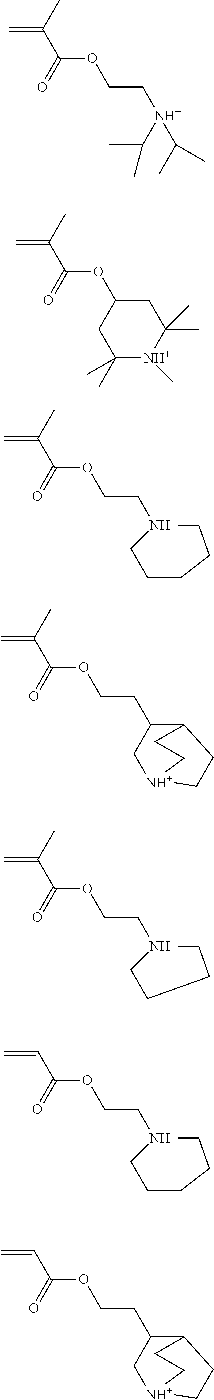

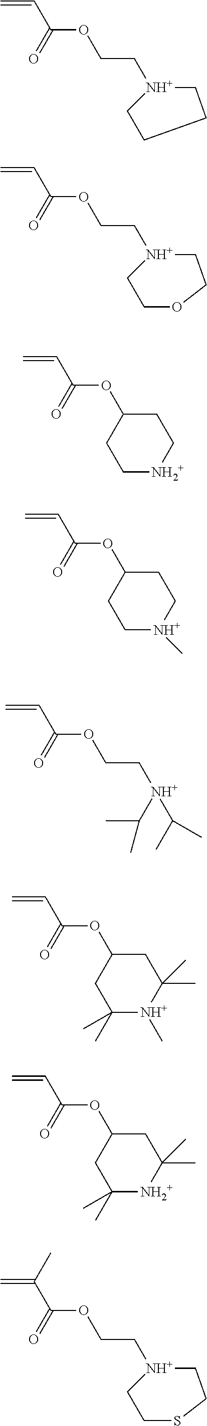

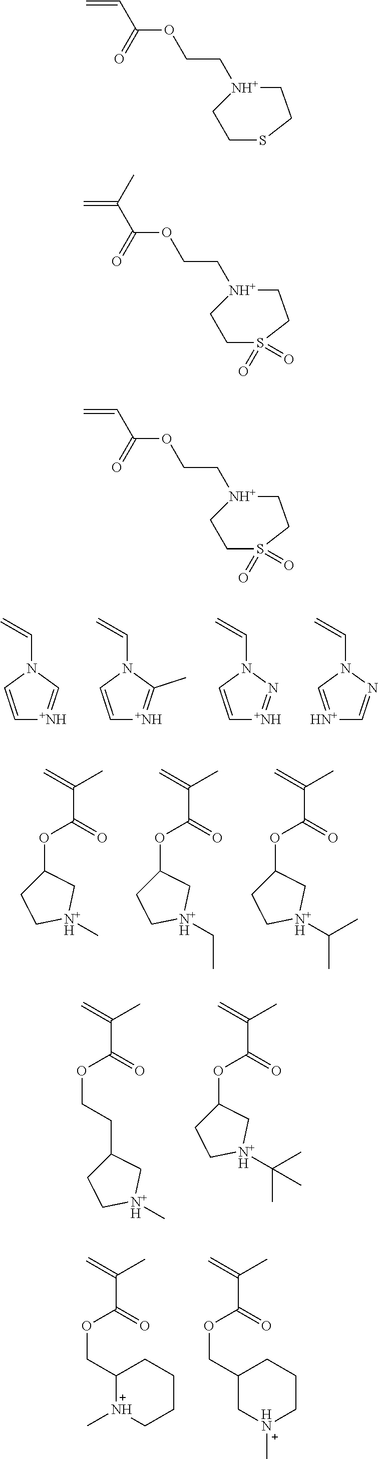

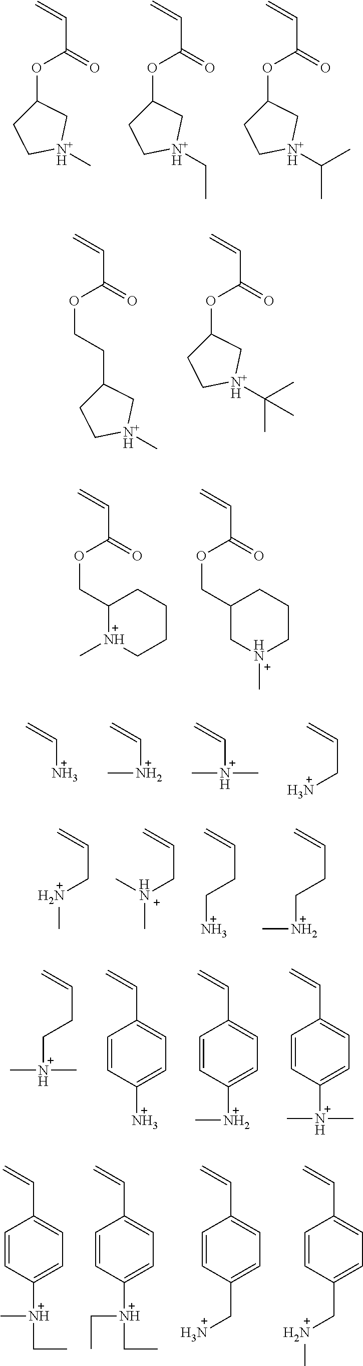

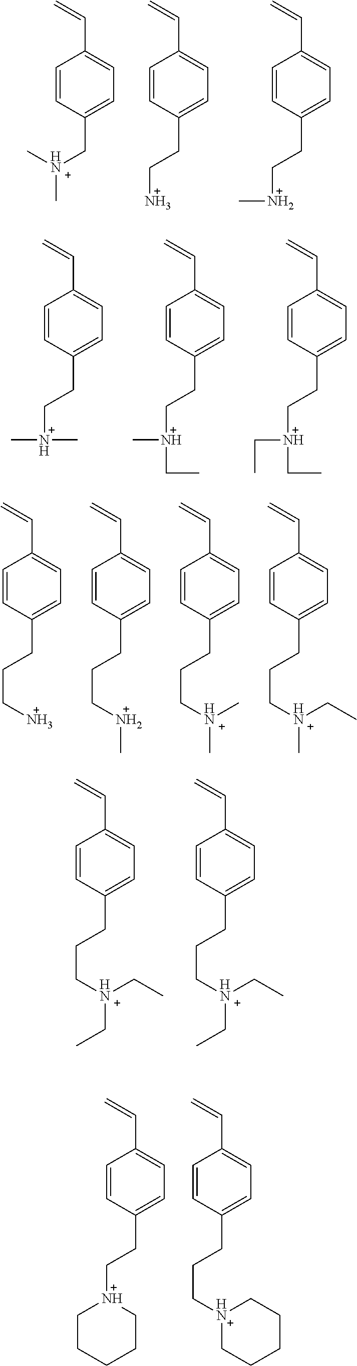

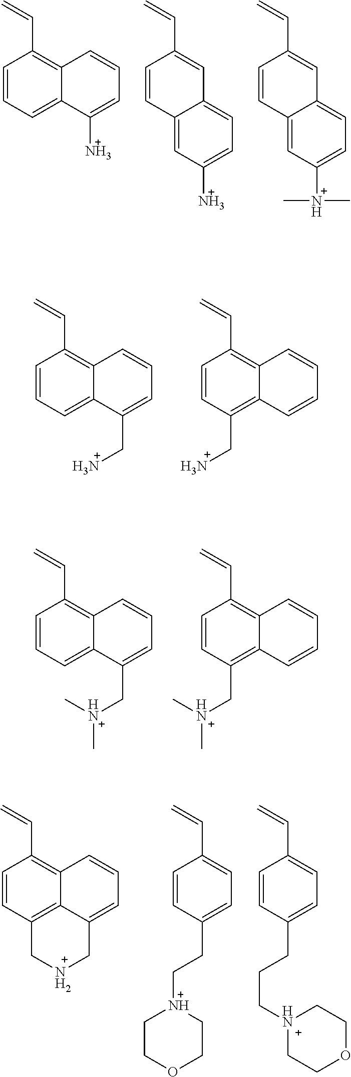

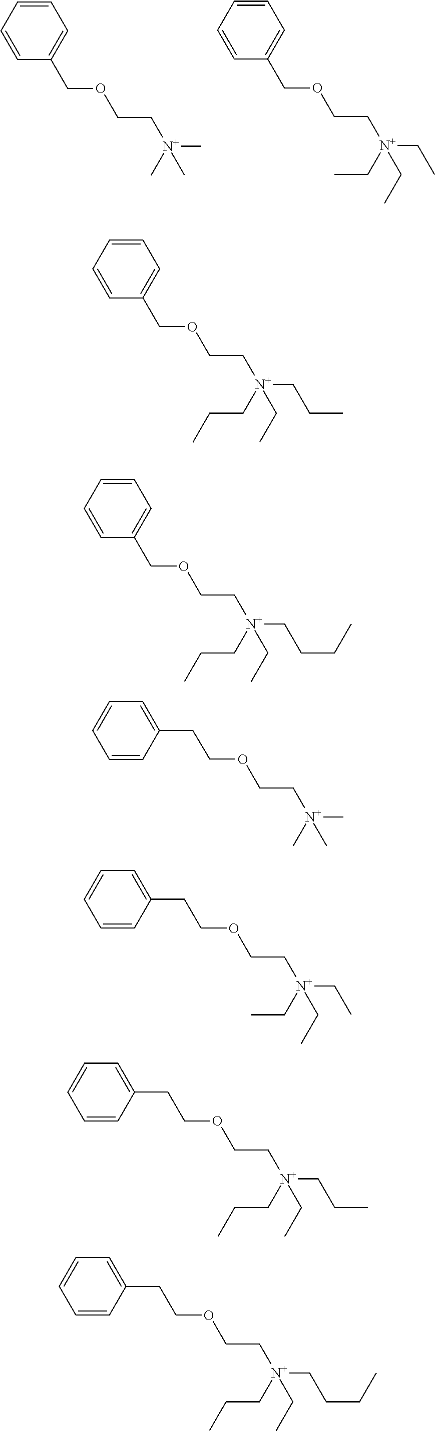

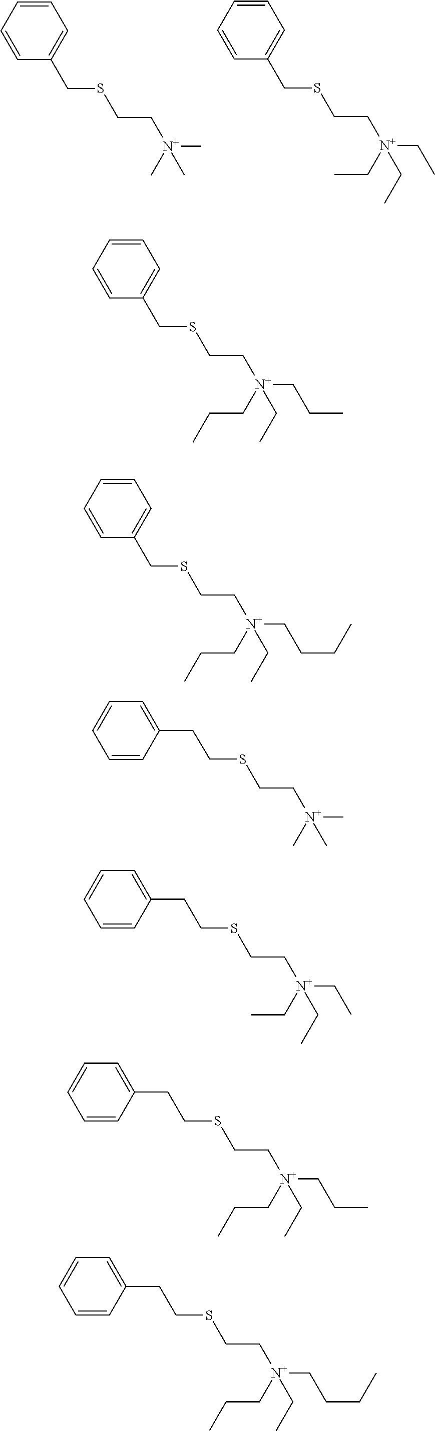

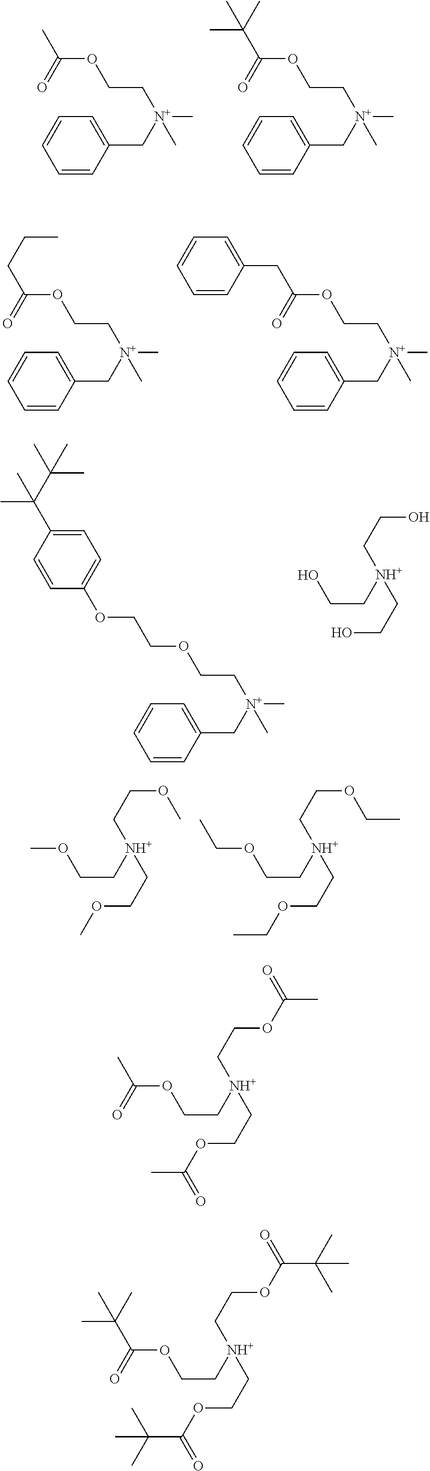

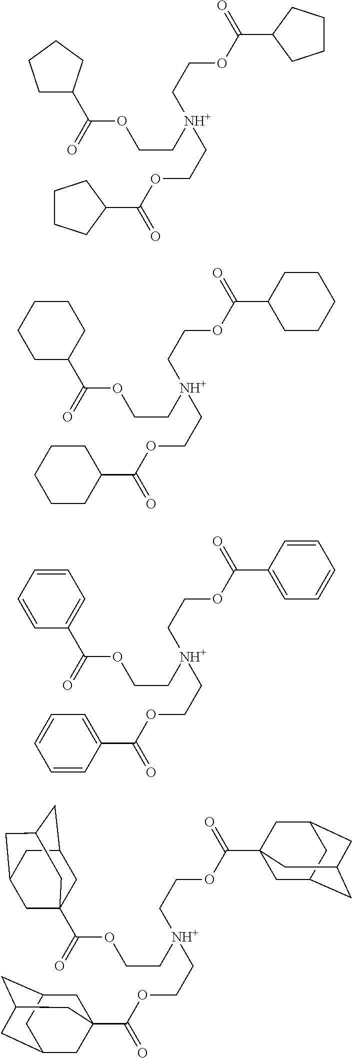

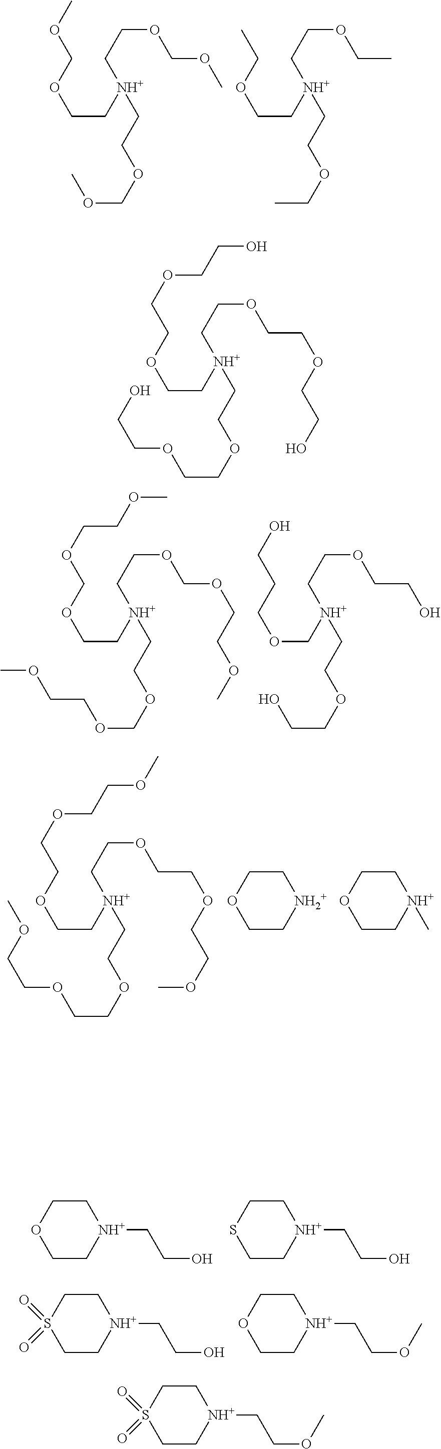

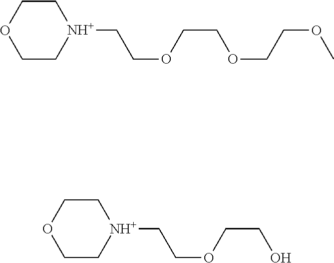

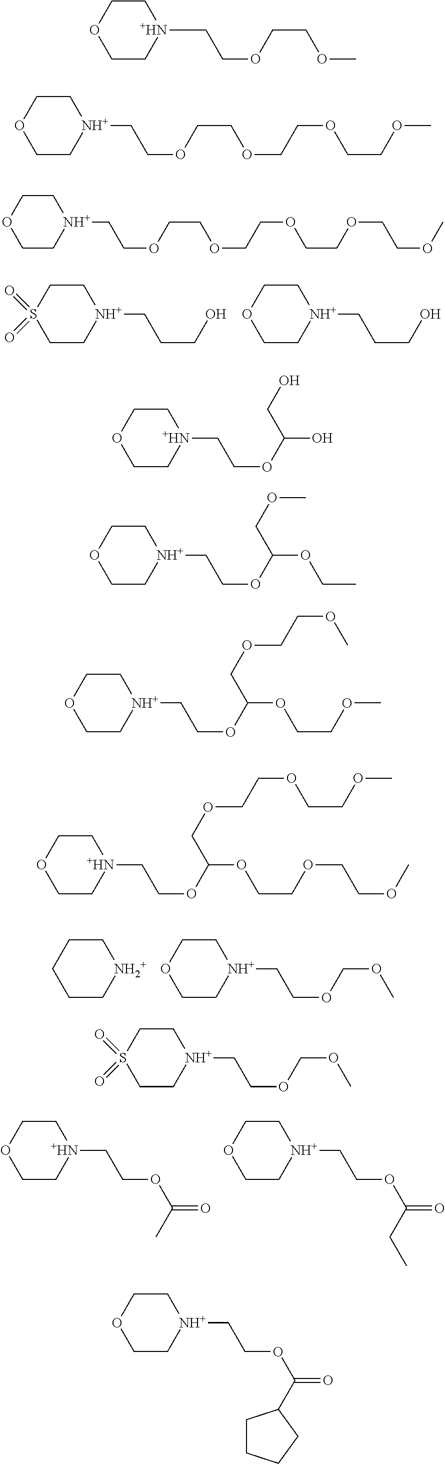

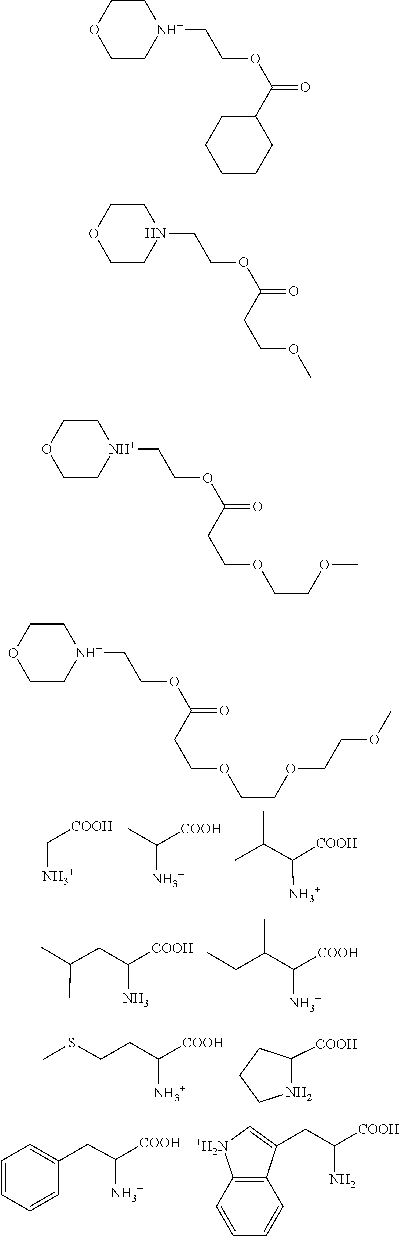

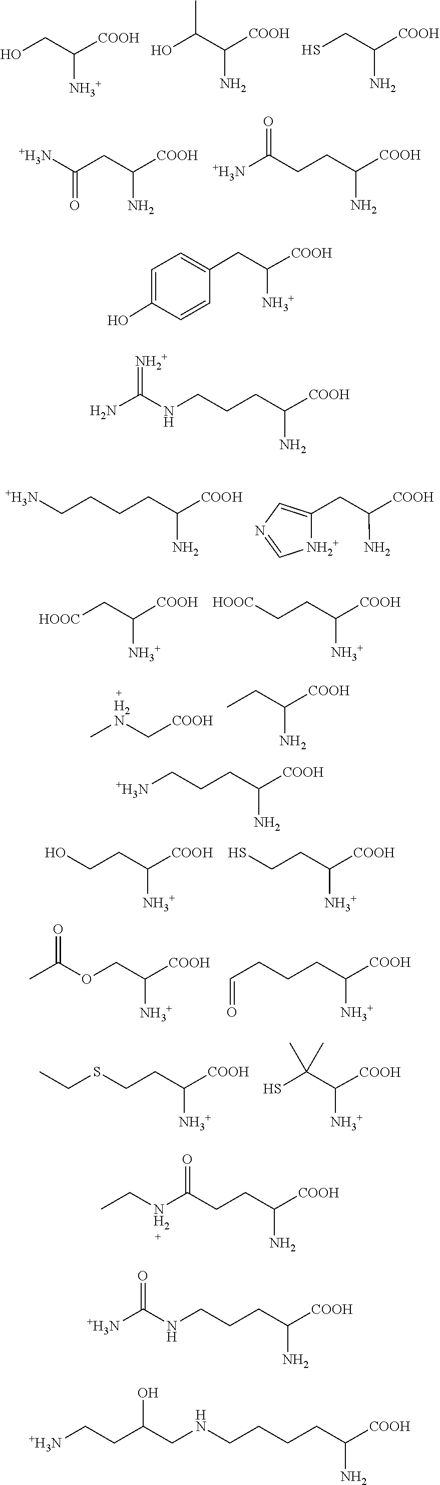

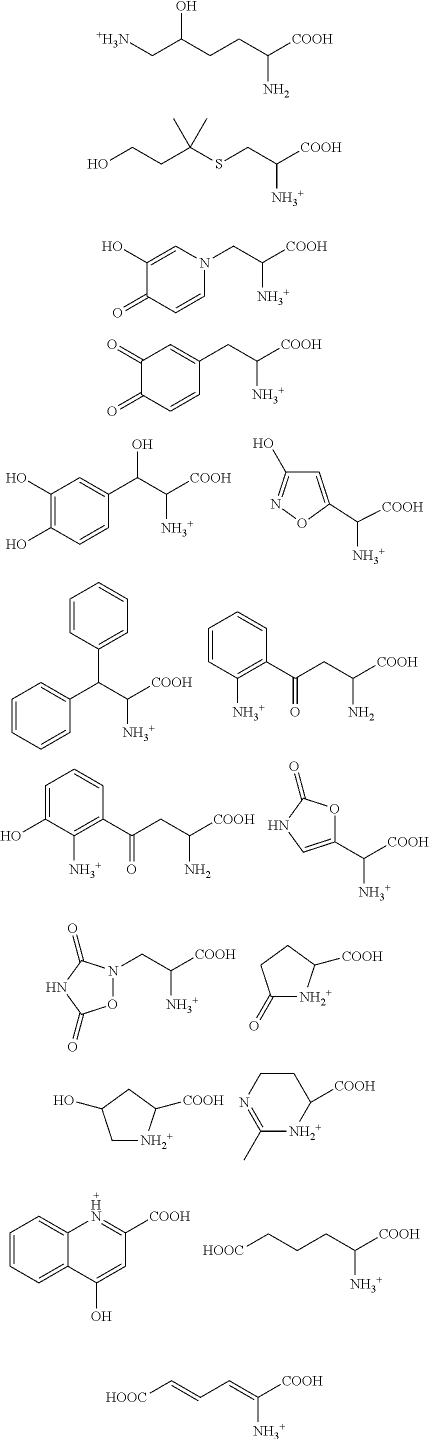

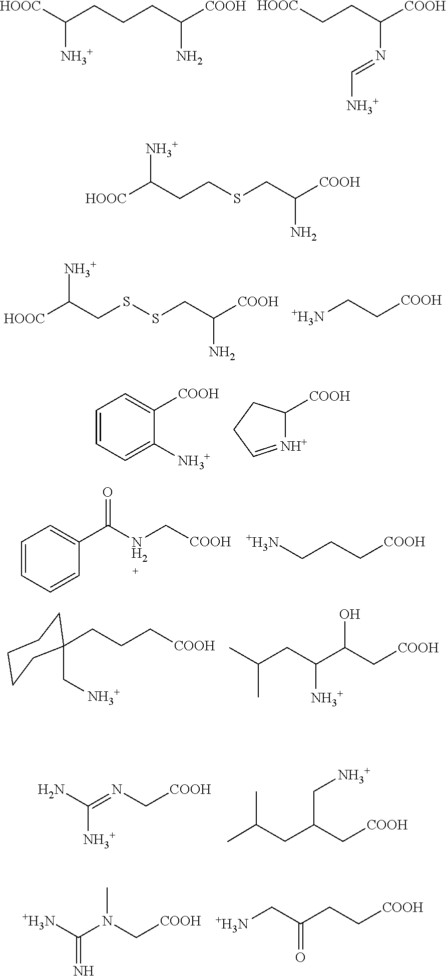

As one method for synthesizing an ionic polymer which the layer (B) contains, ionic polymer material can be obtained, for example, by a method in which desired monomer (s) among the monomers to give the repeating units-A1 to -A7, -b, -c, -d, -e, -f, -g and -h undergo heat polymerization in an organic solvent to which a radical polymerization initiator is added.

Examples of the organic solvents used in the polymerization include toluene, benzene, tetrahydrofuran, diethyl ether, dioxane, cyclohexane, cyclopentane, methyl ethyl ketone, γ-butyrolactone, etc.

Examples of the radical polymerization initiator include 2,2′-azobisisobutyronitrile (AIBN), 2,2′-azobis(2,4-dimethylvaleronitrile), dimethyl 2,2′-azobis(2-methylpropionate), benzoyl peroxide, lauroyl peroxide, etc.

The reaction temperature is preferably 50 to 80° C., and the reaction time is preferably 2 to 100 hours, more preferably 5 to 20 hours.

In the ionic polymer which the layer (B) contains, the number of the monomers which give the repeating units-A1 to -A7 may be one or more.

Further, when two or more kind of monomers which give the repeating units-A1 to -A7 are used, each monomer may be copolymerized at random, or may be in a block basis.

Further, the monomers to give the repeating units-A1 to -A7, -b, -c, -d, -e, -f, -g and -h, may be copolymerized at random, or in a block basis.

In general, the random copolymerization by a radical polymerization is conducted by mixing a monomer to be copolymerized and a radical polymerization initiator and heating them. In case where a first monomer starts polymerization under the radical polymerization initiator and then the second monomer was added, the polymer has a polymerized structure of the first monomer in one end and a polymerized structure of the second monomer in another end. However, in this case, the middle portion of the polymer includes both repeating units from the first monomer and ones from the second monomer, and its structure is different from one of a blocked copolymer. A living radical polymerization technique is preferably used when a blocked copolymer is formed by a radical polymerization.

In living radical polymerization technique called RAFT polymerization (Reversible Addition Fragmentation chain Transfer polymerization), because a radical at the terminal of a polymer is always active, by starting a polymerization of the first monomer and then adding the second monomer at the timing when the first monomer is used up, it is possible to form a diblock copolymer having both a block of a repeating unit of the first monomer and a block of a repeating unit of the second monomer. Further, by starting a polymerization of the first monomer, adding the second monomer at the timing when the first monomer is used up, and then adding the third monomer, it is possible to form a triblock polymer.

The RAFT polymerization has a characteristic of forming a polymer with narrow dispersity, which means a narrow distribution of molecular weights (dispersity). Especially when monomers are added at a time and subjected to RAFT polymerization, polymer having a narrower distribution of molecular weights is formed.

RAFT polymerization needs a chain transfer agent. Specific examples of the chain transfer agent include 2-cyano-2-propylbenzothioate, 4-cyano-4-phenylcarbonothioylthiopentanoic acid, 2-cyano-2-propyldodecyltrithiocarbonate, 4-cyano-4-[(dodecylsulfanylthiocarbonyl) sulfanyl]pentanoic acid, 2-(dodecylthiocarbonothioylthio)-2-methylpropanoic acid, cyanomethyl dodecyl thiocarbonate, cyanomethyl methyl(phenyl) carbamothioate, bis(thiobenzoyl)disulfide, and bis(dodecylsulfanylthiocarbonyl)disulfide. Among these, 2-cyano-2-propylbenzothioate is preferable especially.

A polymer contained in the layer (B) has a weight-average molecular weight in the range of 1,000 to 500,000, preferably in the range of 2,000 to 200,000. When the weight-average molecular weight is less than 1,000, heat resistance is poor, and it is possible that a residue remained on the skin after being peeling off. Meanwhile, when the weight-average molecular weight is more than 500,000, its viscosity increases, its workability decrease, and its solubility in organic solvents decreases.

Not that the weight-average molecular weight (Mw) is a value measured in terms of polyethylene oxide, polyethylene glycol, or polystyrene, by gel permeation chromatography (GPC) using dimethylformamide (DMF) and tetrahydrofuran (THF) as a solvent.

As disclosed in JP 2021-164630 A, it is possible by reactions to introduce a radically reactive group such as (meth)acrylate group, or styrene group into a hydroxy group, a carboxy group, an oxirane group, an oxetane group, or an isocyanate group, etc. of ionic polymer after the polymerization.

Here, ratios of repeating units-A1 to -A7, -b, -c, -d, -e, -f, -g, and -h, in an ionic polymer contained in the layer (B) are: 0≤a1≤1.0, 0≤a2≤1.0, 0≤a3≤1.0, 0≤a4≤1.0, 0≤a5≤1.0, 0≤a6≤1.0, 0≤a7≤1.0, 0<a1+a2+a3+a4+a5+a6+a7≤1.0, 0≤b<1.0, 0≤c<1.0, 0≤d<1.0, 0≤e<1.0, 0≤f<1.0, 0≤g<1.0, 0≤h<1.0; preferably 0≤a1≤1.0, 0≤a2≤1.0, 0≤a3≤1.0, 0≤a4≤1.0, 0≤a5≤1.0, 0≤a6≤1.0, 0≤a7≤1.0, 0.1≤a1+a2+a3+a4+a5+a6+a7≤1.0, 0≤b≤0.8, 0≤c≤0.8, 0≤d≤0.8, 0≤e≤0.8, 0≤f≤0.8, 0≤g≤0.8, 0≤h≤0.8; and more preferably 0≤a1≤1.0, 0≤a2≤1.0, 0≤a3≤1.0, 0≤a4≤1.0, 0≤a5≤1.0, 0≤a6≤1.0, 0≤a7≤1.0, 0.2≤a1+a2+a3+a4+a5+a6+a7≤1.0, 0<b≤0.7, 0≤c≤0.7, 0≤d≤0.7, 0≤e≤0.7, 0≤f≤0.7, 0≤g≤0.7, 0≤h≤0.7. a1 to a7, “b”, “c”, “d”, “e”, “f”, “g”, and “h”, are a ratio of repeating units-A1 to -A7, -b, -c, -d, -e, -f, -g, and -h respectively.

### Other Components

### (Surfactants)

In the present invention, a surfactant may be added in order to increase wettability of a solution containing metal nanowire, an electro-conductive paste containing conductive particles, or a bio-electrode composition, against a material to be processed such as a substrate. Examples of the surfactant include a nonionic, cationic, and anionic surfactants. Specifically, the examples include nonionic surfactants such as polyoxyethylene alkyl ether, polyoxyethylene alkylphenyl ether, polyoxyethylene carboxylate, sorbitan ester, and polyoxyethylene sorbitan ester; cationic surfactants such as alkyltrimethylammonium chloride, and alkylbenzylammonium chloride; anionic surfactants such as alkyl or alkylaryl sulfates, alkyl or alkylaryl sulfonates, and dialkyl sulfosuccinate; and zwitterionic surfactants such as amino acid type and betaine type, etc. The surfactant is preferably contained in an amount of 50 to 0.1 parts by mass, more preferably 20 to 1 parts by mass, based on 100 parts by mass of the ionic polymer.

### [Resin (C)]

The Resin (C) blended in a composition to form the ionic polymer containing layer of the inventive bio-electrode is a component to prevent elution of the ionic polymer and to maintain adhesiveness with the electro-conductive wiring. Components contained in the layer (B) preferably contain at least one resin (C) selected from the group consisting of (meth)acrylate resin, (meth)acrylamide resin, urethane resin, polyurethane (meth)acrylate, polyvinyl alcohol, polyvinylpyrrolidone, polyoxazoline, polyglycerin, polyglycerin-modified silicone, polyglycerin (meth)acrylate, cellulose, polyethylene glycol, and polypropylene glycol. Resin (C) is preferably contained in an amount of 1000 to 5 parts by mass, more preferably 500 to 10 parts by mass, based on 100 parts by mass of the ionic polymer.

### [Optional Component]

Compositions to form the ionic polymer containing layer of the inventive bio-electrode can contain optional components such as an ionic additive, a silicone compound having a polyglycerin structure, an organic solvent, etc.

### [Ionic Additives]

An ionic additive can be added to composition to form the ionic polymer containing layer of the inventive bio-electrode to increase ionic conductivity. In light of biocompatibility, examples of the ionic additives include sodium chloride, potassium chloride, calcium chloride, saccharin, acesulfame potassium, and salts disclosed in JP 2018-044147 A, JP 2018-059050 A, JP 2018-059052 A, or JP 2018-130534 A.

Ammonium salts of fluorosulfonic acid, fluoroimide acid, and fluoromethide acid are known as ionic liquids. Specifically, it is possible to add ionic liquids described in Trulove C, Mantz R. 2003. Ionic Liquids in Synthesis, Chapter 3.6: Electrochemical Properties of Ionic Liquids. The ionic additive is preferably contained in an amount of 100 to 1 parts by mass, more preferably 50 to 5 parts by mass, based on 100 parts by mass of the ionic polymer.

### [Silicone Compound Having a Polyglycerin Structure]

In composition to form the ionic polymer containing layer of the inventive bio-electrode, in order to improve moisture retaining property and sensitivity to ion released from skin and ionic conductivity, it is possible to add a silicone compound having a polyglycerin structure. The silicone compound having a polyglycerin structure is preferably contained in an amount of 0.01 to 100 parts by mass, more preferably 0.5 to 60 parts by mass, based on 100 parts by mass of the ionic polymer. Further, the silicone compound having a polyglycerin structure may be used alone, or in a mixture of 2 or more.

Examples of such a silicone compound having a polyglycerin structure include those disclosed in JP 2021-115458 A.

When such a silicone compound having a polyglycerin structure are contained, it is possible to exhibit more excellent moisture retaining property, and consequently to have a bio-electrode composition to form the ionic polymer containing layer having more excellent sensitivity to ion released from skin.

### [(D) Organic Solvent]

Specific examples of the organic solvent include: ketone solvents, such as cyclohexanone, cyclopentanone, 2-octanone, 2-nonanone, 2-heptanone, 3-heptanone, 4-heptanone, 2-hexanone, 3-hexanone, diisobutyl ketone, methylcyclohexanone, and methyl n-pentyl ketone; alcohol solvents, such as 3-methoxybutanol, 3-methyl-3-methoxybutanol, 1-methoxy-2-propanol, and 1-ethoxy-2-propanol; ether solvents, such as propylene glycol monomethyl ether, ethylene glycol monomethyl ether, propylene glycol monoethyl ether, ethylene glycol monoethyl ether, propylene glycol dimethyl ether, diethylene glycol monomethyl ether, diethylene glycol monoethyl ether, diethylene glycol monopropyl ether, diethylene glycol monobutyl ether, diethylene glycol monopentyl ether, diethylene glycol monoheptyl ether, diethylene glycol diethyl ether, diethylene glycol dipropyl ether, diethylene glycol dibutyl ether, diisopropyl ether, diisobutyl ether, diisopentyl ether, di-n-pentyl ether, methyl cyclopentyl ether, methyl cyclohexyl ether, di-n-butyl ether, di-sec-butyl ether, di-sec-pentyl ether, di-tert-amyl ether, di-n-hexyl ether, and anisole; ester solvents, such as propylene glycol monomethyl ether acetate, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactone solvents, such as γ-butyrolactone; etc.

Note that the organic solvent is preferably contained in an amount of 10 to 50,000 parts by mass based on 100 parts by mass of the ionic polymer.

When a repeating unit of the ionic polymer have an oxirane group or an oxetane group, an amine-based crosslinking agent or an imidazole-based crosslinking catalyst can be added. When a repeating unit of the ionic polymer have a carboxy group, amino group, or an imidazole group, a crosslinking agent having an oxirane group or an oxetane group can be added.

For decreasing viscosity or crosslinking of composition to form the ionic polymer containing layer, a (meth)acrylate monomer can be added. For crosslinking, a monomer preferably contains a plurality of (meth)acrylate.

In order to react the (meth)acrylate, a radical generator can be added. The radical generator is a compound which generate a radical by heat of light. Specifically, compounds disclosed in JP 2020-097214 A can be used.

### <Electro-Conductive Layer>

The electro-conductive wiring having a width of 200 μm or less, which is contained in the electro-conductive layer, is preferably a printed pattern formed by an electro-conductive paste including a particle of gold, silver, copper, or nickel, or a fusion layer of a metal nanowire including gold, silver, copper, nickel, or alloy thereof.

When the electro-conductive wiring having a width of 200 μm or less is formed by printing, electro-conductive paste including a particle of gold, silver, copper, or nickel is preferably used. The electro-conductive paste is preferably mixed with an organic solvent or a resin besides an electro-conductive particle such as a particle of gold, silver, copper, or nickel. The electro-conductive particle is particular preferably silver. Compositions of the electro-conductive paste are disclosed specifically in JP 2022-078861 A.

Hereinafter, the inventive bio-electrode will be described in detail with reference to the drawings, but the present invention is not limited thereto.

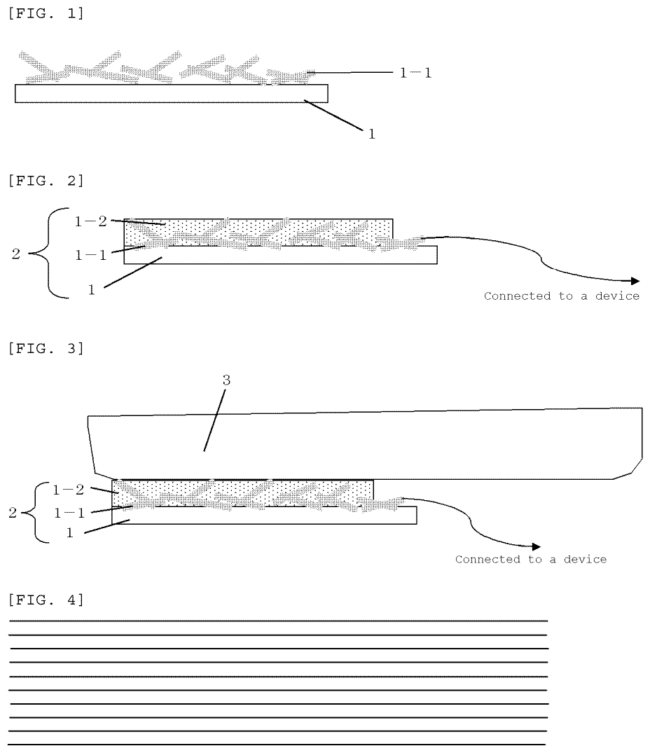

FIG. 1 shows a schematic sectional view showing after the electro-conductive layer 1-1 is formed on a substrate 1. The electro-conductive layer 1-1 of this case is an example using a metal nanowire.

FIG. 2 shows a schematic sectional view of the inventive bio-electrode 2 after the ionic polymer containing layer 1-2 are formed on the electro-conductive layer 1-1 on the substrate 1. All of the surface of the electro-conductive layer 1-1 may be covered by the ionic polymer containing layer 1-2, or the surface of the electro-conductive layer 1-1 may be partially exposed on the surface.

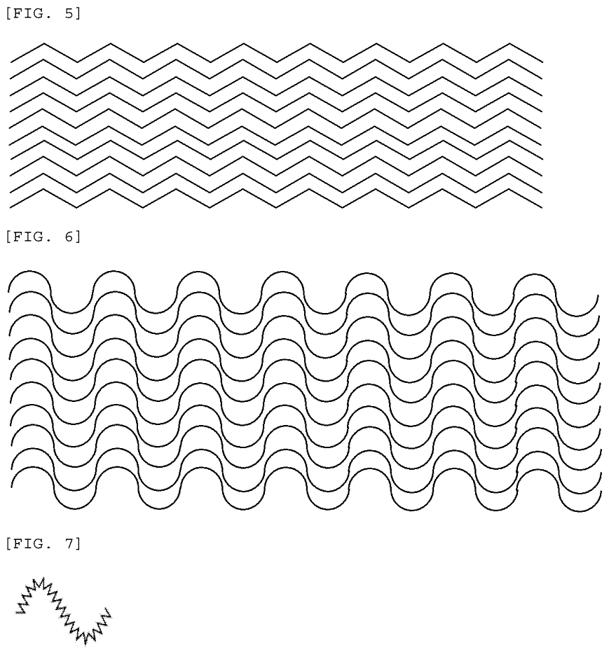

FIG. 3 shows a schematic sectional view of the inventive bio-electrode 2 attached on the human skin 3. One surface of the ionic polymer containing layer 1-2 is in contact with the human skin and another surface is in contact with the electro-conductive layer 1-1.

In the present invention, patterns of the electro-conductive wiring are not limited specifically, but exemplified below. FIG. 4 shows a schematic top view (plan view) of the electro-conductive wiring having a width of 200 μm or less, which is formed in the shape of straight lines by printing. The space between the wiring improves transparency and a plurality of wirings improves electro-conductivity.

FIG. 5 shows a schematic top view of the electro-conductive wiring having a width of 200 μm or less, which is formed in the shape of zigzag line by printing. When the electro-conductive wiring is extended or contracted in a horizontal direction, the wiring in a zigzag pattern has less change in electro-conductivity when extended or contracted.

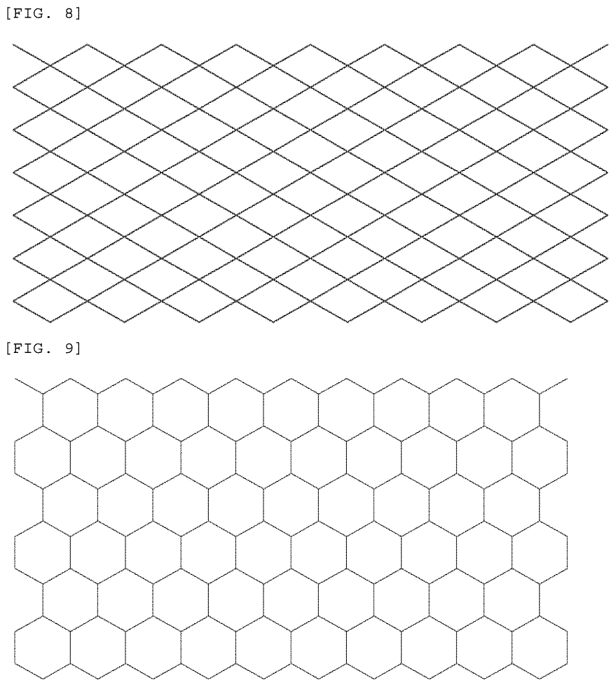

FIG. 6 shows a schematic top view of the electro-conductive wiring having a width of 200 μm or less, which is printed to form the shape of zigzag line having corners rounded (wavy lines). The wiring pattern such a wavy line has less change in electro-conductivity when extended or contracted than wiring patterns having a straight line and a sharp angle.

FIG. 7 shows a schematic top view of electro-conductive wiring having a width of 200 μm or less, which is formed in a combination of two zigzag lines. This wiring pattern has less change in electro-conductivity when extended or contracted than one zigzag wiring pattern having a straight line and a sharp angle as shown in FIG. 5.

The electro-conductive wiring may have a zigzag pattern, not only in horizontal to a substrate as shown in FIGS. 5 to 7, but also in vertical to a substrate as disclosed in JP 2020-107875 A.

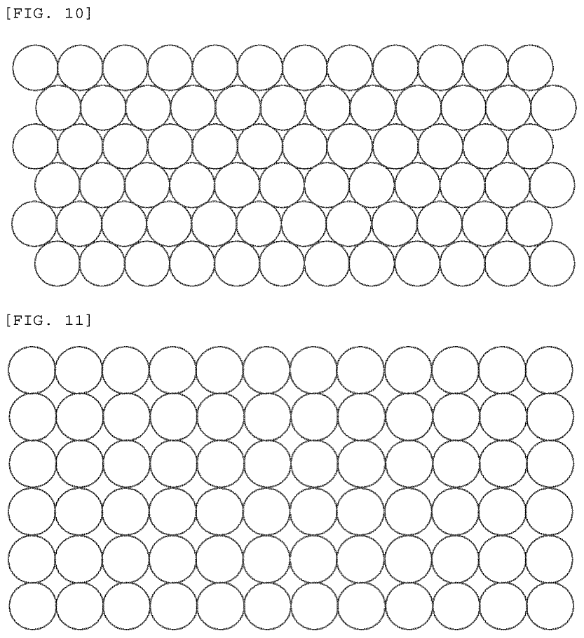

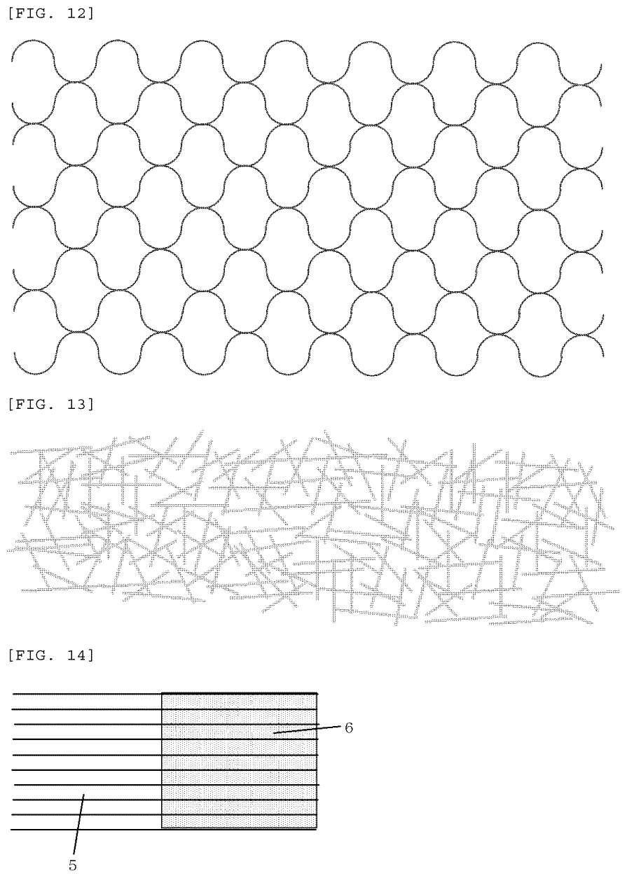

The printed pattern of the electro-conductive wiring may consist of lines not connected each other as shown in FIGS. 4 to 7, or may have a connection wiring to connect neighboring wirings. For example, the patterns can include a reticular pattern (aslant lattice pattern) shown in FIG. 8, a hexagonal pattern shown in FIG. 9, a pattern of connected-circles shown in FIGS. 10 and 11, and a mesh pattern shown in FIG. 12. In addition to these, the patterns can include a Higaki pattern with repeated diagonal rectangles, a Mutsude no Manji pattern that combines hexagons, a Kagome pattern that combines equilateral triangles and straight lines, and a Bishamon tortoiseshell pattern, a Fundo Tsunagi pattern with curved lines crossing diagonally, a Tokkuri Ajiro pattern, and a Hoshishippo pattern with overlapping circles (http://www.natubunko.net/wagara/kotoba09g.html).

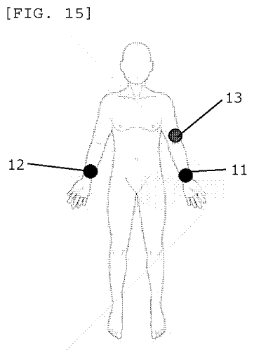

FIG. 13 shows a schematic top view showing the electro-conductive wiring, which is a fusion layer which is made by coating and fusing a solution containing metal nanowires to a substrate. Spaces between metal nanowires improve transparency. Contact between metal nanowires each other improve electro-conductivity when the electro-conductive wiring is extended or contracted.

The thickness of the bio-electrode which is a combination of the Layer (A) and Layer (B) is preferably 1 nm or more and 1 mm or less, more preferably 2 nm or more and 0.5 mm or less.

### <Method for Manufacturing Bio-Electrode>

The present invention provides a method for manufacturing the bio-electrode described above, the method including: forming the layer (A) on a substrate by applying a solution including a metal nanowire, or by printing an electro-conductive paste containing a conductive particle; and forming the layer (B) on the layer (A) by coating a bio-electrode composition including the polymer.

A substrate preferably has flexibility, stretchability, and transparency. A thin film glass has flexibility and high transparency, but the skin is hurt when the glass is broken while attached on the skin. Therefore, resin which cannot be broken is preferable. Examples of resins which can be used as a substrate can include poly(meth)acrylate (PMMA), polyurethane, silicone, PEEK, polyimide, polyolefin, styrene-butadiene rubber, polycarbonate, PET, PEN, PVC, polystyrene, polyethylene, polypropylene, polymethylpentene, and Teflon (registered trademark).

A method to form the electro-conductive layer on a substrate can include: printing an electro-conductive paste containing a conductive particle, a resin, and a solvent as an electro-conductive pattern having a width of 200 μm or less; and coating ink (solution) containing a metal nanowire.

A Method to form the ionic polymer containing layer on the electro-conductive layer can include coating a bio-electrode composition containing the polymer.

A Method to coat the bio-electrode composition containing the polymer on the electro-conductive layer is not specifically limited, but includes direct coating and transferring what is coated on another substrate. In either method, the preferable methods are, for example, dip coating, spray coating, spin coating, roll coating, flow coating, doctor coating, screen printing, flexo printing, gravure printing, inkjet printing, etc.

The same methods as one for coating a bio-electrode composition containing the polymer are used for printing the electro-conductive paste containing an electro-conductive particle, a resin, and a solvent, as an electro-conductive pattern having width of 200 μm or less, and for coating an ink containing a metal nanowire.

To evaporate a solvent and cure the layer, heating is conducted after coating the electro-conductive paste, the ink containing a metal nanowire, and the bio-electrode composition containing the polymer.

Note that a temperature in case of heating after coating a bio-electrode composition containing the polymer is not particularly limited and, for example, temperature around a range of 50 to 250° C. is preferable.

In case of using a silver nanowire etc., the heating temperature after coating the electro-conductive paste or after coating the ink containing a metal nanowire is in the range of 200 to 600° C., in order to fuse silver each other. At this time, to prevent thermal decomposition of a substrate, it is possible to use a flash lamp anneal method which radiate intense UV in a short time.

In case that heating and radiation of light are combined, these may be conducted at the same time, heating may be conducted after light radiation, or vice versa. Further, air-dry may be conducted to evaporate a solvent before heating after coating.

Putting a waterdrop or spraying a surface of the cured ionic polymer containing layer with steam or mist, improves affinity with the skin, and a biological signal can be obtained promptly. To make a small size of waterdrop of the steam or the mist, it is possible to use a water mixed with alcohol. It is also possible to wet the film surface or skin surface by having them touched with absorbent cotton or cloth containing water.

Water to wet the surface of the cured ionic polymer containing layer may contain salt. A water-soluble salt to be mixed with water is preferably selected from the group of sodium salt, potassium salt, calcium salt, magnesium salt, and betaine.

The water-soluble salt can be salts specifically selected from the group of sodium chloride, potassium chloride, calcium chloride, magnesium chloride, saccharin sodium salt, acesulfame potassium, sodium carboxylate, potassium carboxylate, calcium carboxylate, sodium sulfonate, potassium sulfonate, calcium sulfonate, sodium phosphate, potassium phosphate, calcium phosphate, magnesium phosphate, and betaine. Note that the ionic polymer is not included in the water-soluble salt.

More specifically, in addition to the above, the water-soluble salts include sodium acetate, sodium propionate, sodium pivalate, sodium glycolate, sodium butyrate, sodium valerate, sodium caproate, sodium enanthate, sodium caprylate, sodium pelargonate, sodium caprate, sodium undecylate, sodium laurate, sodium tridecylate, sodium myristate, sodium pentadecylate, sodium palmitate, sodium margarate, sodium stearate, sodium benzoate, disodium adipate, disodium maleate, disodium phthalate, sodium 2-hydroxybutyrate, sodium 3-hydroxybutyrate, sodium 2-oxobutyrate, sodium gluconate, sodium methanesulfonate, sodium 1-nonanesulfonate, sodium 1-decanesulfonate, sodium 1-dodecanesulfonate, sodium 1-undecanesulfonate, sodium cocoyl isethionate, sodium lauroylmethylalanine, sodium cocoyl methyl taurate, sodium cocoyl glutamate, sodium cocoyl sarcosine, sodium lauroylmethyltaurate, laumidopropyl betaine, potassium isobutyrate, potassium propionate, potassium pivalate, potassium glycolate, potassium gluconate, potassium methanesulfonate, calcium stearate, calcium glycolate, calcium gluconate, calcium 3-methyl-2-oxobutyrate, calcium methanesulfonate.

Betaine is a generic term for internal salt, specifically compounds in which three methyl groups are added to an amino group of an amino acid, more specifically it includes trimethylglycine, carnitine and proline betaine.

The water-soluble salts can further contain monohydric or polyhydric alcohols having 1 to 4 carbon atoms, and the alcohol is preferably selected from the group of ethanol, isopropyl alcohol, ethylene glycol, diethylene glycol, triethylene glycol, glycerin, polyethylene glycol, polypropylene glycol, polyglycerin, diglycerin, or a silicone compound having a polyglycerin structure.

As a preprocessing process using solution containing a salt, it is possible to wet the cured ionic polymer containing layer by atomization method, waterdrop dispensing method, etc. It is also possible to wet the layer in hot, humid conditions like in a sauna. The wet layer can be covered by a sheet to prevent drying after getting wet. Because the sheet has to be removed immediately before attaching the bio-electrode on the skin, the sheet is coated by a release agent or a removable fluororesin film. A dry electrode covered by a removable sheet are sealed in a bag covered with aluminum, etc. for long-term storage. In order to prevent drying in a bag covered by aluminum, it is preferable to seal water in the bag.

It is effective in term of humidifying skin surface and obtaining a biological signal with high sensitivity and high accuracy in a shorter time, to wipe skin where the bio-electrode is attached with fabric containing water or water containing alcohol such as ethanol, glycerin, etc., or to spray them onto the skin, immediately before attaching the bio-electrode. Wiping with fabric containing water is effective not only in humidifying skin but also in removing oil on the skin surface, and also improve sensitivity to a biological signal.

As has been described above, the inventive method for manufacturing the bio-electrode makes it possible to manufacture the inventive bio-electrode easily at low cost, which is thin, highly transparent, highly sensitive to a biological signal, excellent in biocompatibility, light-weight, capable of preventing significant reduction in electro-conductivity when wetted with water or dried, and comfortable without itching, reddening, nor rash of skin.

### EXAMPLE

Hereinafter, the present invention will be specifically described with reference to Examples and Comparative Examples, but the present invention is not limited thereto.

Ionic polymers 1 to 24 and Comparative ionic polymer 1, which were blended as ionic materials (conductive materials) in bio-electrode composition solutions, were synthesized as follows.

First, 30 mass % cyclopentanone solutions of respective monomers were introduced into a reaction vessel and mixed. The reaction vessel was cooled to −70° ° C. under a nitrogen atmosphere, and vacuum degassing and nitrogen blowing were repeated three times. After raising the temperature to room temperature, 0.01 mol of azobisisobutyronitrile (AIBN) was added as a polymerization initiator based on 1 mol of the entire monomer, and the mixture was reacted for 15 hours after raising the temperature to 60° C. After drying the solvent, the composition of the resulting polymer was identified by ¹H-NMR. The molecular weight (Mw) and the dispersity (Mw/Mn) of the obtained polymer were determined by gel permeation chromatography (GPC) using tetrahydrofuran (THF) as an eluent. In case of ionic polymer 10, 2-(dimethylamino)ethyl methacrylate was added for neutralization, after copolymerization of a monomer having sulfonic acid. In case of ionic polymer 16, a polymer having a pendant with acrylic group was synthesized by reacting a polymerization solution with hydroxyethyl acrylate to form urethane bonds, after copolymerization of a monomer having an isocyanate group. In case of ionic polymer 17, after copolymerizing a monomer having a carboxyl group, glycidyl acrylate was added to the polymerization solution to open the oxirane ring to synthesize a polymer having acrylic groups as a pendant. The solvent was evaporated to obtain a powder. Thus, synthesized Ionic polymers 1 to 24 and Comparative ionic polymer 1 are shown below.

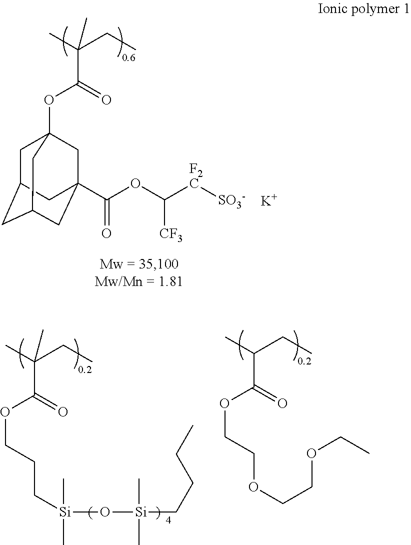

The repeating number in formula shows the average value.

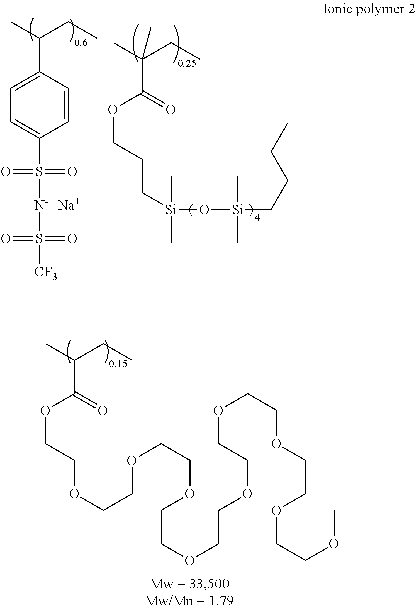

The repeating number in formula shows the average value.

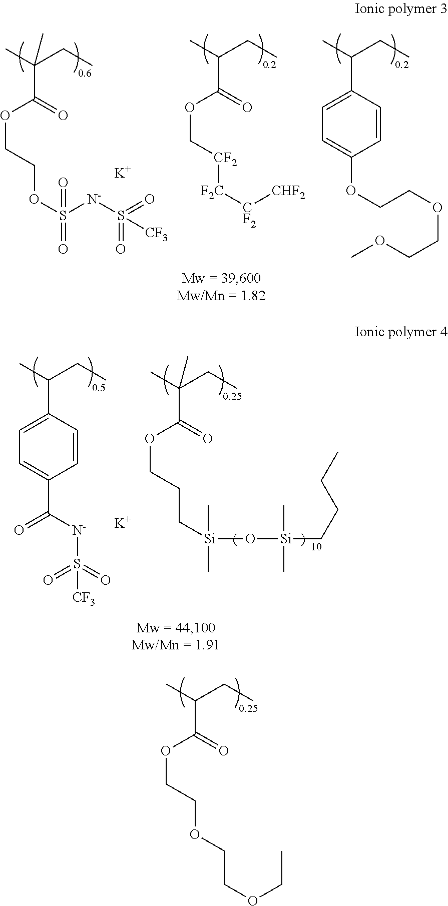

The repeating number in formula shows the average value.

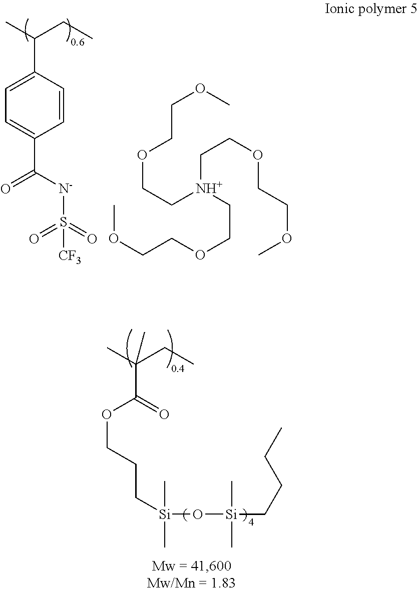

The repeating number in formula shows the average value.

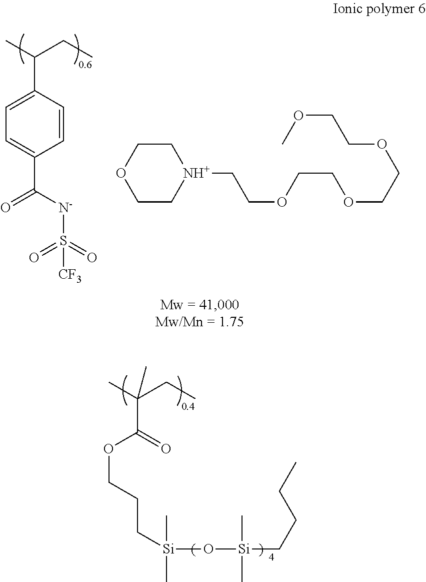

The repeating number in formula shows the average value.

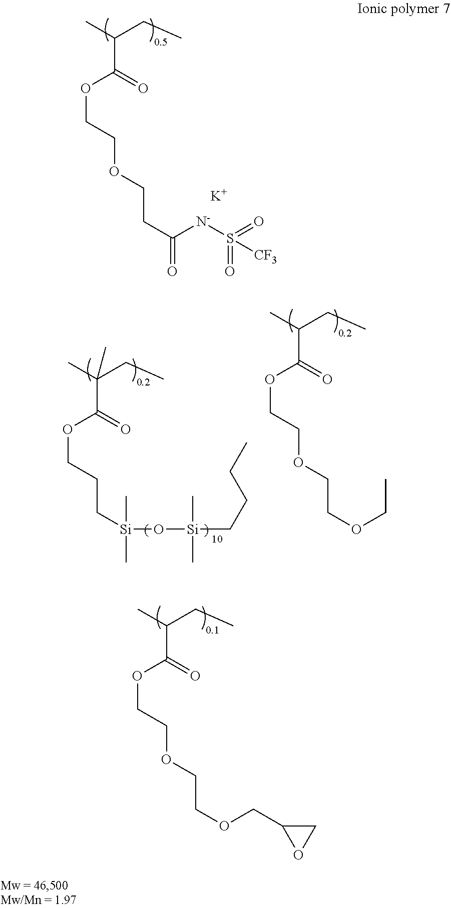

The repeating number in formula shows the average value.

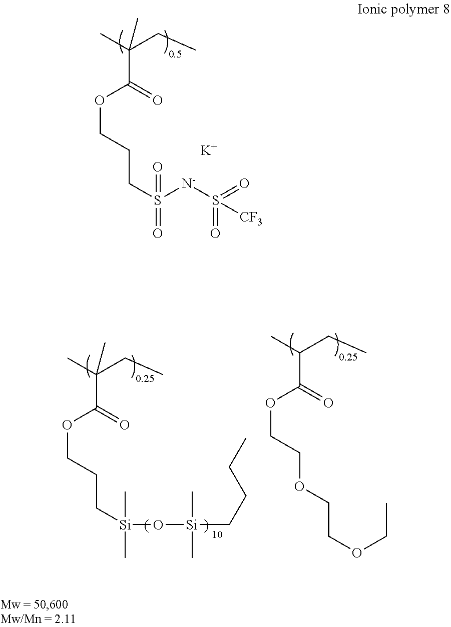

The repeating number in formula shows the average value.

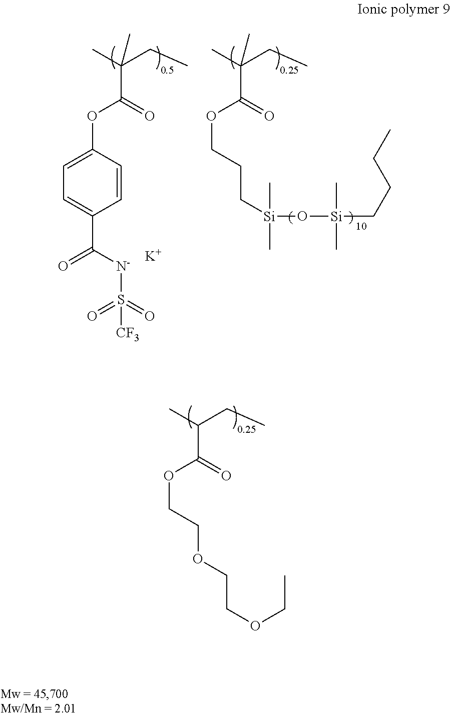

The repeating number in formula shows the average value.

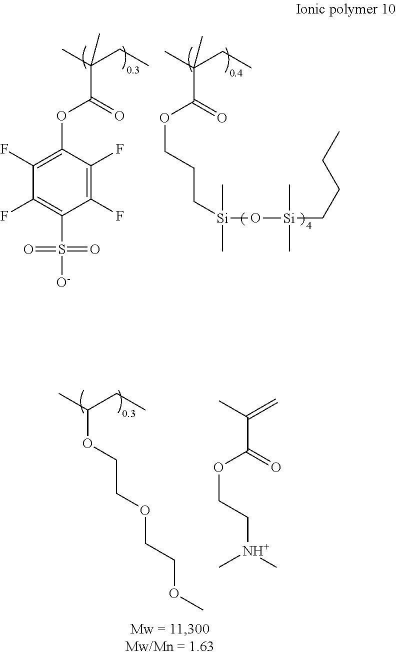

The repeating number in formula shows the average value.

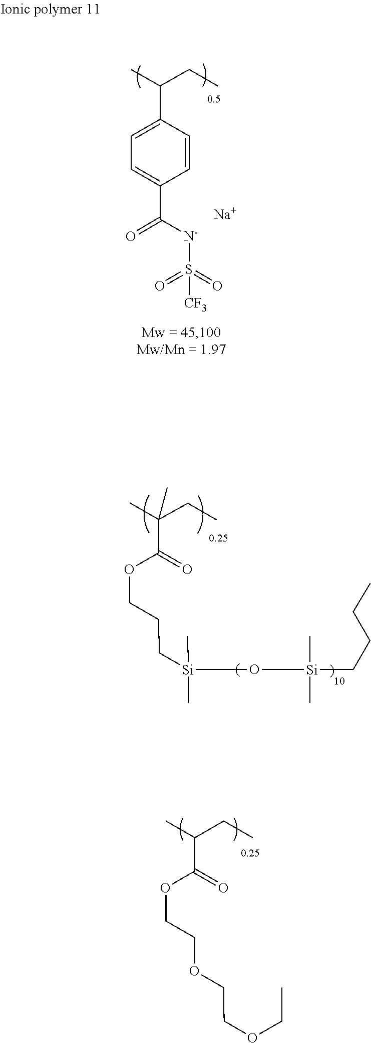

The repeating number in formula shows the average value.

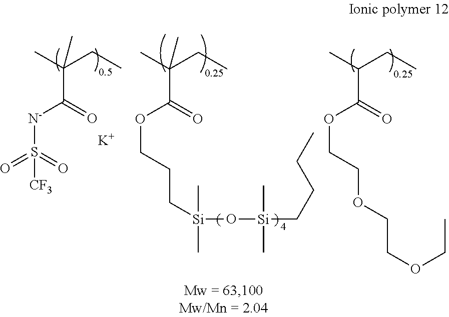

The repeating number in formula shows the average value.

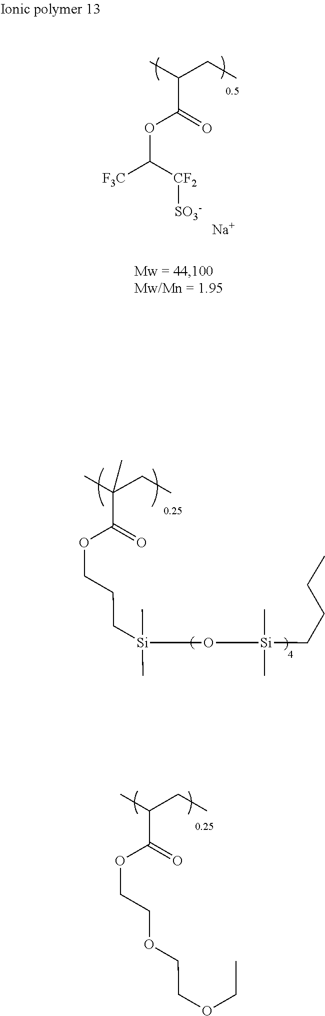

The repeating number in formula shows the average value.

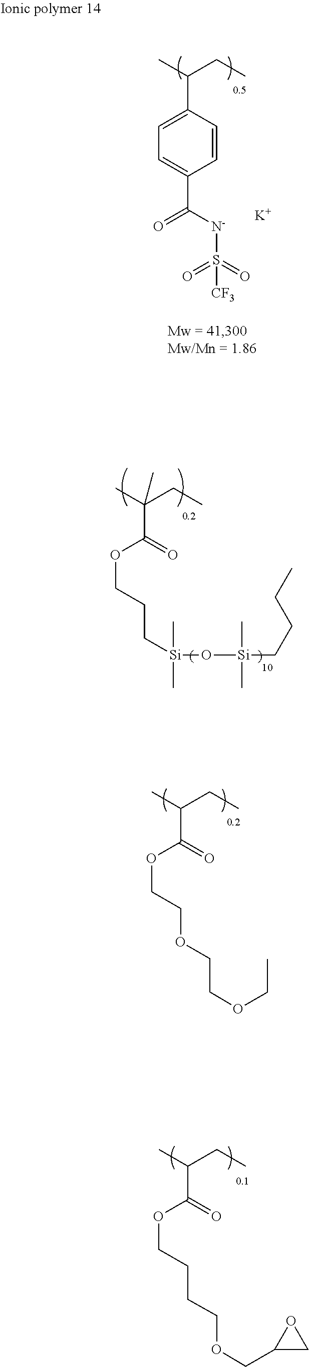

The repeating number in formula shows the average value.

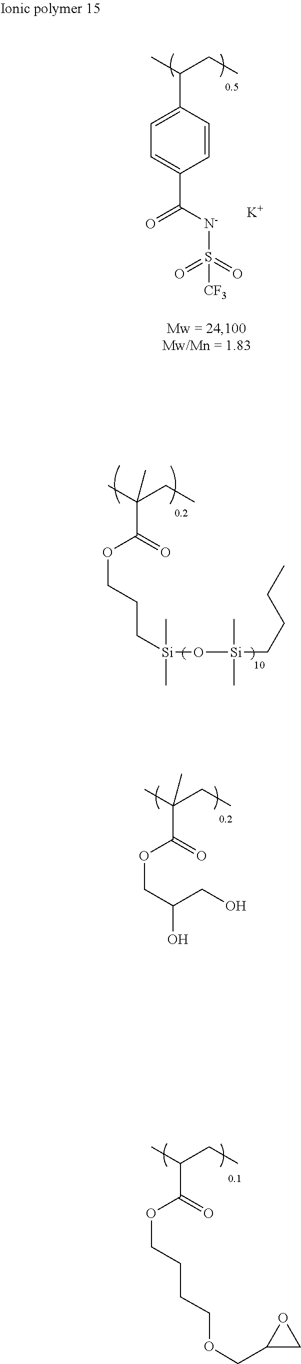

The repeating number in formula shows the average value.

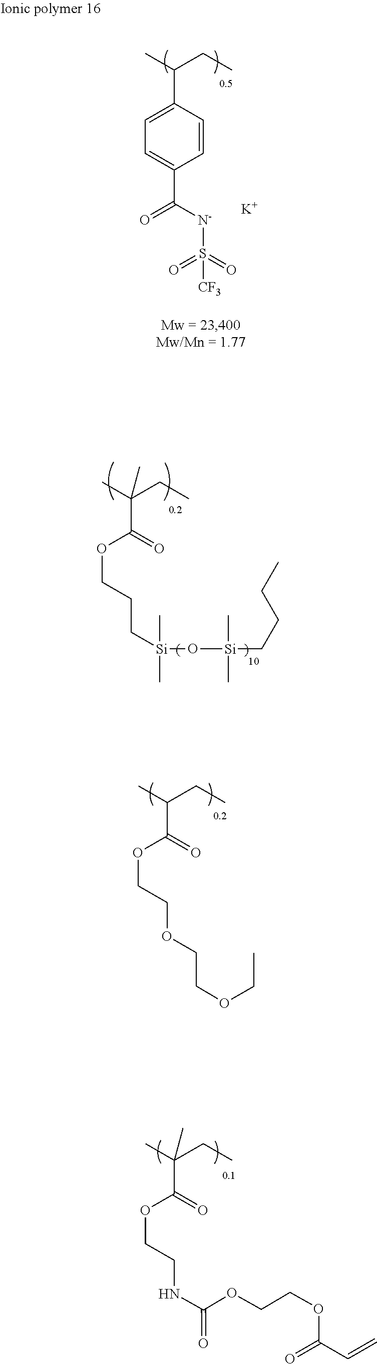

The repeating number in formula shows the average value.

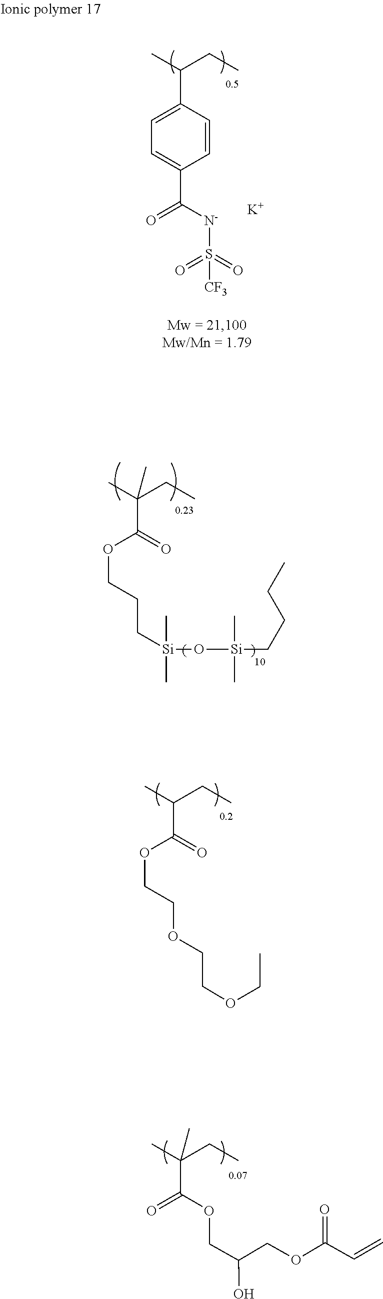

The repeating number in formula shows the average value.

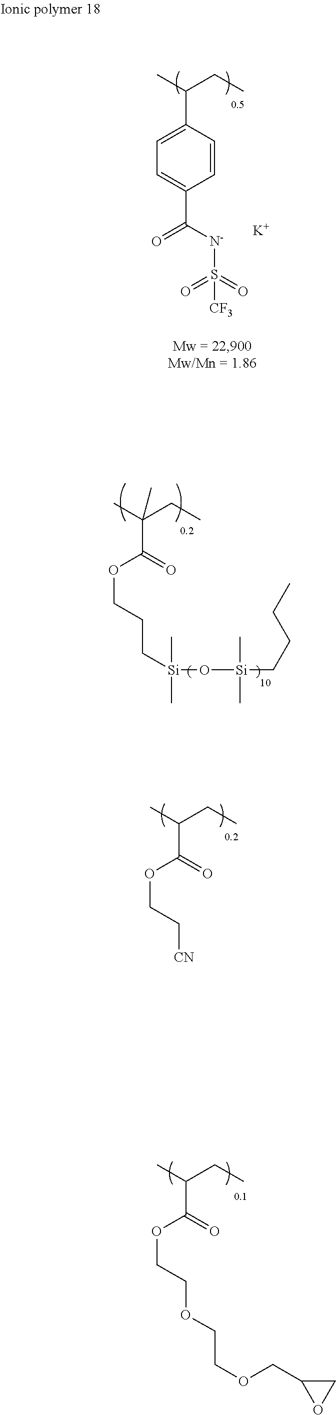

The repeating number in formula shows the average value.

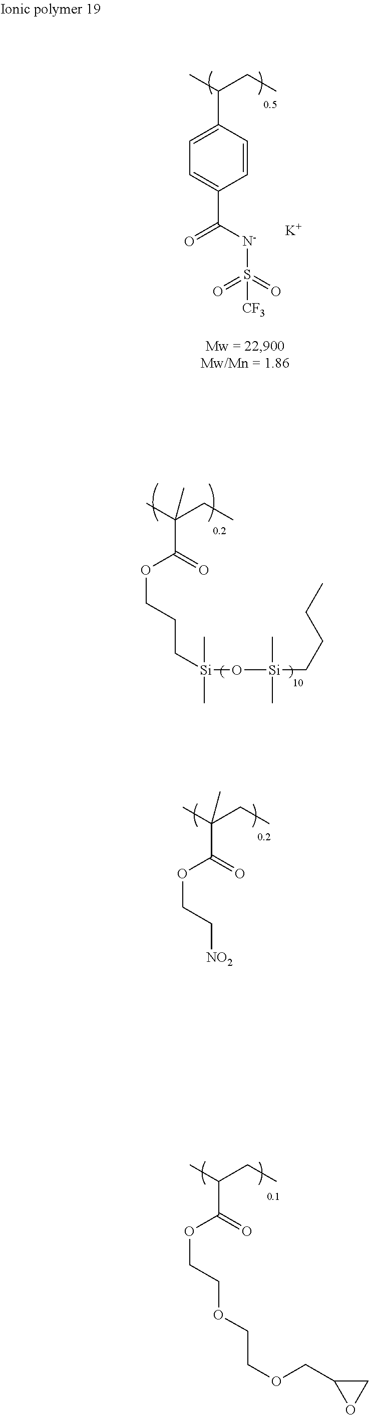

The repeating number in formula shows the average value.

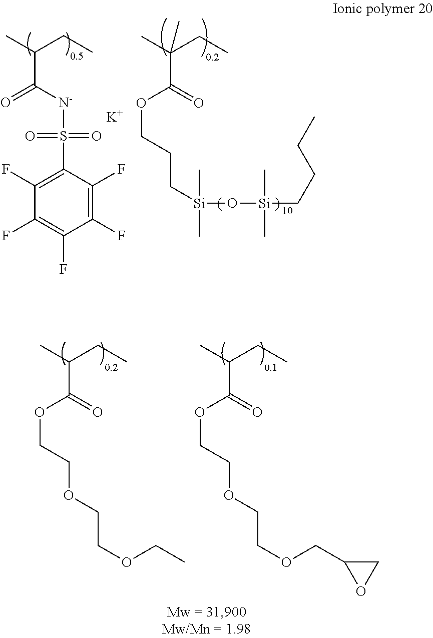

The repeating number in formula shows the average value.

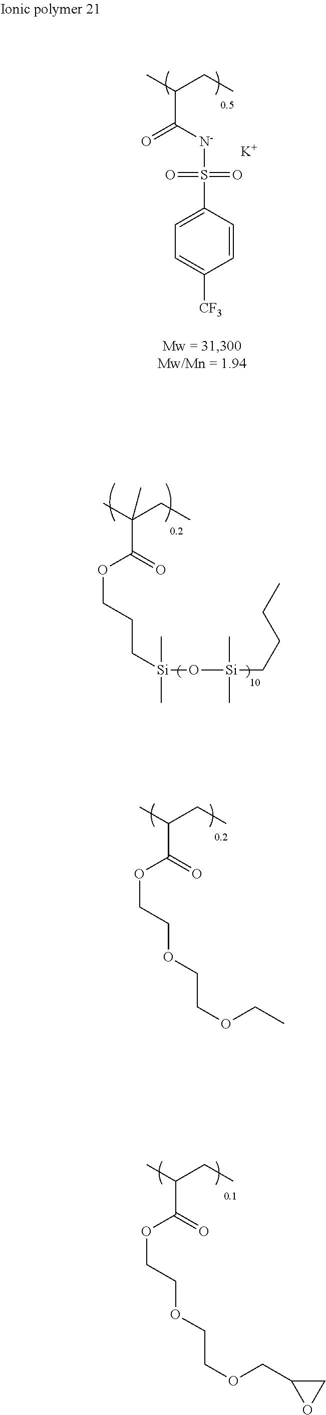

The repeating number in formula shows the average value.

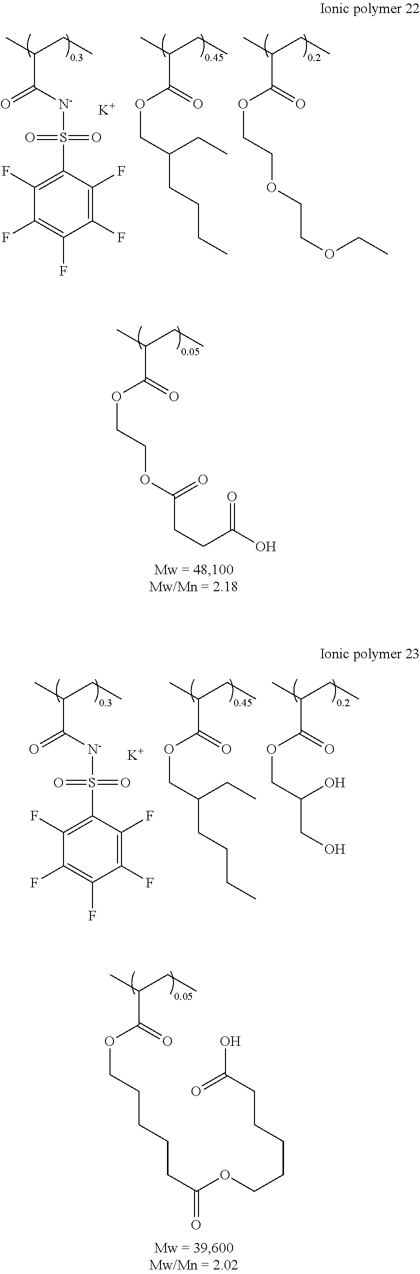

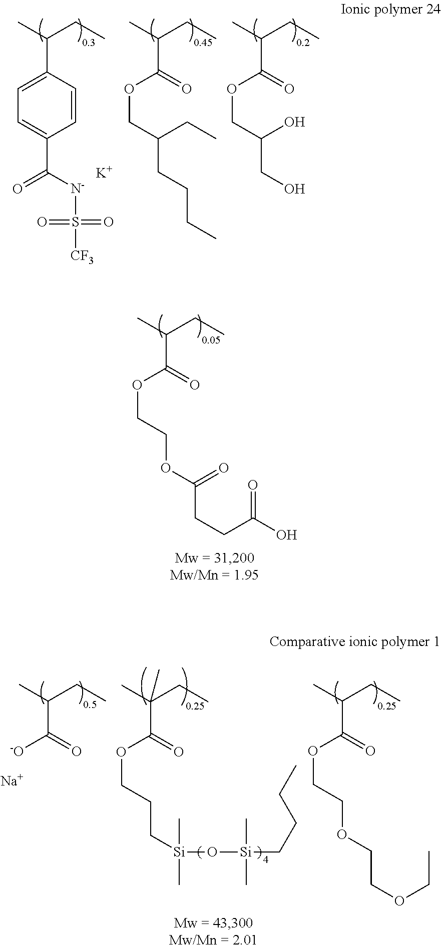

The repeating number in formula shows the average value.

### (Measurement of Thickness of Ionic Polymer Containing Layer)

Ionic polymer solutions shown in Tables 1 and 2 were prepared, applied to a Si substrate by spin coating, baked on a hot plate at 120° C. for 10 minutes, and the film thickness was measured using an optical film thickness meter. The results are shown in Tables 1 and 2.

### (Preparation of Electro-Conductive Substrate)

A polyimide film having a thickness of 25 μm was coated with Dotite FA-333, an electro-conductive paste manufactured by Fujikura Kasei, by screen printing, and baked in an oven at 120° C. for 10 minutes to produce a conductive substrate X having 100 printed straight lines as shown in FIG. 4, which was 4 cm in length, 50 μm in width, and 150 μm in line spacing.

Silver nanowire solution (diameter 60 nm, length 40 μm, concentration 5 mg/mL) manufactured by Sigma-Aldrich was diluted 6 times with pure water, was coated by spin coating on a silicon wafer on which a 25 μm thick polyimide film was placed, and was baked on a hot plate at 250° ° C. for 10 minutes to fuse silver nanowires to produce a conductive substrate Y.

A polyimide film having a thickness of 25 μm was coated with Dotite FA-333, an electro-conductive paste manufactured by Fujikura Kasei, by screen printing, and baked in an oven at 120° ° C. for 10 minutes to produce a comparative conductive substrate having 50 printed straight lines as shown in FIG. 4, which was 4 cm in length, 250 μm in width, and 150 μm in line spacing.

### (Preparation of Bio-Electrode)

Bio-electrodes were prepared based on the combinations shown in Table 3. Specifically, a fluororesin tape with adhesive was pasted on a part of the electro-conductive wiring on the conductive substrates X and Y and the comparative conductive substrate, and an ionic polymer solution was spin-coated on it. It was baked on a hot plate at 120° ° C. for 10 minutes, and the fluororesin tape with adhesive was peeled off.

For polymer solutions 5 to 10, 12, 16, and 17, the ionic polymer containing layer were cured by irradiating with light of 500 mJ/cm² using a 1,000 W xenon lamp in a nitrogen atmosphere.

Conductive substrate on which the ionic polymer containing layer 6 was formed was cut so that both a contact area to the skin and a masked area should be a 2 cm square, as shown in FIG. 14.

### (Measurement of Biological Signals)

A cellophane adhesive tape was attached on the back side of the bio-electrode, and the bio-electrodes were attached to the locations on the arm shown in FIG. 15, which were treated with absorbent cotton moistened with water. The exposed electro-conductive wiring in the masked area was sandwiched by a metal clip, and the metal clip were connected to the ECG measurement device using electro-conductive wiring.

Nexus10 MARKII manufactured by Kissei Comtec Co., Ltd. was used for ECG measuring device. In FIG. 15, the numbered 11 is for a positive electrode, 12 for a negative electrode, and 13 for an earth. Table 3 shows the results.

### (Measurement of Transparency)

The ionic polymer containing layers was formed on the conductive substrate and its transmittance at a wavelength of 600 nm was measured. Difference of measured values between the conductive substrate and a substrate, which was electro-conductive wiring and ionic polymer were not coated, was defined as transmittance of the bio-electrode. Table 3 shows the results.

### [Resin]

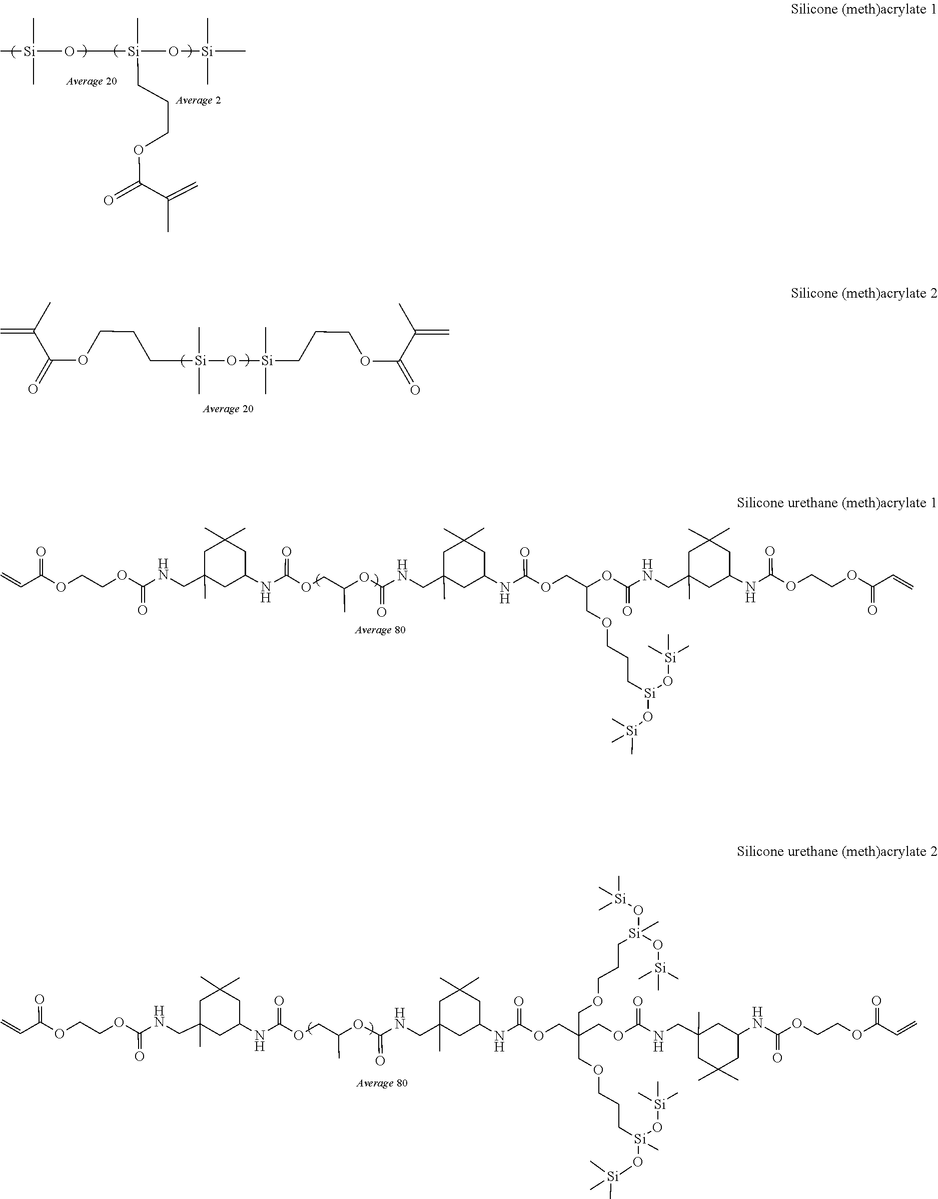

Polyglycerin acrylate: SYntechSA-TE6 (Manufactured by Sakamoto Yakuhin kogyo Co., Ltd.)

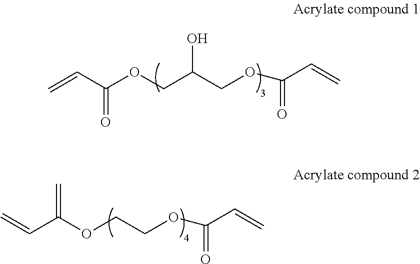

### [Additive]

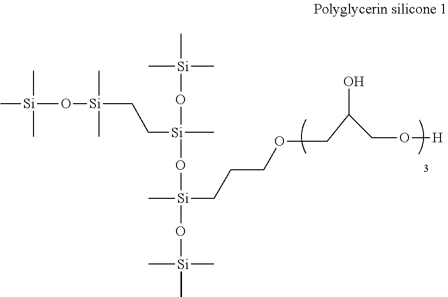 [0209] Irgacure TPO
2-ethyl-4-imidasol

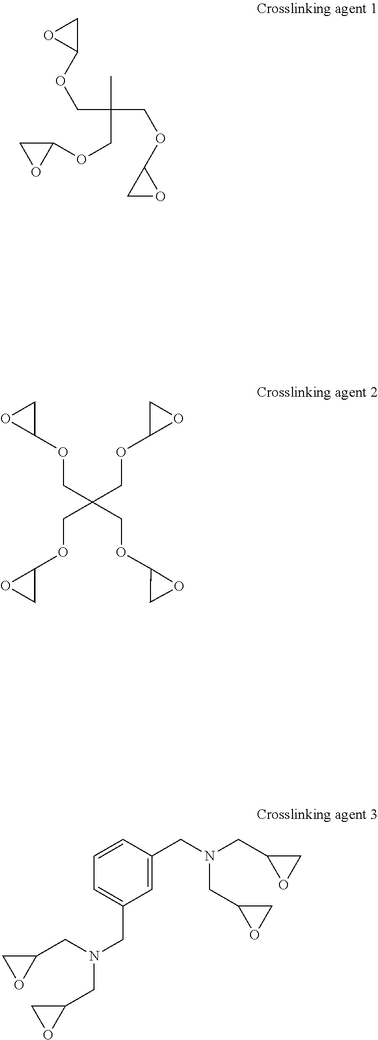 [SMILES: CC(COC1CO1)(COC1CO1)COC1CO1 ; C1OC1OCC(COC1CO1)(COC1CO1)COC1CO1 ; c1cc(CN(CC2CO2)CC2CO2)cc(CN(CC2CO2)CC2CO2)c1]

### [Solvent]

PGEE: Propylene glycol monomethyl ether
BE: diethylene glycol monobutyl ether
GBL: Gamma butyrlactone
cyclopentanone
2-heptanone

TABLE 1

| | | | | | Layer (B) |
|---|---|---|---|---|---|
| Ionic polymer | Ionic polymer | Resin | Solvent | Additive | Thickness |
| solution | (parts by mass) | (parts by mass) | (parts by mass) | (parts by mass) | (μm) |
| Ionic polymer | Ionic polymer | Silicone (meth) | Cyclopentanone | Polyglycerin | 14 |
| solution 1 | 1 (100) | acrylate 1 (10) | (100) | silicone 1 (20) | |
| | | | | Irgacure TPO (1) | |
| Ionic polymer | Ionic polymer | Silicone (meth) | Cyclopentanone | Polyglycerin | 16 |
| solution 2 | 2 (100) | acrylate 2 (10) | (100) | silicone 1 (15) | |
| | | | | Irgacure TPO (1) | |
| Ionic polymer | Ionic polymer | Silicone urethane | PGEE (100) | Polyglycerin | 28 |
| solution 3 | 3 (100) | (meth) acrylate 1 | | silicone 1 (15) | |
| | | (10) | | Irgacure TPO (1) | |
| Ionic polymer | Ionic polymer | — | Cyclopentanone | — | 16 |
| solution 4 | 4 (100) | | (100) | | |
| Ionic polymer | Ionic polymer | Silicone urethane | Cyclopentanone | Irgacure TPO (1) | 31 |
| solution 5 | 5 (100) | (meth) acrylate 2 | (50) | | |
| | | (10) | 2-Heptanone (50) | | |
| Ionic polymer | Ionic polymer | Polyglycerin | Cyclopentanone | Irgacure TPO (1) | 16 |
| solution 6 | 6 (100) | acrylate (10) | (95) BE (5) | | |
| Ionic polymer | Ionic polymer | Polyglycerin | Cyclopentanone | 2-Ethyl-4- | 18 |
| solution 7 | 7 (100) | acrylate (10) | (100) | imidazole (2) | |
| | | | | Irgacure TPO (1) | |
| Ionic polymer | Ionic polymer | Acrylate | Cyclopentanone | Irgacure TPO (1) | 21 |
| solution 8 | 8 (100) | compound 1 (5) | (90) GBL (10) | | |
| Ionic polymer | Ionic polymer | Acrylate | Cyclopentanone | Polyglycerin | 16 |
| solution 9 | 9 (100) | compound 2 (5) | (100) | silicone 1 (5) | |
| | | | | Irgacure TPO (1) | |
| Ionic polymer | Ionic polymer | — | Cyclopentanone | Polyglycerin | 13 |
| solution 10 | 10 (100) | | (100) | silicone 1 (5) | |
| | | | | Irgacure TPO (1) | |
| Ionic polymer | Ionic polymer | — | Cyclopentanone | Polyglycerin | 18 |
| solution 11 | 11 (100) | | (100) | silicone 1 (2) | |
| Ionic polymer | Ionic polymer | — | Cyclopentanone | — | 12 |
| solution 12 | 12 (100) | | (100) | | |
| Ionic polymer | Ionic polymer | — | Cyclopentanone | Polyglycerin | 12 |
| solution 13 | 13 (100) | | (100) | silicone 1 (4) | |
| Ionic polymer | Ionic polymer | — | Cyclopentanone | 2-Ethyl-4- | 13 |
| solution 14 | 14 (100) | | (100) | imidazole (2) | |
| Ionic polymer | Ionic polymer | — | Cyclopentanone | 2-Ethyl-4- | 15 |
| solution 15 | 15 (100) | | (100) | imidazole (2) | |

TABLE 2

| | | | | | Layer (B) |
|---|---|---|---|---|---|
| Ionic polymer | Ionic polymer | Resin | Solvent | Additive | Thickness |
| solution | (parts by mass) | (parts by mass) | (parts by mass) | (parts by mass) | (μm) |
| Ionic polymer | Ionic polymer | — | Cyclopentanone | Polyglycerin | 21 |
| Solution 16 | 16 (100) | | (100) | silicone 1 (4) | |
| | | | | Irgacure TPO (1) | |
| Ionic polymer | Ionic polymer | — | Cyclopentanone | Polyglycerin | 22 |
| solution 17 | 17 (100) | | (100) | silicone 1 (4) | |
| | | | | Irgacure TPO (1) | |
| Ionic polymer | Ionic polymer | — | Cyclopentanone | Polyglycerin | 26 |
| solution 18 | 18 (100) | | (100) | silicone 1 (4) | |
| | | | | 2-Ethyl-4- | |
| | | | | imidasol (2) | |
| Ionic polymer | Ionic polymer | — | Cyclopentanone | Polyglycerin | 20 |
| solution 19 | 19 (100) | | (100) | silicone 1 (4) | |
| | | | | 2-Ethyl-4- | |
| | | | | imidasol (2) | |
| Ionic polymer | Ionic polymer | — | Cyclopentanone | Polyglycerin | 33 |
| solution 20 | 20 (100) | | (100) | silicone 1 (4) | |
| | | | | 2-Ethyl-4- | |
| | | | | imidasol (2) | |
| Ionic polymer | Ionic polymer | — | Cyclopentanone | Polyglycerin | 35 |
| solution 21 | 21 (100) | | (100) | silicone 1 (4) | |
| | | | | 2-Ethyl-4- | |
| | | | | imidasol (2) | |
| Ionic polymer | Ionic polymer | — | Cyclopentanone | Polyglycerin | 36 |
| solution 22 | 22 (100) | | (100) | silicone 1 (4) | |
| | | | | Crosslinking | |
| | | | | agent 1 (0.5) | |
| Ionic polymer | Ionic polymer | — | Cyclopentanone | Crosslinking | 32 |
| solution 23 | 23 (100) | | (100) | agent 2 (0.03) | |
| Ionic polymer | Ionic polymer | — | Cyclopentanone | Crosslinking | 31 |
| solution 24 | 24 (100) | | (100) | agent 3 (0.2) | |
| Comparative | Comparative | — | Cyclopentanone | — | 16 |
| Ionic polymer | Ionic polymer | | (100) | | |
| solution 1 | 1 (100) | | | | |

TABLE 3

| Example/ | | | | |
|---|---|---|---|---|
| Comparative | Ionic polymer | Conductive | Transmittance | ECG |
| example | solution | substrate | at 600 nm (%) | signal |
| Example 1 | Ionic polymer | Conductive | 73 | Good |
| | solution 1 | substrate X | | |
| Example 2 | Ionic polymer | Conductive | 72 | Good |
| | solution 2 | substrate X | | |
| Example 3 | Ionic polymer | Conductive | 71 | Good |
| | solution 3 | substrate X | | |
| Example 4 | Ionic polymer | Conductive | 74 | Good |
| | solution 4 | substrate X | | |
| Example 5 | Ionic polymer | Conductive | 73 | Good |
| | solution 5 | substrate X | | |
| Example 6 | Ionic polymer | Conductive | 73 | Good |
| | solution 6 | substrate X | | |
| Example 7 | Ionic polymer | Conductive | 72 | Good |
| | solution 7 | substrate X | | |
| Example 8 | Ionic polymer | Conductive | 72 | Good |
| | solution 8 | substrate X | | |
| Example 9 | Ionic polymer | Conductive | 73 | Good |
| | solution 9 | substrate X | | |
| Example 10 | Ionic polymer | Conductive | 72 | Good |
| | solution 10 | substrate X | | |
| Example 11 | Ionic polymer | Conductive | 71 | Good |
| | solution 11 | substrate X | | |
| Example 12 | Ionic polymer | Conductive | 72 | Good |
| | solution 12 | substrate X | | |
| Example 13 | Ionic polymer | Conductive | 73 | Good |
| | solution 13 | substrate X | | |
| Example 14 | Ionic polymer | Conductive | 72 | Good |
| | solution 14 | substrate X | | |
| Example 15 | Ionic polymer | Conductive | 70 | Good |
| | solution 15 | substrate X | | |
| Example 16 | Ionic polymer | Conductive | 72 | Good |
| | solution 16 | substrate X | | |
| Example 17 | Ionic polymer | Conductive | 73 | Good |
| | solution 17 | substrate X | | |
| Example 18 | Ionic polymer | Conductive | 72 | Good |
| | solution 18 | substrate X | | |
| Example 19 | Ionic polymer | Conductive | 72 | Good |
| | solution 19 | substrate X | | |
| Example 20 | Ionic polymer | Conductive | 90 | Good |
| | solution 19 | substrate Y | | |
| Example 21 | Ionic polymer | Conductive | 72 | Good |
| | solution 20 | substrate X | | |
| Example 22 | Ionic polymer | Conductive | 71 | Good |
| | solution 21 | substrate X | | |
| Example 23 | Ionic polymer | Conductive | 72 | Good |
| | solution 22 | substrate X | | |
| Example 24 | Ionic polymer | Conductive | 72 | Good |
| | solution 23 | substrate X | | |
| Example 25 | Ionic polymer | Conductive | 73 | Good |
| | solution 24 | substrate X | | |
| Comparative | Comparative | Conductive | 72 | Bad |
| Example 1 | Ionic polymer | substrate X | | |
| | solution 1 | | | |
| Comparative | Ionic polymer | Comparative | 40 | Good |
| Example 2 | solution 1 | conductive | | |
| | | substrate | | |

ECG signal was determined as “Good” when waves PORST of ECG signal were observed, and was determined as “Bad” when waves PORST of ECG signal were not observed.

From the results shown in Table 3, Examples 1 to 25 using the inventive bio-electrode had a good transmittance of 70% or more at a wavelength of 600 nm, and the waves PORST of the ECG signal was observed. From this, the inventive bio-electrode is thin and highly transparent, highly sensitive for biological signals, excellent in biocompatibility, lightweight, capable to be manufactured at low cost, capable of preventing significant reduction in the sensitivity to biological signals even when attached on the skin for a long time and when wetted with water or dried, and comfortable without itching, reddening, nor rash of skin.

On the other hand, in the case of Comparative Example 1 using the comparative ionic polymer solution 1 which does not contain ionic polymer having specific structure, the waves PORST of the ECG signal was not observed. In the case of Comparative Example 2 using the comparative conductive substrate having the electro-conductive wiring of 250 μm width, the transmittance at 600 nm of wavelength was poor.

The present description includes the following embodiments.

[1]: A bio-electrode has layers on a substrate, the layers including:
(A) an electro-conductive layer including an electro-conductive wiring having a width of 200 μm or less; and
(B) an ionic polymer containing layer including a polymer including a repeating unit-a having at least one selected from fluorosulfonic acid, fluorosulfonimide, and N-carbonyl-fluorosulfonamide, and having the weight-average molecular weight in a range of 1,000 to 500,000.
   [2]: The bio-electrode of the above [1], wherein a laminate film, being a combination of the layer (A) and the layer (B), has a visible light transmittance of 50% or more.
   [3]: The bio-electrode of the above [1] or [2], wherein the electro-conductive wiring having a width of 200 μm or less is a printed pattern formed by an electro-conductive paste including a particle of gold, silver, copper, or nickel, or a fusion layer of a metal nanowire including gold, silver, copper, nickel, or alloy thereof.
   [4]: The bio-electrode any one of the above [1] to [3], wherein the repeating unit-a has a partial structure shown by any of the following general formulae (1)-1 to (1)-4,

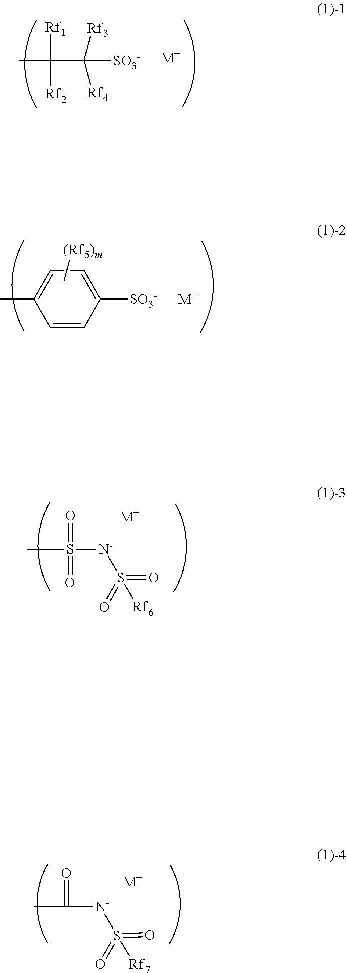

wherein Rf₁ and Rf₂ each represent a hydrogen atom, a fluorine atom, an oxygen atom, a methyl group, or a trifluoromethyl group, provided that when Rf₁ and Rf₂ represent an oxygen atom, the single oxygen atom represented by Rf₁ and Rf₂ bonds to a single carbon atom to form a carbonyl group; Rf₃ and Rf₄ each represent a hydrogen atom, a fluorine atom, or a trifluoromethyl group, and at least one of Rf₁ to Rf₄ is a fluorine atom or a trifluoromethyl group; Rf₅, Rf₆, and Rf₇ each represent a fluorine atom, a trifluoromethyl group, or a linear or branched alkyl group having 1 to 4 carbon atoms, or an aryl group having 6 to 10 carbon atoms and have at least one fluorine atom or trifluoromethyl group; and M⁺ represents an ion selected from the group consisting of an ammonium ion, a sodium ion, and a potassium ion; “m” represents an integer of 1 to 4.
[5]: The bio-electrode composition of the above [4], wherein the repeating unit-a includes at least one selected from the group consisting of repeating units A1 to A7 shown by the following general formula (2),

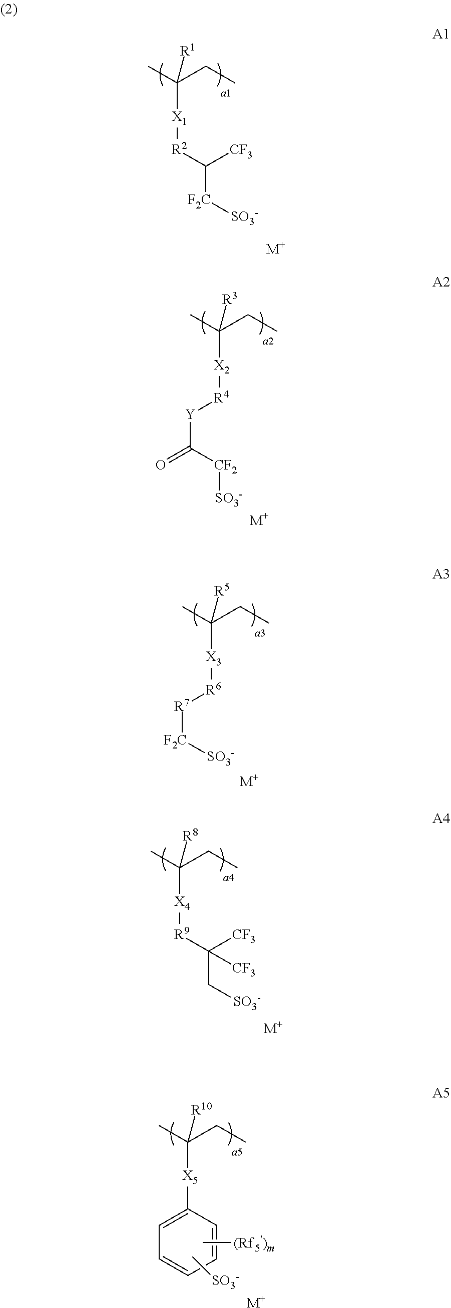

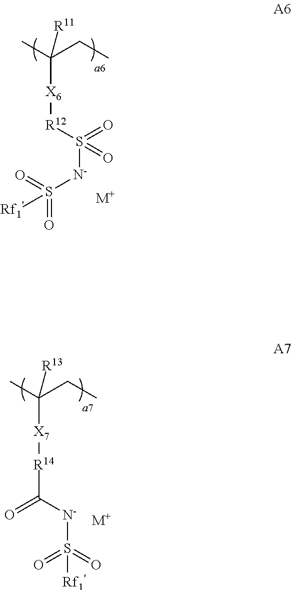

wherein, R¹, R³, R⁵, R⁸, R¹⁰, R¹¹, and R¹³ each independently represent a hydrogen atom or a methyl group; R², R⁴, R⁶, R⁹, R¹², and R¹⁴ each independently represent a single bond or a linear, branched, or cyclic hydrocarbon group having 1 to 13 carbon atoms, the hydrocarbon group optionally having either or both of an ester group and an ether group; R⁷ represents a linear or branched alkylene group having 1 to 4 carbon atoms, and one or two hydrogen atoms in R⁷ are optionally substituted with a fluorine atom; X₁, X₂, X₃, X₄, X₆, and X₇ each independently represent any of a single bond, a phenylene group, a naphthylene group, an ether group, an ester group, and an amide group; X₅ represents any of a single bond, an ether group, and an ester group; Y represents an oxygen atom or a —NR¹⁹— group; R¹⁹ represents any of a hydrogen atom, a linear, branched, or cyclic alkyl group having 1 to 12 carbon atoms, and phenyl group, optionally include one or more group selected from the group consisting of ether group, carbonyl group, ester group, and amide group; and Y forms a ring together with R⁴; Rf₁′ and Rf₅′ each represent a fluorine atom, a trifluoromethyl group, or a linear or branched alkyl group having 1 to 4 carbon atoms, and have at least one fluorine atom; “m” represents an integer of 1 to 4; a1, a2, a3, a4, a5, a6, and a7 satisfy 0≤a1≤1.0, 0≤a2≤1.0, 0≤a3≤1.0, 0≤a4≤1.0, 0≤a5≤1.0, 0≤a6≤1.0, 0≤a7≤1.0, and 0<a1+a2+a3+a4+a5+a6+a7≤1.0; and M⁺ represents an ion selected from the group consisting of an ammonium ion, a sodium ion, and a potassium ion.
[6]: The bio-electrode according of the above [4] or [5], wherein the repeating unit-a includes an ammonium ion shown by the following general formula (3) as an ammonium ion for forming an ammonium salt,

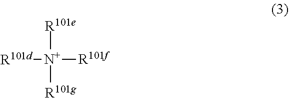

wherein, R^{101d}, R^{101e}, R^{101f}, and R^{101g} each represent a hydrogen atom, a linear, branched, or cyclic alkyl group having 1 to 15 carbon atoms, a linear, branched, or cyclic alkenyl group or alkynyl group having 2 to 12 carbon atoms, or an aromatic group having 4 to 20 carbon atoms, and optionally have one or more selected from the group consisting of an ether group, a carbonyl group, an ester group, a hydroxy group, a carboxy group, an amino group, a nitro group, a sulfonyl group, a sulfinyl group, a halogen atom, and a sulfur atom; and R^{101d} and R^{101e}, or R^{101d}, R^{101e}, and R^{101f}, are optionally bonded to each other together with a nitrogen atom bonded therewith to form a ring in which R^{101d} and R^{101e}, or R^{101d}, R^{101e}, and R^{101f}, represent an alkylene group having 3 to 10 carbon atoms, or to form a heteroaromatic ring having the nitrogen atom in the general formula (3) within the ring.
[7]: The bio-electrode of any one of the above [1] to [6], further including one or more resin (C) selected from the group consisting of (meth)acrylate resin, (meth)acrylamide resin, urethane resin, polyurethane (meth)acrylate, polyvinyl alcohol, polyvinylpyrrolidone, polyoxazoline, polyglycerin, polyglycerin-modified silicone, polyglycerin(meth)acrylate, cellulose, polyethylene glycol, and polypropylene glycol, as a component of the layer (B).
[8]: A method for manufacturing a bio-electrode according to any one of [1] to [7], the method including:
forming the layer (A) on a substrate by applying a solution including a metal nanowire, or by printing an electro-conductive paste containing a conductive particle; and forming the layer (B) on the layer (A) by coating a bio-electrode composition including the polymer.

It should be noted that the present invention is not limited to the above-described embodiments. The embodiments are just examples, and any examples that have substantially the same feature and demonstrate the same functions and effects as those in the technical concept disclosed in claims of the present invention are included in the technical scope of the present invention.

1. A bio-electrode has layers on a substrate, the layers comprising:
(A) an electro-conductive layer comprising an electro-conductive wiring having a width of 200 μm or less; and
(B) an ionic polymer containing layer comprising a polymer comprising a repeating unit-a having at least one selected from fluorosulfonic acid, fluorosulfonimide, and N-carbonyl-fluorosulfonamide, and having the weight-average molecular weight in a range of 1,000 to 500,000.

2. The bio-electrode according to claim 1, wherein a laminate film, being a combination of the layer (A) and the layer (B), has a visible light transmittance of 50% or more.

3. The bio-electrode according to claim 1, wherein the electro-conductive wiring having a width of 200 μm or less is a printed pattern formed by an electro-conductive paste comprising a particle of gold, silver, copper, or nickel, or a fusion layer of a metal nanowire comprising gold, silver, copper, nickel, or alloy thereof.

4. The bio-electrode according to claim 2, wherein the electro-conductive wiring having a width of 200 μm or less is a printed pattern formed by an electro-conductive paste comprising a particle of gold, silver, copper, or nickel, or a fusion layer of a metal nanowire comprising gold, silver, copper, nickel, or alloy thereof.

5. The bio-electrode according to claim 1, wherein the repeating unit-a has a partial structure shown by any of the following general formulae (1)-1 to (1)-4, 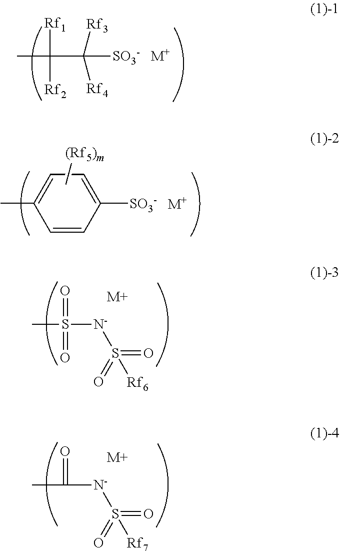
wherein Rf₁ and Rf₂ each represent a hydrogen atom, a fluorine atom, an oxygen atom, a methyl group, or a trifluoromethyl group, provided that when Rf₁ and Rf₂ represent an oxygen atom, the single oxygen atom represented by Rf₁ and Rf₂ bonds to a single carbon atom to form a carbonyl group; Rf₃ and Rf₄ each represent a hydrogen atom, a fluorine atom, or a trifluoromethyl group, and at least one of Rf₁ to Rf₄ is a fluorine atom or a trifluoromethyl group; Rf₅, Rf₆, and Rf₇ each represent a fluorine atom, a trifluoromethyl group, a linear or branched alkyl group having 1 to 4 carbon atoms, or an aryl group having 6 to 10 carbon atoms, and have at least one fluorine atom or trifluoromethyl group; and M⁺ represents an ion selected from the group consisting of an ammonium ion, a sodium ion, and a potassium ion; “m” represents an integer of 1 to 4.

6. The bio-electrode according to claim 2, wherein the repeating unit-a has a partial structure shown by any of the following general formulae (1)-1 to (1)-4, 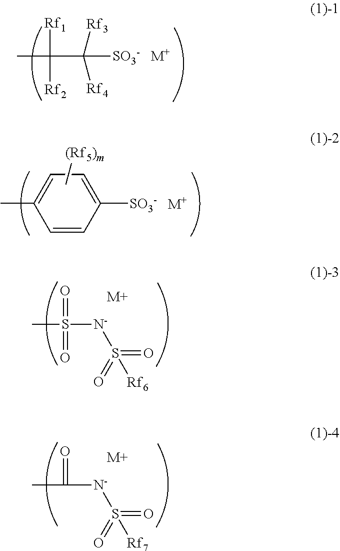
wherein Rf₁ and Rf₂ each represent a hydrogen atom, a fluorine atom, an oxygen atom, a methyl group, or a trifluoromethyl group, provided that when Rf₁ and Rf₂ represent an oxygen atom, the single oxygen atom represented by Rf₁ and Rf₂ bonds to a single carbon atom to form a carbonyl group; Rf₃ and Rf₄ each represent a hydrogen atom, a fluorine atom, or a trifluoromethyl group, and at least one of Rf₁ to Rf₄ is a fluorine atom or a trifluoromethyl group; Rf₅, Rf₆, and Rf₇ each represent a fluorine atom, a trifluoromethyl group, a linear or branched alkyl group having 1 to 4 carbon atoms, or an aryl group having 6 to 10 carbon atoms, and have at least one fluorine atom or trifluoromethyl group; and M⁺ represents an ion selected from the group consisting of an ammonium ion, a sodium ion, and a potassium ion; “m” represents an integer of 1 to 4.

7. The bio-electrode according to claim 3, wherein the repeating unit-a has a partial structure shown by any of the following general formulae (1)-1 to (1)-4, 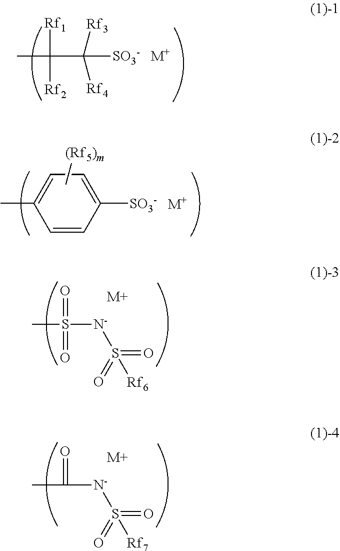
wherein Rf₁ and Rf₂ each represent a hydrogen atom, a fluorine atom, an oxygen atom, a methyl group, or a trifluoromethyl group, provided that when Rf₁ and Rf₂ represent an oxygen atom, the single oxygen atom represented by Rf₁ and Rf₂ bonds to a single carbon atom to form a carbonyl group; Rf₃ and Rf₄ each represent a hydrogen atom, a fluorine atom, or a trifluoromethyl group, and at least one of Rf₁ to Rf₄ is a fluorine atom or a trifluoromethyl group; Rf₅, Rf₆, and Rf₇ each represent a fluorine atom, a trifluoromethyl group, a linear or branched alkyl group having 1 to 4 carbon atoms, or an aryl group having 6 to 10 carbon atoms, and have at least one fluorine atom or trifluoromethyl group; and M⁺ represents an ion selected from the group consisting of an ammonium ion, a sodium ion, and a potassium ion; “m” represents an integer of 1 to 4.

8. The bio-electrode according to claim 4, wherein the repeating unit-a has a partial structure shown by any of the following general formulae (1)-1 to (1)-4, 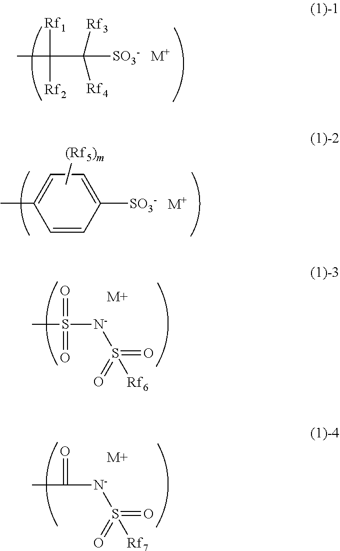
wherein Rf₁ and Rf₂ each represent a hydrogen atom, a fluorine atom, an oxygen atom, a methyl group, or a trifluoromethyl group, provided that when Rf₁ and Rf₂ represent an oxygen atom, the single oxygen atom represented by Rf₁ and Rf₂ bonds to a single carbon atom to form a carbonyl group; Rf₃ and Rf₄ each represent a hydrogen atom, a fluorine atom, or a trifluoromethyl group, and at least one of Rf₁ to Rf₄ is a fluorine atom or a trifluoromethyl group; Rf₅, Rf₆, and Rf₇ each represent a fluorine atom, a trifluoromethyl group, a linear or branched alkyl group having 1 to 4 carbon atoms, or an aryl group having 6 to 10 carbon atoms, and have at least one fluorine atom or trifluoromethyl group; and M⁺ represents an ion selected from the group consisting of an ammonium ion, a sodium ion, and a potassium ion; “m” represents an integer of 1 to 4.

9. The bio-electrode composition according to claim 5, wherein the repeating unit-a comprises at least one selected from the group consisting of repeating units A1 to A7 shown by the following general formula (2), 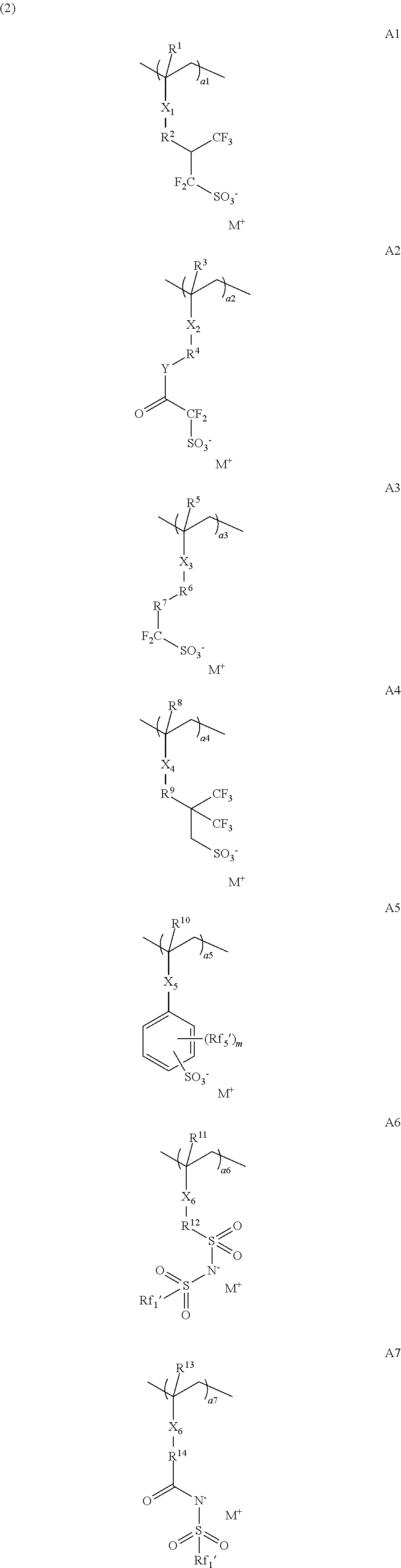
wherein, R¹, R³, R⁵, R⁸, R¹⁰, R¹¹, and R¹³ each independently represent a hydrogen atom or a methyl group; R², R⁴, R⁶, R⁹, R¹², and R¹⁴ each independently represent a single bond or a linear, branched, or cyclic hydrocarbon group having 1 to 13 carbon atoms, the hydrocarbon group optionally having either or both of an ester group and an ether group; R⁷ represents a linear or branched alkylene group having 1 to 4 carbon atoms, and one or two hydrogen atoms in R⁷ are optionally substituted with a fluorine atom; X₁, X₂, X₃, X₄, X₆, and X₇ each independently represent any of a single bond, a phenylene group, a naphthylene group, an ether group, an ester group, and an amide group; X₅ represents any of a single bond, an ether group, and an ester group; Y represents an oxygen atom or a —NR¹⁹— group; R¹⁹ represents any of a hydrogen atom, a linear, branched, or cyclic alkyl group having 1 to 12 carbon atoms, and phenyl group, optionally comprise one or more group selected from the group consisting of ether group, carbonyl group, ester group, and amide group; and Y forms a ring together with R⁴; Rf₁′ and Rf₅′ each represent a fluorine atom, a trifluoromethyl group, or a linear or branched alkyl group having 1 to 4 carbon atoms, and have at least one fluorine atom; “m” represents an integer of 1 to 4; a1, a2, a3, a4, a5, a6, and a7 satisfy 0≤a1≤1.0, 0≤a2≤1.0, 0≤a3≤1.0, 0≤a4≤1.0, 0≤a5≤1.0, 0≤a6≤1.0, 0≤a7≤1.0, and 0<a1+a2+a3+a4+a5+a6+a7≤1.0; and M⁺ represents an ion selected from the group consisting of an ammonium ion, a sodium ion, and a potassium ion.

10. The bio-electrode composition according to claim 6, wherein the repeating unit-a comprises at least one selected from the group consisting of repeating units A1 to A7 shown by the following general formula (2), 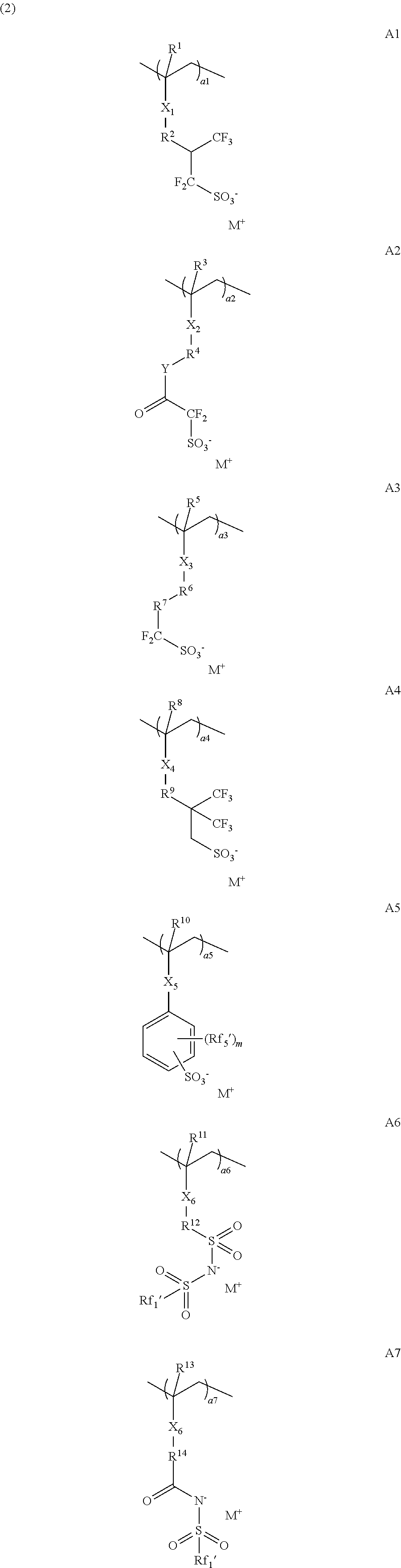
wherein, R¹, R³, R⁵, R⁸, R¹⁰, R¹¹, and R¹³ each independently represent a hydrogen atom or a methyl group; R², R⁴, R⁶, R⁹, R¹², and R¹⁴ each independently represent a single bond or a linear, branched, or cyclic hydrocarbon group having 1 to 13 carbon atoms, the hydrocarbon group optionally having either or both of an ester group and an ether group; R⁷ represents a linear or branched alkylene group having 1 to 4 carbon atoms, and one or two hydrogen atoms in R⁷ are optionally substituted with a fluorine atom; X₁, X₂, X₃, X₄, X₆, and X₇ each independently represent any of a single bond, a phenylene group, a naphthylene group, an ether group, an ester group, and an amide group; X₅ represents any of a single bond, an ether group, and an ester group; Y represents an oxygen atom or a —NR¹⁹— group; R¹⁹ represents any of a hydrogen atom, a linear, branched, or cyclic alkyl group having 1 to 12 carbon atoms, and phenyl group, optionally comprise one or more group selected from the group consisting of ether group, carbonyl group, ester group, and amide group; and Y forms a ring together with R⁴; Rf₁′ and Rf₅′ each represent a fluorine atom, a trifluoromethyl group, or a linear or branched alkyl group having 1 to 4 carbon atoms, and have at least one fluorine atom; “m” represents an integer of 1 to 4; a1, a2, a3, a4, a5, a6, and a7 satisfy 0≤a1≤1.0, 0≤a2≤1.0, 0≤a3≤1.0, 0≤a4≤1.0, 0≤a5≤1.0, 0≤a6≤1.0, 0≤a7≤1.0, and 0<a1+a2+a3+a4+a5+a6+a7≤1.0; and M⁺ represents an ion selected from the group consisting of an ammonium ion, a sodium ion, and a potassium ion.

11. The bio-electrode composition according to claim 7, wherein the repeating unit-a comprises at least one selected from the group consisting of repeating units A1 to A7 shown by the following general formula (2), 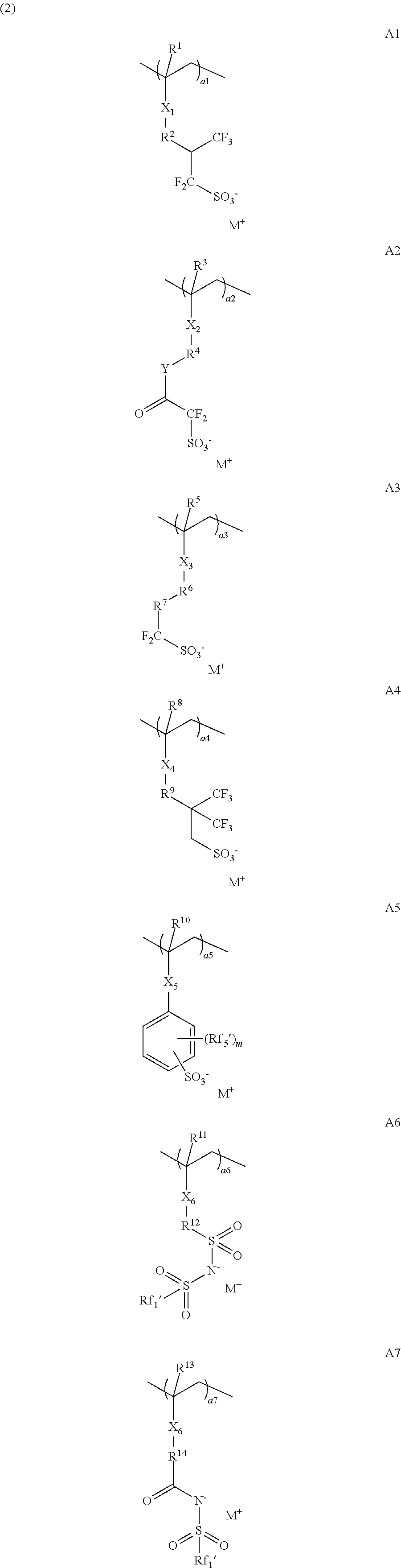
wherein, R¹, R³, R⁵, R⁸, R¹⁰, R¹¹, and R¹³ each independently represent a hydrogen atom or a methyl group; R², R⁴, R⁶, R⁹, R¹², and R¹⁴ each independently represent a single bond or a linear, branched, or cyclic hydrocarbon group having 1 to 13 carbon atoms, the hydrocarbon group optionally having either or both of an ester group and an ether group; R⁷ represents a linear or branched alkylene group having 1 to 4 carbon atoms, and one or two hydrogen atoms in R⁷ are optionally substituted with a fluorine atom; X₁, X₂, X₃, X₄, X₆, and X₇ each independently represent any of a single bond, a phenylene group, a naphthylene group, an ether group, an ester group, and an amide group; X₅ represents any of a single bond, an ether group, and an ester group; Y represents an oxygen atom or a —NR¹⁹— group; R¹⁹ represents any of a hydrogen atom, a linear, branched, or cyclic alkyl group having 1 to 12 carbon atoms, and phenyl group, optionally comprise one or more group selected from the group consisting of ether group, carbonyl group, ester group, and amide group; and Y forms a ring together with R⁴; Rf₁′ and Rf₅′ each represent a fluorine atom, a trifluoromethyl group, or a linear or branched alkyl group having 1 to 4 carbon atoms, and have at least one fluorine atom; “m” represents an integer of 1 to 4; a1, a2, a3, a4, a5, a6, and a7 satisfy 0≤a1≤1.0, 0≤a2≤1.0, 0≤a3≤1.0, 0≤a4≤1.0, 0≤a5≤1.0, 0≤a6≤1.0, 0≤a7≤1.0, and 0<a1+a2+a3+a4+a5+a6+a7≤1.0; and M⁺ represents an ion selected from the group consisting of an ammonium ion, a sodium ion, and a potassium ion.

12. The bio-electrode composition according to claim 8, wherein the repeating unit-a comprises at least one selected from the group consisting of repeating units A1 to A7 shown by the following general formula (2), 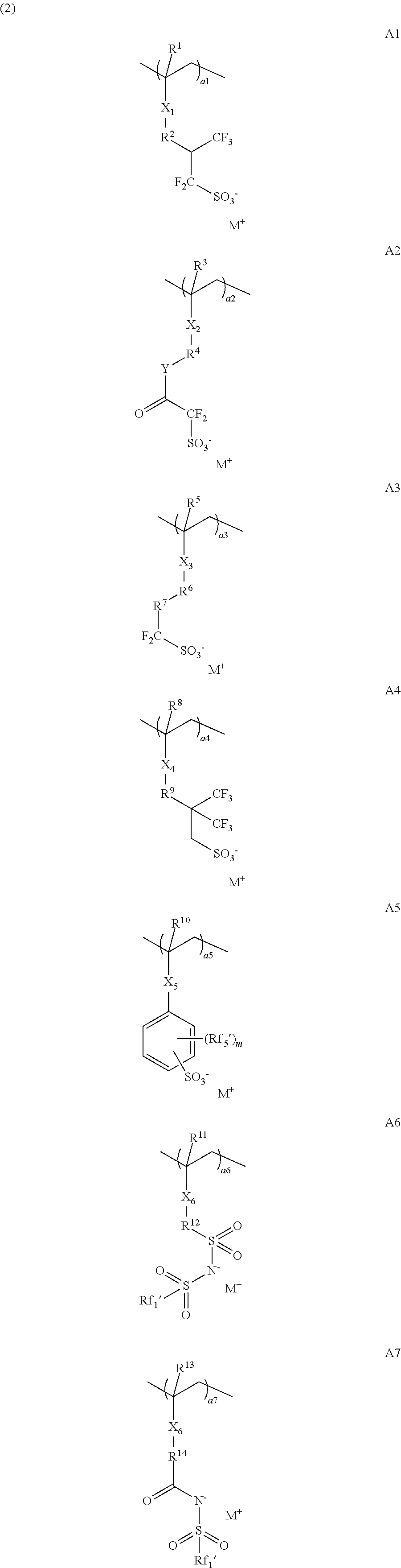
wherein, R¹, R³, R⁵, R⁸, R¹⁰, R¹¹, and R¹³ each independently represent a hydrogen atom or a methyl group; R², R⁴, R⁶, R⁹, R¹², and R¹⁴ each independently represent a single bond or a linear, branched, or cyclic hydrocarbon group having 1 to 13 carbon atoms, the hydrocarbon group optionally having either or both of an ester group and an ether group; R⁷ represents a linear or branched alkylene group having 1 to 4 carbon atoms, and one or two hydrogen atoms in R⁷ are optionally substituted with a fluorine atom; X₁, X₂, X₃, X₄, X₆, and X₇ each independently represent any of a single bond, a phenylene group, a naphthylene group, an ether group, an ester group, and an amide group; X₅ represents any of a single bond, an ether group, and an ester group; Y represents an oxygen atom or a —NR¹⁹— group; R¹⁹ represents any of a hydrogen atom, a linear, branched, or cyclic alkyl group having 1 to 12 carbon atoms, and phenyl group, optionally comprise one or more group selected from the group consisting of ether group, carbonyl group, ester group, and amide group; and Y forms a ring together with R⁴; Rf₁′ and Rf₅′ each represent a fluorine atom, a trifluoromethyl group, or a linear or branched alkyl group having 1 to 4 carbon atoms, and have at least one fluorine atom; “m” represents an integer of 1 to 4; a1, a2, a3, a4, a5, a6, and a7 satisfy 0≤a1≤1.0, 0a2≤1.0, 0≤a3≤1.0, 0≤a4≤1.0, 0≤a5≤1.0, 0≤a6≤1.0, 0≤a7≤1.0, and 0<a1+a2+a3+a4+a5+a6+a7≤1.0; and M⁺ represents an ion selected from the group consisting of an ammonium ion, a sodium ion, and a potassium ion.

13. The bio-electrode according to claim 9, wherein the repeating unit-a comprises an ammonium ion shown by the following general formula (3) as an ammonium ion for forming an ammonium salt, 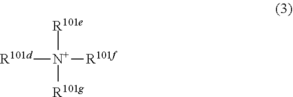
wherein, R^{101d}, R^{101e}, R^{101f}, and R^{101g} each represent a hydrogen atom, a linear, branched, or cyclic alkyl group having 1 to 15 carbon atoms, a linear, branched, or cyclic alkenyl group or alkynyl group having 2 to 12 carbon atoms, or an aromatic group having 4 to 20 carbon atoms, and optionally have one or more selected from the group consisting of an ether group, a carbonyl group, an ester group, a hydroxy group, a carboxy group, an amino group, a nitro group, a sulfonyl group, a sulfinyl group, a halogen atom, and a sulfur atom; and R^{101d} and R^{101e}, or R^{101d}, R^{101e}, and R^{101f}, are optionally bonded to each other together with a nitrogen atom bonded therewith to form a ring in which R^{101d} and R^{101e}, or R^{101d}, R^{101e}, and R^{101f}, represent an alkylene group having 3 to 10 carbon atoms, or to form a heteroaromatic ring having the nitrogen atom in the general formula (3) within the ring.

14. The bio-electrode according to claim 10, wherein the repeating unit-a comprises an ammonium ion shown by the following general formula (3) as an ammonium ion for forming an ammonium salt, 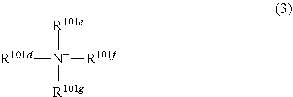
wherein, R^{101d}, R^{101e}, R^{101f}, and R^{101g} each represent a hydrogen atom, a linear, branched, or cyclic alkyl group having 1 to 15 carbon atoms, a linear, branched, or cyclic alkenyl group or alkynyl group having 2 to 12 carbon atoms, or an aromatic group having 4 to 20 carbon atoms, and optionally have one or more selected from the group consisting of an ether group, a carbonyl group, an ester group, a hydroxy group, a carboxy group, an amino group, a nitro group, a sulfonyl group, a sulfinyl group, a halogen atom, and a sulfur atom; and R^{101d} and R^{101e}, or R^{101d}, R^{101e}, and R^{101f}, are optionally bonded to each other together with a nitrogen atom bonded therewith to form a ring in which R^{101d} and R^{101e}, or R^{101d}, R^{101e}, and R^{101f}, represent an alkylene group having 3 to 10 carbon atoms, or to form a heteroaromatic ring having the nitrogen atom in the general formula (3) within the ring.

15. The bio-electrode according to claim 11, wherein the repeating unit-a comprises an ammonium ion shown by the following general formula (3) as an ammonium ion for forming an ammonium salt, 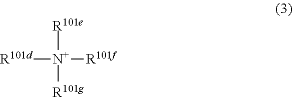
wherein, R^{101d}, R^{101e}, R^{101f}, and R^{101g} each represent a hydrogen atom, a linear, branched, or cyclic alkyl group having 1 to 15 carbon atoms, a linear, branched, or cyclic alkenyl group or alkynyl group having 2 to 12 carbon atoms, or an aromatic group having 4 to 20 carbon atoms, and optionally have one or more selected from the group consisting of an ether group, a carbonyl group, an ester group, a hydroxy group, a carboxy group, an amino group, a nitro group, a sulfonyl group, a sulfinyl group, a halogen atom, and a sulfur atom; and R^{101d} and R^{101e}, or R^{101d}, R^{101e}, and R^{101f}, are optionally bonded to each other together with a nitrogen atom bonded therewith to form a ring in which R^{101d} and R^{101e}, or R^{101d}, R^{101e}, and R^{101f}, represent an alkylene group having 3 to 10 carbon atoms, or to form a heteroaromatic ring having the nitrogen atom in the general formula (3) within the ring.

16. The bio-electrode according to claim 12, wherein the repeating unit-a comprises an ammonium ion shown by the following general formula (3) as an ammonium ion for forming an ammonium salt, 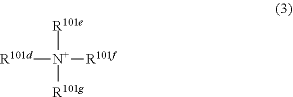
wherein, R^{101d}, R^{101e}, R^{101f}, and R^{101g} each represent a hydrogen atom, a linear, branched, or cyclic alkyl group having 1 to 15 carbon atoms, a linear, branched, or cyclic alkenyl group or alkynyl group having 2 to 12 carbon atoms, or an aromatic group having 4 to 20 carbon atoms, and optionally have one or more selected from the group consisting of an ether group, a carbonyl group, an ester group, a hydroxy group, a carboxy group, an amino group, a nitro group, a sulfonyl group, a sulfinyl group, a halogen atom, and a sulfur atom; and R^{101d} and R^{101e}, or R^{101d}, R^{101e}, and R^{101f}, are optionally bonded to each other together with a nitrogen atom bonded therewith to form a ring in which R^{101d} and R^{101e}, or R^{101d}, R^{101e}, and R^{101f}, represent an alkylene group having 3 to 10 carbon atoms, or to form a heteroaromatic ring having the nitrogen atom in the general formula (3) within the ring.

17. The bio-electrode according to claim 1, further comprising one or more resin (C) selected from the group consisting of (meth)acrylate resin, (meth)acrylamide resin, urethane resin, polyurethane (meth)acrylate, polyvinyl alcohol, polyvinylpyrrolidone, polyoxazoline, polyglycerin, polyglycerin-modified silicone, polyglycerin(meth)acrylate, cellulose, polyethylene glycol, and polypropylene glycol, as a component of the layer (B).

18. The bio-electrode according to claim 2, further comprising one or more resin (C) selected from the group consisting of (meth)acrylate resin, (meth)acrylamide resin, urethane resin, polyurethane (meth)acrylate, polyvinyl alcohol, polyvinylpyrrolidone, polyoxazoline, polyglycerin, polyglycerin-modified silicone, polyglycerin(meth)acrylate, cellulose, polyethylene glycol, and polypropylene glycol, as a component of the layer (B).

19. A method for manufacturing a bio-electrode according to claim 1, the method comprising:
forming the layer (A) on a substrate by applying a solution comprising a metal nanowire, or by printing an electro-conductive paste containing a conductive particle; and forming the layer (B) on the layer (A) by coating a bio-electrode composition comprising the polymer.

20. A method for manufacturing a bio-electrode according to claim 2, the method comprising:
forming the layer (A) on a substrate by applying a solution comprising a metal nanowire, or by printing an electro-conductive paste containing a conductive particle; and forming the layer (B) on the layer (A) by coating a bio-electrode composition comprising the polymer.
